(19) United States
(12) Patent Application Publication
(10) Pub. No.: US 2011/0150775 A1
(43) Pub. Date: Jun. 23, 2011

(54) GENOMIC APPROACHES TO FETAL TREATMENT AND DIAGNOSIS

(75) Inventors: Donna Slonim, North Andover, MA (US); Kirby Johnson, North Attleboro, MA (US); Diana Bianchi, Charlestown, MA (US)

(73) Assignees: TUFTS MEDICAL CENTER, INC.; TUFTS UNIVERSITY

(21) Appl. No.: 12/993,881

(22) PCT Filed: Jun. 1, 2009
(86) PCT No.: PCT/US09/45876

§ 371 Date: Nov. 22, 2010

Related U.S. Application Data
(60) Provisional application No. 61/057,874, filed on Jun. 1, 2008.
(60) Provisional application No. 61/180,904, filed on May 25, 2009.

(51) Int. Cl. A61K 49/00; C40B 30/02; A61K 31/56; A61K 31/137; A61K 31/44; A61K 33/34; A61K 31/19; A61K 31/5415; A61K 31/395; A61K 31/60; A61K 31/4355; A61K 31/196; A61K 31/52; A61K 31/405; A61K 31/352; A61K 31/337; A61K 31/047; A61K 31/55; A61K 31/54; A61K 31/517; A61K 31/167; A61K 31/706; A61K 31/415; A61K 31/566; A61K 31/4164; A61K 31/195; A61K 31/197; A61K 31/201; A61K 31/407; C12Q 1/68; A61P 43/00

(52) U.S. Cl. 424/9.2; 506/8; 514/181; 514/654; 514/356; 424/637; 514/573; 514/227.8; 514/183; 514/165; 514/291; 514/567; 514/263.3; 514/420; 514/456; 514/449; 514/729; 514/217; 514/225.8; 514/252.17; 514/619; 514/43; 514/396; 514/177; 514/399; 514/561; 514/557; 514/560; 514/411; 435/6.11

(57) ABSTRACT

The present invention provides systems for developing and/or testing therapies for prenatal diseases and conditions including Down Syndrome. The present invention also provides diagnostic methods for Down Syndrome involving, in some embodiments, gene expression analyses of fetal RNA and/or detection of expression of particular genes involved in Down Syndrome. Also provided are microarrays and kits useful in prenatal diagnostic applications.

### RELATED APPLICATION INFORMATION

The present application claims benefit of and priority to U.S. provisional applications Ser. Nos. 61/057,874 (filed on Jun. 1, 2008) and 61/180,904 (filed on May 25, 2009), the contents of which are herein incorporated by reference in their entirety.

### GOVERNMENT SUPPORT

This invention was made with U.S. government support under the Eunice Kennedy Shriver National Institute of Child Health and Human Development Award (R01 grant nos. HD042053-06 and R01 HD058880-01 to Diana Bianchi and Donna Slonim respectively). The government of the United States of America has certain rights in the invention.

### BACKGROUND

Genetic disorders and congenital abnormalities (also called birth defects) occur in about 3 to 5% of all live births (Robinson and Linden (1993)). In the United States, birth defects are the leading cause of infant mortality (Anderson et al. (1997)). Genetic disorders and congenital anomalies are associated with enormous medical-care costs and create a heavy psychological and emotional burden on those afflicted and/or their families (Czeizel et al., (1984); Centers for Disease Control (1989); Kaplan (1991); and Cuniff et al. (1995)).

Prenatal diagnosis has become an essential facet of clinical management of pregnancy itself, as well as a critical step toward the detection, prevention, and possible treatment of genetic disorders. Current prenatal diagnostic methods are typically limited to methods that rely on anatomical abnormalities, chromosomal anomalies, and single gene mutations as markers. Often, the biological mechanisms underlying fetal diseases are poorly understood, and therapeutic interventions are lacking.

### SUMMARY

The present invention encompasses the understanding that examination of fetal gene expression may provide an increased understanding of fetal development that may lead to novel therapies and diagnostic methods for fetal diseases and conditions. The present invention encompasses the discovery that genomic profiles (such as, for example, gene expression profiles) provide useful information that enables the design and implementation of therapies for fetal conditions and diseases. Such therapies would include therapies that can be applied prenatally, for example in utero, and/or shortly after birth. By employing genomic approaches, the inventors have created a new dimension to fetal diagnosis and treatment.

The present invention further encompasses the discovery of novel biomarkers for Down Syndrome (also known as Trisomy 21) that may afford diagnostic methods that are less invasive, require less biological material, and/or may be performed at earlier gestational ages than do current diagnostic methods.

In one aspect, provided are methods usefule for identifying therapeutic agents for a fetal disease or condition, comprising the steps of obtaining a reference genomic profile; obtaining a test genomic profile from a sample of amniotic fluid and/or maternal blood, wherein the sample is obtained from a subject suffering from or carrying a fetus suffering from a fetal disease or condition; determining differences between the test genomic profile and the reference genomic profile; inputting the test genomic profile into a first computing machine; accessing a storage repository on a second computing machine, wherein the storage repository contains a set of stored genomic profiles of one or more cell line(s) that have each been contacted with a different agent, wherein each stored genomic profile is mapped to data representing a corresponding agent; generating, by a correlator executing on the first or the second computing machine, a correlation between each stored genomic profile and the test genomic profile; and selecting at least one agent whose corresponding genomic profile has a negative correlation score with the test genomic profile, the selected agent being likely to reduce the differences between the test genomic profile and the reference genomic profile. In some embodiments, the genomic profiles comprise information selected from the group consisting of mRNA levels (i.e., such as those obtained from gene expression profiling experiments), protein expression levels, DNA methylation patterns, metabolite profiles, and combinations thereof.

In some embodiments, the fetal disease or condition is selected from the group consisting of twin-to-twin-transfusion syndrome (TTTS), gastroschisis, Down Syndrome, fetal structural anomalies, fetal congenital heart anomaly, fetal kidney anomalies, neural tube defects, and congenital diaphragmatic hernia. In some embodiments, the fetal disease or condition is Down Syndrome. In some embodiments, inventive methods further comprise testing activity of at least one identified agent for therapeutic effects in a fetal disease or condition. In some such embodiments, the at least one identified agent is tested for medical applications in utero.

In another aspect, the invention provides a method comprising a step administering to a patient suffering from a fetal disease or condition an effective dose of an agent identified by methods of the present invention, such that symptoms of the fetal disease or condition are ameliorated. In some embodiments, the fetal disease or condition is selected from the group consisting of twin-to-twin-transfusion syndrome (TTTS), gastroschisis, Down Syndrome, fetal structural anomalies, fetal congenital heart anomaly, fetal kidney anomalies, neural tube defects, and congenital diaphragmatic hernia. In some embodiments in which the fetal disease or condition is Down Syndrome, the agent is selected from the group consisting of anti-oxidants (e.g., celastrol), ion channel modulators, G-protein signaling modulators, and combinations thereof. In some embodiments, the agent is a calcium channel blocker (e.g., verapamil, felodipine, nifedipine, combinations thereof, etc.). In some embodiments, the agent is selected from the group consisting of copper sulfate, 15-delta prostaglandian J2, blebbistatin, prochlorperazine, 17-dimethylamino-geldanamyc in, butein, nordihydroguaiaretic acid, acetylsalicyclic acid, 51825898, sirolimus, docosahexaenoic acid ethyl ester, diclofenac, mercaptopurine, indometacin, 5279552, 17-allylamino-geldanamycin, rottlerin, paclitaxel, pyrvinium, flufenamic acid, oligomycin, 5114445, resveratrol, Y-27632, carbamazepine, nitrendipine, fluphenazine, 5152487, prazosin, 5140203, cytochalasin B, vorinostate, MG-132, HNMPA-(AM)₃, decitabine, U0125, nocodazole, 5224221, 3-hydroxy-DL-kynurenine, 5162773, oxaprozin, colforsin, exemestane, felodipine, HC toxin, 5213008, dimethyloxalylglycine, 5109870, calmidazolium, 5255229, derivatives thereof, and combinations thereof. In some embodiments, the effective dose of the agent is administered in utero and/or perinatally.

In yet another aspect, the invention provides methods for evaluating efficacy of a treatment for a fetal disease or condition. Such inventive methods comprise steps of (a) hybridizing RNA from an amniotic fluid and/or maternal blood sample from a subject suffering from or carrying a fetus suffering from a fetal disease or condition to at least one polynucleotide probe for at least one predetermined gene such that expression levels of at least one predetermined gene are obtained, wherein the sample is obtained from a subject to which the agent in step (b) has not been administered; (b) administering an agent to a subject suffering from the fetal disease or condition; (c) hybridizing RNA from an amniotic fluid and/or maternal blood sample from a subject suffering from or carrying a fetus suffering from a fetal disease or condition to at least one genetic probe for the same predetermined gene(s) from step (a) such that expression levels of the predetermined gene(s) are obtained, wherein the sample is obtained from a subject to which the agent has been administered; (d) comparing the gene expression levels of the predetermined genes obtained from steps (a) and (c); and (e) determining, based on the comparison, efficacy of the agent as a treatment for the fetal disease or condition. In some embodiments, the fetal disease or condition is selected from the group consisting of twin-to-twin-transfusion syndrome (TTTS), gastroschisis, Down Syndrome, fetal structural anomalies, fetal congenital heart anomaly, fetal kidney anomalies, neural tube defects, and congenital diaphragmatic hernia.

In yet another aspect, the invention provides methods for diagnosing Down Syndrome. In some embodiments, such methods comprise steps of: providing a test sample comprising fetal RNA, wherein the fetal RNA is obtained from amniotic fluid and/or maternal blood obtained from a woman pregnant with a fetus with a known gender and gestational age, and wherein the test sample comprises a plurality of nucleic acid segments labeled with a detectable agent; providing a gene-expression array comprising a plurality of genetic probes, wherein each genetic probe is immobilized to a discrete spot on a substrate surface to form the array; providing a database comprising levels of mRNA expression established for trisomy 21 male and female fetuses at different gestational ages; contacting the array with the test sample under conditions to allow the nucleic acid segments in the sample to specifically hybridize to the genetic probes on the array; determining the binding of individual nucleic acid segments of the test sample to individual genetic probes immobilized on the array to obtain a binding pattern; establishing a gene expression pattern for the fetus; comparing the gene expression pattern of the fetus to the levels of mRNA expression in the database; and providing, based on the comparison, a diagnosis with respect to Down Syndrome.

In some embodiments, such methods comprise steps of: providing an amniotic fluid and/or maternal blood sample from a pregnant woman; hybridizing RNA from the sample to at least ten genetic probes for at least ten genes that are differentially expressed in trisomy 21 fetuses such that expression levels of the at least ten genes are obtained; and determining, based on the expression levels of the at least ten genes, a diagnosis with respect to Down Syndrome.

In yet another aspect, the invention provides gene expression microarrays for use in prenatal diagnostic applications for one or more particular fetal diseases or conditions. In many embodiments, inventive gene expression microarrays comprise a solid substrate, the substrate having a surface, and a plurality of genetic probes, wherein each genetic probe is immobilized to a discrete spot on the surface of the substrate to form an array and wherein at least a subset of the genetic probes comprise sequences from a predetermined set of genes that are differentially expressed in a fetal disease or condition for which prenatal diagnosis is desired. In some embodiments, the subset of the genes represented on the microarray comprises at least ten of the genes listed in Tables 2 and 4. In some embodiments, the fetal disease or condition is selected from the group consisting of twin-to-twin-transfusion syndrome (TTTS), gastroschisis, Down Syndrome, fetal structural anomalies, fetal congenital heart anomaly, fetal kidney anomalies, neural tube defects, and congenital diaphragmatic hernia.

In another aspect, the invention provides kits comprising inventive gene expression microarrays as described above, a database comprising baseline levels of mRNA expression established for karyotypically and developmentally normal male and normal female fetuses at different gestational ages, and instructions for using the array and database. In some embodiments, kits further comprise materials to extract fetal RNA from a sample of amniotic fluid obtained from a pregnant woman. In some embodiments, kits further comprise materials to extract RNA from a sample of blood obtained from a pregnant woman and instructions on how to distinguish fetal RNA from maternal RNA in the sample.

### BRIEF DESCRIPTION OF THE DRAWINGS

FIG. 1 depicts a heatmap showing hierarchical clustering of control (C1-C7; solid red bar) and trisomic (T1-T7; solid blue bar) samples. Clustering is based on data from 409 probe sets (414 from the Individual gene set minus 5 from chromosome 21). If the five genes from chromosome 21 are not removed, the dendrogram looks quite similar. In particular, it similarly separates the control and trisomic samples. Expression values in each row are z-score normalized.

FIG. 2 depicts histograms of fold-changes in gene expression between average expression in Down Syndrome and average expression in controls. (A) Histogram for all 501 probe sets representing genes on chromosome 21. Fold-changes are reported in log-scale (base 2), so that up- and down-regulation appear at equal distance from zero. For example, a value of 1.0 represents 2-fold upregulation in Down Syndrome. The bold vertical line at ≈0.58 corresponds to the 1.5-fold change expected from the gene-dosage hypothesis. (B) Histogram of all probe sets representing genes on chromosomes other than 21, shown on the same scale as in (A).

FIG. 3 is a schematic depicting a putative network of pathways implicated in Down Syndrome. Significantly implicated processes, based on DAVID functional analysis, are shown in boxes. Edges between boxes represent relationships between functional processes such as G protein signaling and disruptions in ion transport that result from oxidative stress, as suggested in references Kourie (1998) (solid lines), Esposito et al. (2008) (dotted lines), Mates et al. (2008) (dashed lines), and Lehotsky et al. (1999) (dash-dotted lines), the contents of each of which are herein incorporated by reference in their entirety. Blue boxes with dashed borders are processes over-represented in the Individual gene set only; purple boxes with thin solid borders are processes over-represented in both the individual and Leading Edge gene sets; and green boxes with thick solid borders are implicated by both the individual and Leading Edge gene sets and by the Connectivity Map.

FIGS. 4A and 4B are block diagrams depicting embodiments of computes that useful in the practice of the invention.

### DEFINITIONS

Throughout the specification, several terms are employed that are defined in the following paragraphs.

As used herein, the terms “about” and “approximately,” in reference to a number, is used herein to include numbers that fall within a range of 20%, 10%, 5%, or 1% in either direction (greater than or less than) the number unless otherwise stated or otherwise evident from the context (except where such number would exceed 100% of a possible value).

As used herein, the term “administer” means giving something (such as, for example a therapy, treatment, compound, and/or dose thereof) to an individual. Routes of administration include, but are not limited to, topical (including epicutaneous, enema, eye drops, ear drops, intranasal, vaginal, etc.), enteral (including oral, feeding tube, rectal, etc.), parenteral (including intravenous, intraarterial, intramuscular, intracardiac, subcutaneous, intraosseous, intradermal, intrathecal, intraperitonael, transdermal, transmucosal, inhalational), epidural, intravitreal. Administration to a subject may result in the therapy, treatment, compound, dose thereof, etc. being applied in utero. In some embodiments, the individual to which something is administered is a pregnant woman. In some embodiments, the individual to which something is administered is a fetus. In some embodiments, administering to a fetus comprises administering to the pregnant woman carrying the fetus.

As used herein, the term “biomarker” refers to its meaning as understood in the art. The term can refer to an indicator that provides information about, among other things, a process, condition, developmental stage, or outcome of interest, e.g., a fetus's diagnosis with respect to Down Syndrome. In general, the value of such an indicator is correlated with a process, condition, developmental stage, or outcome of interest. The term “biomarker” can also refer to a molecule that is the subject of an assay or measurement the result of which provides information about a process, condition, developmental stage, or outcome of interest. For example, an elevated expression level of a particular gene can be an indicator that a subject has a particular condition. The expression level of the gene, an elevated expression level of the gene, and the gene expression product itself, can all be referred to as “biomarkers”.

As used herein, the term “client” or “client agent” when used in reference to a computing environment, may be used interchangeable with any one of the following terms: “client machine(s),” “client(s),” “client computer(s),” “client device(s),” “client computing device(s),” “client node(s),” “endpoint(s),” “endpoint node(s),” or a “second machine.” A client machine can, in some embodiments, be a computing device. A client machine can in some embodiments execute, operate or otherwise provide an application that can be any one of the following: software; a program; executable instructions; a web browser; a web-based client; a client-server application; a thin-client computing client; an ActiveX control; a Java applet; software related to voice over internet protocol (VoIP) communications like a soft IP telephone; an application for streaming video and/or audio; an application for facilitating real-time-data communications; a HTTP client; a FTP client; an Oscar client; a Telnet client; or any other type and/or form of executable instructions capable of executing on client machine. In some embodiments, the client machine is be a virtual machine 102C such as those manufactured by XenSolutions, Citrix Systems, IBM, VMware, or any other virtual machine able to implement the methods and systems described herein.

As used herein, the term “complementary” refers to nucleic acid sequences that base-pair according to the standard Watson-Crick complementary rules, or that are capable of hybridizing to a particular nucleic acid segment under relatively stringent conditions. Nucleic acid polymers are optionally complementary across only portions of their entire sequences.

As used herein, the term “differentially expressed” in reference to genes refers to the state of having a different expression pattern or level depending on the type of cell, tissue, and/or sample, from which the gene expression products are derived. “Differentially expressed” genes may be upregulated or downregulated in the cell, tissue, and/or samples as compared to controls. For example, a gene that is upregulated in samples obtained from a subject suffering from Down Syndrome as compared to a subject who is not can be said to be “differentially expressed.” As another example, a gene that is downregulated in samples from a subject that has undergone a developmental transition as compared to a subject who has not can also be said to be “differentially expressed.”

As used herein, the term “Down Syndrome” (also known as “Down's Syndrome” and “trisomy 21”) has its meaning as known in the art and refers to a disorder that results from extra genetic material from all or part of human chromosome 21.

As used herein, the term “effective dose” is used interchangeably with the term “effective amount” and refers to any dose or amount of a compound, composition, therapeutic agent, etc. that is sufficient to fulfill its intended purpose(s), i.e., a desired biological or medicinal response in a tissue or subject. For example, in certain embodiments of the present invention, the purpose(s) may be: to slow down or stop the progression, aggravation, or deterioration of the symptoms of a fetal disease or condition, to bring about amelioration of the symptoms of the fetal disease or condition, and/or to cure the fetal disease or condition.

As used herein, the terms “fluorophore”, “fluorescent moiety”, “fluorescent label”, “fluorescent dye” and “fluorescent labeling moiety” are used herein interchangeably. They refer to a molecule which, in solution and upon excitation with light of appropriate wavelength, emits light back. Numerous fluorescent dyes of a wide variety of structures and characteristics are suitable for use in the practice of this invention. Similarly, methods and materials are known for fluorescently labeling nucleic acids (see, for example, Haugland (1994)). In choosing a fluorophore, it is preferred that the fluorescent molecule absorbs light and emits fluorescence with high efficiency (i.e., high molar absorption coefficient and fluorescence quantum yield, respectively) and is photostable (i.e., it does not undergo significant degradation upon light excitation within the time necessary to perform the analysis).

As used herein, the term “gene” refers to a discrete nucleic acid sequence responsible for a discrete cellular product and/or performing one or more intracellular or extracellular functions. In some embodiments, the term “gene” refers to a nucleic acid that includes a portion encoding a protein and optionally encompasses regulatory sequences, such as promoters, enhancers, terminators, and the like, which are involved in the regulation of expression of the protein encoded by the gene of interest. Such gene and regulatory sequences may be derived from the same natural source, or may be heterologous to one another. In some embodiments, a gene does not encode proteins but rather provide templates for transcription of functional RNA molecules such as tRNAs, rRNAs, etc. Alternatively or additionally, in some embodiments, a gene may define a genomic location for a particular event/function, such as the binding of proteins and/or nucleic acids.

As used herein, the term “gene expression” refers to the conversion of the information, contained in a gene, into a gene product. A gene product can be the direct transcriptional product of a gene (e.g., mRNA, tRNA, rRNA, antisense RNA, ribozyme structural RNA or any other type of RNA), or the product of subsequent downstream processing events (e.g., splicing, RNA processing, translation). In some embodiments, a gene product is a protein produced by translation of an mRNA. In some embodiments, gene products are RNAs that are modified by processes such as capping, polyadenylation, methylation, and editing, proteins post-translationally modified, and proteins modified by, for example, methylation, acetylation, phosphorylation, ubiquitination, ADP-ribosylation, myristilation, and glycosylation.

As used herein, the term “gene expression array” refers to an array comprising a plurality of genetic probes immobilized on a substrate surface that can be used for quantitation of mRNA expression levels. In the context of the present invention, the term “array-based gene expression analysis” is used to refer to methods of gene expression analysis that use gene-expression arrays. The term “genetic probe”, as used herein, refers to a nucleic acid molecule of known sequence, which has its origin in a defined region of the genome and can be a short DNA sequence (or oligonucleotide), a PCR product, or mRNA isolate. Genetic probes are gene-specific DNA sequences to which nucleic acids from a test sample of amniotic fluid RNA are hybridized. Genetic probes specifically bind (or specifically hybridize) to nucleic acid of complementary or substantially complementary sequence through one or more types of chemical bonds, usually through hydrogen bond formation.

As used herein, the phrases “genomic profile” and “genomic signature” are used interchangeable to refer to a genome-wide profile in a given cell, cell type, tissue, individual, sample, condition, disease state, etc. A genomic profile or genomic signature is the genome-wide equivalent of a “molecular signature” and may refer to, among other things, a gene expression profile, a protein expression profile, a DNA methylation pattern, metabolite profiles, etc.

As used herein, the term “gestational age” refers to age of an embryo, fetus, or fetus as calculated from the first day of the mother's last menstrual period. In humans, the gestational age may count the period of time from about two weeks before fertilization takes place.

As used herein, the term “isolated” when applied to RNA means a molecule of RNA or a portion thereof, which (1) by virtue of its origin or manipulation, is separated from at least some of the components with which it was previously associated; or (2) was produced or synthesized by the hand of man.

As used herein, the terms “labeled”, “labeled with a detectable agent” and “labeled with a detectable moiety” are used interchangeably. They are used to specify that a nucleic acid molecule or individual nucleic acid segments from a sample can be visualized, for example, following binding (i.e., hybridization) to genetic probes. In hybridization methods, samples of nucleic acid segments may be detectably labeled before the hybridization reaction or a detectable label may be selected that binds to the hybridization product. Preferably, the detectable agent or moiety is selected such that it generates a signal which can be measured and whose intensity is related to the amount of hybridized nucleic acids. In array-based methods, the detectable agent or moiety is also preferably selected such that it generates a localized signal, thereby allowing spatial resolution of the signal from each spot on the array. Methods for labeling nucleic acid molecules are well known in the art (see below for a more detailed description of such methods). Labeled nucleic acid fragments can be prepared by incorporation of or conjugation to a label, that is directly or indirectly detectable by spectroscopic, photochemical, biochemical, immunochemical, electrical, optical, or chemical means. Suitable detectable agents include, but are not limited to: various ligands, radionuclides, fluorescent dyes, chemiluminescent agents, microparticles, enzymes, colorimetric labels, magnetic labels, and haptens. Detectable moieties can also be biological molecules such as molecular beacons and aptamer beacons.

As used herein, the term “messenger RNA” or “mRNA” refers a form of RNA that serves as a template for protein biosynthesis. In many embodiments, the amount of a particular mRNA (i.e., having a particular sequence, and originating from a particular same gene) reflects the extent to which the gene encoding the mRNA has been “expressed.”

As used herein, the terms “microarray,” “array” and “biochip” are used interchangeably and refer to an arrangement, on a substrate surface, of multiple nucleic acid molecules of known sequences. Each nucleic acid molecule is immobilized to a “discrete spot” (i.e., a defined location or assigned position) on the substrate surface. The term “microarray” more specifically refers to an array that is miniaturized so as to require microscopic examination for visual evaluation. Arrays used in the methods of the invention are preferably microarrays.

As used herein, the terms “nucleic acid” and “nucleic acid molecule” are used herein interchangeably. They refer to a deoxyribonucleotide or ribonucleotide polymer in either single- or double-stranded form, and unless otherwise stated, encompass known analogs of natural nucleotides that can function in a similar manner as naturally occurring nucleotides. The terms encompass nucleic acid-like structures with synthetic backbones, as well as amplification products.

As used herein, the term “oligonucleotide” refers to usually short strings of DNA or RNA to be used as hybridizing probes or nucleic acid molecule array elements. These short stretches of sequence are often chemically synthesized. The size of the oligonucleotide depends on the function or use of the oligonucleotides. When used in microarrays for hybridization, oligonucleotides can comprise natural nucleic acid molecules or synthesized nucleic acid molecules and comprise between 5 and 150 nucleotides, preferably between about 15 and about 100 nucleotides, more preferably between 15 and 30 nucleotides and most preferably, between 18 and 25 nucleotides complementary to mRNA.

As used herein, the term “prenatal disease or condition” refers to any disease or condition that can affect fetuses. The term “prenatal disease or condition” encompasses diseases or conditions that have symptoms that manifest during fetal development and/or result in detectable changes at prenatal stages. Thus, for example, Down Syndrome, which affects adults, is considered a “prenatal disease or condition” because the syndrome results in detectable changes at prenatal stages.

As used herein, the term “RNA transcript” refers to the product resulting from transcription of a DNA sequence. When the RNA transcript is the original, unmodified product of a RNA polymerase catalyzed transcription, it is referred to as the primary transcript. An RNA transcript that has been processed (e.g., spliced, etc.) will differ in sequence from the primary transcript; a fully processed transcript is referred to as a “mature” RNA. The term “transcription” refers to the process of copying a DNA sequence of a gene into an RNA product, generally conducted by a DNA-directed RNA polymerase using the DNA as a template. A processed RNA transcript that is translated into protein is often called a messenger RNA (mRNA).

As used herein, the term “statistically significant number” refers to a number of samples (analyzed or to be analyzed) that is large enough to provide reliable data.

As used herein, the terms “subject” and “individual” are used herein interchangeably. They refer to a human or another animal (e.g., mouse, rat, rabbit, dog, cat, cattle, swine, sheep, horse, or primate) that can be afflicted with or is susceptible to a disease, disorder, condition, or complication (e.g., Down Syndrome) but may or may not have the disease or disorder. In many embodiments, the subject is a human being. In many embodiments, the subject is a fetus. In some embodiments, the subject is a newborn.

As used herein, the term “suffering from” is used to describe subjects that have been diagnosed as having a particular disease or condition, whether or not the subject is experiencing symptoms typical of that disease or condition.

As used herein, the term “susceptible” means having an increased risk for and/or a propensity for something, i.e., a condition such as twin-to-twin transfusion syndrome (TTTS), gastroschisis, congenital diaphragmatic hernia, and/or Down Syndrome. The term takes into account that an individual “susceptible” for a condition may never be diagnosed with the condition.

The terms “therapeutic agent” and “drug” are used herein interchangeably. They refer to a bioactive substance, molecule, compound, agent, factor or composition effective in the treatment of a disease or clinical condition.

As used herein, the term “treatment” characterizes a method or process that is aimed at (1) delaying or preventing the onset of a disease or condition; (2) slowing down or stopping the progression, aggravation, or deterioration of one or more symptoms of the disease or condition; (3) bringing about ameliorations of the symptoms of the disease or condition; (4) reducing the severity or incidence of the disease or condition; or (5) curing the disease or condition. A treatment may be administered prior to the onset of the disease, for a prophylactic or preventive action. Alternatively or additionally, the treatment may be administered after initiation of the disease or condition, for a therapeutic action.

As used herein, the term “trisomy 21” describes the karyotypic condition in humans and in human cells of having an extra copy of chromosome 21. “Trisomy 21” is often used interchangeably with “Down Syndrome.” As used herein, the term “trisomy 21” fetus refers to a fetus that has undergone karyotyping and has been diagnosed as having an extra copy of chromosome 21.

### DETAILED DESCRIPTION OF CERTAIN EMBODIMENTS

As mentioned above, the present invention provides technologies for developing and/or evaluating therapies for fetal diseases and conditions and for diagnosis of fetal diseases and conditions such as Down Syndrome.

### I. Fetal RNA

Fetal RNA can be obtained from biological samples such as amniotic fluid or maternal blood from pregnant women.

### Amniotic Fluid Sample

Notwithstanding the well-known instability of RNA, fetal RNA survives in amniotic fluid in amounts and in a condition appropriate for analysis. In some embodiments, inventive methods involve providing or obtaining a sample of amniotic fluid obtained from a pregnant woman. Amniotic fluid is generally collected by amniocentesis, in which a long needle is inserted in the mother's lower abdomen into the amniotic cavity inside the uterus to withdraw a certain volume of amniotic fluid.

For prenatal diagnosis, most amniocenteses are performed between the 14^{th} and 20^{th} weeks of pregnancy and the volume of amniotic fluid withdrawn is about 10 to 30 mL. Traditionally, the most common indications for amniocentesis include: advanced maternal age (typically set, in the US, at 35 years or more at the estimated time of delivery), previous child with a birth defect or genetic disorder, parental chromosomal rearrangement, family history of late-onset disorders with genetic components, recurrent miscarriages, positive maternal serum screening test (Multiple Marker Screening) documenting increased risk of fetal neural tube defects and/or fetal chromosomal abnormality, and abnormal fetal ultrasound examination (for example, revealing signs known to be associated with fetal aneuploidy). However, the amount and type of information that may be obtained from an amniotic fluid sample according to the present invention may support a change in standard operating procedure, such that amniocentesis is considered or performed in any pregnancy.

Amniocentesis is also performed for therapeutic purposes. In such cases, large amounts of amniotic fluid (>1 L) are removed (amnioreduction) to correct polyhydramnios (i.e., an excess of amniotic fluid surrounding the fetus). Polyhydramnios can represent a danger because of an increased risk of premature rupture of the membranes, and may also be a sign of birth defect or other medical problems such as gestational diabetes or fetal hydrops. Polyhydramnios is also observed in multiple gestations. Twin-to-twin transfusion syndrome (TTTS) is defined sonographically as the combined presence of an excess of amniotic fluid in one sac and an insufficiency of amniotic fluid in the other sac. In TTTS, the goal of the amnioreduction is to attempt to decrease the likelihood of miscarriage or preterm labor by reducing the amniotic fluid volume in the sac of the recipient twin.

In the context of the present invention, samples of amniotic fluid may be obtained after standard or therapeutic amniocentesis. In conventional amniocentesis procedures, fetal cells present in the amniotic fluid are isolated by centrifugation and grown in culture for chromosome analysis, biochemical analysis, and/or molecular biological analysis. Centrifugation also produces a supernatant sample (herein termed “remaining amniotic material”), which is usually stored at −20° C. as a back-up in case of assay failure. Aliquots of this supernatant may also be used for additional assays such as determination of alpha-fetoprotein and acetyl cholinesterase levels. After a certain period of time, the frozen supernatant sample is typically discarded. In aminoreductions, the entire sample of amniotic fluid withdrawn is discarded. The standard protocol followed by the Cytogenetics Laboratory at Tufts Medical Center (Boston, Mass.) provides the Applicants with fresh and frozen samples of amniotic fluid (from therapeutic amniocenteses) and fresh samples of remaining amniotic material (from diagnostic amniocenteses).

### Maternal Blood

Maternal blood can be obtained more readily than amniotic fluid and contains fetal and placental mRNAs. The term “maternal blood” is used to refer to blood from the pregnant woman (as opposed to blood from the fetus). In some embodiments, inventive methods involve providing or obtaining a sample of maternal blood obtained from a pregnant woman. Blood samples may, for example, be whole blood samples, that is, samples that are not separated into components such as plasma or serum. Fetal transcripts found in whole blood differ from those found in plasma. Blood samples can be drawn using standard techniques well known in the art such as venipuncture.

### Isolation of Fetal RNA

In the practice of methods of the invention, fetal RNA may be isolated from a sample of amniotic fluid obtained from a pregnant woman. Isolation may be carried out by any suitable method of RNA isolation or extraction.

In certain embodiments, fetal RNA is obtained by treating a sample of amniotic fluid such that fetal RNA present in the amniotic fluid sample is extracted. In certain embodiments, fetal RNA is extracted after removal of substantially all or some of the cell populations present in the sample of amniotic fluid. The cell populations may be removed from the sample by any suitable method, for example, by centrifugation. More than one centrifugation step may be performed to ensure that substantially all cell populations have been removed. In some embodiments, the cell populations are removed within two hours of obtaining the sample. In some embodiments, the cell populations are removed immediately after obtaining the sample of amniotic fluid.

When substantially all cell populations are removed from the sample, amniotic fluid fetal RNA consists essentially of cell-free fetal RNA. When extracted from a sample of remaining amniotic material obtained by centrifugation, fetal RNA comprises cell-free fetal RNA as well as fetal RNA from the cells still present in the remaining material.

Fetal RNA may also be obtained by isolating cells from the sample of amniotic fluid, optionally cultivating these isolated cells, and extracting RNA from the cells. In such cases, amniotic fluid fetal RNA consists essentially of fetal RNA from the cultured cells.

In some embodiments of the invention, fetal RNA is obtained from whole maternal blood, which contains a mixture of maternal mRNAs as well as fetal and placental mRNAs. In such embodiments, fetal and/or placental RNA is not isolated from maternal mRNAs. Rather, certain sets of transcripts are known to be expressed only from fetuses and/or by the placenta. (See, e.g., Maron et al. (2007), the entire contents of which are herein incorporated by reference.) Such fetal biomarkers allow analyses of fetal mRNAs within a sample also containing maternal mRNAs. In some such embodiments, computational methods for decomposing signals from different sources are used to help to distinguish fetal RNA from maternal RNA.

In some embodiments, before isolation or extraction of fetal RNA, the sample of amniotic fluid material or whole maternal blood is stored for a certain period of time under suitable storage conditions. In some embodiments, suitable storage conditions comprise temperatures ranging between about 10° C. to about −220° C., inclusive. In some embodiments, samples are stored at about 4° C., at about −10° C., at about −20° C., at about −70° C., or at about −80° C. In some embodiments, samples are stored for less than about 28 days. In some embodiments, samples are stored for more than about twenty-four hours. In some embodiments, before freezing, an RNase inhibitor, which prevents degradation of fetal RNA by RNases (i.e., ribonucleases), is added to the sample. In some embodiments, the RNase inhibitor is added within two hours of obtaining the sample. In some embodiments, the RNAse inhibitor is added within one hour of obtaining the sample. In some embodiments, the RNAse inhibitor is added within thirty minutes of obtaining the sample. In some embodiments, the RNAse inhibitor is added within ten minutes of obtaining the sample. In some embodiments, the RNAse inhibitor is added within five minutes of obtaining the sample. In some embodiments, the RNAse inhibitor is added within two minutes of obtaining the sample. In some embodiments, the RNase inhibitor is added immediately after obtaining the sample. In some embodiments, before RNA extraction, the frozen sample is thawed at 37° C. and mixed with a vortex.

In some embodiments, the sample is frozen (e.g., flash-frozen in liquid nitrogen and dry ice), stored, and thawed; then RNAse inhibitor is added after thawing. In some such embodiments, the RNase inhibitor is added within two hours of thawing. In some embodiments, the RNAse inhibitor is added within one hour of thawing. In some embodiments, the RNAse inhibitor is added within thirty minutes of thawing. In some embodiments, the RNAse inhibitor is added within ten minutes of thawing. In some embodiments, the RNAse inhibitor is added within five minutes of thawing. In some embodiments, the RNAse inhibitor is added within two minutes of thawing.

The most commonly used RNase inhibitor is a natural protein derived from human placenta that specifically (and reversibly) binds RNases (Blackburn et al. (1977)). RNase inhibitors are commercially available, for example, from Ambion (Austin, Tex.; as SUPERase•In™), Promega, Inc. (Madison, Wis.; as rRNasin® Ribonuclease Inhibitor) and Applied Biosystems (Framingham, Mass.). In general, precautions for preventing RNases contaminations in RNA samples, which are well known in the art and include the use of gloves, of certified RNase-free reagents and ware, of specifically treated water and of low temperatures, as well as routine decontamination and the like, are used in the practice of the methods of the invention.

For amniotic fluid samples, isolating fetal RNA may include treating the sample such that fetal RNA present in the sample is extracted and made available for analysis. Any suitable isolation method that results in extracted amniotic fluid fetal RNA may be used in the practice of the invention. For maternal whole blood sample, fetal RNA may be extracted together with maternal RNA, but some fetal mRNAs will be distinguishable from maternal RNA (Maron et al. 2007).

In order to obtain the most accurate assessment of the fetus, it may be desirable to minimize artifacts from manipulation processes. Therefore, the number of extraction and modification steps is in some embodiments kept as low as possible.

Methods of RNA extraction are well known in the art (see, for example, Sambrook et al., (1989)). Most methods of RNA isolation from bodily fluids or tissues are based on the disruption of the tissue in the presence of protein denaturants to quickly and effectively inactivate RNases. Generally, RNA isolation reagents comprise, among other components, guanidinium thiocyanate and/or beta-mercaptoethanol, which are known to act as RNase inhibitors (Chirgwin et al. (1979)). Isolated total RNA is then further purified from the protein contaminants and concentrated by selective ethanol precipitations, phenol/chloroform extractions followed by isopropanol precipitation (see, for example, Chomczynski and Sacchi (1987)) or cesium chloride, lithium chloride or cesium trifluoroacetate gradient centrifugations (see, for example, Glisin et al. (1974); and Stern and Newton (1986)).

In certain methods of the invention, for example those wherein fetal RNA is subjected to a gene-expression analysis, it may be desirable to isolate mRNA from total RNA in order to allow the detection of even low level messages (Alberts et al. (1994)).

Purification of mRNA from total RNA typically relies on the poly(A) tail present on most mature eukaryotic mRNA species. Several variations of isolation methods have been developed based on the same principle. In a first approach, a solution of total RNA is passed through a column containing oligo(dT) or d(U) attached to a solid cellulose matrix in the presence of high concentrations of salts to allow the annealing of the poly(A) tail to the oligo(dT) or d(U). The column is then washed with a lower salt buffer to remove and release the poly(A) mRNAs. In a second approach, a biotinylated oligo(dT) primer is added to the solution of total RNA and used to hybridize to the 3′ poly(A) region of the mRNAs. The hybridization products are captured and washed at high stringency using streptavidin coupled to paramagnetic particles and a magnetic separation stand. The mRNA is eluted from the solid phase by the simple addition of ribonuclease-free deionized water. Other approaches do not require the prior isolation of total RNA. For example, uniform, superparamagnetic, polystyrene beads with oligo(dT) sequences covalently bound to the surface may be used to isolate mRNA directly by specific base pairing between the poly(A) residues of mRNA and the oligo(dT) sequences on the beads. Furthermore, the oligo(dT) sequence on the beads may also be used as a primer for the reverse transcriptase to subsequently synthesize the first strand of cDNA. Alternatively, new methods or improvements of existing methods for total RNA or mRNA isolation, preparation and purification may be devised by one skilled in the art and used in the practice of the methods of the invention.

Numerous different and versatile kits can be used to extract RNA (i.e., total RNA or mRNA) from bodily fluids and are commercially available from, for example, Ambion, Inc. (Austin, Tex.), Amersham Biosciences (Piscataway, N.J.), BD Biosciences Clontech (Palo Alto, Calif.), BioRad Laboratories (Hercules, Calif.), Dynal Biotech Inc. (Lake Success, N.Y.), Epicentre Technologies (Madison, Wis.), Gentra Systems, Inc. (Minneapolis, Minn.), GIBCO BRL (Gaithersburg, Md.), Invitrogen Life Technologies (Carlsbad, Calif.), MicroProbe Corp. (Bothell, Wash.), Organon Teknika (Durham, N.C.), Promega, Inc. (Madison, Wis.), and Qiagen Inc. (Valencia, Calif.). For example, the RNAprotect Amniotic fluid Kit (Qiagen) may be used to extract fetal RNA from amniotic fluid. Similarly, the QIAamp DNA Blood Mini Kit (Qiagen), QIAamp DNA Blood Maxi Kit (Qiagen), and PaxGene blood RNA kit (PreAnalytiX) can be used to extract RNA from maternal whole blood. User Guides that describe in great detail the protocol to be followed are usually included in all these kits. Sensitivity, processing time and cost may be different from one kit to another. One of ordinary skill in the art can easily select the kit(s) most appropriate for a particular situation.

### Amplification of Extracted RNA

In certain embodiments, RNA (for example, fetal RNA from amniotic fluid or fetal and maternal RNA from whole maternal blood) is amplified before being analyzed. In some embodiments, before analysis, the RNA is converted, by reverse-transcriptase, into complementary DNA (cDNA), which, optionally, may, in turn, be converted into complementary RNA (cRNA) by transcription.

Amplification methods are well known in the art (see, for example, Kimmel and Berger (1987); Sambrook et al. (1989); Ausubel (Ed.) (2002); and U.S. Pat. Nos. 4,683,195; 4,683,202 and 4,800,159). Standard nucleic acid amplification methods include: polymerase chain reaction (or PCR, see, for example, Innis (Ed.) (1990); and Innis (Ed.) (1995); and ligase chain reaction (or LCR, see, for example, Landegren et al. (1988); and Barringer et al. (1990)).

Methods for transcribing RNA into cDNA are also well known in the art. Reverse transcription reactions may be carried out using non-specific primers, such as an anchored oligo-dT primer, or random sequence primers, or using a target-specific primer complementary to the RNA for each genetic probe being monitored, or using thermostable DNA polymerases (such as avian myeloblastosis virus reverse transcriptase or Moloney murine leukemia virus reverse transcriptase). Other methods include transcription-based amplification system (TAS) (see, for example, Kwoh et al. (1989)), isothermal transcription-based systems such as Self-Sustained Sequence Replication (3SR) (see, for example, Guatelli et al. (1990)), and Q-beta replicase amplification (see, for example, Smith et al. (1997); and Burg et al. (1996)).

The cDNA products resulting from these reverse transcriptase methods may serve as templates for multiple rounds of transcription by the appropriate RNA polymerase (for example, by nucleic acid sequence based amplification or NASBA, see, for example, Kievits et al. (1991); and Greijer et al. (2001)). Transcription of the cDNA template rapidly amplifies the signal from the original target mRNA.

These methods as well as others (either known or newly devised by one skilled in the art) may be used in the practice of the invention.

Amplification can also be used to quantify the amount of extracted RNA (see, for example, U.S. Pat. No. 6,294,338). Alternatively or additionally, amplification using appropriate oligonucleotide primers can be used to label cell-free RNA prior to analysis (see below). Suitable oligonucleotide amplification primers can easily be selected and designed by one skilled in the art.

### Labeling of RNA

In certain embodiments, RNA (for example, after amplification, or after conversion to cDNA or to cRNA) is labeled with a detectable agent or moiety before being analyzed. The role of a detectable agent is to facilitate detection of fetal RNA or to allow visualization of hybridized nucleic acid fragments (e.g., nucleic acid fragments bound to genetic probes). In some embodiments, the detectable agent is selected such that it generates a signal which can be measured and whose intensity is related to the amount of labeled nucleic acids present in the sample being analyzed. In array-based analysis methods, the detectable agent is also in some embodiments selected such that it generates a localized signal, thereby allowing spatial resolution of the signal from each spot on the array.

The association between the nucleic acid molecule and detectable agent can be covalent or non-covalent. Labeled nucleic acid fragments can be prepared by incorporation of or conjugation to a detectable moiety. Labels can be attached directly to the nucleic acid fragment or indirectly through a linker. Linkers or spacer arms of various lengths are known in the art and are commercially available, and can be selected to reduce steric hindrance, or to confer other useful or desired properties to the resulting labeled molecules (see, for example, Mansfield et al. (1995)).

Methods for labeling nucleic acid molecules are well-known in the art. For a review of labeling protocols, label detection techniques and recent developments in the field, see, for example, Kricka (2002); van Gijlswijk et al. (2001); and Joos et al. (1994). Standard nucleic acid labeling methods include: incorporation of radioactive agents, direct attachment of fluorescent dyes (see, for example, Smith et al. (1985)) or of enzymes (see, for example, Connoly and Rider (1985)); chemical modifications of nucleic acid fragments making them detectable immunochemically or by other affinity reactions (see, for example, Broker et al., (1978); Bayer et al. (1980); Langer et al. (1981); Richardson et al. (1983); Brigati et al. (1983); Tchen et al. (1984); Landegent et al. (1984); and Hopman et al. (1987)); and enzyme-mediated labeling methods, such as random priming, nick translation, PCR and tailing with terminal transferase (for a review on enzymatic labeling, see, for example, Temsamani and Agrawal (1996)). More recently developed nucleic acid labeling systems include, but are not limited to: ULS (Universal Linkage System; see, for example, Wiegant et al. (1999)), photoreactive azido derivatives (see, for example, Neves et al. (2000)), and alkylating agents (see, for example, Sebestyen et al. (1998)).

Any of a wide variety of detectable agents can be used in the practice of the present invention. Suitable detectable agents include, but are not limited to: various ligands, radionuclides (such as, for example, ³²P, ³⁵S, ³H, ¹⁴C, ¹²⁵I, ¹³¹I and the like); fluorescent dyes (for specific exemplary fluorescent dyes, see below); chemiluminescent agents (such as, for example, acridinium esters, stabilized dioxetanes and the like); microparticles (such as, for example, quantum dots, nanocrystals, phosphors and the like); enzymes (such as, for example, those used in an ELISA, i.e., horseradish peroxidase, beta-galactosidase, luciferase, alkaline phosphatase); colorimetric labels (such as, for example, dyes, colloidal gold and the like); magnetic labels (such as, for example, Dynabeads™); and biotin, dioxigenin or other haptens and proteins for which antisera or monoclonal antibodies are available.

In certain embodiments, fetal amniotic fluid RNA (after amplification, or conversion to cDNA or to cRNA) is fluorescently labeled. Numerous known fluorescentlabeling moieties of a wide variety of chemical structures and physical characteristics are suitable for use in the practice of this invention. Suitable fluorescent dyes include, but are not limited to: Cy-3™, Cy-5™, Texas red, FITC, phycoerythrin, rhodamine, fluorescein, fluorescein isothiocyanine, carbocyanine, merocyanine, styryl dye, oxonol dye, BODIPY dye (i.e., boron dipyrromethene difluoride fluorophore, see, for example, C. S. Chen et al., J. Org. Chem. 2000, 65: 2900-2906; Chen et al. (2000); U.S. Pat. Nos. 4,774,339; 5,187,288; 5,227,487; 5,248,782; 5,614,386; 5,994,063; and 6,060,324), and equivalents, analogues, derivatives or combinations of these molecules. Similarly, methods and materials are known for linking or incorporating fluorescent dyes to biomolecules such as nucleic acids (see, for example, Haugland (1994)). Fluorescent labeling dyes as well as labeling kits are commercially available from, for example, Amersham Biosciences, Inc. (Piscataway, N.J.), Molecular Probes, Inc. (Eugene, Oreg.), and New England Biolabs, Inc. (Beverly, Mass.).

Favorable properties of fluorescent labeling agents to be used in the practice of the invention include high molar absorption coefficient, high fluorescence quantum yield, and photostability. Some labeling fluorophores exhibit absorption and emission wavelengths in the visible (i.e., between 400 and 750 nm) rather than in the ultraviolet range of the spectrum (i.e., lower than 400 nm).

In some embodiments, RNA (for example, after amplification or conversion to cDNA or cRNA) is made detectable through one of the many variations of the biotin-avidin system, which are well known in the art. Biotin RNA labeling kits are commercially available, for example, from Roche Applied Science (Indianapolis, Ind.) Perkin Elmer (Boston, Mass.), and NuGEN (San Carlos, Calif.).

Detectable moieties can also be biological molecules such as molecular beacons and aptamer beacons. Molecular beacons are nucleic acid molecules carrying a fluorophore and a non-fluorescent quencher on their 5′ and 3′ ends. In the absence of a complementary nucleic acid strand, the molecular beacon adopts a stem-loop (or hairpin) conformation, in which the fluorophore and quencher are in close proximity to each other, causing the fluorescence of the fluorophore to be efficiently quenched by FRET (i.e., fluorescence resonance energy transfer). Binding of a complementary sequence to the molecular beacon results in the opening of the stem-loop structure, which increases the physical distance between the fluorophore and quencher thus reducing the FRET efficiency and allowing emission of a fluorescence signal. The use of molecular beacons as detectable moieties is well-known in the art (see, for example, Sokol et al., (1998); and U.S. Pat. Nos. 6,277,581 and 6,235,504). Aptamer beacons are similar to molecular beacons except that they can adopt two or more conformations (see, for example, Kaboev et al. (2000); Yamamoto et al. (2000); Hamaguchi et al. (2001); Poddar and Le (2001)).

A “tail” of normal or modified nucleotides may also be added to nucleic acid fragments for detectability purposes. A second hybridization with nucleic acid complementary to the tail and containing a detectable label (such as, for example, a fluorophore, an enzyme or bases that have been radioactively labeled) allows visualization of the nucleic acid fragments bound to the array (see, for example, system commercially available from Enzo Biochem Inc., New York, N.Y.).

The selection of a particular nucleic acid labeling technique may depend on the situation and may be governed by several factors, such as the ease and cost of the labeling method, the quality of sample labeling desired, the effects of the detectable moiety on the hybridization reaction (e.g., on the rate and/or efficiency of the hybridization process), the nature of the detection system to be used, the nature and intensity of the signal generated by the detectable label, and the like.

II. Analysis of RNA from Amniotic Fluid or Whole Maternal Blood

According to the present invention, RNA such as fetal RNA from amniotic fluid or RNA from whole maternal blood can be analyzed to obtain information regarding fetal gene expression. In certain embodiments, analyzing the RNA comprises determining the quantity, concentration or sequence composition of RNA.

RNA may be analyzed by any of a variety of methods. Methods of analysis of RNA are well-known in the art (see, for example, Sambrook et al. (1989); and Ausubel (Ed.) (2002)).

For example, the quantity and concentration of RNA extracted from amniotic fluid or whole maternal blood samples may be evaluated by UV spectroscopy, wherein the absorbance of a diluted RNA sample is measured at 260 and 280 nm (Wilfinger et al. (1997)). Quantitative measurements may also be carried out using certain fluorescent dyes, such as, for example, RiboGreen® (commercially available from Molecular Probes, Eugene, Oreg.), which exhibit a large fluorescence enhancement when bound to nucleic acids. RNA labeled with these fluorescent dyes can be detected using standard fluorometers, fluorescence microplate reader or filter fluorometers. Another method for analyzing quantity and quality of RNA samples is through use of a BioAnalyzer (commercially available from Agilent Technologies, Foster City, Calif.), which separates charged biological molecules (such as nucleic acids) using microfluidic technologies and then a laser to excite intercalating fluorescent dyes.

RNA may also be analyzed through sequencing. For example, RNase T1, which cleaves single-stranded RNA specifically at the 3′-side of guanosine residues in a two-step mechanism, may be used to digest denatured RNA. Partial digestion of 3′ or 5′ labeled RNA with this enzyme thus generates a ladder of G residues. The cleavage can be monitored by radioactive (Ikehara et al. (1986)) and photometric (Grunert et al. (1993)) detection systems, by zymogram assay (Bravo et al. (1994)), agar diffusion test (Quaas et al. (1989)), lanthan assay (Anfinsen et al. (1954)) or methylene blue test (Greiner-Stoeffele et al. (1996)) or by fluorescence correlation spectroscopy (Korn et al. (2000)).

Other methods for analyzing RNA include northern blots, wherein the components of the RNA sample being analyzed are resolved by size prior to detection thereby allowing identification of more than one species simultaneously, and slot/dot blots, wherein unresolved mixtures are used.

In certain embodiments, analyzing the RNA comprises submitting the extracted RNA to a gene-expression analysis. In some embodiments, this includes the simultaneous analysis of multiple genes.

For example, analysis of RNA may include detecting the presence of and/or quantitating a RNA transcribed from a gene known or suspected to be involved in a fetal anomaly such as congenital diaphragmatic hernia or condition such as Down Syndrome. In some embodiments, such genes are differentially expressed in the fetal disease or condition.

In analyses carried out to detect the presence or absence of RNA transcribed from a specific gene, the detection may be performed by any of a variety of physical, immunological and biochemical methods. Such methods are well-known in the art, and include, for example, protection from enzymatic degradation such as 51 analysis and RNase protection assays, in which hybridization to a labeled nucleic acid probe is followed by enzymatic degradation of single-stranded regions of the probe and analysis of the amount and length of probe protected from degradation.

In some embodiments of the invention, real time RT-PCR methods are employed that allow quantification of RNA transcripts and viewing of the increase in amount of nucleic acid as it is amplified. The TaqMan assay, a quenched fluorescent dye system, may also be used to quantitate targeted mRNA levels (see, for example Livak et al. (1995)).

In some embodiments of the invention involving methods that allow quantification of RNA transcripts (such as real time RT-PCR), expression housekeeping genes are used as normalization controls. Examples of housekeeping genes include GAPDH, 18S rRNA, beta-actin, cyclophilin, tubulin, etc.

Other methods are based on the analysis of cDNA derived from mRNA, which is less sensitive to degradation than RNA and therefore easier to handle. These methods include, but are not limited to, sequencing cDNA inserts of an expressed sequence tag (EST) clone library (see, for example, Adams et al. (1991)) and serial analysis of gene expression (or SAGE), which allows quantitative and simultaneous analysis of a large number of transcripts (see, for example, U.S. Pat. No. 5,866,330; V. E. Velculescu et al. (1995); and Zhang et al. (1997)). These two methods survey the whole spectrum of mRNA in a sample rather than focusing on a predetermined set.

Other methods of analysis of cDNA derived from mRNA include reverse transcriptase-mediated PCR (RT-PCR) gene expression assays. These methods are directed at specific target gene products and allow the qualitative (non-quantitative) detection of transcripts of very low abundance (see, for example, Su et al. (1997)). A variation of these methods, called competitive RT-PCR, in which a known amount of exogenous template is added as internal control, has been developed to allow quantitative measurements (see, for example, Beker-Andre and Hahlbrock (1989); Wang et al. (1989); and Gilliland et al. (1990)).

mRNA analysis may also be performed by differential display reverse transcriptase PCR (DDRT-PCR; see, for example, Liang and Pardee (1992)) or RNA arbitrarily primed PCR (RAP-CPR; see, for example, Welsh et al. (1992); and McClelland et al. (1993)). In these methods, RT-PCR fingerprint profiles of transcripts are generated by random priming and differentially expressed genes appear as changes in the fingerprint profiles between two samples. Identification of a differentially expressed gene requires further manipulation (i.e., the appropriate band of the gel must be excised, subcloned, sequenced and matched to a gene in a sequence database).

### III. Array-Based Gene Expression Analysis of Fetal RNA

In certain embodiments, the methods of the invention include submitting fetal amniotic fluid RNA or RNA from whole maternal blood to an array-based gene expression analysis.

### Array-Based Gene Expression Analysis

Traditional molecular biology methods, such as most of those described above, typically assess one gene per experiment, which significantly limits the overall throughput and prevents gaining a broad picture of gene function. Technologies based on DNA array or microarray (also called gene expression microarray), which were developed more recently, offer the advantage of allowing the monitoring of thousands of genes simultaneously through identification of sequence (gene/gene mutation) and determination of gene expression level (abundance) of genes (see, for example, Marshall and Hodgson (1998); Ramsay (1998); Ekins and Chu (1999); and Lockhart and Winzeler (2000)).

In a gene expression experiment, labeled cDNA or cRNA targets derived from the mRNA of an experimental sample are hybridized to nucleic acid probes immobilized to a solid support. By monitoring the amount of label associated with each DNA location, it is possible to infer the abundance of each mRNA species represented.

There are two standard types of DNA microarray technology in terms of the nature of the arrayed DNA sequence. In the first format, probe cDNA sequences (typically 500 to 5,000 bases long) are immobilized to a solid surface and exposed to a plurality of targets either separately or in a mixture. In the second format, oligonucleotides (typically 20-80-mer oligos) or peptide nucleic acid (PNA) probes are synthesized either in situ (i.e., directly on-chip) or by conventional synthesis followed by on-chip attachment, and then exposed to labeled samples of nucleic acids.

The analyzing step in the methods of the invention can be performed using any of a variety of methods, means and variations thereof for carrying out array-based gene expression analysis. Array-based gene expression methods are known in the art and have been described in numerous scientific publications as well as in patents (see, for example, Schena et al. (1995); Schena et al. (1996); and Chen et al. (1998); U.S. Pat. Nos. 5,143,854; 5,445,934; 5,807,522; 5,837,832; 6,040,138; 6,045,996; 6,284,460; and 6,607,885)

In the practice of the present invention, these methods as well as other methods known in the art for carrying out array-based gene expression analysis may be used as described or modified such that they allow fetal mRNA levels of gene expression to be evaluated.

### Test Genomic Profiles

In many embodiments, a test genomic profile comprising information about at least a subset of genes in a given genome for a test sample is used. In some embodiments, the test genomic profile contains information about at least 10, 20, 30, 40, 50, 60, 70, 80, 90, 100, 110, 120, 130, 140, 150, 160, 170, 180, 190, 200, 220, 240, 260, 280, 300, 320, 340, 360, 380, 400, 420, 440, 460, 480, 500, 550, 600, 650, 700, 750, 800, 850, 900, 950, 1000, 1100, 1200, 1300, 1400, 1500, 1600, 1700, 1800, 1900, 2000, 2200, 2400, 2600, 2800, 3000, 3200, 3400, 3600, 3800, 4000, 4200, 4400, 4600, 4800, 5000, 5200, 5400, 5600, 5800, 6000, 6500, 7000, 7500, 8000, 8500, 9000, 9500, 10000, 11000, 12000, 13000, 14000, 15000, 16000, 17000, 18000, 19000, 20000, or more genes.

In some embodiments, the test genomic profile is a test gene expression profile, i.e., a set of RNA levels for a plurality of genes. RNA levels can be, for example, obtained by analyzing RNA using an array-based gene expression method.

In some embodiments, RNA to be analyzed by an array-based gene expression method (e.g. “test sample RNA”) is isolated from a sample of amniotic fluid as described above. In some embodiments, test sample RNA is isolated from a sample of maternal blood. In many embodiments, the subject from whom test sample RNA is obtained (i.e., the “test subject”) is a pregnant woman carrying a fetus having or suspected of having a fetal disease or condition, such as Down Syndrome. (Down Syndrome and other fetal diseases or conditions are described herein). In some embodiments, the subject from whom test sample RNA is obtained is a fetus having or suspected of having a fetal disease or condition.

A test sample of RNA to be used in the methods of the invention may include a plurality of nucleic acid fragments labeled with a detectable agent.

The extracted RNA may be amplified, reverse-transcribed, labeled, fragmented, purified, concentrated and/or otherwise modified prior to the gene-expression analysis. Techniques for the manipulation of nucleic acids are well-known in the art, see, for example, J. Sambrook et al. (1989), Innis (Ed.) (1990); Tijssen (1993); M. A. Innis (Ed.) (1995), Academic Press: New York, N.Y.; and Ausubel (Ed.) (2002).

In certain embodiments, in order to improve the resolution of the array-based gene expression analysis, the nucleic acid fragments of the test sample are less then 500 bases long, in some embodiments less than about 200 bases long. The use of small fragments significantly increases the reliability of the detection of small differences or the detection of unique sequences.

Methods of RNA fragmentation are known in the art and include: treatment with ribonucleases (e.g., RNase T1, RNase V1 and RNase A), sonication (see, for example, Deininger (1983)), mechanical shearing, and the like (see, for example, Sambrook et al. (1989); Tijssen (1993); Ordahl et al. (1976); Oefner et al. (1996); Thorstenson et al. (1998)). Random enzymatic digestion of the RNA leads to fragments containing as low as 25 to 30 bases.

Fragment size of the nucleic acid segments in the test sample may be evaluated by any of a variety of techniques, such as, for example, electrophoresis (see, for example, Siles and Collier (1997)) or matrix-assisted laser desorption/ionization time-of-flight mass spectrometry (see, for example, Chiu et al. (2000)).

In the practice of the methods of the invention, the test sample of fetal amniotic fluid RNA is labeled before analysis. Suitable methods of nucleic acid labeling with detectable agents have been described in detail above.

Prior to hybridization, the labeled nucleic acid fragments of the test sample may be purified and concentrated before being resuspended in the hybridization buffer. Columns such as Microcon 30 columns may be used to purify and concentrate samples in a single step. Alternatively or additionally, nucleic acids may be purified using a membrane column (such as a Qiagen column) or Sephadex G50 and precipitated in the presence of ethanol.

### Reference Genomic Profiles

In many embodiments, a test genomic profile is compared against a reference genomic profile comprising information about at least a subset of genes in a given genome for a reference sample. In some embodiments, a reference genomic profile contains information about at least 10, 20, 30, 40, 50, 60, 70, 80, 90, 100, 110, 120, 130, 140, 150, 160, 170, 180, 190, 200, 220, 240, 260, 280, 300, 320, 340, 360, 380, 400, 420, 440, 460, 480, 500, 550, 600, 650, 700, 750, 800, 850, 900, 950, 1000, 1100, 1200, 1300, 1400, 1500, 1600, 1700, 1800, 1900, 2000, 2200, 2400, 2600, 2800, 3000, 3200, 3400, 3600, 3800, 4000, 4200, 4400, 4600, 4800, 5000, 5200, 5400, 5600, 5800, 6000, 6500, 7000, 7500, 8000, 8500, 9000, 9500, 10000, 11000, 12000, 13000, 14000, 15000, 16000, 17000, 18000, 19000, 20000, or more genes.

The reference genomic profile can comprise, for example, a value or set of values related to the amount and/or pattern of gene expression in a reference sample. In some embodiments, the reference genomic profile is a reference gene expression profile, i.e., a set of RNA levels for a plurality of genes. RNA levels can be, for example, obtained by analyzing RNA using an array-based gene expression method as described herein.

In some embodiments, the value or set of values for the reference is obtained from experiments performed on reference samples and/or using a reference subject. For example, reference values can be obtained from experiments on samples derived from comparable samples, such as amniotic fluid and/or maternal whole blood from pregnant women carrying fetuses that are not suffering from a condition or disease from which the test subject suffers.

In some embodiments, the reference sample is obtained from a fetus of the same gestational and/or developmental age as the test sample, or from a pregnant woman carrying such a fetus. In some embodiments, the reference sample is obtained from a fetus of the same gender, or from a pregnant woman carrying such a fetus. In some embodiments, the reference sample is obtained from a fetus that shares one or more attributes in common with the fetus from which the test sample is obtained, or from a pregnant woman carrying such a fetus.

In some embodiments, the reference sample is obtained from the same subject who provided the test sample, except at a different point in time and/or in a different stage as the subject was when the test sample was obtained. For example, the reference sample can be obtained from the same individual who is (and/or whose fetus is) at a different stage of development, at a different stage of the disease or condition, and/or at a different stage with respect to treatment (e.g., before treatment, and the commencement of treatment, during the treatment regimen, after treatment etc.). Alternatively or additionally, in embodiments wherein the subject is a pregnant woman, the reference sample can be obtained from the same woman when she was pregnant with another fetus that was not suffering from the particular disease or condition.

In some embodiments, a reference value or set of reference values may be determined, for example, by calculations, using algorithms, and/or from previously acquired and/or archived data.

In some embodiments, a reference expression profile is compiled from gene expression data obtained from more than one reference sample. For example, gene expression values for one gene (or for a particular subset of genes) may be obtained from data obtained from one reference sample or a set of reference samples, while gene expression values for another gene or for another particular subset of genes (which may or may not overlap the other particular subset of genes) may be obtained from another reference sample or set of reference samples. Alternatively or additionally, a gene expression value for one or more particular gene(s) in the reference expression profile may be averaged from a set of values obtained from more than one reference sample.

### Gene-Expression Hybridization Arrays

Any of a variety of arrays may be used in the practice of the present invention. Investigators can either rely on commercially available arrays or generate their own. Methods of making and using arrays are well known in the art (see, for example, Kern and Hampton (1997); Schummer et al. (1997); Solinas-Toldo et al. (1997); Johnston (1998); Bowtell (1999); Watson and Akil (1999); Freeman et al. (2000); Lockhart and Winzeler (2000); Cuzin (2001); Zarrinkar et al. (2001); Gabig and Wegrzyn (2001); and Cheung et al. (2001); see also, for example, U.S. Pat. Nos. 5,143,854; 5,434,049; 5,556,752; 5,632,957; 5,700,637; 5,744,305; 5,770,456; 5,800,992; 5,807,522; 5,830,645; 5,856,174; 5,959,098; 5,965,452; 6,013,440; 6,022,963; 6,045,996; 6,048,695; 6,054,270; 6,258,606; 6,261,776; 6,277,489; 6,277,628; 6,365,349; 6,387,626; 6,458,584; 6,503,711; 6,516,276; 6,521,465; 6,558,907; 6,562,565; 6,576,424; 6,587,579; 6,589,726; 6,594,432; 6,599,693; 6,600,031; and 6,613,893).

Arrays comprise a plurality of genetic probes immobilized to discrete spots (i.e., defined locations or assigned positions) on a substrate surface. Gene arrays used in accordance with some embodiments of the invention contain probes representing a comprehensive set of genes across the genome. In some such embodiments, the genes represented by the probes do not represent any particular subset of genes, and/or may be a random assortment of genes. In some embodiments of the invention, the gene arrays comprise a particular subset or subsets of genes. The subsets of genes may be represent particular classes of genes of interest. For example, an array comprising probes for developmental genes may be used in order to focus analyses on developmental genes. In such embodiments using arrays having particular subsets, more than one class of genes of interest may be represented on the same array.

Substrate surfaces suitable for use in the present invention can be made of any of a variety of rigid, semi-rigid or flexible materials that allow direct or indirect attachment (i.e., immobilization) of genetic probes to the substrate surface. Suitable materials include, but are not limited to: cellulose (see, for example, U.S. Pat. No. 5,068,269), cellulose acetate (see, for example, U.S. Pat. No. 6,048,457), nitrocellulose, glass (see, for example, U.S. Pat. No. 5,843,767), quartz or other crystalline substrates such as gallium arsenide, silicones (see, for example, U.S. Pat. No. 6,096,817), various plastics and plastic copolymers (see, for example, U.S. Pat. Nos. 4,355,153; 4,652,613; and 6,024,872), various membranes and gels (see, for example, U.S. Pat. No. 5,795,557), and paramagnetic or supramagnetic microparticles (see, for example, U.S. Pat. No. 5,939,261). When fluorescence is to be detected, arrays comprising cyclo-olefin polymers may in some embodiments be used (see, for example, U.S. Pat. No. 6,063,338).

The presence of reactive functional chemical groups (such as, for example, hydroxyl, carboxyl, amino groups and the like) on the material can be exploited to directly or indirectly attach genetic probes to the substrate surface. Methods for immobilizing genetic probes to substrate surfaces to form an array are well-known in the art.

More than one copy of each genetic probe may be spotted on the array (for example, in duplicate or in triplicate). This arrangement may, for example, allow assessment of the reproducibility of the results obtained. Related genetic probes may also be grouped in probe elements on an array. For example, a probe element may include a plurality of related genetic probes of different lengths but comprising substantially the same sequence. Alternatively, a probe element may include a plurality of related genetic probes that are fragments of different lengths resulting from digestion of more than one copy of a cloned piece of DNA. A probe element may also include a plurality of related genetic probes that are identical fragments except for the presence of a single base pair mismatch. An array may contain a plurality of probe elements. Probe elements on an array may be arranged on the substrate surface at different densities.

Array-immobilized genetic probes may be nucleic acids that contain sequences from genes (e.g., from a genomic library), including, for example, sequences that collectively cover a substantially complete genome or a subset of a genome (for example, the array may contain only human genes that are expressed throughout development). Genetic probes may be long cDNA sequences (500 to 5,000 bases long) or shorter sequences (for example, 20-80-mer oligonucleotides). The sequences of the genetic probes are those for which gene expression levels information is desired. Additionally or alternatively, the array may comprise nucleic acid sequences of unknown significance or location. Genetic probes may be used as positive or negative controls (for example, the nucleic acid sequences may be derived from karyotypically normal genomes or from genomes containing one or more chromosomal abnormalities; alternatively or additionally, the array may contain perfect match sequences as well as single base pair mismatch sequences to adjust for non-specific hybridization).

Techniques for the preparation and manipulation of genetic probes are well-known in the art (see, for example, J. Sambrook et al. (1989); Innis (Ed.) (1990); Tijssen (1993); Innis (Ed.) (1995); and Ausubel (Ed.) (2002)).

Long cDNA sequences may be obtained and manipulated by cloning into various vehicles. They may be screened and re-cloned or amplified from any source of genomic DNA. Genetic probes may be derived from genomic clones including mammalian and human artificial chromosomes (MACs and HACs, respectively, which can contain inserts from ˜5 to 400 kilobases (kb)), satellite artificial chromosomes or satellite DNA-based artificial chromosomes (SATACs), yeast artificial chromosomes (YACs; 0.2-1 Mb in size), bacterial artificial chromosomes (BACs; up to 300 kb); P1 artificial chromosomes (PACs; ˜70-100 kb) and the like.

Genetic probes may also be obtained and manipulated by cloning into other cloning vehicles such as, for example, recombinant viruses, cosmids, or plasmids (see, for example, U.S. Pat. Nos. 5,266,489; 5,288,641 and 5,501,979).

In some embodiments, genetic probes are synthesized in vitro by chemical techniques well-known in the art and then immobilized on arrays. Such methods are especially suitable for obtaining genetic probes comprising short sequences such as oligonucleotides and have been described in scientific articles as well as in patents (see, for example, Narang et al. (1979); Brown et al. (1979); Belousov et al. (1997); Guschin et al. (1997); Blommers et al. (1994); and Frenkel et al. (1995); see also for example, U.S. Pat. No. 4,458,066).

For example, oligonucleotides may be prepared using an automated, solid-phase procedure based on the phosphoramidite approach. In such a method, each nucleotide is individually added to the 5-end of the growing oligonucleotide chain, which is attached at the 3′-end to a solid support. The added nucleotides are in the form of trivalent 3′-phosphoramidites that are protected from polymerization by a dimethoxytrityl (or DMT) group at the 5-position. After base-induced phosphoramidite coupling, mild oxidation to give a pentavalent phosphotriester intermediate and DMT removal provides a new site for oligonucleotide elongation. The oligonucleotides are then cleaved off the solid support, and the phosphodiester and exocyclic amino groups are deprotected with ammonium hydroxide. These syntheses may be performed on commercial oligo synthesizers such as the Perkin Elmer/Applied Biosystems Division DNA synthesizer.

Methods of attachment (or immobilization) of oligonucleotides on substrate supports have been described (see, for example, Maskos and Southern (1992); Matson et al. (1995); Lipshutz et al. (1999); Rogers et al. (1999); Podyminogin et al. (2001); Belosludtsev et al. (2001)).

Oligonucleotide-based arrays have also been prepared by synthesis in situ using a combination of photolithography and oligonucleotide chemistry (see, for example, Pease et al., (1994); Lockhart et al. (1996); Singh-Gasson et al. (1999); Pirrung et al. (2001); McGall et al., (2001); Barone et al. (2001); Butler et al. (2001); Nuwaysir et al. (2002)). The chemistry for light-directed oligonucleotide synthesis using photolabile protected 2′-deoxynucleoside phosphoramites has been developed by Affymetrix Inc. (Santa Clara, Calif.) and is well known in the art (see, for example, U.S. Pat. Nos. 5,424,186 and 6,582,908).

An alternative to custom arraying of genetic probes is to rely on commercially available arrays and micro-arrays. Such arrays have been developed, for example, by Affymetrix Inc. (Santa Clara, Calif.), Illumina, Inc. (San Diego, Calif.), Spectral Genomics, Inc. (Houston, Tex.), and Vysis Corporation (Downers Grove, Ill.).

In some embodiments of the invention, provided are gene expression arrays for use in prenatal diagnostic applications. Such arrays are generally custom-made such that the genes represented on the array include at least a subset of genes that are known to be or suspected of being differentially expressed in a particular fetal disease or condition for which prenatal diagnosis is desirable. For example, a gene expression array for use in diagnosing Down Syndrome would include genetic probes for genes that are differentially expressed in trisomy 21 fetuses as discussed in Examples 2 and 3 of the present application.

### Hybridization

In the methods of the invention, the gene expression array may be contacted with the test sample under conditions wherein the nucleic acid fragments in the sample specifically hybridize to the genetic probes immobilized on the array.

The hybridization reaction and washing step(s), if any, may be carried out under any of a variety of experimental conditions. Numerous hybridization and wash protocols have been described and are well-known in the art (see, for example, Sambrook et al. (1989); Tijssen (1993); and Anderson (Ed.) (1999)). Methods of the invention may be carried out by following known hybridization protocols, by using modified or optimized versions of known hybridization protocols or newly developed hybridization protocols as long as these protocols allow specific hybridization to take place.

The term “specific hybridization” refers to a process in which a nucleic acid molecule preferentially binds, duplexes, or hybridizes to a particular nucleic acid sequence under stringent conditions. In the context of the present invention, this term more specifically refers to a process in which a nucleic acid fragment from a test sample preferentially binds (i.e., hybridizes) to a particular genetic probe immobilized on the array and to a lesser extent, or not at all, to other immobilized genetic probes of the array. Stringent hybridization conditions are sequence dependent. The specificity of hybridization increases with the stringency of the hybridization conditions; reducing the stringency of the hybridization conditions results in a higher degree of mismatch being tolerated.

The hybridization and/or wash conditions may be adjusted by varying different factors such as the hybridization reaction time, the time of the washing step(s), the temperature of the hybridization reaction and/or of the washing process, the components of the hybridization and/or wash buffers, the concentrations of these components as well as the pH and ionic strength of the hybridization and/or wash buffers.

In certain embodiments, the hybridization and/or wash steps are carried out under very stringent conditions. In other embodiments, the hybridization and/or wash steps are carried out under moderate to stringent conditions. In still other embodiments, more than one washing steps are performed. For example, in order to reduce background signal, a medium to low stringency wash is followed by a wash carried out under very stringent conditions.

As is well known in the art, the hybridization process may be enhanced by modifying other reaction conditions. For example, the efficiency of hybridization (i.e., time to equilibrium) may be enhanced by using reaction conditions that include temperature fluctuations (i.e., differences in temperature that are higher than a couple of degrees). An oven or other devices capable of generating variations in temperatures may be used in the practice of the methods of the invention to obtain temperature fluctuation conditions during the hybridization process.

It is also known in the art that hybridization efficiency is significantly improved if the reaction takes place in an environment where the humidity is not saturated. Controlling the humidity during the hybridization process provides another means to increase the hybridization sensitivity. Array-based instruments usually include housings allowing control of the humidity during all the different stages of the experiment (i.e., pre-hybridization, hybridization, wash and detection steps).

Additionally or alternatively, a hybridization environment that includes osmotic fluctuation may be used to increase hybridization efficiency. Such an environment where the hyper-/hypo-tonicity of the hybridization reaction mixture varies may be obtained by creating a solute gradient in the hybridization chamber, for example, by placing a hybridization buffer containing a low salt concentration on one side of the chamber and a hybridization buffer containing a higher salt concentration on the other side of the chamber

### Highly Repetitive Sequences

In the practice of the methods of the invention, the array may be contacted with the test sample under conditions wherein the nucleic acid segments in the sample specifically hybridize to the genetic probes on the array. As mentioned above, the selection of appropriate hybridization conditions will allow specific hybridization to take place. In certain cases, the specificity of hybridization may further be enhanced by inhibiting repetitive sequences.

In certain embodiments, repetitive sequences present in the nucleic acid fragments are removed or their hybridization capacity is disabled. By excluding repetitive sequences from the hybridization reaction or by suppressing their hybridization capacity, one prevents the signal from hybridized nucleic acids to be dominated by the signal originating from these repetitive-type sequences (which are statistically more likely to undergo hybridization). Failure to remove repetitive sequences from the hybridization or to suppress their hybridization capacity results in non-specific hybridization, making it difficult to distinguish the signal from the background noise.

Removing repetitive sequences from a mixture or disabling their hybridization capacity can be accomplished using any of a variety of methods well-known to those skilled in the art. These methods include, but are not limited to, removing repetitive sequences by hybridization to specific nucleic acid sequences immobilized to a solid support (see, for example, Brison et al. (1982)); suppressing the production of repetitive sequences by PCR amplification using adequate PCR primers; or inhibiting the hybridization capacity of highly repeated sequences by self-reassociation (see, for example, Britten et al. (1974)).

In some embodiments, the hybridization capacity of highly repeated sequences is competitively inhibited by including, in the hybridization mixture, unlabeled blocking nucleic acids. The unlabeled blocking nucleic acids, which are mixed to the test sample before the contacting step, act as a competitor and prevent the labeled repetitive sequences from binding to the highly repetitive sequences of the genetic probes, thus decreasing hybridization background. In certain embodiments, for example when cDNA derived from fetal mRNA is analyzed, the unlabeled blocking nucleic acids are Human Cot-1 DNA. Human Cot-1 DNA is commercially available, for example, from Gibco/BRL Life Technologies (Gaithersburg, Md.).

### Binding Detection and Data Analysis

In some embodiments, inventive methods include determining the binding of individual nucleic acid fragments of the test sample to individual genetic probes immobilized on the array in order to obtain a binding pattern. In array-based gene expression, determination of the binding pattern is carried out by analyzing the labeled array which results from hybridization of labeled nucleic acid segments to immobilized genetic probes.

In certain embodiments, determination of the binding includes: measuring the intensity of the signals produced by the detectable agent at each discrete spot on the array.

Analysis of the labeled array may be carried out using any of a variety of means and methods, whose selection will depend on the nature of the detectable agent and the detection system of the array-based instrument used.

In certain embodiments, the detectable agent comprises a fluorescent dye and the binding is detected by fluorescence. In other embodiments, the sample of RNA is biotin-labeled and after hybridization to immobilized genetic probes, the hybridization products are stained with a streptavidin-phycoerythrin conjugate and visualized by fluorescence. Analysis of a fluorescently labeled array usually comprises: detection of fluorescence over the whole array, image acquisition, quantitation of fluorescence intensity from the imaged array, and data analysis.

Methods for the detection of fluorescent labels and the creation of fluorescence images are well known in the art and include the use of “array reading” or “scanning” systems, such as charge-coupled devices (i.e., CCDs). Any known device or method, or variation thereof can be used or adapted to practice the methods of the invention (see, for example, Hiraoka et al. (1987); Aikens et al. (1989); Divane et al. (1994); Jalal et al. (1998); and Cheung et al. (1999); see also, for example, U.S. Pat. Nos. 5,539,517; 5,790,727; 5,846,708; 5,880,473; 5,922,617; 5,943,129; 6,049,380; 6,054,279; 6,055,325; 6,066,459; 6,140,044; 6,143,495; 6,191,425; 6,252,664; 6,261,776 and 6,294,331).

Commercially available microarrays scanners are typically laser-based scanning systems that can acquire one (or more) fluorescent image (such as, for example, the instruments commercially available from PerkinElmer Life and Analytical Sciences, Inc. (Boston, Mass.), Virtek Vision, Inc. (Ontario, Canada) and Axon Instruments, Inc. (Union City, Calif.)). Arrays can be scanned using different laser intensities in order to ensure the detection of weak fluorescence signals and the linearity of the signal response at each spot on the array. Fluorochrome-specific optical filters may be used during the acquisition of the fluorescent images. Filter sets are commercially available, for example, from Chroma Technology Corp. (Rockingham, Vt.).

In some embodiments, a computer-assisted imaging system capable of generating and acquiring fluorescence images from arrays such as those described above, is used in the practice of the methods of the invention. One or more fluorescent images of the labeled array after hybridization may be acquired and stored.

In some embodiments, a computer-assisted image analysis system is used to analyze the acquired fluorescent images. Such systems allow for an accurate quantitation of the intensity differences and for an easier interpretation of the results. A software for fluorescence quantitation and fluorescence ratio determination at discrete spots on an array is usually included with the scanner hardware. Softwares and/or hardwares are commercially available and may be obtained from, for example, BioDiscovery (El Segundo, Calif.), Imaging Research (Ontario, Canada), Affymetrix, Inc. (Santa Clara, Calif.), Applied Spectral Imaging Inc. (Carlsbad, Calif.); Chroma Technology Corp. (Brattleboro, Vt.); Leica Microsystems, (Bannockburn, Ill.); and Vysis Inc. (Downers Grove, Ill.). Other softwares are publicly available (e.g., MicroArray Image Analysis, and Combined Expression Data and Sequence Analysis (http://rana.lbl.gov); Chiang et al. (2001); a system written in R and available through the Bioconductor project (http://www.bioconductor.org); a Java-based TM4 software system available from the Institute for Genomic Research (http://www.tigr.org/software); and a Web-based system developed at Lund University (http://base.thep.lu.se)).

Accurate determination of fluorescence intensities requires normalization and determination of the fluorescence ratio baseline (Brazma and Vilo (2000)). Data reproducibility may be assessed by using arrays on which genetic probes are spotted in duplicate or triplicate. Baseline thresholds may also be determined using global normalization approaches (Kerr et al. (2000)). Other arrays include a set of maintenance genes which shows consistent levels of expression over a wide variety of tissues and allows the normalization and scaling of array experiments.

In the practice of the methods of the invention, any of a large variety of bioinformatics and statistical methods may be used to analyze data obtained by array-based gene expression analysis. Such methods are well known in the art (for a review of essential elements of data acquisition, data processing, data analysis, data mining and of the quality, relevance and validation of information extracted by different bioinformatics and statistical methods, see, for example, Watson et al. (1998); Duggan et al. (1999); Bassett et al. (1999); Hess et al. (2001); Marcotte and Date (2001); Weinstein et al. (2002); Dewey (2002); Butte (2002); Tamames et al. (2002); Xiang et al. (2003).

In gene expression array experiments, quantitative readouts of expression levels are typically provided. Typically, after normalization of data, genes having at least a 1.5-fold differences (i.e. a ratio of about 1.5) in expression levels between test and control samples may be considered “differentially expressed.” In some embodiments of the invention, genes considered to be differentially expressed show at least two-fold, at least five-fold, at least ten-fold, at least 15-fold, at least 20-fold, or at least 25-fold different expression levels compared to controls. (It is to be understood that the fold different expression levels can be determined in either direction, i.e., the expression levels for the test sample may be at least 1.5-fold higher or 1.5-fold lower than expression levels for the control sample.) In some embodiments, differential expression is assessed with respect to statistical significance for individual genes or groups of genes; in such embodiments, the fold-change may be lower, e.g., 1.4-fold, 1.3-fold, 1.2-fold, or 1.1-fold or even lower.

It will be appreciated that both the fold-difference cutoff for being considered differentially expressed varies depending on several factors which may include, for example, the type of samples used, the quantity and quality of the RNA sample, the power of the statistical analyses, the type of genes of interest, etc. In some embodiments, a lower cutoff ratio (i.e. −fold difference) is used, e.g., ratios of about 1.4, or about 1.3. In some embodiments, a higher cutoff ratio than about 1.5 is used, e.g., about 2.0, about 2.5, about 3.0, about 3.5, about 4.0, about 4.5, about 5.0, etc.

In some embodiments of the invention, gene expression data is analyzed at an individual level such that individual genes that are differentially expressed are identified. In some embodiments of the invention, gene expression data is analyzed by gene sets. Sets of genes may be grouped together based on location such as on a chromosomal band. For example, a set of genes on Chr21q22, a chromosomal region involved in Down Syndrome, may be analyzed together. Individual gene analyses and/or gene set analyses may identify functional groups and/or gene pathways involved in a particular fetal disease or condition. For further description of analytical methods used in the practice of the invention, see Examples 2 and 3 of the present application.

In some embodiments of the invention, gene expression data is fed into software programs to generate protein networks that may be involved in a particular fetal condition or disease. Protein network analyses may facilitate the design and/or development of novel fetal treatment approaches (see Examples 10 and 11 of the present application).

IV. Methods of Identifying Therapeutic Agents, Regimens, and/or Compounds

In one aspect, the invention provides methods for identifying therapeutic agents and/or regimens for a fetal disease or condition. In some embodiments, such methods comprise steps of: obtaining a reference genomic profile; obtaining a test genomic profile from a sample of amniotic fluid and/or maternal blood, wherein the sample is obtained from a subject suffering from or carrying a fetus suffering from a fetal disease or condition; determining differences between the test genomic profile and the reference genomic profile; inputting the test genomic profile into a first computing machine; accessing a storage repository on a second computing machine, wherein the storage repository contains a set of stored genomic profiles of one or more cell line(s) that have each been contacted with a different agent, wherein each genomic profile is mapped to data representing a corresponding agent; generating, by a correlator executing on the first or second computing machine, a correlation between each stored genomic profile and the test genomic profile; and selecting at least one agent whose corresponding genomic profile has a negative correlation score with the test genomic profile, the selected agent being likely to reduce the differences between the test genomic profile and the reference genomic profile.

### Genomic Profiles

Characteristics of reference genomic profiles and test genomic profiles are described herein. (See section III: Array-Based Gene Expression Analysis of Fetal RNA). In some embodiments, obtaining a reference genomic profile and/or a test genomic profile comprises creating the genomic profile using methods as described herein. In some embodiments, a reference genomic profile and/or test genomic profile is obtained from another source (e.g., a clinical laboratory, a research laboratory, a commercial service). Test genomic profiles are obtained from a sample of amniotic fluid using methods as described herein.

Genomic profiles generally comprise information about at least a subset of genes (and/or gene products) in a given genome. In some embodiments, genomic profiles comprise information about at least 10, 20, 30, 40, 50, 60, 70, 80, 90, 100, 110, 120, 130, 140, 150, 160, 170, 180, 190, 200, 220, 240, 260, 280, 300, 320, 340, 360, 380, 400, 420, 440, 460, 480, 500, 550, 600, 650, 700, 750, 800, 850, 900, 950, 1000, 1100, 1200, 1300, 1400, 1500, 1600, 1700, 1800, 1900, 2000, 2200, 2400, 2600, 2800, 3000, 3200, 3400, 3600, 3800, 4000, 4200, 4400, 4600, 4800, 5000, 5200, 5400, 5600, 5800, 6000, 6500, 7000, 7500, 8000, 8500, 9000, 9500, 10000, 11000, 12000, 13000, 14000, 15000, 16000, 17000, 18000, 19000, 20000, or more genes.

In some embodiments, the genomic profiles comprise information selected from the group consisting of mRNA levels (e.g., obtained from gene expression profiling experiments), protein expression levels, DNA methylation patterns, metabolite profiles, and combinations thereof.

Differences between the test genomic profile and the reference genomic profile can be determined using any of a variety of methods known in the art, such as, but not limited to, analytical and/or bioinformatics methods as discussed herein (see, for example, “Binding Detection and Data Analysis” in section III: “Array-Based Gene Expression Analysis of Fetal RNA”). In some embodiments, differences are determined using algorithms, functions, and/or scripts executing on one or more computing machines as described herein. In some embodiments, differences are determined visually and/or manually, e.g., by an individual. Not every point of difference between the test genomic profile and the reference genomic profile needs to be determined, though such a determination is contemplated and included in some embodiments of the invention. As would be understood by one of ordinary skill in the art, the determined differences generally provide an overall picture of differences across the genome and may guide an understanding of what genetic pathways may be disrupted in the test sample as compared to the reference sample. In some embodiments, a single difference is determined. In some embodiments, a plurality of differences is determined. In some embodiments at least 5, 10, 20, 30, 40, 50, 60, 70, 80, 90, 100, 110, 120, 130, 140, 150, 160, 170, 180, 190, 200, or more differences are determined

In accordance with provided methods, test genomic profiles are inputted into a first computing machine. By “inputting” it is meant that the test genomic profile, data representation(s) thereof, or data representation(s) of a subset of information contained in the test genomic profile, is entered into the first computing machine. By “data representations,” it is meant that the information in the test genomic profile may be summarized, abstracted, and/or represented in a different way (e.g., using numbers, symbols, code(s), binary numbers, etc.) before being inputted into the computing machine. In some embodiments, only a subset of information contained in the test genomic profile is inputted into the first computing machine. In some such embodiments, the subset of information comprises information deemed relevant (e.g., by a research or clinician, as determined by an algorithm, etc.) In some embodiments, inputting involves use of one or more inputting devices as described below. (See “computing machines.”)

In some embodiments, one or more names of genes that whose expression or other state is altered in the test genomic profile is inputted into the first computing machine, and the correlation step involves generating a correlation factor between the genes and the stored genomic profiles.

Although not required, in some embodiments, the reference genomic profile is also inputted into a computing machine, which may or may not be the same as the first computing machine into which the test genomic profile is inputted.

The fetal disease or condition may be any fetal disease or condition for which a therapeutic agent is desired. In some embodiments, the fetal disease or condition is selected from the group consisting of twin-to-twin-transfusion syndrome (TTTS), gastroschisis, Down Syndrome, fetal structural anomalies, fetal congenital heart anomaly, fetal kidney anomalies, neural tube defects, and congenital diaphragmatic hernia. In some such embodiments, the fetal disease or condition is Down Syndrome. In some embodiments, methods for identifying therapeutic agents further comprise testing the selected agent for medical applications in utero. It may be desirable, for example, to apply a therapy prenatally to the fetus and/or perinatally. In instances where prenatal therapy is desired, it may be advantageous to test the efficacy and safety of the therapeutic agent in utero, which would allow medical intervention before birth.

### Storage Repositories

The storage repository (in some embodiments, known as a “reference database”) on the second computing machine contains a set of stored genomic profiles. In some embodiments, the stored genomic profiles are of one or more cell line(s) that have each been contacted with a different agent. Thus, in such embodiments, each stored genomic profile corresponds to a particular agent. In such embodiments, each stored genomic profile is mapped to data representing a corresponding agent in the storage repository, such that it is possible to determine which agent, when contacted to the one or more cell line(s), corresponds to a given stored genomic profile.

The one or more cell lines can be any of a variety of cell lines known in the art, such as those used in biomedical and/or clinical research. These included without limitation cancer cell lines such as, for example, MCF7 (human breast cancer epithelial cells), PC3 (human prostate cancer epithelial cells), HL60 (human leukemia cells), SKMEL5 (human melanoma cells), etc. Generally, any cell line that is generated from a biological sample and/or organism may be suitable for use in the invention, so long as the cells in the cell line contain a genome that is comparable to that of the test and/or reference sample. Generally, the cell line(s) are obtained from a species that is the same as that from which the test and/or reference sample is obtained. For example, if the test genomic profile is to be obtained from a human biological sample, the cell line(s) used would likely be a human cell line.

The different agents can comprise any number of compounds, small molecules, drug candidates, nucleic acid agents, etc. The different agents may comprise all or a subset of the compounds, small molecules, etc. in a library or collection (e.g., historical collections of compounds and/or libraries from diversity-oriented syntheses). In some embodiments, the different agents comprise bioactive small molecules. In some embodiments, the different agents comprise agents in one or more classes of small molecules, e.g., histone deacetylase (HDAC) inhibitors, estrogens, phenothiazines, etc.

For example, a storage repository amenable for use in accordance with methods of the invention may comprise stored genomic profiles from a reference database comprising mRNA levels known as the “Connectivity Map,” which is publicly available. (See Lamb et al. (2006), the entire contents of which are herein incorporated by reference in their entirety.) The Connectivity Map comprises a reference collection of gene-expression profiles from cultured human cells treated with bioactive small molecules along with pattern-matching software that allows connections between small molecules, genes, diseases, and drugs to be found. Other storage repositories can also be used in accordance with the invention. Such storage repositories may include genomic profiles comprising information such as DNA methylation patterns, protein expression profiles, metabolite profiles, or combinations thereof. Information from more than one such database may be combined for use in inventive methods.

Storage repositories may be located on one or more computing machines as described below. Typically, storage repositories are located on one or more main memory components, although they can alternatively or additionally be located on a subsidiary memory component (such as, but not limited to, an external disk or drive in communication with the second computing machine).

The storage repository may be accessed in any of a variety of ways. In some embodiments, the storage repository is accessed via a bus (e.g., a system bus) within the second computing machine. In some embodiments, the storage repository is accessed via a netwok as described below (see “Computing machines”).

### Correlating and Selecting

Correlation scores generally give an indication of the degree to which two variables are associated. In some embodiments, the correlations score ranges from −1 to +1 and may be known as a “correlation coefficient.” In some such embodiments, a positive correlation score denotes a positive correlation, a negative correlation score denotes a negative correlation (also known as an “inverse correlation”), a correlation score of zero denotes no correlation, and the magnitude of the correlation score is an indication of the strength of the correlation. For example, in some such embodiments wherein the correlation score ranges from −1 to +1, the greater the magnitude of the correlation score, the greater the strength of the correlation (whether it is positive or negative). Thus, in such embodiments, the closer a negative correlation score is to −1, the stronger the negative correlation is, whereas the closer a negative correlation score is to 0, the weaker the negative correlation is. Similarly, in such embodiments, the closer a positive correlation score is to +1, the stronger the positive correlation is, whereas the closer a positive correlation score is to 0, the weaker the positive correlation is.

The correlation score can be generated using a correlator, which can execute on the first or second computing machine or both. The correlator may, in some embodiments, be a function, script, algorithm, computer program, software, etc. that employs a computational method to determine the correlation score between the test genomic profile and each stored genomic profile. For the purposes of computing the correlation score, in some embodiments, each datum of information in the test genomic profile and/or stored genomic profile is represented by a number. (For example, the number may correspond to gene expression values, fold-gene expression as compared to a control or reference, extent of methylation, extent of deacetylation, fold-protein expression as compared to a control or reference, etc.). Computational methods to compare genomic profiles (e.g., to determine a correlation score) are known in the art and include without limitation, nonparametric rank-based pattern-matching strategies such as those based on the Kolmogorov-Smirnov statistic (See, e.g., Hollander and Wolfe, Nonparametric Statistic Methods. Wiley, New York, ed. 2, 1999, pp. 178-185, the contents of which are herein incorporated by reference in their entirety.)

Selecting at least one agent in many embodiments comprises selecting an agent whose corresponding stored genomic profile has a strong (or “high”) negative correlation score with the test genomic profile. Such an agent may be deemed likely to reduce the differences between the test genomic profile and the reference genomic profile and may be desirable candidates and therapeutic drugs for the fetal disease or condition. In some embodiments, the strong negative correlation score is a correlation coefficient less than −0.4, −0.5, −0.6, −0.7, −0.8, −0.9, or less.

In some embodiments, an agent whose corresponding stored genomic profile has a strong (or “high”) positive correlation score with the test genomic profile. Such an agent may be deemed likely to mimic the test genomic profile, and may, for example, be useful in creating a model (e.g., an animal model and/or model in a cell line) of the disease or condition. In some embodiments, the strong positive correlation score is a positive coefficient greater than +0.4, +0.5, +0.6, +0.7, +0.8, +0.9, or more.

Selecting may be accomplished using for example, a function, script, algorithm, computer program, software, etc. executing on a computing machine, such as the first or second computing machine described herein. In some embodiments, selecting is accomplished without using a computing machine. In some embodiments, selecting is accomplished manually, e.g., an individual may scan a list of correlation scores and determine which agent(s) are to be selected.

In some embodiments, methods further comprise a step of testing activity of the selected agent (e.g., a candidate compound) in a model for the fetal disease or condition. Suitable models for the fetal disease or condition include, but are not limited to, animal models, in vitro cell culture assays, etc.

### Computing Machines

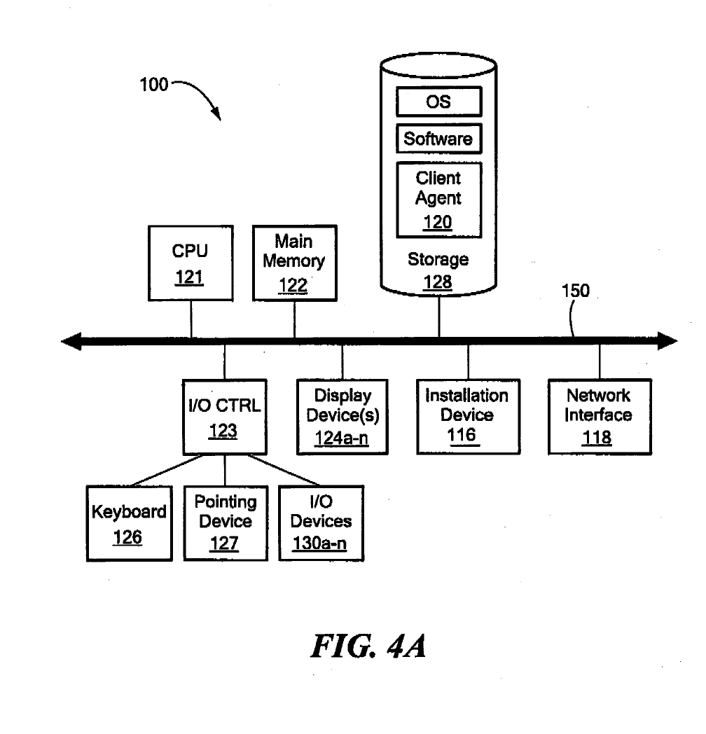

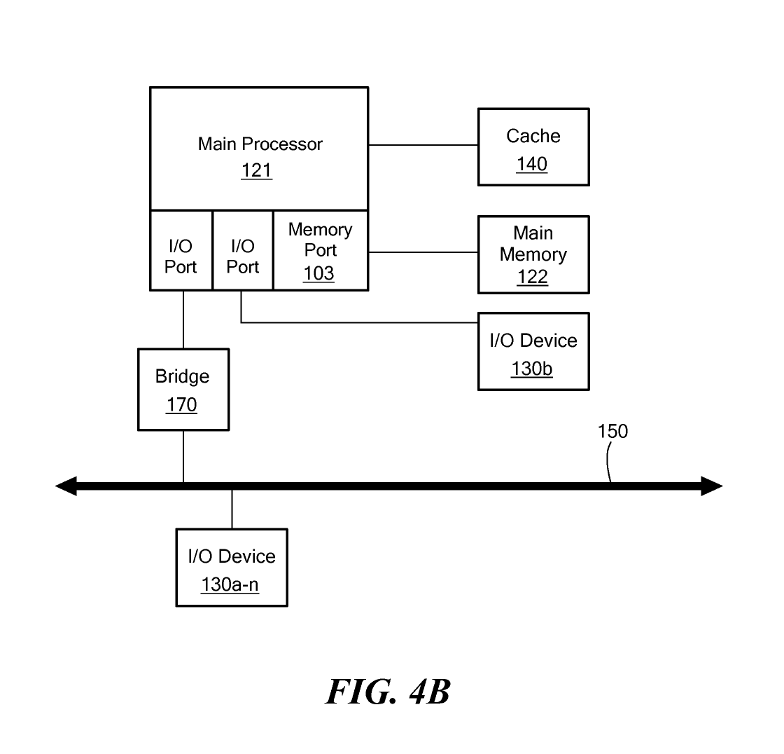

The first and second computing machines may be the same or different machine and may each comprise any type of computing device, such as a computer, network device or appliance capable of communicating on any type and form of network and performing the operations described herein. FIGS. 4A and 4B depict block diagrams of a computing device useful in practicing the invention. As shown in FIGS. 4A and 4B, a computing device includes a central processing unit 121 and a main memory unit 122. As shown FIG. 4A, a computing device 100 may include a storage device 128, an installation device 116, a network interface 118, an input/output controller 123, one or more display device(s) 124a-n, a keyboard 126, and a pointing device 127 (such as a mouse). The storage device may include, without limitation, an operating system, software, and a client agent (120).

As shown in FIG. 4B, each computing device may also include additional optional elements, such as a memory port 103, a bridge 170, one or more input/output devices 130a-130n (generally referred to using reference numeral 130), and a cache memory 140 in communication with the central processing unit 121.

The central processing unit is any logic circuitry that responds to and processes instructions fetched from the main memory unit. In many embodiments, the central processing unit comprises a microprocessor unit, such as those manufactured by Intel Corporation (Mountain View, Calif.), those manufactured by Motorola Corporation (Schaumburg, Ill.), those manufactured by Transmeta Corporation (Santa Clara, Calif.), the RS/6000 processor, those manufactured by International Business Machines (White Plains, N.Y.), and/or those manufactured by Advanced Micro Devices (Sunnyvale, Calif.). The computing device may be based on any of these processors, or any other processor capable of operating as described herein.

Main memory may comprise one or more memory chips capable of storing data and allowing any storage location to be directly accessed by the microprocessor, such as Static random access memory (SRAM), Burst SRAM or SynchBurst SRAM (BSRAM), Dynamic random access memory (DRAM), Fast Page Mode DRAM (FPM DRAM), Enhanced DRAM (EDRAM), Extended Data Output RAM (EDO RAM), Extended Data Output DRAM (EDO DRAM), Burst Extended Data Output DRAM (BEDO DRAM), Enhanced DRAM (EDRAM), synchronous DRAM (SDRAM), JEDEC SRAM, PC100 SDRAM, Double Data Rate SDRAM (DDR SDRAM), Enhanced SDRAM (ESDRAM), SyncLink DRAM (SLDRAM), Direct Rambus DRAM (DRDRAM), and/or Ferroelectric RAM (FRAM). The main memory may be based on any of the above described memory chips, or any other available memory chips capable of operating as described herein. In the embodiment shown in FIG. 4A, the central processor communicates with the main memory via a system bus 150 (described in more detail below). FIG. 4B depicts an embodiment of a computing device in which the processor communicates directly with main memory via a memory port 103. For example, in FIG. 4B, the main memory may be DRDRAM.

FIG. 4B depicts an embodiment in which the main processor 121 communicates directly with cache memory 40 via a secondary bus, sometimes referred to as a backside bus. In some embodiments, the main processor communicates with cache memory using the system bus 150. Cache memory typically has a faster response time than main memory and is typically provided by SRAM, BSRAM, or EDRAM. In the embodiment shown in FIG. 4A, the processor central processing unit 121 communicates with various input/output devices 130 via a local system bus 150. Various buses may be used to connect the central processing unit to any of the I/O devices, including a VESA VL bus, an ISA bus, an EISA bus, a MicroChannel Architecture (MCA) bus, a PCI bus, a PCI-X bus, a PCI-Express bus, or a NuBus. For embodiments in which the input/output device is a video display, the processor may use an Advanced Graphics Port (AGP) to communicate with the display. FIG. 4B depicts an embodiment of a computer in which the central processing unit communicates directly with input/output device 130b via HYPERTRANSPORT, RAPIDIO, or INFINIBAND communications technology. FIG. 4B also depicts an embodiment in which local busses and direct communication are mixed: the processor communicates with input/output device 130a using a local interconnect bus while communicating with I/O device 130b directly.

In some embodiments, the first computing machine is the same as the second computing machine.

In some embodiments, the first computing machine is different than the second computing machine. In some such embodiments, the first computing machine and second computing machine are connected via a network (e.g., a local-area network (LAN) (such as a company Intranet), a metropolitan area network (MAN), and/or a wide area network (WAN) (such as the Internet or the World Wide Web)). In some embodiments, the first computing machine and second computing machine are connected via more than one network. In some embodiments, the network comprises a private network. In some embodiments, the network comprises a public network.

Any type and/or form of network may be used to connect the first and second computing machines in embodiments wherein they are different. Networks compatible for use in accordance with the invention include, but are not limited to, any of the following: a point to point network, a broadcast network, a wide area network, a local area network, a telecommunications network, a data communication network, a computer network, an ATM (Asynchronous Transfer Mode) network, a SONET (Synchronous Optical Network) network, a SDH (Synchronous Digital Hierarchy) network, a wireless network and a wireline network. In some embodiments, the network comprises a wireless link, such as an infrared channel or satellite band. The topology of the network may comprise a bus, star, and/or ring network topology. The network may be of any network topology known to those ordinarily skilled in the art capable of supporting the operations described herein. In some embodiments, the network comprise mobile telephone networks utilizing any protocol or protocols used to communicate among mobile devices, including AMPS, TDMA, CDMA, GSM, GPRS or UMTS. In some embodiments, different types of data may be transmitted via different protocols. In other embodiments, the same types of data may be transmitted via different protocols.

In some embodiments, the first and/or second computing machine may comprise multiple, logically-grouped machines, which may or may not be remote from each other. In some such embodiments, a logical group of remote machines may be referred to as a server farm. In some embodiments, the remote machines are geographically dispersed. In some embodiments, a server farm may be a single entity. In some embodiments, the server farm comprises a plurality of server farms. In some embodiments, remote machines within each server farm are heterogeneous (e.g., one or more of the remote machines can operate according to one type of operating system platform (e.g., WINDOWS NT, manufactured by Microsoft Corp. of Redmond, Wash.), while one or more of the other remote machines can operate according to another type of operating system platform (e.g., Unix or Linux)).

In some embodiments, a remote machine within a server farm is not physically proximate to another remote machine in the same server farm. Thus, a group of remote machines logically grouped as a server farm may be interconnected using, for example, a wide-area network (WAN) connection or a metropolitan-area network (MAN) connection. For example, a server farm may include remote machines physically located in different continents or different regions of a continent, country, state, city, campus, or room. In some embodiments, data transmission speeds between remote machines in the server farm are increased by using a local-area network (LAN) and/or other direct connection to connect the remote machines.

In some embodiments, a remote machine is a file server, application server, web server, proxy server, appliance, network appliance, gateway, application gateway, gateway server, virtualization server, deployment server, SSL VPN server, firewall, or combination thereof. In some embodiments, a remote machine provides a remote authentication dial-in user service (referred to as a RADIUS server). In some embodiments, a remote machine is a blade server. In some embodiments, a remote machine executes a virtual machine providing, to a user or client computer, access to a computing environment.

In some embodiments, a client communicates with a remote machine. In some such embodiments, the client communicates directly with one of the remote machines in a server farm. In some embodiments, the remote machine receives requests from the client, forwards the requests to a second remote machine, and responds to the request by the client with a response to the request from the remote machine.

Any of a wide variety of input/output (I/O) devices 130a-130n may be present in the computing device 100. Input devices include, without limitation, keyboards, mice, trackpads, trackballs, microphones, and drawing tablets. Output devices include, without limitation, video displays, speakers, inkjet printers, laser printers, and dye-sublimation printers. The I/O devices may be controlled by an I/O controller 123 as shown in FIG. 4A. The I/O controller may control one or more I/O devices such as a keyboard 126 and a pointing device 127, e.g., a mouse or optical pen. Furthermore, an I/O device may also provide storage and/or an installation medium 116 for the computing device 100. In some embodiments, the computing device 100 may provide USB connections (not shown) to receive handheld USB storage devices (such as, for example, the USB Flash Drive line of devices manufactured by Twintech Industry, Inc. of Los Alamitos, Calif.).

Referring again to FIG. 4A, the computing device 100 may support any suitable installation device 116, such as a floppy disk drive for receiving floppy disks such as 3.5-inch, 5.25-inch disks or ZIP disks, a CD-ROM drive, a CD-R/RW drive, a DVD-ROM drive, tape drives of various formats, USB device, hard-drive, or any other device suitable for installing software and programs. The computing device 100 may further comprise a storage device, such as one or more hard disk drives or redundant arrays of independent disks, for storing an operating system and other related software, and for storing application software programs such as any program related to the client agent 120. Optionally, any of the installation devices 116 could also be used as the storage device. Alternatively or additionally, the operating system and the software can be run from a bootable medium, for example, a bootable CD, such as KNOPPIX, a bootable CD for GNU/Linux that is available as a GNU/Linux distribution from knoppix.net.

The computing device 100 may include a network interface 118 to interface to the network 104 through a variety of connections including, but not limited to, standard telephone lines, LAN or WAN links (e.g., 802.11, T1, T3, 56 kb, X.25, SNA, DECNET), broadband connections (e.g., ISDN, Frame Relay, ATM, Gigabit Ethernet, Ethernet-over-SONET), wireless connections, or some combination of any or all of the above. Connections can be established using a variety of communication protocols (e.g., TCP/IP, IPX, SPX, NetBIOS, Ethernet, ARCNET, SONET, SDH, Fiber Distributed Data Interface (FDDI), RS232, IEEE 802.11, IEEE 802.11a, IEEE 802.11b, IEEE 802.11g, CDMA, GSM, WiMax and direct asynchronous connections). In some embodiments, the computing device 100 communicates with other computing devices 100′ via any type and/or form of gateway or tunneling protocol such as Secure Socket Layer (SSL) or Transport Layer Security (TLS), and/or the Citrix Gateway Protocol manufactured by Citrix Systems, Inc. of Ft. Lauderdale, Fla. The network interface 118 may comprise a built-in network adapter, network interface card, PCMCIA network card, card bus network adapter, wireless network adapter, USB network adapter, modem, or any other device suitable for interfacing the computing device 100 to any type of network capable of communication and performing the operations described herein.

In some embodiments, the computing device 100 may comprise or be connected to multiple display devices 124a-124n, which each may be of the same or different type and/or form. As such, any of the I/O devices 130a-130n and/or the I/O controller 123 may comprise any type and/or form of suitable hardware, software, or combination of hardware and software to support, enable or provide for the connection and use of multiple display devices 124a-124n by the computing device 100. For example, the computing device 100 may include any type and/or form of video adapter, video card, driver, and/or library to interface, communicate, connect or otherwise use the display devices 124a-124n. In some embodiments, a video adapter may comprise multiple connectors to interface to multiple display devices 124a-124n. In some embodiments, the computing device 100 may include multiple video adapters, with each video adapter connected to one or more of the display devices 124a-124n. In some embodiments, any portion of the operating system of the computing device 100 may be configured for using multiple displays 124a-124n. In some embodiments, one or more of the display devices 124a-124n may be provided by one or more other computing devices, such as computing devices 100a and 100b connected to the computing device 100, for example, via a network. These embodiments may include any type of software designed and constructed to use another computer's display device as a second display device 124a for the computing device 100. One ordinarily skilled in the art will recognize and appreciate the various ways and embodiments that a computing device 100 may be configured to have multiple display devices 124a-124n.

In some embodiments, an I/O device 130 may be a bridge between the system bus 150 and an external communication bus, such as a USB bus, an Apple Desktop Bus, an RS-232 serial connection, a SCSI bus, a FireWire bus, a FireWire 800 bus, an Ethernet bus, an AppleTalk bus, a Gigabit Ethernet bus, an Asynchronous Transfer Mode bus, a HIPPI bus, a Super HIPPI bus, a SerialPlus bus, a SCl/LAMP bus, a FibreChannel bus, or a Serial Attached small computer system interface bus.

A computing device 100 of the sort depicted in FIGS. 4A and 4B typically operates under the control of operating systems, which control scheduling of tasks and access to system resources. The computing device 100 can be running any operating system such as any of the versions of the MICROSOFT WINDOWS operating systems, the different releases of the Unix and Linux operating systems, any version of the MAC OS for Macintosh computers, any embedded operating system, any real-time operating system, any open source operating system, any proprietary operating system, any operating systems for mobile computing devices, and/or any other operating system capable of running on the computing device and performing the operations described herein. Typical operating systems include, but are not limited to: WINDOWS 3.x, WINDOWS 95, WINDOWS 98, WINDOWS 2000, WINDOWS NT 3.51, WINDOWS NT 4.0, WINDOWS CE, WINDOWS XP, and WINDOWS VISTA, all of which are manufactured by Microsoft Corporation of Redmond, Wash.; MAC OS, manufactured by Apple Inc., of Cupertino, Calif.; OS/2, manufactured by International Business Machines of Armonk, N.Y.; Linux, a freely-available operating system distributed by Caldera Corp. of Salt Lake City, Utah; and any type and/or form of a Unix operating system.

### V. Methods of Treatment

In another aspect, the invention provides a method of treating a fetal disease or condition comprising administering to a patient suffering from a fetal disease or condition an effective dose of a compound or therapeutic agent identified by methods of the present invention, such that symptoms of the fetal disease or condition are ameliorated.

The fetal disease or condition may be selected from the group consisting of twin-to-twin-transfusion syndrome (TTTS), gastroschisis, Down Syndrome, fetal structural anomalies, fetal congenital heart anomaly, fetal kidney anomalies, neural tube defects, and congenital diaphragmatic hernia. In some embodiments in which the fetal disease or condition is Down Syndrome, the compound is selected from the group consisting of anti-oxidants, ion channel modulators, G-protein signaling modulators, and combinations thereof. It is proposed, without wishing to be bound by any particular theory, that anti-oxidants (e.g., celastrol) and ion channel modulators such as calcium channel blockers (e.g., verapamil, felodipine, nifedipine, combinations thereof, etc.) may be beneficial in the treatment of Down Syndrome, as suggested by gene expression data presented in Examples 2-4 of the present application.

In some embodiments, the compound is selected from the group consisting of copper sulfate, 15-delta prostaglandian J2, blebbistatin, prochlorperazine, 17-dimethylamino-geldanamycin, butein, nordihydroguaiaretic acid, acetylsalicyclic acid, 51825898, sirolimus, docosahexaenoic acid ethyl ester, diclofenac, mercaptopurine, indometacin, 5279552, 17-allylamino-geldanamycin, rottlerin, paclitaxel, pyrvinium, flufenamic acid, oligomycin, 5114445, resveratrol, Y-27632, carbamazepine, nitrendipine, fluphenazine, 5152487, prazosin, 5140203, cytochalasin B, vorinostate, MG-132, HNMPA-(AM)₃, decitabine, U0125, nocodazole, 5224221, 3-hydroxy-DL-kynurenine, 5162773, oxaprozin, colforsin, exemestane, felodipine, HC toxin, 5213008, dimethyloxalylglycine, 5109870, calmidazolium, 5255229, derivatives thereof, and combinations thereof (See Example 5.)

In some embodiments, the effective dose of the compound is administered in utero and/or perinatally. In some embodiments, the individual to which something is administered is a pregnant woman. In some embodiments, the individual to which something is administered is a fetus. In some embodiments, administering to a fetus comprises administering to the pregnant woman carrying the fetus.

### VI. Methods of Evaluating Treatments

In some aspects, the invention provides methods for evaluating the efficacy of a treatment for a fetal disease or condition. It may be desirable to evaluate the efficacy and/or necessity of a currently used treatment, for example, to distinguish between subgroups of patients with particular diseases or conditions that may respond differently to a particular treatment. In some embodiments, the treatment is a novel treatment being developed for use in routine prenatal care.

In some aspects, the invention provides methods for identifying therapeutic agents for a fetal disease or condition. Therapies are sorely lacking for many diseases and conditions affecting fetuses (such as Down Syndrome). Even for fetal diseases and conditions for which there are available therapies, existing interventions are often only available after birth or at very late stages in fetal development, which may be too late to be beneficial.

Methods for evaluating efficacy of a treatment generally comprise hybridizing RNA from an amniotic fluid and/or maternal blood sample from a subject suffering from or carrying a fetus suffering from a fetal disease or condition to at least one polynucleotide probe for at least one predetermined gene such that expression levels of at least one predetermined gene are obtained, wherein the sample is obtained from a subject to which the agent in step (b) has not been administered; (b) administering an agent to a subject suffering from the fetal disease or condition; (c) hybridizing RNA from an amniotic fluid and/or maternal blood sample from a subject suffering from or carrying a fetus suffering from a fetal disease or condition to at least one genetic probe for the same predetermined gene(s) from step (a) such that expression levels of the predetermined gene(s) are obtained, wherein the sample is obtained from a subject to which the agent has been administered; (d) comparing the gene expression levels of the predetermined genes obtained from steps (a) and (c); and (e) determining, based on the comparison, efficacy of the agent as a treatment for the fetal disease or condition.

Treatments can be evaluated and/or therapeutic agents can be identified for any of a variety of fetal diseases or conditions using inventive methods described above. These include fetal anomalies such as gastroschisis, diaphragmatic hernia, fetal congenital heart anomaly, fetal kidney anomalies, etc.; chromosomal abnormalities such as Down Syndrome, etc.; and fetal functional abnormalities such as twin to twin transfusion syndrome (TTTS), neural tube defects, etc.

### VII. Diagnostic Methods

In some aspects, the invention provides methods for diagnosing Down Syndrome. Some inventive diagnostic methods involve performing gene-expression analyses as described herein.

In some embodiments, diagnostic methods comprise providing an amniotic fluid and/or maternal blood sample from a pregnant woman; hybridizing RNA from the sample to at least ten genetic probes for at least ten genes that are differentially expressed in trisomy 21 fetuses such that expression levels of the at least ten genes are obtained; and determining, based on the expression levels of the at least ten genes, a diagnosis with respect to Down Syndrome.

The fetal RNA is obtained from a biological sample (such as, for example, amniotic fluid or maternal whole blood) from a woman pregnant with a fetus with a known gender and gestational age. Gene expression array experiments are then performed on the fetal RNA, and the resulting gene expression pattern for the fetus is compared against established gene expression profiles of sex-matched and gestationally age-matched fetuses that are karyotypically and developmentally normal. (Gene expression profiles for normal fetuses would be obtained from a database of mRNA expression levels established for male and female fetuses at different gestational ages.) The comparison of the test sample's gene expression profile with that from the database of data is then used as a basis for determining a diagnosis of Down Syndrome.

In some embodiments, the expression of genes of the sample is compared against expression profiles of sex-matched and gestationally age-matched trisomy 21 fetuses. (Gene expression for trisomy 21 fetuses may be, for example, obtained from a database of mRNA expression levels established for male and female trisomy 21 fetuses at different gestational ages.) In such embodiments, similarities between the gene expression of the sample and that of the reference trisomy 21 data are positive indicators for a diagnosis of Down Syndrome.

Some inventive diagnostic methods involve detecting expression of a particular gene or subset of genes that are known to be differentially expressed in trisomy 21 fetuses. In such methods, a biological sample (such as, for example, amniotic fluid or maternal whole blood) from a pregnant woman is provided. Expression of at least one gene that is differentially expressed in trisomy 21 fetuses is then detected in the biological sample, and a determination is made based on the detected expression with respect to a diagnosis of Down Syndrome.

In some embodiments of the invention, a custom microarray is used for performing gene expression profiling experiments. Such custom microarrays contain genetic probes for at least a subset of genes that are differentially expressed in trisomy 21 fetuses.

The diagnosis in inventive diagnostic methods can be, for example that the fetus has or does not have Down Syndrome, that the fetus is at risk for developing Down Syndrome, that the fetus is in a particular stage of developing Down Syndrome, that the fetus is likely to develop particular disorders related to Down Syndrome, that the fetus may or may not be responsive to particular therapeutic interventions, etc.

### VIII. Kits

Inventive kits are provided that may be used in prenatal diagnostic applications. Such kits comprise gene expression microarrays that are designed to contain genetic probes for at least a subset of differentially expressed genes associated with a particular fetal disease or condition (as described herein in the “Gene expression microarrays” section). Such kits also comprise a database, or information about how to access a database, comprising baseline levels of mRNA expression established for karyotypically and developmentally normal male and normal female fetuses at different gestational ages. Instructions for using the gene expression arrays in conjunction with the database for diagnostic purposes are also included in inventive kits. In some embodiments, inventive kits include materials for extracting RNA from samples. In some such embodiments, materials are provided that allow extraction of RNA from amniotic fluid samples. In some embodiments, materials are provided that allow extraction of RNA from maternal whole blood samples as well as instructions on how to distinguish fetal RNA transcripts from maternal RNA transcripts.

### IX. Fetal Diseases and Conditions

It will be understood by those of ordinary skill in the art that inventive methods, microarrays, and reagents can be used in the development and evaluation of treatments for and/or diagnosis of a variety of fetal disorders. These include, but are not limited to, fetal anomalies such as gastroschisis, diaphragmatic hernia, fetal congenital heart anomaly, fetal kidney anomalies, etc.; chromosomal abnormalities such as Down Syndrome, etc.; and fetal functional abnormalities such as twin to twin transfusion syndrome (TTTS), neural tube defects, etc. For illustrative purposes, a subset of these diseases and conditions are described in further detail below.

Down Syndrome (also known as Trisomy 21) is a disorder caused by the presence of an extra copy of genetic material on chromosome 21 in humans. Trisomy 21 is the most common liveborn fetal autosomal aneuploidy. Down Syndrome patients have shortened life expectancy and reduced fertility. Most Down Syndrome patients exhibit mild to moderate mental retardation. The biological mechanisms underlying Down Syndrome are poorly understood, and fetal therapeutic interventions are lacking. Understanding gene expression profiles of trisomy 21 fetuses may shed light on genetic mechanisms underlying Down Syndrome and may lead to therapies and/or to novel biomarkers for prenatal diagnosis.

Gastroschisis and CDH are relatively common malformations that are easily detected on sonographic examination, and can be associated with significant postnatal morbidity. For both conditions, amniocentesis may be offered as part of the initial diagnostic work-up.

Gastroschisis is a common birth defect characterized by a fissure in the abdominal wall, usually accompanied by protrusion of the viscera. This condition is currently detected with maternal serum screening and confirmed by ultrasound examination. Though it is no longer a lethal disease, short- and long-term morbidity of this condition can be significant, and the degree of intestinal damage is highly variable. In gastroschisis, the typical appearance of exteriorized intestine is a thickened, foreshortened mesentery and stiff, inflamed bowel loops covered with a “peel” of thick pseudomembranes. The degree of peel is variable, and a subset of patients hardly exhibits any bowel wall inflammation at all.

It remains unclear what factors influence clinical severity and/or outcome. It has been suggested that the duration of exposure of the intestinal loops to amniotic fluid may correlate with the degree of damage, though this has not been confirmed. Postnatal recovery of intestinal function and length of hospitalization is shorter in full-term infants than in pre- or near-term ones, contradicting the notion that amniotic fluid is noxious in this condition. Other suggested factors include the diameter of the abdominal wall defect and the degree of mesenteric constriction, though studies have yielded inconclusive results.

While the majority of neonates with gastroschisis will have a return of normal bowel function within weeks, a substantial minority of patients will experience prolonged intestinal dysfunction lasting months and requiring prolonged parenteral nutrition. These infants are at significant risk of developing central venous line-associated sepsis, TPN-related cholestasis, and liver disease. It is not yet possible to antenatally stratify patients with gastroschisis and to predict which fetuses will follow a protracted course after birth.

Congenital diaphragmatic hernia (CDH) is a condition that can easily be diagnosed prenatally. Despite significant advances in postnatal management, CDH is still associated with high morbidity rates, and survival for the most severe forms continues to be poor. The biology of this condition is still largely unknown, but its genetic basis is becoming increasingly recognized.

### EXAMPLES

The following examples describe some of the preferred modes of making and practicing the present invention. However, it should be understood that these examples are for illustrative purposes only and are not meant to limit the scope of the invention. Furthermore, unless the description in an Example is presented in the past tense, the text, like the rest of the specification, is not intended to suggest that experiments were actually performed or data were actually obtained.

### Example 1

### Fetal mRNA Extraction from Amniotic Fluid

This Example demonstrates the successful extraction and amplification of cell-free fetal mRNA from both fresh and frozen residual amniotic fluid samples. Amniotic fluid samples were initially collected for routine diagnostic purposes; the supernatant is usually discarded following karyotype analysis, while in therapeutic amniocentesis the entire sample is discarded. In a cytogenetics laboratory, samples were spun at 350×g for 10 minutes to remove cells for culture. Samples were centrifuged again at 13,000×g either upon receipt in the case of fresh samples, or immediately after thawing in the case of frozen samples. This ensured that the extracted RNA was truly extracellular.

RNA was extracted using the Qiagen Viral RNA mini kit. Sample starting volumes were typically 420 μL. Synthetic poly-A RNA (15-25 μg) was added to the sample during extraction as a carrier. RNA was concentrated into a final volume of 60 μL.

Initially mRNA was extracted from frozen samples, and was present at a concentration between 500 and 1000 pg/mL. To test whether RNA was degraded by the freeze/thaw process and/or the time lapse between drawing and freezing the sample, frozen samples were thawed and two 420 μL aliquots were drawn; one for immediate processing and one that was kept at 4° C. for three hours before being subjected to RNA extraction. In all cases, there was a significant loss of amplifiable RNA over the three-hour period. Nevertheless, if the amniotic fluid was frozen immediately after acquisition, more RNA was recovered from the frozen sample as compared to the fresh sample.

From these preliminary experiments, it appears that the extracellular RNA present in amniotic fluid at the time of sample acquisition degrades over time. However, there also appears to be an increase in extracellular RNA from lysis and degradation of amniocytes, either over time or from the freezing and thawing of a sample. To obtain the most accurate assessment of extracellular RNA, it is suggested that samples be cleared of all cells as soon as possible after being drawn. It is suggested that samples then be processed immediately or subjected to the addition of RNAse inhibitor and frozen at −80° C.

### Example 2

### Preliminary Gene Expression Profiling of Down Syndrome Fetuses

In this Example, gene expression differences were analyzed between samples from second trimester fetuses with Down Syndrome (DS, also known as trisomy 21) and euploid gestational age-matched controls. Differentially expressed genes were identified by two different methods: one analyzing individual gene differences and another analyzing sets of genes. Such analyses may yield information useful in diagnosis and treatment of fetal diseases, and/or the understanding of fetal development.

### Materials and Methods

Ten mL of residual amniotic fluid (AF) was obtained from women undergoing fetal genetic testing between 16 and 21 weeks of gestation.

RNA was extracted from 8 samples from trisomy 21 fetuses and from 12 euploid samples using the RNeasy® Maxi Kit (QIAGEN). cDNA was synthesized from extracted RNA, amplified, biotin labeled, and hybridized to Human Genome U133 Plus 2.0 Microarrays (Affymetrix). Arrays were scanned with a GeneArray Scanner and analyzed using GeneChip Microarray Suite 5.0 (Affymetrix). The Bioconductor tool set in the R statistical computing and graphics software environment (http://www.r-project.org/) and Gene Set Enrichment Analysis (GSEA) tool (Subramanian et al. (2005) the contents of which are herein incorporated by reference in their entirety) from the Broad Institute were used to determine if concordant gene expression differences are present in trisomy 21 fetuses compared to normal controls. Onto-Express (Khatri et al. (2002), the contents of which are herein incorporated by reference in their entirety) was used to determine significantly over-represented Gene Ontology (GO) annotations among the gene lists.

### Results

Twenty-three genes were significantly differentially expressed in trisomy 21 fetuses compared to controls. Two of the 23 genes are on chromosome 21. Over-represented functional categories among the list of genes include apoptosis, integrin-mediated signaling and multicellular organismal development. Only one chromosomal band was significantly differentially expressed as a group: the critical region of chromosome 21 (q22). These genes include those that encode potassium and chloride ion binding and transport proteins, as well as those related to heart contraction and nervous system development.

### Discussion/Conclusion

These results show that microarray profiling is useful for the evaluation of abnormal gene expression in the early development of fetuses with trisomy 21. A set of genes, including those related to heart and nervous system development and function, were differentially expressed in fetuses with DS compared to controls. Gene expression profiling of trisomy 21 fetuses early in gestation may contribute to a better understanding of developmental abnormalities associated with this condition.

### Example 3

### Gene Expression Profiling of Down Syndrome Fetuses

In this Example, expanded gene expression analyses of trisomy 21 fetuses were performed to identify more genes and further characterize dysregulated genes in Down Syndrome. Similar to the analyses in Example 2, expression differences were analyzed by examining individual genes as well as sets of genes.

### Materials and Methods

### Samples

Residual amniotic fluid (AF) supernatant samples were obtained from women in their second trimester of pregnancy who were undergoing fetal genetic testing for routine clinical indications. All samples were anonymous, although the karyotype results and gestational ages were known. Samples were stored at −80° C. until RNA extraction. The initial study set consisted of AF samples with the following confirmed metaphase karyotypes: 47,XX,+21 (n=4); 47,XY,+21 (n=5); 46, XX (n=6); and 46, XY (n=6). Gestational ages of fetuses ranged from about 15 weeks to about 22 weeks.

RNA Extraction and cDNA Synthesis and Amplification

RNA was extracted from amniotic fluid samples from trisomy 21 fetuses and from age- and sex-matched karyotypically normal controls. RNA was extracted using a commercially available kit (RNeasy® Maxi Kit, QIAGEN) with some modification. Samples were thawed and homogenized in TRIzol LS reagent (Invitrogen) to permit complete dissociation of nucleoprotein complexes. After homogenization, samples were combined with chloroform to allow separation into organic and aqueous phases. The aqueous phases of each sample were then passed through RNeasy® columns and then processed according to the remaining steps of the manufacturer's protocol for the RNAeasy® Maxi Kit. (For 10 mL of AF, 30 mL of TRIzol LS reagent and 8 mL of chloroform was used.)

RNA was precipitated using 3M NaOAc and 100% ethanol, and 80% ethanol was added after 4 h incubation at −20° C.

cDNA was synthesized from extracted RNA and then amplified and purified using the WT-Ovation™ Pico RNA Amplification System (NuGEN) and the DNA Clean & Concentrator-25 (Zymo Research) according to the manufacturer's instructions. The WT-Ovation™ Pico RNA Amplification System is specifically designed for the uniform amplification of low starting quantities of RNA (500 pg to 50 ng). In this system, amplification is initiated both at the 3′ end and randomly throughout the whole transcriptome to enable amplification of intact mRNA as well as non-poly(A) transcripts and compromised RNA samples. Double-stranded cDNA was synthesized through a two-step process, using both random and poly(T) primers and reverse transcriptase in the first strand step, and DNA polymerase to generate double stranded cDNA in the second step.

Unamplified cDNA was purified using Agencourt RNAClean® magnetic beads and then amplified using the SPIA™ Amplification Protocol (NuGEN) according to manufacturer's instructions. SPIA™ amplification uses DNA/RNA chimeric primers, DNA polymerase and RNAse H in a homogenous isothermal assay. RNAse H is used to degrade RNA in the DNA/RNA heteroduplex at the 5′ end of the first cDNA strand, which results in the exposure of a DNA sequence that is available for binding of a chimeric primer. DNA polymerase then initiates replication at the 3′ end of the primer, displacing the existing forward strand. The RNA portion at the 5′ end of the newly synthesized strand is again removed by RNase H, exposing part of the unique priming site for initiation of the next round of cDNA synthesis. The process is repeated, leading to up to 15,000 fold amplification of cDNA that is complementary to the original RNA.

Amplified cDNA was purified using Zymo-Spin II Columns (Zymo Research). The quality and quantity of amplified cDNA was measured on the Agilent Bioanalyzer 2100 Expert software (Agilent) with the RNA 6000 Nano kit (Agilent).

### Fragmentation, Labeling, and Hybridization

cDNA was then biotin labeled and fragmented using the FL-Ovation cDNA Biotin Module V2 (NuGEN) according to the manufacturer's instructions. At least 5 μg of biotin labeled and fragmented cDNA suitable for hybridization to the Affymetrix GeneChip® Human Genome U133 Plus 2.0 Array was obtained. (Affymetrix GeneChip® Human Genome U133 Plus 2.0 Arrays allow analysis of over 47,000 transcripts and variants derived from over 38,500 human genes.) Arrays were washed, stained with streptavidin-phycoerythrin, scanned with the GeneArray Scanner, and analyzed using the GeneChip Microarray Suite 5.0 (Affymetrix, Santa Clara, Calif.). Array quality was assessed in R (version 2.7.2) using the simpleaffy package in BioConductor (version 1.7; www.bioconductor.org). Three arrays with scaling factors above 22 and fewer than 15% present calls were discarded.

Seven samples from DS fetuses remained: 5 males and 2 females. Five gender-matched controls were matched within 4 days of gestational age of the corresponding DS samples; the other two were collected 10 and 12 days earlier than the respective DS samples. A total of 7 DS and 7 matched controls were further analyzed. (See Table 1.)

TABLE 1
Sample pairings for gene expression comparisons

| | | |
|---|---|---|
| | | Age |
| Karyotype | Sample | (weeks’ gestation) |

Trisomy 21 sample

| | | |
|---|---|---|
| 47XY + 21 | 1 | 17 1/7 |
| | 2 | 19 5/7 |
| | 3 | 21 1/7 |
| | 4 | 16 4/7 |
| | 5 | 19 6/7 |
| 47XX + 21 | 6 | 17 2/7 |
| | 7 | 21 4/7 |

Matched control sample

| | | |
|---|---|---|
| 46XY | 1 | 17 2/7 |
| | 2 | 18 2/7 |
| | 3 | 20 4/7 |
| | 4 | 17 0/7 |
| | 5 | 18 1/7 |
| 46XX | 6 | 17 5/7 |
| | 7 | 21 3/7 |

### Analysis and Statistics

Normalization was performed using the three step command from the AffyPLM package in BioConductor, using ideal mismatch for background/signal adjustment, quantile normalization, and the Tukey biweight summary method (Gentleman et al. (2005), the contents of which are herein incorporated by reference in their entirety.

This summary method includes a logarithmic transformation, improving normality of the data. Identification of individual differentially-expressed genes was performed via two-sided, paired t-tests using the multtest package in BioConductor, with the Benjamini-Hochberg adjustment for multiple testing (Benjamini and Hochberg (1995), the contents of which are herein incorporated by reference in their entirety).

Gene Set Enrichment Analysis (GSEA) (Subramanian et al. (2005), the contents of which are herein incorporated by reference in their entirety) was performed using GSEA software v. 2.0 and MSigDB version 2.4. This analysis identifies consistent differential expression of sets of genes defined in the MSigDB database. We examined both the functional, curated gene sets (MSigDB collection c2) and gene sets defined by chromosomal bands (MSigDB collection c1), but only the chromosomal band analysis yielded sets that were significant with a false discovery rate (FDR) below 0.05. The full results of the chromosomal band analysis appear in Table 3.

TABLE 3
Full GSEA results showing significance of differential
expression in DS vs. euploid, by chromosomal band

| | FDR |
|---|---|
| NAME | q-val |
| CHR10P11 | 1.000 |
| CHR10P12 | 0.930 |
| CHR10P13 | 1.000 |
| CHR10P14 | 1.000 |
| CHR10P15 | 1.000 |
| CHR10Q11 | 0.927 |
| CHR10Q21 | 1.000 |
| CHR10Q22 | 1.000 |
| CHR10Q23 | 1.000 |
| CHR10Q24 | 0.948 |
| CHR10Q25 | 1.000 |
| CHR10Q26 | 1.000 |
| CHR11P11 | 1.000 |
| CHR11P13 | 0.933 |
| CHR11P14 | 0.919 |
| CHR11P15 | 0.975 |
| CHR11Q11 | 0.971 |
| CHR11Q12 | 1.000 |
| CHR11Q13 | 0.950 |
| CHR11Q14 | 1.000 |
| CHR11Q21 | 1.000 |
| CHR11Q22 | 0.989 |
| CHR11Q23 | 1.000 |
| CHR11Q24 | 1.000 |
| CHR11Q25 | 1.000 |
| CHR12P11 | 0.963 |
| CHR12P12 | 1.000 |
| CHR12P13 | 1.000 |
| CHR12Q | 1.000 |
| CHR12Q12 | 0.989 |
| CHR12Q13 | 0.972 |
| CHR12Q14 | 1.000 |
| CHR12Q15 | 0.970 |
| CHR12Q21 | 1.000 |
| CHR12Q22 | 1.000 |
| CHR12Q23 | 0.952 |
| CHR12Q24 | 0.958 |
| CHR13Q12 | 0.992 |
| CHR13Q13 | 0.931 |
| CHR13Q14 | 1.000 |
| CHR13Q21 | 1.000 |
| CHR13Q32 | 0.556 |
| CHR13Q33 | 0.908 |
| CHR13Q34 | 0.092 |
| CHR14Q11 | 1.000 |
| CHR14Q12 | 0.984 |
| CHR14Q13 | 1.000 |
| CHR14Q21 | 0.946 |
| CHR14Q22 | 1.000 |
| CHR14Q23 | 1.000 |
| CHR14Q24 | 1.000 |
| CHR14Q31 | 0.755 |
| CHR14Q32 | 1.000 |
| CHR15Q11 | 0.939 |
| CHR15Q13 | 0.967 |
| CHR15Q14 | 0.959 |
| CHR15Q15 | 0.942 |
| CHR15Q21 | 1.000 |
| CHR15Q22 | 0.954 |
| CHR15Q23 | 1.000 |
| CHR15Q24 | 0.882 |
| CHR15Q25 | 1.000 |
| CHR15Q26 | 0.982 |
| CHR16P11 | 0.973 |
| CHR16P12 | 0.980 |
| CHR16P13 | 0.963 |
| CHR16Q12 | 1.000 |
| CHR16Q13 | 0.992 |
| CHR16Q21 | 0.964 |
| CHR16Q22 | 0.933 |
| CHR16Q23 | 0.976 |
| CHR16Q24 | 1.000 |
| CHR17P11 | 0.948 |
| CHR17P12 | 0.977 |
| CHR17P13 | 0.972 |
| CHR17Q11 | 1.000 |
| CHR17Q12 | 1.000 |
| CHR17Q21 | 0.953 |
| CHR17Q22 | 1.000 |
| CHR17Q23 | 0.972 |
| CHR17Q24 | 0.965 |
| CHR17Q25 | 0.918 |
| CHR18P11 | 1.000 |
| CHR18Q11 | 0.999 |
| CHR18Q12 | 0.983 |
| CHR18Q21 | 0.952 |
| CHR18Q22 | 1.000 |
| CHR18Q23 | 0.962 |
| CHR19P12 | 1.000 |
| CHR1P13 | 1.000 |
| CHR1P21 | 0.910 |
| CHR1P22 | 1.000 |
| CHR1P31 | 0.901 |
| CHR1P32 | 0.969 |
| CHR1P33 | 0.965 |
| CHR1P34 | 0.987 |
| CHR1P35 | 1.000 |
| CHR1P36 | 0.994 |
| CHR1Q12 | 0.995 |
| CHR1Q21 | 0.984 |
| CHR1Q22 | 0.957 |
| CHR1Q23 | 1.000 |
| CHR1Q24 | 1.000 |
| CHR1Q25 | 0.951 |
| CHR1Q31 | 0.961 |
| CHR1Q32 | 1.000 |
| CHR1Q41 | 0.947 |
| CHR1Q42 | 1.000 |
| CHR1Q43 | 1.000 |
| CHR1Q44 | 0.931 |
| CHR20P11 | 1.000 |
| CHR20P12 | 0.970 |
| CHR20P13 | 0.945 |
| CHR20Q11 | 1.000 |
| CHR20Q12 | 1.000 |
| CHR20Q13 | 1.000 |
| CHR21Q11 | 1.000 |
| CHR21Q21 | 0.957 |
| CHR21Q22 | 0.006 |
| CHR22Q11 | 0.972 |
| CHR22Q12 | 1.000 |
| CHR22Q13 | 1.000 |
| CHR2P11 | 1.000 |
| CHR2P12 | 0.948 |
| CHR2P13 | 0.827 |
| CHR2P14 | 1.000 |
| CHR2P15 | 0.982 |
| CHR2P16 | 0.967 |
| CHR2P21 | 0.998 |
| CHR2P22 | 0.994 |
| CHR2P23 | 0.877 |
| CHR2P24 | 1.000 |
| CHR2P25 | 1.000 |
| CHR2Q11 | 0.938 |
| CHR2Q12 | 0.784 |
| CHR2Q13 | 0.887 |
| CHR2Q14 | 1.000 |
| CHR2Q21 | 0.938 |
| CHR2Q22 | 1.000 |
| CHR2Q23 | 1.000 |
| CHR2Q24 | 1.000 |
| CHR2Q31 | 0.984 |
| CHR2Q32 | 1.000 |
| CHR2Q33 | 0.952 |
| CHR2Q34 | 1.000 |
| CHR2Q35 | 1.000 |
| CHR2Q36 | 0.958 |
| CHR2Q37 | 0.991 |
| CHR3P14 | 1.000 |
| CHR3P21 | 1.000 |
| CHR3P22 | 0.963 |
| CHR3P24 | 1.000 |
| CHR3P25 | 0.995 |
| CHR3P26 | 1.000 |
| CHR3Q12 | 0.954 |
| CHR3Q13 | 0.930 |
| CHR3Q21 | 0.960 |
| CHR3Q22 | 1.000 |
| CHR3Q23 | 1.000 |
| CHR3Q24 | 0.996 |
| CHR3Q25 | 1.000 |
| CHR3Q26 | 1.000 |
| CHR3Q27 | 0.944 |
| CHR3Q28 | 0.941 |
| CHR3Q29 | 1.000 |
| CHR4P12 | 0.862 |
| CHR4P14 | 1.000 |
| CHR4P15 | 0.982 |
| CHR4P16 | 1.000 |
| CHR4Q12 | 0.751 |
| CHR4Q13 | 0.986 |
| CHR4Q21 | 1.000 |
| CHR4Q22 | 0.991 |
| CHR4Q23 | 0.964 |
| CHR4Q24 | 1.000 |
| CHR4Q25 | 1.000 |
| CHR4Q26 | 1.000 |
| CHR4Q27 | 1.000 |
| CHR4Q28 | 1.000 |
| CHR4Q31 | 0.988 |
| CHR4Q32 | 0.942 |
| CHR4Q34 | 0.789 |
| CHR4Q35 | 0.976 |
| CHR5P12 | 0.956 |
| CHR5P13 | 0.881 |
| CHR5P14 | 0.901 |
| CHR5P15 | 0.965 |
| CHR5Q11 | 1.000 |
| CHR5Q12 | 1.000 |
| CHR5Q13 | 0.976 |
| CHR5Q14 | 1.000 |
| CHR5Q15 | 1.000 |
| CHR5Q21 | 1.000 |
| CHR5Q22 | 1.000 |
| CHR5Q23 | 0.977 |
| CHR5Q31 | 1.000 |
| CHR5Q32 | 0.976 |
| CHR5Q33 | 1.000 |
| CHR5Q34 | 0.984 |
| CHR5Q35 | 0.905 |
| CHR6P12 | 0.927 |
| CHR6P21 | 0.954 |
| CHR6P22 | 1.000 |
| CHR6P23 | 0.994 |
| CHR6P24 | 1.000 |
| CHR6P25 | 0.935 |
| CHR6Q13 | 1.000 |
| CHR6Q14 | 0.984 |
| CHR6Q15 | 1.000 |
| CHR6Q16 | 0.955 |
| CHR6Q21 | 1.000 |
| CHR6Q22 | 0.992 |
| CHR6Q23 | 1.000 |
| CHR6Q24 | 0.980 |
| CHR6Q25 | 1.000 |
| CHR6Q27 | 0.993 |
| CHR7P11 | 1.000 |
| CHR7P12 | 0.996 |
| CHR7P13 | 1.000 |
| CHR7P14 | 1.000 |
| CHR7P15 | 0.980 |
| CHR7P21 | 0.828 |
| CHR7P22 | 0.990 |
| CHR7Q11 | 1.000 |
| CHR7Q21 | 1.000 |
| CHR7Q22 | 1.000 |
| CHR7Q31 | 0.990 |
| CHR7Q32 | 0.978 |
| CHR7Q33 | 0.935 |
| CHR7Q34 | 0.924 |
| CHR7Q35 | 1.000 |
| CHR7Q36 | 1.000 |
| CHR8P11 | 1.000 |
| CHR8P12 | 0.844 |
| CHR8P21 | 0.981 |
| CHR8P22 | 1.000 |
| CHR8P23 | 0.991 |
| CHR8Q11 | 1.000 |
| CHR8Q12 | 0.979 |
| CHR8Q13 | 0.961 |
| CHR8Q21 | 0.997 |
| CHR8Q22 | 1.000 |
| CHR8Q23 | 1.000 |
| CHR8Q24 | 1.000 |
| CHR9P13 | 1.000 |
| CHR9P21 | 0.976 |
| CHR9P22 | 0.982 |
| CHR9P24 | 0.941 |
| CHR9Q21 | 1.000 |
| CHR9Q22 | 1.000 |
| CHR9Q31 | 0.945 |
| CHR9Q32 | 1.000 |
| CHR9Q33 | 1.000 |
| CHR9Q34 | 0.973 |
| CHRXP11 | 1.000 |
| CHRXP21 | 0.934 |
| CHRXP22 | 0.901 |
| CHRXQ13 | 0.950 |
| CHRXQ21 | 0.931 |
| CHRXQ22 | 1.000 |
| CHRXQ23 | 1.000 |
| CHRXQ24 | 1.000 |
| CHRXQ25 | 0.994 |
| CHRXQ26 | 0.844 |
| CHRXQ27 | 0.963 |
| CHRXQ28 | 0.962 |
| CHRYP11 | 1.000 |
| CHRYQ11 | 0.958 |

To identify the most differentially expressed genes from these statistically significant gene sets, we chose the “leading edge subset,” a group of the most-upregulated genes in the gene set (Subramanian et al. (2005)). Specifically, the leading edge subset of a gene set contains the genes that contribute the most to the set's enrichment score (ES), a statistic reflecting the degree to which a gene set is over-represented at the top or bottom of a list of genes ranked by their differential expression.

Hierarchical clustering was performed in R, using complete-linkage hierarchical clustering (the hclust function in the stats package), and heatmaps created via the heatmap.2 function in the gplots package, using the “scale=‘row’” option to z-score normalize the rows.

### Results

Using paired t-tests, two sets of genes were identified as being significantly and consistently differentially expressed between trisomy 21 samples and their euploid controls. One set, the “Individual gene set,” comprised 414 probes (see Table 2) whose individual expression levels were significantly different via paired t-tests (adjusted p-value <0.5) in samples matched for sex and gestational age.

TABLE 2
Individual genes significantly and consistently
differentially expressed in trisomy 21 samples

| | | | Entrez | Chromosomal |
|---|---|---|---|---|
| Probe Set ID | Gene Symbol | Gene Title | Gene ID | Location |
| 1552461_at | FAM46D | family with sequence similarity 46, | 169966 | Xq21.1 |
| | | member D | | |
| 1552531_a_at | NLRP11 | NLR family, pyrin domain containing | 204801 | 19q13.42 |
| | | 11 | | |
| 1552564_at | NUDT9P1 | nudix (nucleoside diphosphate linked | 119369 | 10q23.32 |
| | | moiety X)-type motif 9 pseudogene 1 | | |
| 1552703_s_at | CASP1 /// COP1 | caspase 1, apoptosis-related cysteine | 114769 | 11q23 |
| | | peptidase (interleukin 1, beta, | /// 834 | |
| | | convertase) /// caspase-1 dominant- | | |
| | | negative inhibitor pseudo-ICE | | |
| 1552925_at | PCDH10 | protocadherin 10 | 57575 | 4q28.3 |
| 1553172_at | ZNF777 | zinc finger protein 777 | 27153 | 7q36.1 |
| 1553322_s_at | TEAD1 | TEA domain family member 1 (SV40 | 7003 | 11p15.2 |
| | | transcriptional enhancer factor) | | |
| 1553450_s_at | — | MRNA; cDNA DKFZp434C1427 | — | — |
| | | (from clone DKFZp434C1427) | | |
| 1554188_at | C11orf53 | chromosome 11 open reading frame 53 | 341032 | 11q23.1 |
| 1554345_a_at | GIN1 | gypsy retrotransposon integrase 1 | 54826 | 5q21.1 |
| 1554681_a_at | MGC50722 | hypothetical MGC50722 | 399693 | — |
| 1554690_a_at | TACC1 | transforming, acidic coiled-coil | 6867 | 8p11 |
| | | containing protein 1 | | |
| 1554740_a_at | IPP | intracisternal A particle-promoted | 3652 | 1p34-p32 |
| | | polypeptide | | |
| 1554778_at | PHLDB2 | pleckstrin homology-like domain, | 90102 | 3q13.2 |
| | | family B, member 2 | | |
| 1554821_a_at | ZBED1 | zinc finger, BED-type containing 1 | 9189 | Xp22.33; Yp11 |
| 1554890_a_at | TIA1 | TIA1 cytotoxic granule-associated | 7072 | 2p13 |
| | | RNA binding protein | | |
| 1554957_at | — | — | — | — |
| 1555176_at | — | — | — | — |
| 1555349_a_at | ITGB2 | integrin, beta 2 (complement | 3689 | 21q22.3 |
| | | component 3 receptor 3 and 4 subunit) | | |
| 1555393_s_at | C21orf67 | chromosome 21 open reading frame 67 | 84536 | 21q22.3 |
| 1555404_a_at | DUOXA1 | dual oxidase maturation factor 1 | 90527 | 15q21.1 |
| 1556017_at | NBEAL2 | neurobeachin-like 2 | 23218 | 3p21.31 |
| 1556037_s_at | HHIP | hedgehog interacting protein | 64399 | 4q28-q32 |
| 1556236_at | — | CDNA clone IMAGE: 5265178 | — | — |
| 1556261_a_at | — | CDNA FLJ40252 fis, clone | — | — |
| | | TESTI2024299 | | |
| 1556282_at | FGFR1OP2 | FGFR1 oncogene partner 2 | 26127 | 12p11.23 |
| 1556496_a_at | — | Full length insert cDNA clone | — | — |
| | | ZD79H01 | | |
| 1557248_at | ZNF587 | Zinc finger protein 587 | 84914 | 19q13.43 |
| 1557300_s_at | — | CDNA FLJ34138 fis, clone | — | — |
| | | FCBBF3011003 | | |
| 1557558_s_at | MATN1 | Matrilin 1, cartilage matrix protein | 4146 | 1p35 |
| 1557787_at | — | CDNA clone IMAGE: 4839194 | — | — |
| 1558000_at | ARID5B | AT rich interactive domain 5B | 84159 | 10q21.2 |
| | | (MRF1-like) | | |
| 1558166_at | MGC16275 | hypothetical protein MGC16275 | 85001 | 17q25.2 |
| 1558185_at | CLLU1 | chronic lymphocytic leukemia up- | 574028 | 12q22 |
| | | regulated 1 | | |
| 1558208_at | — | Full length insert cDNA clone | — | — |
| | | ZC66E08 | | |
| 1559611_at | TMEM75 | transmembrane protein 75 | 641384 | 8q24.21 |
| 1559633_a_at | CHRM3 /// | cholinergic receptor, muscarinic 3 /// | 1131 /// | 1q43 |
| | LOC730413 | similar to cholinergic receptor, | 730413 | |
| | | muscarinic 3 | | |
| 1559702_at | — | Clone IMAGE: 120631 mRNA | — | — |
| | | sequence | | |
| 1560431_at | PGM5P1 | phosphoglucomutase 5 pseudogene 1 | 653394 | 9q12 |
| 1560587_s_at | PRDX5 | peroxiredoxin 5 | 25824 | 11q13 |
| 1560630_at | — | CDNA clone IMAGE: 4838137 | — | — |
| 1560752_at | FBXW2 | F-box and WD repeat domain | 26190 | 9q34 |
| | | containing 2 | | |
| 1560997_at | — | Full length insert cDNA clone | — | — |
| | | YW24A03 | | |
| 1561185_at | CLONE795723 | hypothetical transcript 795723 | 64592 | Yp11.2 |
| 1561240_at | — | MRNA; cDNA DKFZp434C122 (from | — | — |
| | | clone DKFZp434C122) | | |
| 1561365_at | NRP1 | Neuropilin 1 | 8829 | 10p12 |
| 1562153_a_at | PVT1 | Pvt1 oncogene homolog, MYC | 5820 | 8q24 |
| | | activator (mouse) | | |
| 1562633_at | RMST | rhabdomyosarcoma 2 associated | 196475 | 12q23.1\|12q21 |
| | | transcript (non-protein coding) | | |
| 1563283_at | — | CDNA clone IMAGE: 4828909 | — | — |
| 1563620_at | BTRC | beta-transducin repeat containing | 8945 | 10q24.32 |
| 1563845_at | LOC202134 | hypothetical protein LOC202134 | 202134 | 5q35.2 |
| 1565537_at | NKX1-1 | NK1 homeobox 1 | 54729 | 4p16.3 |
| 1565621_at | CTGLF2 | Centaurin, gamma-like family, | 729092 | 10q22.2 |
| | | member 2 | | |
| 1565628_at | — | Full length insert cDNA clone | — | — |
| | | ZD55G10 | | |
| 1565735_at | — | CDNA FLJ39816 fis, clone | — | — |
| | | SPLEN2010119 | | |
| 1565748_at | — | CDNA FLJ32177 fis, clone | — | — |
| | | PLACE6001294 | | |
| 1566163_at | — | MRNA; cDNA DKFZp686L0519 | — | — |
| | | (from clone DKFZp686L0519) | | |
| 1566168_at | LOC729986 /// | hypothetical protein LOC729986 /// | 729986 | 7q36.1 |
| | LOC730340 | hypothetical protein LOC730340 | /// | |
| | | | 730340 | |
| 1566987_s_at | LOC729074 | Hypothetical protein LOC729074 | 729074 | — |
| 1568687_s_at | LOC158381 | ATPase, Class I, type 8B family | 158381 | 9p13.3 |
| | | pseudogene | | |
| 1568949_at | LOC729822 | similar to phosphatidylinositol transfer | 729822 | 17q24.2 |
| | | protein, cytoplasmic 1 | | |
| 1569025_s_at | FAM13A1 | family with sequence similarity 13, | 10144 | 4q22.1 |
| | | member A1 | | |
| 1569376_s_at | — | CDNA clone IMAGE: 4297546 | — | — |
| 1569794_at | — | CDNA clone IMAGE: 4824066 | — | — |
| 1569826_at | — | CDNA clone IMAGE: 4822782 | — | — |
| 1569955_at | — | Homo sapiens, clone | — | — |
| | | IMAGE: 4097490, mRNA | | |
| 1570064_at | — | CDNA clone IMAGE: 5266039 | — | — |
| 1570093_at | — | Clone pp8142 unknown mRNA | — | — |
| 1570102_at | — | Homo sapiens, clone | — | — |
| | | IMAGE: 5208312, mRNA | | |
| 1570505_at | ABCB4 | ATP-binding cassette, sub-family B | 5244 | 7q21.1 |
| | | (MDR/TAP), member 4 | | |
| 200710_at | ACADVL | acyl-Coenzyme A dehydrogenase, | 37 | 17p13-p11 |
| | | very long chain | | |
| 200783_s_at | STMN1 | stathmin 1/oncoprotein 18 | 3925 | 1p36.1-p35 |
| 200838_at | CTSB | cathepsin B | 1508 | 8p22 |
| 200956_s_at | SSRP1 | structure specific recognition protein 1 | 6749 | 11q12 |
| 200991_s_at | SNX17 | sorting nexin 17 | 9784 | 2p23-p22 |
| 201079_at | SYNGR2 | synaptogyrin 2 | 9144 | 17q25.3 |
| 201819_at | SCARB1 | scavenger receptor class B, member 1 | 949 | 12q24.31 |
| 201959_s_at | MYCBP2 | MYC binding protein 2 | 23077 | 13q22 |
| 202332_at | CSNK1E | casein kinase 1, epsilon | 1454 | 22q13.1 |
| 202415_s_at | HSPBP1 | hsp70-interacting protein | 23640 | 19q13.42 |
| 202510_s_at | TNFAIP2 | tumor necrosis factor, alpha-induced | 7127 | 14q32 |
| | | protein 2 | | |
| 202638_s_at | ICAM1 | intercellular adhesion molecule 1 | 3383 | 19p13.3-p13.2 |
| | | (CD54), human rhinovirus receptor | | |
| 202695_s_at | STK17A | serine/threonine kinase 17a | 9263 | 7p12-p14 |
| 202898_at | SDC3 | syndecan 3 | 9672 | 1pter-p22.3 |
| 202931_x_at | BIN1 | bridging integrator 1 | 274 | 2q14 |
| 203022_at | RNASEH2A | ribonuclease H2, subunit A | 10535 | 19p13.13 |
| 203045_at | NINJ1 | ninjurin 1 | 4814 | 9q22 |
| 203099_s_at | CDYL | chromodomain protein, Y-like | 9425 | 6p25.1 |
| 203164_at | SLC33A1 | solute carrier family 33 (acetyl-CoA | 9197 | 3q25.31 |
| | | transporter), member 1 | | |
| 203167_at | TIMP2 | TIMP metallopeptidase inhibitor 2 | 7077 | 17q25 |
| 203799_at | CD302 | CD302 molecule | 9936 | 2q24.2 |
| 204014_at | DUSP4 | dual specificity phosphatase 4 | 1846 | 8p12-p11 |
| 204029_at | CELSR2 | cadherin, EGF LAG seven-pass G-type | 1952 | 1p21 |
| | | receptor 2 (flamingo homolog, | | |
| | | Drosophila) | | |
| 204118_at | CD48 | CD48 molecule | 962 | 1q21.3-q22 |
| 204177_s_at | KLHL20 | kelch-like 20 (Drosophila) | 27252 | 1q24.1-q24.3 |
| 204182_s_at | ZBTB43 | zinc finger and BTB domain | 23099 | 9q33-q34 |
| | | containing 43 | | |
| 204300_at | PET112L | PET112-like (yeast) | 5188 | 4q27-q28 |
| 204385_at | KYNU | kynureninase (L-kynurenine | 8942 | 2q22.2 |
| | | hydrolase) | | |
| 204708_at | MAPK4 | mitogen-activated protein kinase 4 | 5596 | 18q12-q21 |
| 204913_s_at | SOX11 | SRY (sex determining region Y)-box | 6664 | 2p25 |
| | | 11 | | |
| 205075_at | SERPINF2 | serpin peptidase inhibitor, clade F | 5345 | 17p13 |
| | | (alpha-2 antiplasmin, pigment | | |
| | | epithelium derived factor), member 2 | | |
| 205380_at | PDZK1 | PDZ domain containing 1 | 5174 | 1q21 |
| 205388_at | TNNC2 | troponin C type 2 (fast) | 7125 | 20q12-q13.11 |
| 205527_s_at | GEMIN4 | gem (nuclear organelle) associated | 50628 | 17p13 |
| | | protein 4 | | |
| 205655_at | MDM4 | Mdm4, transformed 3T3 cell double | 4194 | 1q32 |
| | | minute 4, p53 binding protein (mouse) | | |
| 205665_at | TSPAN9 | tetraspanin 9 | 10867 | 12p13.33-p13.32 |
| 205774_at | F12 | coagulation factor XII (Hageman | 2161 | 5q33-qter |
| | | factor) | | |
| 205797_s_at | TCP11L1 | t-complex 11 (mouse)-like 1 | 55346 | 11p13 |
| 205885_s_at | ITGA4 | integrin, alpha 4 (antigen CD49D, | 3676 | 2q31.3 |
| | | alpha 4 subunit of VLA-4 receptor) | | |
| 205900_at | KRT1 | keratin 1 (epidermolytic | 3848 | 12q12-q13 |
| | | hyperkeratosis) | | |
| 205938_at | PPM1E | protein phosphatase 1E (PP2C domain | 22843 | 17q22 |
| | | containing) | | |
| 206035_at | REL | v-rel reticuloendotheliosis viral | 5966 | 2p13-p12 |
| | | oncogene homolog (avian) | | |
| 206074_s_at | HMGA1 | high mobility group AT-hook 1 | 3159 | 6p21 |
| 206153_at | CYP4F11 | cytochrome P450, family 4, subfamily | 57834 | 19p13.1 |
| | | F, polypeptide 11 | | |
| 206180_x_at | ZNF747 | zinc finger protein 747 | 65988 | 16p11.2 |
| 206190_at | GPR17 | G protein-coupled receptor 17 | 2840 | 2q21 |
| 206213_at | WNT10B | wingless-type MMTV integration site | 7480 | 12q13 |
| | | family, member 10B | | |
| 206286_s_at | TDGF1 /// TDGF3 | teratocarcinoma-derived growth factor | 6997 /// | 3p21.31 /// |
| | | 1 /// teratocarcinoma-derived growth | 6998 | Xq22.3 |
| | | factor 3, pseudogene | | |
| 206413_s_at | TCL1B | T-cell leukemia/lymphoma 1B | 9623 | 14q32.1 |
| 206426_at | MLANA | melan-A | 2315 | 9p24.1 |
| 206563_s_at | OPRL1 | opiate receptor-like 1 | 4987 | 20q13.33 |
| 206765_at | KCNJ2 | potassium inwardly-rectifying channel, | 3759 | 17q23.1-q24.2 |
| | | subfamily J, member 2 | | |
| 206775_at | CUBN | cubilin (intrinsic factor-cobalamin | 8029 | 10p12.31 |
| | | receptor) | | |
| 206972_s_at | GPR161 | G protein-coupled receptor 161 | 23432 | 1q24.2 |
| 206973_at | PPFIA2 | protein tyrosine phosphatase, receptor | 8499 | 12q21.31 |
| | | type, f polypeptide (PTPRF), | | |
| | | interacting protein (liprin), alpha 2 | | |
| 207253_s_at | UBN1 | ubinuclein 1 | 29855 | 16p13.3 |
| 207374_at | PLSCR2 | phospholipid scramblase 2 | 57047 | 3q24 |
| 207566_at | MR1 | major histocompatibility complex, | 3140 | 1q25.3 |
| | | class I-related | | |
| 207934_at | RFPL1 | ret finger protein-like 1 | 5988 | 22q12.2 |
| 207968_s_at | MEF2C | myocyte enhancer factor 2C | 4208 | 5q14 |
| 207976_at | KLHL18 | kelch-like 18 (Drosophila) | 23276 | 3p21.31 |
| 208039_at | — | — | — | — |
| 208255_s_at | FKBP8 | FK506 binding protein 8, 38 kDa | 23770 | 19p12 |
| 208334_at | NDST4 | N-deacetylase/N-sulfotransferase | 64579 | 4q25-q26 |
| | | (heparan glucosaminyl) 4 | | |
| 208357_x_at | CSH1 | chorionic somatomammotropin | 1442 | 17q24.2 |
| | | hormone 1 (placental lactogen) | | |
| 208389_s_at | SLC1A2 | solute carrier family 1 (glial high | 6506 | 11p13-p12 |
| | | affinity glutamate transporter), | | |
| | | member 2 | | |
| 208476_s_at | FRMD4A | FERM domain containing 4A | 55691 | 10p13 |
| 208570_at | WNT1 | wingless-type MMTV integration site | 7471 | 12q13 |
| | | family, member 1 | | |
| 208688_x_at | EIF3B | eukaryotic translation initiation factor | 8662 | 7p22.2 |
| | | 3, subunit B | | |
| 208698_s_at | NONO | non-POU domain containing, octamer- | 4841 | Xq13.1 |
| | | binding | | |
| 208710_s_at | AP3D1 | adaptor-related protein complex 3, | 8943 | 19p13.3 |
| | | delta 1 subunit | | |
| 208884_s_at | LOC730429 /// | ubiquitin protein ligase E3 component | 51366 /// | 8q22 |
| | UBR5 | n-recognin 5 /// similar to E3 ubiquitin | 730429 | |
| | | protein ligase, HECT domain | | |
| | | containing, 1 | | |
| 209163_at | CYB561 | cytochrome b-561 | 1534 | 17q11-qter |
| 209574_s_at | C18orf1 | chromosome 18 open reading frame 1 | 753 | 18p11.2 |
| 209960_at | HGF | hepatocyte growth factor (hepapoietin | 3082 | 7q21.1 |
| | | A; scatter factor) | | |
| 210034_s_at | LOC388907 /// | ribosomal protein L5 /// similar to | 388907 | 1p22.1 /// |
| | LOC642146 /// | ribosomal protein L5 | /// 6125 | 22q13.2 |
| | LOC647436 /// | | /// | |
| | RPL5 | | 642146 | |
| | | | /// | |
| | | | 647436 | |
| 210156_s_at | PCMT1 | protein-L-isoaspartate (D-aspartate) O- | 5110 | 6q24-q25 |
| | | methyltransferase | | |
| 210297_s_at | MSMB | microseminoprotein, beta- | 4477 | 10q11.2 |
| 210330_at | SGCD | sarcoglycan, delta (35 kDa dystrophin- | 6444 | 5q33-q34 |
| | | associated glycoprotein) | | |
| 210644_s_at | LAIR1 | leukocyte-associated immunoglobulin- | 3903 | 19q13.4 |
| | | like receptor 1 | | |
| 210981_s_at | GRK6 | G protein-coupled receptor kinase 6 | 2870 | 5q35 |
| 211016_x_at | HSPA4 | heat shock 70 kDa protein 4 | 3308 | 5q31.1-q31.2 |
| 211064_at | ZNF493 | zinc finger protein 493 | 284443 | 19p12 |
| 211180_x_at | RUNX1 | runt-related transcription factor 1 | 861 | 21q22.3 |
| | | (acute myeloid leukemia 1; aml1 | | |
| | | oncogene) | | |
| 211194_s_at | TP63 | tumor protein p63 | 8626 | 3q28 |
| 211208_s_at | CASK | calcium/calmodulin-dependent serine | 8573 | Xp11.4 |
| | | protein kinase (MAGUK family) | | |
| 211212_s_at | ORC5L | origin recognition complex, subunit 5- | 5001 | 7q22.1 |
| | | like (yeast) | | |
| 211422_at | TRPM3 | transient receptor potential cation | 80036 | 9q21.11-q21.12 |
| | | channel, subfamily M, member 3 | | |
| 211571_s_at | VCAN | versican | 1462 | 5q14.3 |
| 211740_at | ICA1 | islet cell autoantigen 1, 69 kDa | 3382 | 7p22 |
| 211771_s_at | POU2F2 | POU class 2 homeobox 2 | 5452 | 19q13.2 |
| 211925_s_at | PLCB1 | phospholipase C, beta 1 | 23236 | 20p12 |
| | | (phosphoinositide-specific) | | |
| 212057_at | KIAA0182 | KIAA0182 | 23199 | 16q24.1 |
| 212684_at | ZNF3 | zinc finger protein 3 | 7551 | 7q22.1 |
| 212793_at | DAAM2 | dishevelled associated activator of | 23500 | 6p21.2 |
| | | morphogenesis 2 | | |
| 213038_at | RNF19B | ring finger protein 19B | 127544 | 1p35.1 |
| 213250_at | CCDC85B | Coiled-coil domain containing 85B | 11007 | 11q12.1 |
| 213361_at | TDRD7 | tudor domain containing 7 | 23424 | 9q22.33 |
| 213442_x_at | SPDEF | SAM pointed domain containing ets | 25803 | 6p21.3 |
| | | transcription factor | | |
| 213444_at | LOC643641 | hypothetical protein LOC643641 | 643641 | 7q36.1 |
| 213753_x_at | EIF5A | eukaryotic translation initiation factor | 1984 | 17p13-p12 |
| | | 5A | | |
| 213835_x_at | GTPBP3 | GTP binding protein 3 (mitochondrial) | 84705 | 19p13.11 |
| 213893_x_at | LOC441259 /// | postmeiotic segregation increased 2- | 441259 | 7q11-q22 /// |
| | LOC729299 /// | like 5 /// PMS2 postmeiotic | /// 5383 | 7q11.23 |
| | LOC729453 /// | segregation increased 2 (S. cerevisiae)- | /// | |
| | LOC730313 /// | like /// similar to postmeiotic | 729299 | |
| | LOC730324 /// | segregation increased 2-like 2 | /// | |
| | PMS2L5 | | 729453 | |
| | | | /// | |
| | | | 730313 | |
| | | | /// | |
| | | | 730324 | |
| 213949_s_at | DOHH | Deoxyhypusine | 83475 | 19p13.3 |
| | | hydroxylase/monooxygenase | | |
| 214013_s_at | TBC1D1 | TBC1 (tre-2/USP6, BUB2, cdc16) | 23216 | 4p14 |
| | | domain family, member 1 | | |
| 214420_s_at | CYP2C9 | Cytochrome P450, family 2, subfamily | 1559 | 10q24 |
| | | C, polypeptide 9 | | |
| 214595_at | KCNG1 | potassium voltage-gated channel, | 3755 | 20q13 |
| | | subfamily G, member 1 | | |
| 214752_x_at | FLNA | filamin A, alpha (actin binding protein | 2316 | Xq28 |
| | | 280) | | |
| 214883_at | THRA | thyroid hormone receptor, alpha | 7067 | 17q11.2 |
| | | (erythroblastic leukemia viral (v-erb-a) | | |
| | | oncogene homolog, avian) | | |
| 214914_at | FAM13C1 | family with sequence similarity 13, | 220965 | 10q21.1 |
| | | member C1 | | |
| 214915_at | ZNF362 | Zinc finger protein 362 | 149076 | 1p35.1 |
| 214955_at | TMPRSS6 | transmembrane protease, serine 6 | 164656 | 22q12.3 |
| 215112_x_at | MCF2L2 | MCF.2 cell line derived transforming | 23101 | 3q27.1 |
| | | sequence-like 2 | | |
| 215319_at | ATP8B3 | ATPase, class I, type 8B, member 3 | 148229 | 19p13.3 |
| 215673_at | KIAA1655 | KIAA1655 protein | 85370 | — |
| 215766_at | GSTA1 | Glutathione S-transferase A1 | 2938 | 6p12.1 |
| 216007_at | — | Clone 24457 mRNA sequence | — | — |
| 216242_x_at | POLR2J2 /// | polymerase (RNA) II (DNA directed) | 246721 | 7p13 /// 7q22.1 |
| | POLR2J3 /// | polypeptide J, 13.3 kDa pseudogene /// | /// | |
| | POLR2J4 | DNA directed RNA polymerase II | 548644 | |
| | | polypeptide J-related /// RPB11b2 | /// 84820 | |
| | | protein | | |
| 216251_s_at | TTLL12 | tubulin tyrosine ligase-like family, | 23170 | 22q13.31 |
| | | member 12 | | |
| 216406_at | — | — | — | — |
| 216669_at | — | — | — | — |
| 216723_at | — | MRNA; cDNA DKFZp434D179 (from | — | — |
| | | clone DKFZp434D179) | | |
| 216889_s_at | HNF4A | hepatocyte nuclear factor 4, alpha | 3172 | 20q12-q13.1 |
| 217326_x_at | IL23A | Interleukin 23, alpha subunit p19 | 51561 | 12q13.2 |
| 217333_at | KRT18P44 | keratin 18 pseudogene 44 | 139748 | Xq25 |
| 217340_at | LOC645452 | similar to 60S ribosomal protein L21 | 645452 | 6p22.2 |
| 217415_at | POLR2A | polymerase (RNA) II (DNA directed) | 5430 | 17p13.1 |
| | | polypeptide A, 220 kDa | | |
| 217621_at | — | — | — | — |
| 218070_s_at | GMPPA | GDP-mannose pyrophosphorylase A | 29926 | 2q35 |
| 218416_s_at | FLJ20489 | hypothetical protein FLJ20489 | 55652 | 12q13.11 |
| 218429_s_at | FLJ11286 | hypothetical protein FLJ11286 | 55337 | 19p13.2 |
| 218475_at | HTF9C | HpaII tiny fragments locus 9C | 27037 | 22q11.1-22q13\| |
| | | | | 22q11.21 |
| 218493_at | C16orf33 | chromosome 16 open reading frame 33 | 79622 | 16p13.3 |
| 218557_at | NIT2 | nitrilase family, member 2 | 56954 | 3q12.2 |
| 218585_s_at | DTL | denticleless homolog (Drosophila) | 51514 | 1q32.1-q32.2 |
| 218714_at | PRR14 | proline rich 14 | 78994 | 16p11.2 |
| 218744_s_at | PACSIN3 | protein kinase C and casein kinase | 29763 | 11p12-p11.12 |
| | | substrate in neurons 3 | | |
| 218831_s_at | FCGRT | Fc fragment of IgG, receptor, | 2217 | 19q13.3 |
| | | transporter, alpha | | |
| 218980_at | FHOD3 | formin homology 2 domain containing 3 | 80206 | 18q12 |
| 219419_at | C18orf22 | chromosome 18 open reading frame 22 | 79863 | 18q23 |
| 219568_x_at | SOX18 | SRY (sex determining region Y)-box | 54345 | 20q13.33 |
| | | 18 | | |
| 219650_at | ERCC6L | excision repair cross-complementing | 54821 | Xq13.1 |
| | | rodent repair deficiency, | | |
| | | complementation group 6-like | | |
| 219683_at | FZD3 | frizzled homolog 3 (Drosophila) | 7976 | 8p21 |
| 219919_s_at | SSH3 | slingshot homolog 3 (Drosophila) | 54961 | 11q13.1 |
| 219960_s_at | UCHL5 | ubiquitin carboxyl-terminal hydrolase | 51377 | 1q32 |
| | | L5 | | |
| 220120_s_at | EPB41L4A | erythrocyte membrane protein band | 64097 | 5q22.2 |
| | | 4.1 like 4A | | |
| 220129_at | SOHLH2 | spermatogenesis and oogenesis | 54937 | 13q13.3 |
| | | specific basic helix-loop-helix 2 | | |
| 220233_at | FBXO17 | F-box protein 17 | 115290 | 19q13.2 |
| 220364_at | FLJ11235 | hypothetical protein FLJ11235 | 54508 | 5q22.2 |
| 220434_at | ADCK4 | aarF domain containing kinase 4 | 79934 | 19q13.2 |
| 221066_at | RXFP3 | relaxin/insulin-like family peptide | 51289 | 5p15.1-p14 |
| | | receptor 3 | | |
| 221114_at | AMBN | ameloblastin (enamel matrix protein) | 258 | 4q21 |
| 221517_s_at | MED17 | mediator complex subunit 17 | 9440 | 11q14 |
| 221667_s_at | HSPB8 | heat shock 22 kDa protein 8 | 26353 | 12q24.23 |
| 221766_s_at | FAM46A | family with sequence similarity 46, | 55603 | 6q14 |
| | | member A | | |
| 221920_s_at | SLC25A37 | solute carrier family 25, member 37 | 51312 | 8p21.2 |
| 222307_at | LOC282997 | hypothetical protein LOC282997 | 282997 | 10q25.3 |
| 222389_s_at | WAC | WW domain containing adaptor with | 51322 | — |
| | | coiled-coil | | |
| 222556_at | ALG5 | asparagine-linked glycosylation 5 | 29880 | 13q13.3 |
| | | homolog (S. cerevisiae, dolichyl- | | |
| | | phosphate beta-glucosyltransferase) | | |
| 222603_at | ERMP1 | endoplasmic reticulum | 79956 | 9p24 |
| | | metallopeptidase 1 | | |
| 222756_s_at | ARRB1 | arrestin, beta 1 | 408 | 11q13 |
| 222826_at | PLDN | pallidin homolog (mouse) | 26258 | 15q21.1 |
| 223103_at | STARD10 | StAR-related lipid transfer (START) | 10809 | 11q13 |
| | | domain containing 10 | | |
| 223122_s_at | SFRP2 | secreted frizzled-related protein 2 | 6423 | 4q31.3 |
| 223296_at | SLC25A33 | solute carrier family 25, member 33 | 84275 | 1p36.22 |
| 223357_s_at | MTIF3 | mitochondrial translational initiation | 219402 | 13q12.2 |
| | | factor 3 | | |
| 223523_at | TMEM108 | transmembrane protein 108 | 66000 | 3q21 |
| 223709_s_at | WNT10A | wingless-type MMTV integration site | 80326 | 2q35 |
| | | family, member 10A | | |
| 223792_at | ZNF2 | zinc finger protein 2 | 7549 | 2q11.2 |
| 223906_s_at | TEX101 | testis expressed 101 | 83639 | 19q13.31 |
| 224108_at | — | — | — | — |
| 224122_at | — | — | — | — |
| 224330_s_at | MRPL27 | mitochondrial ribosomal protein L27 | 51264 | 17q21.3-q22 |
| 224683_at | FBXO18 | F-box protein, helicase, 18 | 84893 | 10p15.1 |
| 224881_at | VKORC1L1 | vitamin K epoxide reductase complex, | 154807 | 7q11.21 |
| | | subunit 1-like 1 | | |
| 225215_s_at | MTRF1L | mitochondrial translational release | 54516 | 6q25-q26 |
| | | factor 1-like | | |
| 225484_at | TSGA14 | testis specific, 14 | 95681 | 7q32 |
| 225830_at | PDZD8 | PDZ domain containing 8 | 118987 | 10q25.3-q26.11 |
| 225858_s_at | BIRC4 | baculoviral IAP repeat-containing 4 | 331 | Xq25 |
| 225939_at | EIF4E3 | eukaryotic translation initiation factor | 317649 | 3p14 |
| | | 4E family member 3 | | |
| 226013_at | TRAK1 | trafficking protein, kinesin binding 1 | 22906 | 3p25.3-p24.1 |
| 226114_at | ZNF436 | zinc finger protein 436 | 80818 | 1p36 |
| 226125_at | — | CDNA clone IMAGE: 4346813 | — | — |
| 226200_at | VARS2 | valyl-tRNA synthetase 2, | 57176 | — |
| | | mitochondrial (putative) | | |
| 226295_at | ITFG2 | integrin alpha FG-GAP repeat | 55846 | 12p13.33 |
| | | containing 2 | | |
| 226410_at | C16orf84 | chromosome 16 open reading frame 84 | 348180 | 16q24.3 |
| 226623_at | PHYHIPL | phytanoyl-CoA 2-hydroxylase | 84457 | 10q11 |
| | | interacting protein-like | | |
| 226986_at | WIPI2 | WD repeat domain, phosphoinositide | 26100 | 7p22.1 |
| | | interacting 2 | | |
| 226995_at | C21orf86 | Chromosome 21 open reading frame | 257103 | 21q22.3 |
| | | 86 | | |
| 227420_at | TNFAIP8L1 | tumor necrosis factor, alpha-induced | 126282 | 19p13.3 |
| | | protein 8-like 1 | | |
| 227449_at | EPHA4 | EPH receptor A4 | 2043 | 2q36.1 |
| 227694_at | C1orf201 | chromosome 1 open reading frame 201 | 90529 | 1p36.11 |
| 227928_at | C12orf48 | chromosome 12 open reading frame 48 | 55010 | 12q23.2 |
| 228375_at | IGSF11 | immunoglobulin superfamily, member | 152404 | 3q13.32 |
| | | 11 | | |
| 228403_at | C9orf165 | chromosome 9 open reading frame 165 | 375704 | 9p13.3 |
| 228712_at | WNK1 | WNK lysine deficient protein kinase 1 | 65125 | 12p13.3 |
| 228831_s_at | GNG7 | guanine nucleotide binding protein (G | 2788 | 19p13.3 |
| | | protein), gamma 7 | | |
| 229035_s_at | KLHDC4 | kelch domain containing 4 | 54758 | 16q24.3 |
| 229131_at | — | — | — | — |
| 229164_s_at | ABTB1 | ankyrin repeat and BTB (POZ) domain | 80325 | 3q21 |
| | | containing 1 | | |
| 229402_at | SAMD13 | sterile alpha motif domain containing | 148418 | 1p31.1 |
| | | 13 | | |
| 229408_at | HDAC5 | histone deacetylase 5 | 10014 | 17q21 |
| 229438_at | FAM20C | Family with sequence similarity 20, | 56975 | 7p22.3 |
| | | member C | | |
| 229444_at | LOC729776 | hypothetical protein LOC729776 | 729776 | 6p21.31 |
| 230009_at | FAM118B | family with sequence similarity 118, | 79607 | 11q24.2 |
| | | member B | | |
| 230043_at | MUC20 | mucin 20, cell surface associated | 200958 | 3q29 |
| 230239_at | ROCK1 | Rho-associated, coiled-coil containing | 6093 | 18q11.1 |
| | | protein kinase 1 | | |
| 230447_at | — | CDNA FLJ30539 fis, clone | — | — |
| | | BRAWH2001255 | | |
| 230492_s_at | RP5-1022P6.2 | hypothetical protein KIAA1434 | 56261 | 20p12.3 |
| 230571_at | — | Transcribed locus | — | — |
| 230917_at | — | CDNA FLJ45450 fis, clone | — | — |
| | | BRSTN2002691 | | |
| 230966_at | IL4I1 | interleukin 4 induced 1 | 259307 | 19q13.3-q13.4 |
| 230988_at | — | Transcribed locus | — | — |
| 231147_at | — | Transcribed locus | — | — |
| 231337_at | LOC730124 | hypothetical protein LOC730124 | 730124 | 2q22.3 |
| 231338_at | C15orf55 | chromosome 15 open reading frame 55 | 256646 | 15q14 |
| 231548_at | FOXO3 | Forkhead box O3 | 2309 | 6q21 |
| 231643_s_at | — | Transcribed locus | — | — |
| 231713_s_at | ELP2 | elongation protein 2 homolog (S. cerevisiae) | 55250 | 18q12.2 |
| 231954_at | DKFZP434I0714 | hypothetical protein DKFZP434I0714 | 54553 | 4q31.23 |
| 232063_x_at | FARSB | phenylalanyl-tRNA synthetase, beta | 10056 | 2q36.1 |
| | | subunit | | |
| 232187_at | PALMD | palmdelphin | 54873 | 1p22-p21 |
| 232213_at | PELI1 | Pellino homolog 1 (Drosophila) | 57162 | 2p13.3 |
| 232243_at | MCPH1 | microcephalin 1 | 79648 | 8p23.1 |
| 232326_at | C8orf56 | chromosome 8 open reading frame 56 | 157556 | 8q22.3 |
| 232422_at | RP11-151A6.2 | hypothetical protein BC004360 | 87769 | 13q32.3 |
| 232725_s_at | MS4A6A | membrane-spanning 4-domains, | 64231 | 11q12.1 |
| | | subfamily A, member 6A | | |
| 232772_at | LOC221272 | hypothetical protein LOC221272 | 221272 | 6q22.1 |
| 232802_at | SYT8 | synaptotagmin VIII | 90019 | 11p15.5 |
| 232833_at | — | Clone 24425 mRNA sequence | — | — |
| 232841_at | — | CDNA: FLJ23097 fis, clone | — | — |
| | | LNG07418 | | |
| 232857_at | POLR3H | polymerase (RNA) III (DNA directed) | 171568 | 22q13.2 |
| | | polypeptide H (22.9 kD) | | |
| 233105_at | — | CDNA: FLJ22627 fis, clone HSI06152 | — | — |
| 233318_at | — | — | — | — |
| 233351_at | — | Clone IMAGE: 1542282, mRNA | — | — |
| | | sequence | | |
| 233404_at | — | CDNA FLJ13756 fis, clone | — | — |
| | | PLACE3000365 | | |
| 233467_s_at | TSPAN32 | tetraspanin 32 | 10077 | 11p15.5 |
| 233543_s_at | CCDC98 | coiled-coil domain containing 98 | 84142 | 4q21.21-q21.23 |
| 233622_x_at | — | MRNA; cDNA DKFZp761A219 (from | — | — |
| | | clone DKFZp761A219) | | |
| 233722_at | — | Clone FLB3107 | — | — |
| 233754_x_at | ZNF71 | zinc finger protein 71 | 58491 | 19q13.4 |
| 233824_at | — | CDNA: FLJ21428 fis, clone | — | — |
| | | COL04203 | | |
| 233937_at | GGNBP2 | gametogenetin binding protein 2 | 79893 | 17q12 |
| 234067_at | — | CDNA FLJ10214 fis, clone | — | — |
| | | HEMBA1006530 | | |
| 234251_at | — | — | — | — |
| 234322_at | — | CDNA: FLJ21248 fis, clone | — | — |
| | | COL01235 | | |
| 234472_at | GALNT13 | UDP-N-acetyl-alpha-D- | 114805 | 2q23.3-q24.1 |
| | | galactosamine:polypeptide N- | | |
| | | acetylgalactosaminyltransferase 13 | | |
| | | (GalNAc-T13) | | |
| 234480_at | DKFZP761C1711 | Hypothetical protein | 57796 | — |
| | | DKFZp761C1711 | | |
| 234481_at | — | — | — | — |
| 234544_at | ARHGEF12 | Rho guanine nucleotide exchange | 23365 | 11q23.3 |
| | | factor (GEF) 12 | | |
| 234592_at | — | CDNA: FLJ23060 fis, clone | — | — |
| | | LNG04601 | | |
| 234701_at | ANKRD11 | ankyrin repeat domain 11 | 29123 | 16q24.3 |
| 234741_at | ATP2B2 | ATPase, Ca++ transporting, plasma | 491 | 3p25.3 |
| | | membrane 2 | | |
| 234827_at | — | MRNA; cDNA DKFZp564M0463 | — | — |
| | | (from clone DKFZp564M0463) | | |
| 234898_at | — | — | — | — |
| 235004_at | RBM24 | RNA binding motif protein 24 | 221662 | 6p22.3 |
| 235017_s_at | — | MRNA; cDNA DKFZp564E143 (from | — | — |
| | | clone DKFZp564E143) | | |
| 235098_at | PEX26 | peroxisome biogenesis factor 26 | 55670 | 22q11.21 |
| 235123_at | — | Transcribed locus | — | — |
| 235343_at | — | — | — | — |
| 235473_at | MED6 | Mediator complex subunit 6 | 10001 | 14q24.2 |
| 235596_at | — | Transcribed locus | — | — |
| 235788_at | — | Transcribed locus | — | — |
| 235792_x_at | PIK3C2A | phosphoinositide-3-kinase, class 2, | 5286 | 11p15.5-p14 |
| | | alpha polypeptide | | |
| 235815_at | TSHZ2 | teashirt zinc finger homeobox 2 | 128553 | 20q13.2 |
| 235885_at | P2RY12 | purinergic receptor P2Y, G-protein | 64805 | 3q24-q25 |
| | | coupled, 12 | | |
| 235955_at | MARVELD2 | MARVEL domain containing 2 | 153562 | 5q13.2 |
| 236098_at | — | CDNA clone IMAGE: 4557810 | — | — |
| 236185_at | NHLRC2 | NHL repeat containing 2 | 374354 | 10q25.3 |
| 236340_at | — | Transcribed locus, strongly similar to | — | — |
| | | XP_940794.1 PREDICTED: | | |
| | | hypothetical protein XP_940794 | | |
| | | [Homo sapiens] | | |
| 236388_at | — | ELISC-1 | — | — |
| 236613_at | RBM25 | RNA binding motif protein 25 | 58517 | 14q24.3 |
| 236713_at | ACTR1A | ARP1 actin-related protein 1 homolog | 10121 | 10q24.32 |
| | | A, centractin alpha (yeast) | | |
| 236766_at | — | Transcribed locus | — | — |
| 236805_at | C9orf96 | chromosome 9 open reading frame 96 | 169436 | 9q34.2 |
| 236834_at | SCFD2 | sec1 family domain containing 2 | 152579 | 4q12 |
| 236869_at | — | Transcribed locus | — | — |
| 236905_at | LOC731139 | hypothetical protein LOC731139 | 731139 | — |
| 237093_at | — | — | — | — |
| 237182_at | LOC653479 | Similar to mitochondrial ribosomal | 653479 | 17q21.32 |
| | | protein L45 | | |
| 237191_x_at | — | Transcribed locus | — | — |
| 237311_at | — | Transcribed locus | — | — |
| 237548_at | — | Transcribed locus | — | — |
| 237665_at | — | — | — | — |
| 237760_at | — | Homo sapiens, clone | — | — |
| | | IMAGE: 5169349, mRNA | | |
| 237905_at | KRT25 | keratin 25 | 147183 | 17q21.2 |
| 237941_at | — | Transcribed locus | — | — |
| 237942_at | — | Transcribed locus, strongly similar to | — | — |
| | | NP_060189.2 SNF related kinase | | |
| | | [Homo sapiens] | | |
| 238037_at | LMLN | leishmanolysin-like (metallopeptidase | 89782 | 3q29 |
| | | M8 family) | | |
| 238045_at | TMEM65 | transmembrane protein 65 | 157378 | 8q24.13 |
| 238093_at | — | CDNA FLJ40445 fis, clone | — | — |
| | | TESTI2040297 | | |
| 238276_at | — | Transcribed locus | — | — |
| 238505_at | ADPRH | ADP-ribosylarginine hydrolase | 141 | 3q13.31-q13.33 |
| 238581_at | GBP5 | guanylate binding protein 5 | 115362 | 1p22.2 |
| 238801_at | RBM33 | RNA binding motif protein 33 | 155435 | 7q36.3 |
| 238904_at | — | MRNA; cDNA DKFZp547A0515 | — | — |
| | | (from clone DKFZp547A0515) | | |
| 238941_at | TRIM50 | Tripartite motif-containing 50 | 135892 | 7q11.23 |
| 239345_at | SLC19A3 | solute carrier family 19, member 3 | 80704 | 2q37 |
| 239357_at | — | — | — | — |
| 239716_at | — | Transcribed locus | — | — |
| 239719_at | CD109 | CD109 molecule | 135228 | 6q13 |
| 239834_at | — | Full length insert cDNA clone | — | — |
| | | YR23D07 | | |
| 240171_at | — | Transcribed locus | — | — |
| 240172_at | ERGIC2 | ERGIC and golgi 2 | 51290 | 12p11.22 |
| 240221_at | CSNK1A1 | Casein kinase 1, alpha 1 | 1452 | 5q32 |
| 240270_x_at | — | CDNA FLJ26264 fis, clone | — | — |
| | | DMC05506 | | |
| 240293_at | LOC283152 | hypothetical protein LOC283152 | 283152 | 11q23.3 |
| 240567_at | — | CDNA clone IMAGE: 4096591 | — | — |
| 240612_at | — | — | — | — |
| 240646_at | GIMAP8 | GTPase, IMAP family member 8 | 155038 | 7q36.1 |
| 240786_at | NOTCH4 | Notch homolog 4 (Drosophila) | 4855 | 6p21.3 |
| 240787_at | — | — | — | — |
| 240900_at | C7orf50 | chromosome 7 open reading frame 50 | 84310 | 7p22.3 |
| 240945_at | — | Transcribed locus, strongly similar to | — | — |
| | | XP_527560.1 PREDICTED: similar to | | |
| | | transitin [Pan troglodytes] | | |
| 240966_at | SH3TC2 | SH3 domain and tetratricopeptide | 79628 | 5q32 |
| | | repeats 2 | | |
| 240998_at | — | Transcribed locus | — | — |
| 241067_at | — | Transcribed locus | — | — |
| 241177_at | — | Transcribed locus | — | — |
| 241448_at | — | CDNA FLJ36228 fis, clone | — | — |
| | | THYMU2001139 | | |
| 241707_at | LOC388630 | similar to C05G5.5 | 388630 | 1p33 |
| 241712_at | CANX | Calnexin | 821 | 5q35 |
| 241849_at | — | Transcribed locus | — | — |
| 242342_at | — | CDNA FLJ40823 fis, clone | — | — |
| | | TRACH2011093 | | |
| 242426_at | NRG4 | neuregulin 4 | 145957 | 15q24.2 |
| 242447_at | LOC285382 | hypothetical gene supported by | 285382 | 3q27.2 |
| | | AK091454 | | |
| 242718_at | — | Transcribed locus | — | — |
| 242868_at | — | Transcribed locus | — | — |
| 242913_at | CLIC6 | chloride intracellular channel 6 | 54102 | 21q22.12 |
| 243118_at | C6orf182 | chromosome 6 open reading frame 182 | 285753 | 6q21 |
| 243142_at | — | Transcribed locus | — | — |
| 243160_at | — | Transcribed locus | — | — |
| 243414_at | PPIL2 | Peptidylprolyl isomerase (cyclophilin)- | 23759 | 22q11.21 |
| | | like 2 | | |
| 243636_s_at | — | Transcribed locus | — | — |
| 243723_at | — | Transcribed locus | — | — |
| 244004_at | — | Transcribed locus | — | — |
| 244029_at | — | CDNA FLJ42228 fis, clone | — | — |
| | | THYMU2041252 | | |
| 244116_at | — | Transcribed locus | — | — |
| 244220_at | — | — | — | — |
| 244258_at | — | Transcribed locus | — | — |
| 244283_x_at | — | Transcribed locus | — | — |
| 244440_at | — | — | — | — |
| 244820_at | — | Transcribed locus | — | — |
| 36994_at | ATP6V0C | ATPase, H+ transporting, lysosomal | 527 | 16p13.3 |
| | | 16 kDa, V0 subunit c | | |
| AFFX-CreX- | — | — | — | — |
| 3_at | | | | |

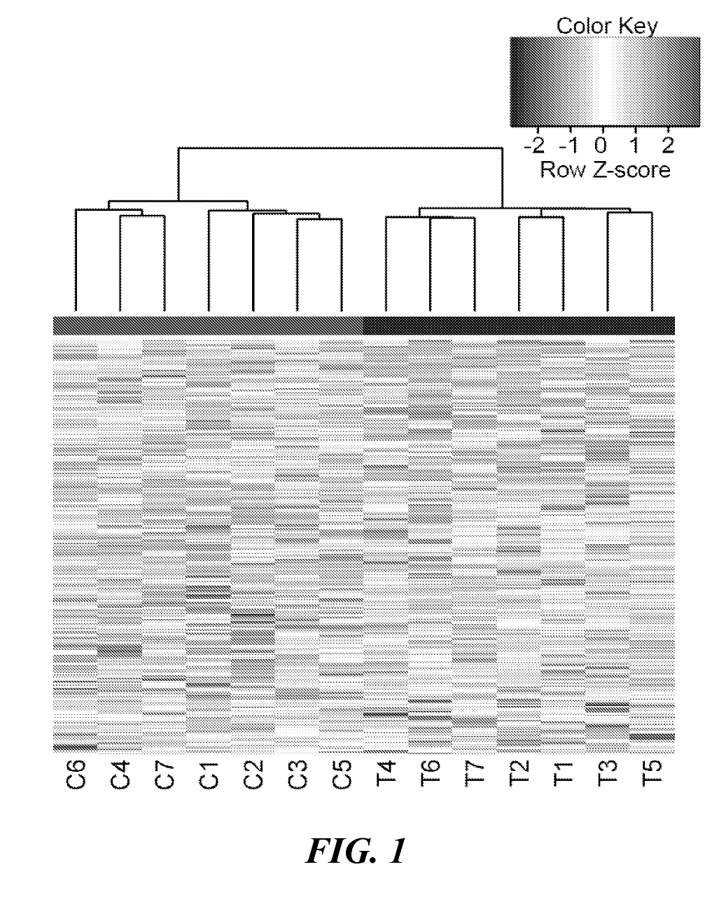

Only five probe sets among the Individual gene set were located on chromosome 21, corresponding to the genes CLIC6, ITGB2, RUNX1, and two open reading frames (ORFs) of unknown function (C21orf67, C21orf86). Four of these five were up-regulated in DS; the exception was RUNX1, which was down-regulated in the DS samples. In the full Individual gene set, 224 (54%) of the genes were up-regulated and 190 (46%) were down-regulated. There was widespread differential expression between trisomic and euploid fetuses, and clustering based on these genes alone, excluding chromosome 21 genes, is sufficient to separate the euploid and trisomic samples (FIG. 1).

The second set was identified by Gene Set Enrichment Analysis (Subramanian et al. (2005)) (GSEA). A single chromosomal band, chromosome 21, band 22, was identified as having genes that were significantly up-regulated as a group (false discovery rate [FDR] q-value=0.006) in DS fetuses. For functional analysis (see Example 4), the 82-gene “Leading Edge” subset of the genes GSEA identified from band chr21q22 was selected (see Methods and Table 4). Only three genes (CLIC6, RUNX1, and C21orf87) are common to both the Individual and Leading Edge gene sets.

TABLE 4
Leading edge subset genes on Chr21q22 that are
differentially expressed in trisomy 21 fetuses

| Gene identifier | Gene name |
|---|---|
| ADARB1 | adenosine deaminase, RNA-specific, B1 (RED1 |
| | homolog rat) |
| B3GALT5 | UDP-Gal:betaGlcNAc beta 1,3-galactosyltransferase, |
| | polypeptide 5 |
| BACE2 | beta-site APP-cleaving enzyme 2 |
| BRWD1 | bromodomain and WD repeat domain containing 1 |
| C21ORF121 | chromosome 21 open reading frame 121 |
| C21ORF130 | chromosome 21 open reading frame 130 |
| C21ORF22 | chromosome 21 open reading frame 22 |
| C21ORF25 | chromosome 21 open reading frame 25 |
| C21ORF29 | chromosome 21 open reading frame 29 |
| C21ORF33 | chromosome 21 open reading frame 33 |
| C21ORF45 | chromosome 21 open reading frame 45 |
| C21ORF51 | chromosome 21 open reading frame 51 |
| C21ORF56 | chromosome 21 open reading frame 56 |
| C21ORF59 | chromosome 21 open reading frame 59 |
| C21ORF66 | chromosome 21 open reading frame 66 |
| C21ORF7 | chromosome 21 open reading frame 7 |
| C21ORF77 | chromosome 21 open reading frame 77 |
| C21ORF84 | chromosome 21 open reading frame 84 |
| C21ORF86 | Chromosome 21 open reading frame 86 |
| C21ORF89 | chromosome 21 open reading frame 89 |
| C21ORF9 | chromosome 21 open reading frame 9 |
| C21ORF90 | chromosome 21 open reading frame 90 |
| CBR1 | carbonyl reductase 1 |
| CHAF1B | chromatin assembly factor 1, subunit B (p60) |
| CLDN14 | claudin 14 |
| CLIC6 | chloride intracellular channel 6 |
| CRYAA | crystallin, alpha A |
| CRYZL1 | crystallin, zeta (quinone reductase)-like 1 |
| CSTB | cystatin B (stefin B) |
| DIP2A | DIP2 disco-interacting protein 2 homolog A (Drosophila) |
| DONSON | downstream neighbor of SON |
| DSCR3 | Down syndrome critical region gene 3 |
| DSCR6 | Down syndrome critical region gene 6 |
| DYRK1A | dual-specificity tyrosine-(Y)-phosphorylation regulated |
| | kinase 1A |
| GART | phosphoribosylglycinamide formyltransferase, |
| | phosphoribosylglycinamide synthetase, |
| | phosphoribosylaminoimidazole synthetase |
| H2BFS | H2B histone family, member S /// histone cluster 1, H2bk |
| HLCS | holocarboxylase synthetase (biotin-(proprionyl- |
| | Coenzyme A-carboxylase (ATP-hydrolysing)) ligase) |
| HMGN1 | high-mobility group nucleosome binding domain 1 |
| IFNAR1 | interferon (alpha, beta and omega) receptor 1 |
| IFNAR2 | interferon (alpha, beta and omega) receptor 2 |
| IFNGR2 | interferon gamma receptor 2 (interferon gamma |
| | transducer 1) |
| ITSN1 | intersectin 1 (SH3 domain protein) |
| KCNE1 | potassium voltage-gated channel, Isk-related family, |
| | member 1 |
| KCNE2 | potassium voltage-gated channel, Isk-related family, |
| | member 2 |
| KRTAP10-12 | keratin associated protein 10-12 |
| KRTAP13-1 | keratin associated protein 13-1 |
| KRTAP19-1 | keratin associated protein 19-1 |
| LOC284837 | hypothetical protein LOC284837 |
| LRRC3 | leucine rich repeat containing 3 |
| MCM3APAS | minichromosome maintenance complex component 3 |
| | associated protein antisense |
| MX2 | myxovirus (influenza virus) resistance 2 (mouse) |
| NDUFV3 | NADH dehydrogenase (ubiquinone) flavoprotein 3, |
| | 10 kDa |
| OLIG2 | oligodendrocyte lineage transcription factor 2 |
| PCNT | pericentrin (kendrin) |
| PCP4 | Purkinje cell protein 4 |
| PDE9A | phosphodiesterase 9A |
| PDXK | pyridoxal (pyridoxine, vitamin B6) kinase |
| PFKL | phosphofructokinase, liver |
| PIGP | phosphatidylinositol glycan anchor biosynthesis, class P |
| PLAC4 | placenta-specific 4 |
| POFUT2 | protein O-fucosyltransferase 2 |
| PRDM15 | PR domain containing 15 |
| PRMT2 | protein arginine methyltransferase 2 |
| PWP2H | PWP2 periodic tryptophan protein homolog (yeast) |
| RIPK4 | receptor-interacting serine-threonine kinase 4 |
| RUNX1 | runt-related transcription factor 1 (acute myeloid |
| | leukemia 1; aml1 oncogene) |
| SH3BGR | SH3 domain binding glutamic acid-rich protein |
| SIM2 | single-minded homolog 2 (Drosophila) |
| SLC19A1 | solute carrier family 19 (folate transporter), member 1 |
| SNF1LK | SNF1-like kinase |
| SOD1 | superoxide dismutase 1, soluble (amyotrophic lateral |
| | sclerosis 1 (adult)) |
| SON | SON DNA binding protein |
| TFF2 | trefoil factor 2 (spasmolytic protein 1) |
| TFF3 | trefoil factor 3 (intestinal) |
| TMEM1 | transmembrane protein 1 |
| TMEM50B | transmembrane protein 50B |
| TMPRSS2 | transmembrane protease, serine 2 |
| TRPM2 | transient receptor potential cation channel, subfamily M, |
| | member 2 |
| TSGA2/RSPH1 | radial spoke head 1 homolog (Chlamydomonas)/ |
| | meichroacidin |
| TTC3 | tetratricopeptide repeat domain 3 |
| UBE2G2 | ubiquitin-conjugating enzyme E2G 2 (UBC7 homolog, |
| | yeast) |
| ZNF295 | zinc finger protein 295 |

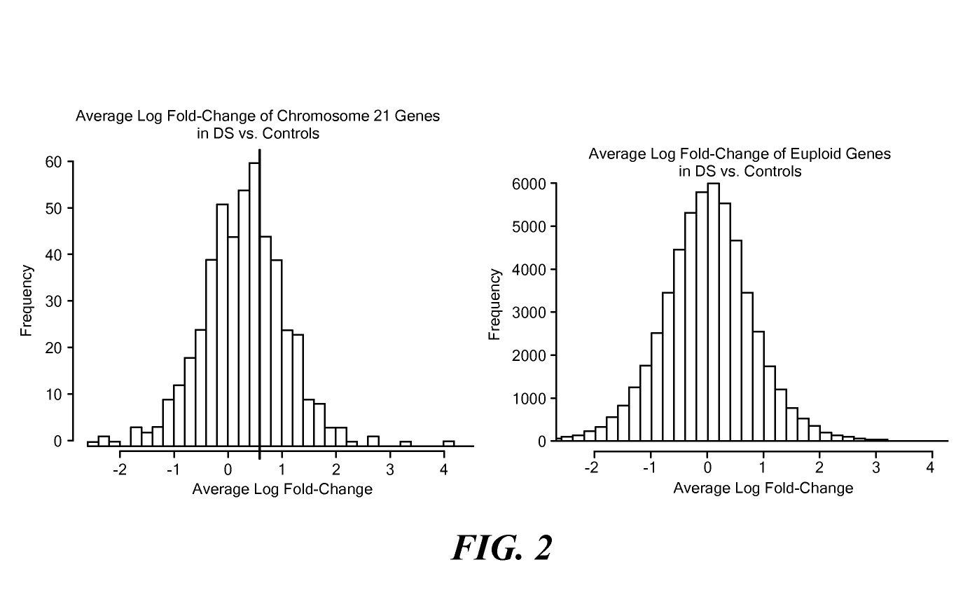

To quantify the extent of differential expression of the known trisomic genes, changes in expression levels of all chromosome 21 probes were examined on microarrays. For each probe set, the fold-change between its average expression level in the DS samples and its average expression in the controls was computed. A histogram of these changes is shown in FIG. 2a, in which a bold vertical line marks the 1.5-fold up-regulation expected given the increased gene dosage. Overexpression was seen for 65% of the chromosome 21 probes (325 of 501). The mean and median fold-changes were 1.44 and 1.25, respectively, but the range is quite large (from 5-fold down-regulation to 16-fold up-regulation in DS). In contrast, only 50.4% (27,299 of 54,174) of the probes from chromosomes other than 21 are higher in DS (FIG. 2b); this frequency of overexpression is significantly different from the frequency of overexpression seen on chromosome 21 (χ2 test, p<0.0006).

### Example 4

### Functional Analyses of Differentially Expressed Genes in Down Syndrome Fetuses

In this Example, genes identified in Example 3 as being differentially expressed in Down Syndrome fetuses were subject to functional analyses in order to examine possible mechanisms underlying the disease.

### Materials and Methods

Functional analysis of gene lists was performed in DAVID (Dennis et al. (2003), the contents of which are herein incorporated in their entirety), using the Panther functional annotation classes (Thomas et al. (2003), the contents of which are herein incorporated in their entirety) in addition to the default pathway selections, which include Gene Ontology (GO) terms (Ashburner et al. (2000), the contents of which are herein incorporated in their entirety), pathways defined from the KEGG (Kanehisa et al. (2000), the contents of which are herein incorporated in their entirety) and BioCarta (www.biocarta.com) databases, InterPro protein families (Apweiler et al. (2000), the contents of which are herein incorporated in their entirety), and Protein Information Resource keywords (Barker et al. (2000), the contents of which are herein incorporated in their entirety). DAVID's EASE score rather than the more stringent Benjamini-Hochberg FDR cutoff was used for DAVID results because the FDR adjustment assumes independence of the functional pathways (which, in practice, overlap heavily by design), and adjusting for multiple testing in such cases is controversial (Gentleman (20040. Therefore, to reduce the possibility of false-positive associations, only those functional processes represented in the DAVID output for both the Individual and Leading Edge gene sets were focused upon.

### Results

Pathway analysis of the Individual and Leading Edge gene sets identified in Example 3 was performed in the Database for Annotation, Visualization, and Integrated Discovery (DAVID). All functional annotations were examined with a modified Fisher exact p-value (the “EASE” score) below 0.1 (see Materials and Methods). The full DAVID results for the two gene sets appear in Tables 5 and 6. Several consistent patterns in differential expression were observed in both the Individual and the Leading Edge gene sets (Table 7). Because of the limited overlap between these two sets and the size of the functional groups considered, the two gene sets can be seen as providing largely independent confirmation of the importance of these functional processes in DS. Therefore, in this Example, only functional processes implicated by both gene sets were focused upon. Using this criterion, the following functions appear to be disrupted in DS (Table 7): oxidative stress, ion transport, G-protein signaling, immune and stress response, circulatory system functions, cell structure, sensory perception, and several developmental processes.

### Discussion

Although there is very little overlap between the gene lists from the two sets (414 individual gene list and 84-leading edge subset), the gene lists tend to implicate the same processes. Without wishing to be bound by any particular theory, the inventors suggest that several of the implicated functional groups of genes may be amenable to a single explanation. For example, reactive oxygen species (especially hydrogen peroxide) are known to disrupt ion transport mechanisms, leading to problems with signal transduction through cell membranes, leading to cellular dysfunction (possibly including structural membrane problems) and pathological symptoms. (See, e.g., Kourie et al. (1998), the contents of which are herein incorporated by reference in their entirety). Kourie et al. point out that oxidative stress can act both directly on ion transport genes and pathways or indirectly by targeting membrane phospholipids.

Based on studies in adult patients, oxidative stress is known to play a role in Down Syndrome as well as in Alzheimer's disease (Zana et al. (2007)). Data described in this Example consistently support a role for oxidative stress in Down Syndrome fetuses. For example, significant expression differences were observed in a few genes involved in phospholipid biology, many genes involved in ion transport, a few genes involved in heart muscle physiology, and some DNA damage repair genes. Without wishing to be bound by any particular theory, the inventors propose that data presented in this Example suggest that there is an oxidative stress response present before birth.

Data presented in this Example also support a role for G-proteins in Down Syndrome. Though a role for G-protein dependent pathways has been suggested by the literature (see, e.g., Best et al. (2007) and Lumbreras et al. (2006)), the data presented here suggest a wider role for G-protein signaling than had been appreciated before.

Immune response genes that were differentially expressed include three interferon receptors on chromosome 21 and other genes involved in broader processes. Genes involved in developmental processes and sensory perception also appear to be misregulated in trisomy 21 samples.

TABLE 5
Full DAVID results for the Individual Gene Set
Annotation Category/Term/Count/%/P-value (EASE)/List Total/Pop Hits/Pop Total/
Fold Enrichment/Benjamini-Hochberg FDR
Genes

```
PANTHER_MF_ALL MF00213: Non-receptor serine/threonine protein kinase 91 21.06%
      8.74E−07 354 4578 29414 1.651643947 2.09E−04
221667_s_at, 204029_at, 215319_at, 212057_at, 200783_s_at, 1570505_at, 237548_at,
232422_at, 1563620_at, 207566_at, 211571_s_at, 214955_at, 218557_at, 238801_at,
243636_s_at, 216889_s_at, 200956_s_at, 205885_s_at, 215112_x_at, 1556017_at, 227694_at,
205774_at, 205380_at, 223103_at, 220233_at, 208570_at, 244029_at, 208357_x_at, 230571_at,
206074_s_at, 227928_at, 230239_at, 234481_at, 226410_at, 236613_at, 211016_x_at,
1552703_s_at, 204118_at, 203799_at, 244440_at, 206426_at, 1552925_at, 217340_at,
211925_s_at, 221066_at, 237942_at, 231338_at, 203099_s_at, 206213_at, 214883_at,
202332_at, 207374_at, 239719_at, 205655_at, 240900_at, 229035_s_at, 240221_at,
1569025_s_at, 211740_at, 229402_at, 1554778_at, 218070_s_at, 207934_at, 210034_s_at,
214420_s_at, 208039_at, 208710_s_at, 1560997_at, 206180_x_at, 214914_at, 229438_at,
202695_s_at, 230966_at, 226125_at, 228712_at, 213444_at, 237093_at, 210981_s_at,
206190_at, 232725_s_at, 211212_s_at, 1557300_s_at, 206413_s_at, 210156_s_at, 232187_at,
204708_at, 229131_at, 221766_s_at, 235473_at, 215766_at, 1554188_at,
PANTHER_MF_ALL MF00042: Nucleic acid binding 142 32.87% 3.26E−06 354
      8438  29414 1.39829772 3.89E−04
212057_at, 216251_s_at, 235098_at, 232422_at, 217340_at, 207566_at, 211571_s_at,
1568949_at, 200956_s_at, 205885_s_at, 227694_at, 223122_s_at, 1561365_at, 229408_at,
1554345_a_at, 205388_at, 217621_at, 207253_s_at, 205665_at, 230571_at, 206074_s_at,
206286_s_at, 242426_at, 236613_at, 223906_s_at, 224330_s_at, 232857_at, 204118_at,
219650_at, 209960_at, 213753_x_at, 217340_at, 221066_at, 1560587_s_at, 218585_s_at,
219683_at, 207968_s_at, 204913_s_at, 206213_at, 226295_at, 1555404_a_at, 214883_at,
1570093_at, 231954_at, 219960_s_at, 230009_at, 208698_s_at, 239719_at, 237905_at,
36994_at, 240966_at, 225215_s_at, 229444_at, 232063_x_at, 218714_at, 236766_at,
208884_s_at, 225939_at, 226200_at, 224683_at, 218070_s_at, 208688_x_at, 233937_at,
217621_at, 210034_s_at, 213442_x_at, 208710_s_at, 206180_x_at, 208334_at, 227449_at,
213361_at, 217340_at, 211212_s_at, 1558208_at, 234741_at, 239357_at, 235885_at, 218475_at,
216242_x_at, 236098_at, 243160_at, 1569794_at, 226623_at, 203164_at, 218429_s_at,
218557_at, 216889_s_at, 215112_x_at, 206035_at, 205774_at, 231548_at, 206972_s_at,
208357_x_at, 226986_at, 217340_at, 242868_at, 237182_at, 235788_at, 219568_x_at,
224122_at, 206426_at, 200710_at, 233722_at, 241849_at, 1558000_at, 211925_s_at,
219919_s_at, 231147_at, 225858_s_at, 217340_at, 214915_at, 205655_at, 1554890_a_at,
240900_at, 213893_x_at, 1552564_at, 1557558_s_at, 236905_at, 238941_at, 211740_at,
1553322_s_at, 1554778_at, 203022_at, 206153_at, 221517_s_at, 223357_s_at, 217415_at,
220120_s_at, 1556037_s_at, 214914_at, 206563_s_at, 208389_s_at, 1560752_at, 205938_at,
240172_at, 1555393_s_at, 206190_at, 1556282_at, 1566163_at, 239345_at, 239716_at,
229131_at, 217340_at,
PANTHER_BP_ALL BP00104: G-protein mediated signaling 81 18.75% 3.86E−06
      354 4056 29414 1.659349614 8.50E−04
236805_at, 243160_at, 215319_at, 233543_s_at, 235098_at, 214013_s_at, 1554681_a_at,
234701_at, 235792_x_at, 205885_s_at, 215112_x_at, 238045_at, 201819_at, 204182_s_at,
222389_s_at, 1561365_at, 236185_at, 233467_s_at, 229408_at, 205388_at, 217621_at,
208476_s_at, 206972_s_at, 205665_at, 208357_x_at, 202415_s_at, 209163_at, 236613_at,
242426_at, 242868_at, 211016_x_at, 204385_at, 205527_s_at, 205075_at, 219650_at,
1558000_at, 1552925_at, 211925_s_at, 221066_at, 231338_at, 219919_s_at, 223792_at,
203099_s_at, 208255_s_at, 225858_s_at, 226114_at, 213835_x_at, 239719_at, 211208_s_at,
216669_at, 240966_at, 240900_at, 236905_at, 229444_at, 218070_s_at, 207934_at,
210034_s_at, 234544_at, 238505_at, 208710_s_at, 223357_s_at, 1552461_at, 211422_at,
212793_at, 208389_s_at, 211771_s_at, 224108_at, 240172_at, 226013_at, 206190_at,
222556_at, 1558208_at, 206413_s_at, 210156_s_at, 235885_at, 239716_at, 228831_s_at,
206973_at, 235473_at, 236098_at, 217340_at,
PANTHER_BP_ALL BP00044: mRNA transcription regulation 167 38.66% 4.15E−06
      354 10414 29414 1.332446327 4.57E−04
224881_at, 215319_at, 220434_at, 200783_s_at, 212057_at, 235098_at, 240171_at, 237548_at,
211571_s_at, 1557248_at, 234701_at, 244820_at, 1568949_at, 200956_s_at, 205885_s_at,
222389_s_at, 1561365_at, 234251_at, 236185_at, 229408_at, 208570_at, 206074_s_at,
238093_at, 209163_at, 236613_at, 242426_at, 201959_s_at, 213753_x_at, 219650_at,
1565748_at, 217340_at, 244220_at, 231338_at, 223792_at, 1560587_s_at, 218585_s_at,
219683_at, 208255_s_at, 207968_s_at, 204913_s_at, 214883_at, 202332_at, 201079_at,
213835_x_at, 1570093_at, 239719_at, 211208_s_at, 242913_at, 237905_at, 240966_at,
225215_s_at, 1555349_a_at, 240221_at, 1569025_s_at, 232063_x_at, 218714_at, 236766_at,
208884_s_at, 225939_at, 226200_at, 218070_s_at, 233937_at, 234544_at, 213442_x_at,
240646_at, 208710_s_at, 237311_at, 1552461_at, 233318_at, 206180_x_at, 234480_at,
228375_at, 227449_at, 213444_at, 206775_at, 204300_at, 211212_s_at, 1557300_s_at,
1558208_at, 234741_at, 239357_at, 218493_at, 232187_at, 218475_at, 215766_at, 214595_at,
221920_s_at, 236805_at, 243160_at, 1570505_at, 1569794_at, 214013_s_at, 226623_at,
218429_s_at, 218744_s_at, 214955_at, 238801_at, 243636_s_at, 216889_s_at, 1556017_at,
206035_at, 1553172_at, 204182_s_at, 205774_at, 231548_at, 220233_at, 1554740_a_at,
208357_x_at, 227928_at, 235004_at, 218980_at, 202415_s_at, 230239_at, 240786_at,
226410_at, 211016_x_at, 204385_at, 235788_at, 219568_x_at, 1554690_a_at, 205075_at,
228403_at, 233722_at, 233754_x_at, 1558000_at, 220364_at, 211925_s_at, 213038_at,
231147_at, 226114_at, 214915_at, 205655_at, 229035_s_at, 210330_at, 227420_at, 236905_at,
238941_at, 211740_at, 1559633_a_at, 1553322_s_at, 1554778_at, 203022_at, 221517_s_at,
204177_s_at, 214420_s_at, 238505_at, 1556037_s_at, 211422_at, 212684_at, 218416_s_at,
234472_at, 208389_s_at, 211194_s_at, 214752_x_at, 228712_at, 213250_at, 1554821_a_at,
240172_at, 1556282_at, 210297_s_at, 211064_at, 239716_at, 229131_at, 221114_at,
1552531_a_at, 206973_at, 235473_at, 207976_at,
PANTHER_MF_ALL MF00224: KRAB box transcription factor 112 25.93% 6.43E−06
      354 6298 29414 1.477631676 5.12E−04
221667_s_at, 215319_at, 220434_at, 216251_s_at, 1557248_at, 234701_at, 244820_at,
1568949_at, 1568687_s_at, 227694_at, 222389_s_at, 1561365_at, 234251_at, 202931_x_at,
233467_s_at, 236185_at, 207253_s_at, 206074_s_at, 238093_at, 236613_at, 235955_at,
201959_s_at, 205797_s_at, 1553450_s_at, 211180_x_at, 236388_at, 239834_at, 209960_at,
1565748_at, 221066_at, 1560587_s_at, 223792_at, 218585_s_at, 208255_s_at, 243414_at,
239719_at, 211208_s_at, 237905_at, 240221_at, 231713_s_at, 1569025_s_at, 208884_s_at,
236766_at, 226200_at, 233937_at, 237311_at, 208710_s_at, 240646_at, 233318_at, 200838_at,
213361_at, 206775_at, 1565628_at, 222556_at, 211212_s_at, 234741_at, 239357_at, 218493_at,
1558208_at, 216242_x_at, 215766_at, 243160_at, 203164_at, 238276_at, 218744_s_at,
238801_at, 214955_at, 216889_s_at, 243636_s_at, 240612_at, 1553172_at, 1556017_at,
201819_at, 204182_s_at, 205774_at, 206972_s_at, 235004_at, 227928_at, 243118_at,
240786_at, 226410_at, 211016_x_at, 235788_at, 230447_at, 1552703_s_at, 233722_at,
233754_x_at, 241849_at, 1558000_at, 213038_at, 231147_at, 226114_at, 232213_at, 219419_at,
206413_s_at, 214915_at, 205655_at, 229035_s_at, 213893_x_at, 1559633_a_at, 214420_s_at,
238505_at, 212684_at, 202695_s_at, 230966_at, 211771_s_at, 213250_at, 1554821_a_at,
1556282_at, 220129_at, 230492_s_at, 211064_at, 206973_at,
PANTHER_BP_ALL BP00060: Protein metabolism and modification 68 15.74% 7.55E−06
      354 3257 29414 1.734770308 5.53E−04
236805_at, 221667_s_at, 212057_at, 233351_at, 237548_at, 217340_at, 1563620_at, 203164_at,
218744_s_at, 1557248_at, 214955_at, 205885_s_at, 205774_at, 1554345_a_at, 202415_s_at,
226986_at, 230239_at, 217340_at, 211016_x_at, 235955_at, 201959_s_at, 237182_at,
1552703_s_at, 224330_s_at, 235788_at, 1552703_s_at, 205075_at, 203167_at, 209960_at,
213753_x_at, 217340_at, 237942_at, 213038_at, 219919_s_at, 208255_s_at, 243414_at,
230988_at, 206213_at, 217340_at, 232802_at, 219960_s_at, 211208_s_at, 225215_s_at,
238941_at, 232063_x_at, 208884_s_at, 226200_at, 225939_at, 208688_x_at, 207934_at,
210034_s_at, 223357_s_at, 238037_at, 202695_s_at, 204014_at, 200838_at, 228712_at,
227449_at, 217340_at, 237093_at, 210981_s_at, 205938_at, 222603_at, 1565628_at,
1558208_at, 210156_s_at, 204708_at, 217340_at,
SP_PIR_KEYWORDS alternative splicing 120 27.78% 1.70E−05 275 5530
      17599 1.388709518 0.017996105
220434_at, 212057_at, 233351_at, 240171_at, 237548_at, 211571_s_at, 1568949_at, 227694_at,
222389_s_at, 202931_x_at, 233467_s_at, 229408_at, 235343_at, 206074_s_at, 222826_at,
240998_at, 235955_at, 223906_s_at, 232857_at, 211180_x_at, 236388_at, 203799_at,
209960_at, 236834_at, 213753_x_at, 244220_at, 231338_at, 237942_at, 203099_s_at,
219683_at, 207968_s_at, 243414_at, 214883_at, 213835_x_at, 1570093_at, 219960_s_at,
239719_at, 242913_at, 216669_at, 240966_at, 229444_at, 231713_s_at, 1569025_s_at,
229402_at, 225939_at, 233937_at, 208688_x_at, 217621_at, 234544_at, 208710_s_at,
238037_at, 206180_x_at, 234480_at, 222756_s_at, 229164_s_at, 1565628_at, 1557300_s_at,
234741_at, 239357_at, 210156_s_at, 243142_at, 218475_at, 214595_at, 236098_at,
221920_s_at, 243160_at, 236805_at, 1570505_at, 1563620_at, 238276_at, 202898_at,
214955_at, 216889_s_at, 1553172_at, 201819_at, 206972_s_at, 208357_x_at, 227928_at,
202415_s_at, 235004_at, 226986_at, 240786_at, 225484_at, 1552703_s_at, 200710_at,
233722_at, 241849_at, 1558000_at, 211925_s_at, 213038_at, 219919_s_at, 230988_at,
219419_at, 232802_at, 1554890_a_at, 210644_s_at, 238941_at, 1554778_at, 221517_s_at,
214420_s_at, 1556037_s_at, 211422_at, 206563_s_at, 234472_at, 230966_at, 211194_s_at,
209574_s_at, 214752_x_at, 228712_at, 1560752_at, 211771_s_at, 210981_s_at, 205938_at,
206190_at, 1556282_at, 1566163_at, 232725_s_at, 210297_s_at, 239716_at, 221114_at,
1552531_a_at,
GOTERM_MF_ALL GO: 0005488~binding 235 54.40% 2.04E−05 270 13054
      16968 1.131334797 0.056929477
221667_s_at, 204029_at, 212057_at, 200783_s_at, 233351_at, 233543_s_at, 240171_at,
237548_at, 1557248_at, 1568687_s_at, 202638_s_at, 1561365_at, 1554345_a_at, 205388_at,
208476_s_at, 206286_s_at, 222826_at, 242426_at, 223906_s_at, 201959_s_at, 204118_at,
203799_at, 209960_at, 219650_at, 217340_at, 244220_at, 221066_at, 218585_s_at, 219683_at,
207968_s_at, 213835_x_at, 1570093_at, 240966_at, 235815_at, 1555349_a_at, 240221_at,
226200_at, 224683_at, 207934_at, 208688_x_at, 210034_s_at, 217621_at, 213442_x_at,
237311_at, 208710_s_at, 203045_at, 238037_at, 234480_at, 200838_at, 213361_at, 241448_at,
222756_s_at, 206775_at, 1565628_at, 1565537_at, 234741_at, 239357_at, 216242_x_at,
214595_at, 1570505_at, 214013_s_at, 218744_s_at, 216889_s_at, 240612_at, 1553172_at,
206035_at, 1556017_at, 1554740_a_at, 202415_s_at, 218980_at, 235004_at, 242868_at,
211016_x_at, 237182_at, 204385_at, 235788_at, 219568_x_at, 244440_at, 1554690_a_at,
200710_at, 205075_at, 233754_x_at, 241849_at, 225858_s_at, 226114_at, 207374_at,
1565735_at, 205900_at, 205655_at, 213893_x_at, 236905_at, 238941_at, 211740_at,
1553322_s_at, 1559633_a_at, 206153_at, 214420_s_at, 238505_at, 223357_s_at, 217415_at,
220120_s_at, 206563_s_at, 229438_at, 211194_s_at, 214752_x_at, 1560752_at, 211771_s_at,
213250_at, 210981_s_at, 205938_at, 1554821_a_at, 239716_at, 236713_at, 204708_at,
229131_at, 1552531_a_at, 225830_at, 215319_at, 235098_at, 211571_s_at, 244820_at,
1568949_at, 200956_s_at, 205885_s_at, 200991_s_at, 222389_s_at, 223122_s_at, 234251_at,
202931_x_at, 229408_at, 206765_at, 208570_at, 207253_s_at, 206074_s_at, 209163_at,
236613_at, 240998_at, 232857_at, 211180_x_at, 205527_s_at, 236388_at, 239834_at,
213753_x_at, 237942_at, 223792_at, 203099_s_at, 208255_s_at, 204913_s_at, 223296_at,
202332_at, 214883_at, 219960_s_at, 239719_at, 208698_s_at, 211208_s_at, 242913_at,
216669_at, 36994_at, 225215_s_at, 231713_s_at, 232063_x_at, 208884_s_at, 213949_s_at,
225939_at, 218831_s_at, 234544_at, 240646_at, 208039_at, 233318_at, 206180_x_at,
212793_at, 227449_at, 213444_at, 229164_s_at, 226013_at, 211212_s_at, 204300_at,
210156_s_at, 206413_s_at, 1558208_at, 218475_at, 236098_at, 221920_s_at, 236805_at,
1569794_at, 1563620_at, 202898_at, 238801_at, 235792_x_at, 201819_at, 204182_s_at,
205380_at, 231548_at, 220233_at, 208357_x_at, 227928_at, 230239_at, 240786_at,
1552703_s_at, 1552703_s_at, 233722_at, 203167_at, 1558000_at, 1552925_at, 211925_s_at,
213038_at, 216007_at, 230988_at, 231147_at, 214915_at, 1554890_a_at, 210330_at,
1557558_s_at, 203022_at, 221517_s_at, 238581_at, 204177_s_at, 1556037_s_at, 211422_at,
1560997_at, 212684_at, 234472_at, 202695_s_at, 226125_at, 228712_at, 237093_at, 240172_at,
222603_at, 206190_at, 1566163_at, 239345_at, 230492_s_at, 211064_at, 206973_at, 235473_at,
207976_at,
PANTHER_BP_ALL BP00071: Proteolysis 95 21.99% 5.44E−05 354 5357
      29414 1.473508973 0.002985006
236805_at, 220434_at, 212057_at, 233543_s_at, 216251_s_at, 240171_at, 214013_s_at,
1563620_at, 211571_s_at, 234701_at, 1557248_at, 214955_at, 238801_at, 235792_x_at,
201819_at, 1568687_s_at, 1553172_at, 202638_s_at, 222389_s_at, 234251_at, 1561365_at,
236185_at, 233467_s_at, 205388_at, 223103_at, 217621_at, 207253_s_at, 208476_s_at,
208357_x_at, 227928_at, 235004_at, 243118_at, 226410_at, 242426_at, 242868_at,
1552703_s_at, 235788_at, 211180_x_at, 1552703_s_at, 1559611_at, 205075_at, 203167_at,
209960_at, 219650_at, 220364_at, 231338_at, 213038_at, 216007_at, 203099_s_at,
207968_s_at, 223709_s_at, 1555404_a_at, 226114_at, 232213_at, 207374_at, 219419_at,
1570093_at, 219960_s_at, 1565735_at, 239719_at, 213893_x_at, 240221_at, 238941_at,
1569025_s_at, 236766_at, 208884_s_at, 229402_at, 1554778_at, 218831_s_at, 218070_s_at,
207934_at, 233937_at, 217621_at, 234544_at, 1552461_at, 214914_at, 208334_at, 230966_at,
208389_s_at, 211194_s_at, 214752_x_at, 228712_at, 211771_s_at, 1560752_at, 213361_at,
1554821_a_at, 226013_at, 222603_at, 239345_at, 232725_s_at, 1558208_at, 218493_at,
221766_s_at, 206973_at, 221920_s_at,
GOTERM_MF_ALL GO: 0005515~protein binding 146 33.80% 9.72E−05 270
      7212 16968 1.272225304 0.130570601
221667_s_at, 225830_at, 200783_s_at, 212057_at, 233543_s_at, 233351_at, 235098_at,
237548_at, 244820_at, 200956_s_at, 205885_s_at, 202638_s_at, 200991_s_at, 222389_s_at,
223122_s_at, 1561365_at, 202931_x_at, 229408_at, 206765_at, 208570_at, 208476_s_at,
206074_s_at, 206286_s_at, 222826_at, 242426_at, 201959_s_at, 232857_at, 204118_at,
211180_x_at, 205527_s_at, 236388_at, 239834_at, 219650_at, 209960_at, 213753_x_at,
244220_at, 237942_at, 218585_s_at, 219683_at, 207968_s_at, 202332_at, 214883_at,
219960_s_at, 239719_at, 208698_s_at, 211208_s_at, 216669_at, 36994_at, 1555349_a_at,
231713_s_at, 208884_s_at, 218831_s_at, 207934_at, 208688_x_at, 210034_s_at, 217621_at,
234544_at, 213442_x_at, 208710_s_at, 237311_at, 233318_at, 203045_at, 212793_at,
234480_at, 200838_at, 213361_at, 213444_at, 241448_at, 222756_s_at, 226013_at,
229164_s_at, 206775_at, 1565628_at, 211212_s_at, 1558208_at, 206413_s_at, 210156_s_at,
234741_at, 239357_at, 216242_x_at, 214595_at, 1569794_at, 1563620_at, 202898_at,
218744_s_at, 235792_x_at, 216889_s_at, 201819_at, 206035_at, 204182_s_at, 205380_at,
231548_at, 220233_at, 1554740_a_at, 208357_x_at, 202415_s_at, 218980_at, 230239_at,
240786_at, 242868_at, 237182_at, 1552703_s_at, 235788_at, 1552703_s_at, 244440_at,
1554690_a_at, 205075_at, 203167_at, 241849_at, 1552925_at, 213038_at, 216007_at,
230988_at, 231147_at, 225858_s_at, 1565735_at, 205900_at, 205655_at, 210330_at,
1557558_s_at, 236905_at, 238941_at, 211740_at, 1553322_s_at, 221517_s_at, 204177_s_at,
217415_at, 220120_s_at, 1556037_s_at, 1560997_at, 211194_s_at, 226125_at, 214752_x_at,
228712_at, 1560752_at, 213250_at, 237093_at, 1554821_a_at, 240172_at, 206190_at,
1566163_at, 239716_at, 236713_at, 1552531_a_at, 206973_at, 235473_at, 207976_at,
KEGG_PATHWAY hsa04310: Wnt signaling pathway 12 2.78% 1.21E−04
      82 151 4214 4.083992893 0.024022934
223122_s_at, 240221_at, 1565735_at, 211925_s_at, 212793_at, 1563620_at, 208570_at,
219683_at, 223709_s_at, 206213_at, 230239_at, 202332_at,
PANTHER_BP_ALL BP00142: Ion transport 52 12.04% 1.29E−04 354 2494
      29414 1.732438077 0.005658208
215319_at, 232422_at, 214955_at, 240612_at, 1553172_at, 227694_at, 204182_s_at,
222389_s_at, 1561365_at, 202931_x_at, 206765_at, 231548_at, 205665_at, 230571_at,
218980_at, 226986_at, 226410_at, 235955_at, 223906_s_at, 239834_at, 206426_at, 219650_at,
219683_at, 232213_at, 201079_at, 202332_at, 214883_at, 211208_s_at, 36994_at, 240966_at,
1555349_a_at, 240221_at, 1559633_a_at, 229402_at, 1553322_s_at, 206153_at, 237311_at,
211422_at, 1560997_at, 234480_at, 228375_at, 240172_at, 226013_at, 206775_at, 1566163_at,
234741_at, 239357_at, 239716_at, 204708_at, 232187_at, 221766_s_at, 214595_at,
221920_s_at,
PANTHER_BP_ALL BP00286: Cell structure 68 15.74% 1.47E−04 354 3585
      29414 1.576052132 0.005379909
236805_at, 243160_at, 215319_at, 1570505_at, 214013_s_at, 226623_at, 232422_at,
211571_s_at, 234701_at, 214955_at, 215112_x_at, 238045_at, 1568687_s_at, 222389_s_at,
202931_x_at, 236185_at, 233467_s_at, 1554740_a_at, 240998_at, 204385_at, 206426_at,
211925_s_at, 219919_s_at, 207968_s_at, 207374_at, 202332_at, 213835_x_at, 1570093_at,
219960_s_at, 239719_at, 237905_at, 205655_at, 240966_at, 240900_at, 210644_s_at,
225215_s_at, 227420_at, 240221_at, 1559633_a_at, 1554778_at, 238581_at, 218070_s_at,
204177_s_at, 208710_s_at, 211422_at, 1560997_at, 214914_at, 212793_at, 234472_at,
234480_at, 208389_s_at, 228375_at, 226125_at, 227449_at, 213444_at, 210981_s_at,
206775_at, 1566163_at, 222556_at, 210156_s_at, 211064_at, 239716_at, 236713_at,
228831_s_at, 204708_at, 236098_at, 207976_at, 221920_s_at,
PANTHER_BP_ALL BP00143: Cation transport 80 18.52% 1.97E−04 354
      4462 29414 1.489742635 0.006158388
243160_at, 215319_at, 200783_s_at, 214955_at, 216889_s_at, 205885_s_at, 240612_at,
201819_at, 1553172_at, 227694_at, 222389_s_at, 1561365_at, 202931_x_at, 1554345_a_at,
206765_at, 231548_at, 208476_s_at, 244029_at, 205665_at, 206286_s_at, 226986_at,
226410_at, 235955_at, 223906_s_at, 231643_s_at, 239834_at, 206426_at, 213753_x_at,
219650_at, 211925_s_at, 226295_at, 1555404_a_at, 214883_at, 202332_at, 213835_x_at,
1570093_at, 219960_s_at, 232802_at, 239719_at, 36994_at, 240966_at, 229035_s_at,
240221_at, 238941_at, 229402_at, 1559633_a_at, 1553322_s_at, 1554778_at, 238581_at,
208688_x_at, 233937_at, 210034_s_at, 237311_at, 223357_s_at, 211422_at, 1560997_at,
234480_at, 208334_at, 228375_at, 200838_at, 228712_at, 241448_at, 210981_s_at, 240172_at,
226013_at, 1565628_at, 1566163_at, 220129_at, 239345_at, 204300_at, 206413_s_at,
210156_s_at, 234741_at, 239357_at, 236713_at, 204708_at, 218475_at, 1552531_a_at,
221766_s_at, 214595_at, 221920_s_at,
SP_PIR_KEYWORDS phosphoprotein 101 23.38% 2.26E−04 275 4705 17599
      1.373779538 0.113361647
221667_s_at, 225830_at, 200783_s_at, 216251_s_at, 233543_s_at, 237548_at, 234701_at,
1568949_at, 200956_s_at, 205885_s_at, 200991_s_at, 222389_s_at, 223122_s_at, 202931_x_at,
229408_at, 208476_s_at, 207253_s_at, 206074_s_at, 222826_at, 236613_at, 240998_at,
235955_at, 211180_x_at, 236388_at, 244220_at, 237942_at, 203099_s_at, 207968_s_at,
208255_s_at, 204913_s_at, 202332_at, 208698_s_at, 242913_at, 1555349_a_at, 240221_at,
1569025_s_at, 208884_s_at, 233937_at, 208688_x_at, 210034_s_at, 217621_at, 234544_at,
237311_at, 208710_s_at, 208039_at, 233318_at, 234480_at, 204014_at, 227449_at,
222756_s_at, 241448_at, 1565628_at, 1557300_s_at, 234741_at, 239357_at, 1558208_at,
214013_s_at, 203164_at, 238276_at, 218744_s_at, 235792_x_at, 216889_s_at, 206035_at,
205380_at, 223103_at, 231548_at, 208357_x_at, 226986_at, 230239_at, 240786_at, 242868_at,
211016_x_at, 225484_at, 244440_at, 233722_at, 1558000_at, 211925_s_at, 219919_s_at,
230988_at, 207374_at, 205900_at, 240900_at, 1554890_a_at, 210644_s_at, 236905_at,
211740_at, 1553322_s_at, 1559633_a_at, 1554778_at, 221517_s_at, 220120_s_at, 217415_at,
206563_s_at, 208389_s_at, 211194_s_at, 202695_s_at, 226125_at, 214752_x_at, 228712_at,
237093_at, 210981_s_at, 206973_at,
PANTHER_MF_ALL MF00108: Protein kinase 40 9.26% 2.47E−04 354
      1790 29414 1.856768614 0.014643064
236805_at, 215319_at, 212057_at, 216251_s_at, 237548_at, 238801_at, 243636_s_at,
200956_s_at, 215112_x_at, 1556017_at, 240221_at, 226200_at, 230239_at, 208710_s_at,
226410_at, 214914_at, 229438_at, 202695_s_at, 204385_at, 230966_at, 228712_at, 227449_at,
206426_at, 237093_at, 210981_s_at, 217340_at, 232725_s_at, 237942_at, 1557300_s_at,
231338_at, 1558208_at, 206413_s_at, 234741_at, 239357_at, 228831_s_at, 204708_at,
229131_at, 1552531_a_at, 221766_s_at, 202332_at, 214595_at,
GOTERM_BP_ALL GO: 0019538~protein metabolic process 85 19.68% 3.62E−04
      251 3691 15360 1.409264055 0.851060507
236805_at, 221667_s_at, 243160_at, 200783_s_at, 216251_s_at, 237548_at, 217340_at,
1563620_at, 1557248_at, 214955_at, 206035_at, 200991_s_at, 205774_at, 220233_at,
206074_s_at, 206286_s_at, 202415_s_at, 230239_at, 240998_at, 211016_x_at, 201959_s_at,
237182_at, 1552703_s_at, 224330_s_at, 235788_at, 1552703_s_at, 209960_at, 241849_at,
213753_x_at, 217340_at, 237942_at, 213038_at, 219919_s_at, 218585_s_at, 243414_at,
208255_s_at, 202332_at, 219960_s_at, 1565735_at, 211208_s_at, 205900_at, 216669_at,
205655_at, 1554890_a_at, 225215_s_at, 1557558_s_at, 1555349_a_at, 240221_at, 232063_x_at,
208884_s_at, 1559633_a_at, 213949_s_at, 225939_at, 226200_at, 224683_at, 208688_x_at,
207934_at, 204177_s_at, 210034_s_at, 238505_at, 208710_s_at, 223357_s_at, 238037_at,
234472_at, 202695_s_at, 211194_s_at, 204014_at, 200838_at, 228712_at, 227449_at,
1560752_at, 237093_at, 210981_s_at, 205938_at, 226013_at, 229164_s_at, 222603_at,
1565628_at, 222556_at, 204300_at, 210156_s_at, 218493_at, 239716_at, 204708_at, 217340_at,
PANTHER_BP_ALL BP00054: tRNA metabolism 14 3.24% 6.51E−04 354
      374 29414 3.11033566 0.017754878
235955_at, 226125_at, 204014_at, 210644_s_at, 205885_s_at, 243636_s_at, 201819_at,
236905_at, 218714_at, 213949_s_at, 1558208_at, 213835_x_at, 1570093_at, 220120_s_at,
GOTERM_BP_ALL GO: 0044267~cellular protein metabolic process 80 18.52% 6.59E−04
      251 3482 15360 1.405978613 0.823245437
236805_at, 221667_s_at, 243160_at, 200783_s_at, 216251_s_at, 237548_at, 217340_at,
1563620_at, 1557248_at, 214955_at, 206035_at, 200991_s_at, 205774_at, 220233_at,
206074_s_at, 206286_s_at, 202415_s_at, 230239_at, 240998_at, 211016_x_at, 201959_s_at,
237182_at, 1552703_s_at, 224330_s_at, 235788_at, 1552703_s_at, 209960_at, 213753_x_at,
217340_at, 237942_at, 213038_at, 219919_s_at, 218585_s_at, 208255_s_at, 243414_at,
202332_at, 219960_s_at, 1565735_at, 211208_s_at, 205900_at, 205655_at, 1554890_a_at,
225215_s_at, 1555349_a_at, 240221_at, 232063_x_at, 208884_s_at, 1559633_a_at,
213949_s_at, 225939_at, 226200_at, 224683_at, 208688_x_at, 207934_at, 204177_s_at,
210034_s_at, 238505_at, 223357_s_at, 238037_at, 234472_at, 202695_s_at, 204014_at,
200838_at, 228712_at, 227449_at, 1560752_at, 237093_at, 210981_s_at, 205938_at, 226013_at,
229164_s_at, 222603_at, 1565628_at, 222556_at, 204300_at, 210156_s_at, 218493_at,
239716_at, 204708_at, 217340_at,
KEGG_PATHWAY hsa04340: Hedgehog signaling pathway 7 1.62% 6.95E−04
      82 57 4214 6.311082585 0.067479156
1556037_s_at, 240221_at, 1563620_at, 208570_at, 223709_s_at, 206213_at, 202332_at,
GOTERM_BP_ALL GO: 0006950~response to stress 33 7.64% 7.34E−04 251
      1081 15360 1.868124173 0.723522817
221667_s_at, 243160_at, 208698_s_at, 233351_at, 240171_at, 237548_at, 205900_at,
214955_at, 216889_s_at, 213893_x_at, 200956_s_at, 1555349_a_at, 205774_at, 208884_s_at,
229408_at, 226200_at, 206153_at, 224683_at, 235004_at, 203045_at, 240998_at, 242868_at,
211016_x_at, 211180_x_at, 200838_at, 205075_at, 209960_at, 1560587_s_at, 235885_at,
228831_s_at, 218585_s_at, 236098_at, 202332_at,
SP_PIR_KEYWORDS protein biosynthesis 11 2.55% 9.14E−04 275 193
      17599 3.64746114 0.277492123
229164_s_at, 217340_at, 232063_x_at, 204300_at, 225939_at, 226200_at, 208688_x_at,
210034_s_at, 225215_s_at, 223357_s_at, 213753_x_at,
PANTHER_BP_ALL BP00063: Protein modification 56 12.96% 0.001009319 354
      3004 29414 1.548955442 0.024382529
236805_at, 212057_at, 214013_s_at, 1557248_at, 244820_at, 235792_x_at, 216889_s_at,
205885_s_at, 243636_s_at, 202931_x_at, 205774_at, 205380_at, 205388_at, 208476_s_at,
227928_at, 243118_at, 242426_at, 226410_at, 204385_at, 244440_at, 220364_at, 211925_s_at,
237942_at, 213038_at, 203099_s_at, 226295_at, 1555404_a_at, 207374_at, 1565735_at,
230009_at, 231713_s_at, 1569025_s_at, 1554778_at, 213949_s_at, 214420_s_at, 237311_at,
208039_at, 220120_s_at, 203045_at, 218416_s_at, 214914_at, 238037_at, 229438_at,
230966_at, 1554957_at, 226125_at, 228712_at, 213444_at, 237093_at, 226013_at, 218493_at,
234741_at, 239357_at, 210156_s_at, 1552531_a_at, 221766_s_at, 214595_at,
GOTERM_BP_ALL GO: 0044260~cellular macromolecule metabolic process 80 18.52%
      0.001042123 251 3534 15360 1.385290755 0.745776661
236805_at, 221667_s_at, 243160_at, 200783_s_at, 216251_s_at, 237548_at, 217340_at,
1563620_at, 1557248_at, 214955_at, 206035_at, 200991_s_at, 205774_at, 220233_at,
206074_s_at, 206286_s_at, 202415_s_at, 230239_at, 240998_at, 211016_x_at, 201959_s_at,
237182_at, 1552703_s_at, 224330_s_at, 235788_at, 1552703_s_at, 209960_at, 213753_x_at,
217340_at, 237942_at, 213038_at, 219919_s_at, 218585_s_at, 208255_s_at, 243414_at,
202332_at, 219960_s_at, 1565735_at, 211208_s_at, 205900_at, 205655_at, 1554890_a_at,
225215_s_at, 1555349_a_at, 240221_at, 232063_x_at, 208884_s_at, 1559633_a_at,
213949_s_at, 225939_at, 226200_at, 224683_at, 208688_x_at, 207934_at, 204177_s_at,
210034_s_at, 238505_at, 223357_s_at, 238037_at, 234472_at, 202695_s_at, 204014_at,
200838_at, 228712_at, 227449_at, 1560752_at, 237093_at, 210981_s_at, 205938_at, 226013_at,
229164_s_at, 222603_at, 1565628_at, 222556_at, 204300_at, 210156_s_at, 218493_at,
239716_at, 204708_at, 217340_at,
PANTHER_BP_ALL BP00064: Protein phosphorylation 47 10.88% 0.001235566
      354 2414 29414 1.617750036 0.026832689
204029_at, 230009_at, 200783_s_at, 212057_at, 1565735_at, 211208_s_at, 235792_x_at,
243636_s_at, 1552564_at, 1569025_s_at, 1561365_at, 205774_at, 213949_s_at, 226200_at,
205388_at, 1554740_a_at, 214420_s_at, 227928_at, 208039_at, 237311_at, 220120_s_at,
203045_at, 236613_at, 214914_at, 229438_at, 204385_at, 230966_at, 226125_at, 204014_at,
228712_at, 244440_at, 237093_at, 206775_at, 244220_at, 1566163_at, 237942_at, 1558208_at,
234741_at, 239357_at, 211064_at, 203099_s_at, 243414_at, 1552531_a_at, 221766_s_at,
226114_at, 236098_at, 215766_at, 207976_at,
PANTHER_BP_ALL BP00193: Developmental processes 42 9.72% 0.001410684
      354 2095 29414 1.665774038 0.027838759
236805_at, 243160_at, 204029_at, 206413_s_at, 226623_at, 232422_at, 205900_at, 216669_at,
234701_at, 243636_s_at, 1557558_s_at, 1568687_s_at, 238941_at, 1569025_s_at, 236766_at,
236185_at, 208570_at, 208357_x_at, 1552461_at, 1556037_s_at, 226410_at, 214914_at,
206563_s_at, 204385_at, 209574_s_at, 230447_at, 235788_at, 205797_s_at, 214752_x_at,
211180_x_at, 213444_at, 232725_s_at, 210297_s_at, 222556_at, 204708_at, 216007_at,
203099_s_at, 221114_at, 243414_at, 221766_s_at, 225858_s_at, 1570093_at,
GOTERM_BP_ALL GO: 0048514~blood vessel morphogenesis 10 2.31%
      0.001608649 251 166 15360 3.686458983 0.815799305
240786_at, 242868_at, 1561365_at, 205900_at, 202510_s_at, 211180_x_at, 219568_x_at,
207968_s_at, 214955_at, 205885_s_at,
INTERPRO IPR013089: Kelch related 6 1.39% 0.002197975 288 57
      17845 6.522295322 0.999997794
204182_s_at, 229164_s_at, 1569794_at, 1554740_a_at, 204177_s_at, 207976_at,
PANTHER_MF_ALL MF00262: Non-motor actin binding protein 66 15.28%
      0.00247099 354 3837 29414 1.42923276 0.111534517
243160_at, 224881_at, 215319_at, 236805_at, 240171_at, 1554681_a_at, 234701_at,
243636_s_at, 216889_s_at, 205885_s_at, 240612_at, 202638_s_at, 1561365_at, 208570_at,
1554740_a_at, 208357_x_at, 227928_at, 218980_at, 230239_at, 243118_at, 242426_at,
240998_at, 204385_at, 201959_s_at, 205527_s_at, 1552703_s_at, 233722_at, 241849_at,
211925_s_at, 216007_at, 218585_s_at, 230988_at, 226295_at, 219419_at, 232802_at,
219960_s_at, 239719_at, 240966_at, 1553322_s_at, 1554778_at, 226200_at, 218831_s_at,
218070_s_at, 204177_s_at, 210034_s_at, 238505_at, 1552461_at, 220120_s_at, 1560997_at,
212793_at, 234480_at, 230966_at, 226125_at, 204014_at, 214752_x_at, 1560752_at, 227449_at,
226013_at, 206775_at, 206190_at, 1556282_at, 210297_s_at, 1557300_s_at, 232187_at,
216242_x_at, 207976_at,
SP_PIR_KEYWORDS cytoplasm 57 13.19% 0.002473566 275 2485 17599
      1.467924639 0.483160037
200783_s_at, 233351_at, 237548_at, 1563620_at, 218744_s_at, 235792_x_at, 1568949_at,
200991_s_at, 202931_x_at, 229408_at, 205380_at, 231548_at, 1554740_a_at, 208476_s_at,
235004_at, 227928_at, 230239_at, 222826_at, 211016_x_at, 204385_at, 205527_s_at,
236388_at, 1552703_s_at, 244440_at, 244220_at, 219919_s_at, 1560587_s_at, 230988_at,
225858_s_at, 202332_at, 1570093_at, 242913_at, 237905_at, 210330_at, 240221_at, 211740_at,
231713_s_at, 232063_x_at, 1554778_at, 210034_s_at, 234544_at, 237311_at, 220120_s_at,
1556037_s_at, 238037_at, 214752_x_at, 228712_at, 213361_at, 205938_at, 229164_s_at,
1565628_at, 1556282_at, 210156_s_at, 236713_at, 206973_at, 215766_at, 236098_at,
GOTERM_BP_ALL GO: 0048646~anatomical structure formation 10 2.31%
      0.002688991 251 179 15360 3.418727325 0.905374754
240786_at, 233318_at, 242868_at, 1561365_at, 234741_at, 239357_at, 205900_at, 202510_s_at,
211180_x_at, 219568_x_at, 214955_at,
GOTERM_BP_ALL GO: 0043412~biopolymer modification 47 10.88% 0.002840542
      251 1877 15360 1.532325679 0.881762947
236805_at, 219960_s_at, 1565735_at, 216251_s_at, 237548_at, 1563620_at, 211208_s_at,
1557248_at, 235792_x_at, 1555349_a_at, 240221_at, 208884_s_at, 1559633_a_at, 213949_s_at,
220233_at, 224683_at, 207934_at, 204177_s_at, 206286_s_at, 238505_at, 230239_at,
240998_at, 234472_at, 202695_s_at, 201959_s_at, 235788_at, 204014_at, 228712_at,
227449_at, 1560752_at, 237093_at, 210981_s_at, 205938_at, 226013_at, 1565628_at,
222556_at, 237942_at, 213038_at, 219919_s_at, 210156_s_at, 218493_at, 239716_at,
204708_at, 218585_s_at, 243414_at, 202332_at, 213835_x_at,
PANTHER_BP_ALL BP00248: Mesoderm development 22 5.09% 0.003293448
      354 905 29414 2.019877017 0.058686967
231954_at, 206563_s_at, 229438_at, 237548_at, 214013_s_at, 204014_at, 202510_s_at,
236388_at, 211180_x_at, 205527_s_at, 227449_at, 1557558_s_at, 201819_at, 1561365_at,
220129_at, 243142_at, 228831_s_at, 218070_s_at, 231147_at, 221766_s_at, 208039_at,
1570093_at,
GOTERM_BP_ALL GO: 0009887~organ morphogenesis 16 3.70% 0.003345954
      251 415 15360 2.359333749 0.889323096
1556037_s_at, 240786_at, 233318_at, 242868_at, 211194_s_at, 205900_at, 202510_s_at,
219568_x_at, 211180_x_at, 214955_at, 205885_s_at, 1561365_at, 234741_at, 239357_at,
221114_at, 219683_at, 207968_s_at,
PANTHER_MF_ALL MF00202: Other miscellaneous function protein 45 10.42%
      0.003353376 354 2402 29414 1.556647709 0.125235552
220434_at, 200783_s_at, 235098_at, 1554681_a_at, 214915_at, 216669_at, 214955_at,
229035_s_at, 238045_at, 240612_at, 227420_at, 229444_at, 222389_s_at, 1569025_s_at,
231713_s_at, 1561365_at, 236185_at, 205380_at, 226200_at, 220233_at, 206286_s_at,
226986_at, 243118_at, 240646_at, 233318_at, 218416_s_at, 206180_x_at, 235955_at,
212793_at, 230447_at, 236388_at, 1560752_at, 222756_s_at, 241448_at, 1554690_a_at,
219650_at, 240172_at, 1565628_at, 1556282_at, 231338_at, 206413_s_at, 232187_at,
218585_s_at, 226295_at, 213835_x_at,
PANTHER_MF_ALL MF00284: Other ligase 15 3.47% 0.003698002 354 510
      29414 2.443835161 0.118820913
218416_s_at, 209574_s_at, 1557248_at, 205655_at, 1552703_s_at, 213444_at, 240172_at,
236905_at, 1554778_at, 204300_at, 226200_at, 218493_at, 218475_at, 205665_at, 220120_s_at,
GOTERM_BP_ALL GO: 0001568~blood vessel development 10 2.31% 0.003726466
      251 188 15360 3.255064847 0.886900998
240786_at, 242868_at, 1561365_at, 205900_at, 202510_s_at, 211180_x_at, 219568_x_at,
207968_s_at, 214955_at, 205885_s_at,
GOTERM_BP_ALL GO: 0006464~protein modification process 45 10.42%
      0.003871296 251 1804 15360 1.526488282 0.869701352
236805_at, 219960_s_at, 1565735_at, 216251_s_at, 237548_at, 1563620_at, 211208_s_at,
1557248_at, 1555349_a_at, 240221_at, 208884_s_at, 1559633_a_at, 213949_s_at, 220233_at,
224683_at, 207934_at, 204177_s_at, 206286_s_at, 238505_at, 230239_at, 240998_at,
234472_at, 202695_s_at, 201959_s_at, 235788_at, 204014_at, 228712_at, 227449_at,
1560752_at, 237093_at, 210981_s_at, 205938_at, 226013_at, 1565628_at, 222556_at,
237942_at, 213038_at, 219919_s_at, 210156_s_at, 218493_at, 239716_at, 204708_at,
218585_s_at, 243414_at, 202332_at,
GOTERM_BP_ALL GO: 0045893~positive regulation of transcription, DNA-dependent
      12 2.78% 0.003954692 251 263 15360 2.792177298 0.849327954
240786_at, 242868_at, 1553322_s_at, 211194_s_at, 231548_at, 221517_s_at, 211180_x_at,
207968_s_at, 206074_s_at, 216889_s_at, 235473_at, 206035_at,
GOTERM_BP_ALL GO: 0001944~vasculature development 10 2.31% 0.004134537
      251 191 15360 3.203938174 0.836997662
240786_at, 242868_at, 1561365_at, 205900_at, 202510_s_at, 211180_x_at, 219568_x_at,
207968_s_at, 214955_at, 205885_s_at,
SP_PIR_KEYWORDS Wnt signaling pathway 7 1.62% 0.004296821 275 98
      17599 4.571168831 0.600705518
223122_s_at, 240221_at, 1563620_at, 208570_at, 219683_at, 223709_s_at, 206213_at,
PANTHER_MF_ALL MF00039: Other transcription factor 27 6.25% 0.004573923
      354 1243 29414 1.804859757 0.127994374
226623_at, 205900_at, 240900_at, 216889_s_at, 205885_s_at, 206035_at, 1561365_at,
1569025_s_at, 1553322_s_at, 1554778_at, 205388_at, 231548_at, 203022_at, 205665_at,
213442_x_at, 220120_s_at, 214914_at, 230966_at, 211194_s_at, 204118_at, 211180_x_at,
227449_at, 206426_at, 211925_s_at, 216242_x_at, 207968_s_at, 1570093_at,
PANTHER_MF_ALL MF00222: Zinc finger transcription factor 49 11.34%
      0.006514236 354 2775 29414 1.467181758 0.159329136
215319_at, 220434_at, 226623_at, 1569794_at, 1557248_at, 244820_at, 216889_s_at,
1568949_at, 243636_s_at, 240612_at, 1553172_at, 227694_at, 204182_s_at, 222389_s_at,
234251_at, 236185_at, 233467_s_at, 1554345_a_at, 206972_s_at, 243118_at, 242426_at,
226410_at, 205797_s_at, 1553450_s_at, 239834_at, 233754_x_at, 233722_at, 1565748_at,
223792_at, 213038_at, 226114_at, 232213_at, 219419_at, 1570093_at, 235815_at, 229035_s_at,
226200_at, 233937_at, 238505_at, 237311_at, 212684_at, 230966_at, 1554821_a_at, 222556_at,
220129_at, 234741_at, 239357_at, 211064_at, 216242_x_at, 221766_s_at,
GOTERM_BP_ALL GO: 0006366~transcription from RNA polymerase II promoter 21
      4.86% 0.006617744 251 669 15360 1.920926161 0.931674736
242868_at, 211194_s_at, 219568_x_at, 211180_x_at, 205655_at, 211771_s_at, 216889_s_at,
206035_at, 226013_at, 231713_s_at, 1566163_at, 1553322_s_at, 229408_at, 1558208_at,
231548_at, 207253_s_at, 221517_s_at, 207968_s_at, 235473_at, 214883_at, 217415_at,
GOTERM_BP_ALL GO: 0045944~positive regulation of transcription from RNA polymerase
II promoter 9 2.08% 0.007001073 251 171 15360 3.220801007
      0.928398772
242868_at, 1553322_s_at, 211194_s_at, 231548_at, 221517_s_at, 211180_x_at, 207968_s_at,
216889_s_at, 235473_at,
INTERPRO IPR013032: EGF-like region 13 3.01% 0.007052949 288 328
      17845 2.455803269 0.999999999
204029_at, 240786_at, 1556037_s_at, 242426_at, 1560997_at, 240171_at, 211571_s_at,
1557558_s_at, 1555349_a_at, 1552925_at, 206775_at, 205774_at, 206286_s_at,
GOTERM_BP_ALL GO: 0045941~positive regulation of transcription13 3.01%
      0.007256039 251 326 15360 2.440300149 0.921981888
240786_at, 242868_at, 211194_s_at, 211180_x_at, 211771_s_at, 216889_s_at, 206035_at,
1553322_s_at, 231548_at, 207968_s_at, 221517_s_at, 206074_s_at, 235473_at,
GOTERM_BP_ALL GO: 0043170~macromolecule metabolic process 138 31.94%
      0.007508649 251 7216 15360 1.170307683 0.915833306
221667_s_at, 200783_s_at, 216251_s_at, 217340_at, 237548_at, 1557248_at, 200956_s_at,
200991_s_at, 234251_at, 229408_at, 1554345_a_at, 207253_s_at, 206074_s_at, 206286_s_at,
236613_at, 240998_at, 201959_s_at, 224330_s_at, 232857_at, 211180_x_at, 205527_s_at,
209960_at, 213753_x_at, 217340_at, 237942_at, 223792_at, 218585_s_at, 203099_s_at,
207968_s_at, 208255_s_at, 243414_at, 204913_s_at, 202332_at, 214883_at, 213835_x_at,
219960_s_at, 208698_s_at, 211208_s_at, 216669_at, 235815_at, 225215_s_at, 1555349_a_at,
240221_at, 231713_s_at, 232063_x_at, 208884_s_at, 213949_s_at, 226200_at, 225939_at,
224683_at, 207934_at, 208688_x_at, 210034_s_at, 217621_at, 213442_x_at, 208710_s_at,
238037_at, 206180_x_at, 204014_at, 200838_at, 227449_at, 213444_at, 226013_at,
229164_s_at, 1565628_at, 1565537_at, 222556_at, 204300_at, 211212_s_at, 1557300_s_at,
1558208_at, 210156_s_at, 218493_at, 236098_at, 236805_at, 243160_at, 1563620_at,
238276_at, 214955_at, 235792_x_at, 216889_s_at, 1553172_at, 206035_at, 204182_s_at,
205774_at, 231548_at, 220233_at, 202415_s_at, 230239_at, 240786_at, 242868_at,
211016_x_at, 237182_at, 1552703_s_at, 235788_at, 219568_x_at, 1552703_s_at, 233722_at,
233754_x_at, 241849_at, 1558000_at, 213038_at, 219919_s_at, 226114_at, 219419_at,
1565735_at, 205900_at, 214915_at, 205655_at, 1554890_a_at, 213893_x_at, 1557558_s_at,
1559633_a_at, 1553322_s_at, 203022_at, 221517_s_at, 204177_s_at, 238505_at, 223357_s_at,
217415_at, 212684_at, 234472_at, 202695_s_at, 211194_s_at, 228712_at, 1560752_at,
211771_s_at, 237093_at, 210981_s_at, 205938_at, 222603_at, 1566163_at, 230492_s_at,
211064_at, 239716_at, 204708_at, 235473_at, 217340_at,
GOTERM_BP_ALL GO: 0031325~positive regulation of cellular metabolic process 15
      3.47% 0.008385303 251 416 15360 2.206558382 0.925910271
240786_at, 242868_at, 1565735_at, 211194_s_at, 211180_x_at, 211771_s_at, 216889_s_at,
206035_at, 1553322_s_at, 231548_at, 221517_s_at, 207968_s_at, 206074_s_at, 206286_s_at,
235473_at,
GOTERM_BP_ALL GO: 0043687~post-translational protein modification 38 8.80%
      0.008568362 251 1522 15360 1.527870123 0.918876771
236805_at, 1565735_at, 219960_s_at, 237548_at, 1563620_at, 211208_s_at, 1557248_at,
1555349_a_at, 240221_at, 208884_s_at, 220233_at, 224683_at, 207934_at, 204177_s_at,
206286_s_at, 238505_at, 230239_at, 240998_at, 202695_s_at, 201959_s_at, 235788_at,
204014_at, 228712_at, 227449_at, 1560752_at, 237093_at, 210981_s_at, 205938_at,
1565628_at, 237942_at, 213038_at, 219919_s_at, 210156_s_at, 239716_at, 204708_at,
218585_s_at, 243414_at, 202332_at,
PANTHER_BP_ALL BP00040: mRNA transcription 100 23.15% 0.008587166 354
      6626 29414 1.254005365 0.135798064
212057_at, 216251_s_at, 235098_at, 211571_s_at, 1557248_at, 244820_at, 200956_s_at,
205885_s_at, 227694_at, 222389_s_at, 1561365_at, 234251_at, 229408_at, 1554345_a_at,
208570_at, 242426_at, 232857_at, 217340_at, 223792_at, 218585_s_at, 219683_at,
208255_s_at, 204913_s_at, 201079_at, 1570093_at, 239719_at, 242913_at, 235815_at,
1555349_a_at, 240221_at, 229402_at, 236766_at, 233937_at, 213442_x_at, 234544_at,
208710_s_at, 240646_at, 206180_x_at, 228375_at, 227449_at, 213361_at, 213444_at,
226013_at, 1557300_s_at, 211212_s_at, 218493_at, 234741_at, 239357_at, 1558208_at,
218475_at, 216242_x_at, 214013_s_at, 226623_at, 1569794_at, 218429_s_at, 216889_s_at,
1556017_at, 204182_s_at, 205774_at, 208357_x_at, 218980_at, 235004_at, 227928_at,
240786_at, 204385_at, 219568_x_at, 1554690_a_at, 233754_x_at, 233722_at, 228403_at,
1552925_at, 1558000_at, 231147_at, 226114_at, 214915_at, 205655_at, 229035_s_at,
211740_at, 1553322_s_at, 1559633_a_at, 206153_at, 221517_s_at, 214420_s_at, 204177_s_at,
238505_at, 217415_at, 1556037_s_at, 211422_at, 214914_at, 218416_s_at, 212684_at,
234472_at, 211194_s_at, 208389_s_at, 214752_x_at, 211771_s_at, 205938_at, 1554821_a_at,
211064_at, 239716_at, 235473_at,
GOTERM_BP_ALL GO: 0042730~fibrinolysis 3 0.69% 0.008808741 251
      9 15360 20.39840637 0.913416023
205774_at, 205900_at, 214955_at,
PANTHER_BP_ALL BP00077: Oxidative phosphorylation 30 6.94% 0.008868851
      354 1508 29414 1.652991953 0.130632511
216251_s_at, 233543_s_at, 235098_at, 211208_s_at, 240966_at, 229444_at, 222389_s_at,
1554778_at, 236185_at, 226200_at, 1554345_a_at, 208570_at, 237311_at, 203045_at,
225484_at, 208389_s_at, 236388_at, 1560752_at, 227449_at, 213753_x_at, 1552925_at,
1565628_at, 220129_at, 211925_s_at, 204300_at, 206413_s_at, 1552531_a_at, 208255_s_at,
206213_at, 1555404_a_at,
GOTERM_BP_ALL GO: 0001525~angiogenesis 8 1.85% 0.008953196 251
      143 15360 3.423508762 0.905824664
240786_at, 242868_at, 1561365_at, 205900_at, 202510_s_at, 211180_x_at, 219568_x_at,
214955_at,
GOTERM_CC_ALL GO: 0009986~cell surface 9 2.08% 0.009037217 271
      171 15857 3.079627112 0.999621754
1556037_s_at, 240786_at, 202638_s_at, 206775_at, 200838_at, 204177_s_at, 206973_at,
206286_s_at, 203167_at,
GOTERM_BP_ALL GO: 0045935~positive regulation of nucleobase, nucleoside, nucleotide
and nucleic acid metabolic process 13 3.01% 0.009141103 251 336 15360
      2.367672168 0.899493612
240786_at, 242868_at, 211194_s_at, 211180_x_at, 211771_s_at, 216889_s_at, 206035_at,
1553322_s_at, 231548_at, 207968_s_at, 221517_s_at, 206074_s_at, 235473_at,
GOTERM_CC_ALL GO: 0019898~extrinsic to membrane 6 1.39% 0.010065592
      271 77 15857 4.559447932 0.987606722
206775_at, 228831_s_at, 208476_s_at, 222756_s_at, 206286_s_at, 220120_s_at,
SP_PIR_KEYWORDS alternative promoter usage 4 0.93% 0.010113736 275
      29 17599 8.827084639 0.835693568
211194_s_at, 230966_at, 228712_at, 216889_s_at,
PANTHER_BP_ALL BP00150: MHCI-mediated immunity 84 19.44% 0.010506288
      354 5445 29414 1.281835302 0.143505519
204029_at, 243160_at, 212057_at, 233543_s_at, 216251_s_at, 240171_at, 226623_at,
218429_s_at, 207566_at, 211571_s_at, 238801_at, 216889_s_at, 215112_x_at, 1568687_s_at,
1553172_at, 222389_s_at, 1561365_at, 205774_at, 236185_at, 233467_s_at, 231548_at,
1554740_a_at, 244029_at, 205665_at, 208357_x_at, 206074_s_at, 218980_at, 230239_at,
242426_at, 242868_at, 211016_x_at, 204385_at, 1553450_s_at, 211180_x_at, 228403_at,
233722_at, 203167_at, 209960_at, 1558000_at, 220364_at, 1552925_at, 213038_at, 201079_at,
1570093_at, 219960_s_at, 239719_at, 211208_s_at, 229035_s_at, 225215_s_at, 218714_at,
208884_s_at, 1554778_at, 226200_at, 218831_s_at, 238581_at, 218070_s_at, 208688_x_at,
210034_s_at, 214420_s_at, 234544_at, 208710_s_at, 220120_s_at, 211422_at, 212684_at,
212793_at, 234480_at, 208334_at, 230966_at, 200838_at, 214752_x_at, 227449_at, 213444_at,
1556282_at, 210156_s_at, 234741_at, 239357_at, 218493_at, 211064_at, 232187_at, 221114_at,
206973_at, 236098_at, 214595_at, 1569826_at, 215766_at,
INTERPRO IPR006210: EGF 10 2.31% 0.011167962 288 226 17845
      2.741672812 1
1556037_s_at, 240786_at, 204029_at, 206775_at, 242426_at, 205774_at, 240171_at,
211571_s_at, 206286_s_at, 1557558_s_at,
GOTERM_BP_ALL GO: 0009611~response to wounding 15 3.47% 0.011206485
      251 431 15360 2.129764007 0.932215478
203045_at, 243160_at, 205900_at, 200838_at, 214955_at, 216889_s_at, 205075_at, 209960_at,
1555349_a_at, 205774_at, 229408_at, 1560587_s_at, 235885_at, 206153_at, 235004_at,
GOTERM_BP_ALL GO: 0006357~regulation of transcription from RNA polymerase II
promoter 16 3.70% 0.011358525 251 476 15360 2.056982155
      0.926430929
242868_at, 211194_s_at, 219568_x_at, 211180_x_at, 205655_at, 216889_s_at, 226013_at,
231713_s_at, 1566163_at, 1553322_s_at, 229408_at, 231548_at, 207253_s_at, 221517_s_at,
207968_s_at, 235473_at,
SP_PIR_KEYWORDS DNA binding 12 2.78% 0.01216951 275 321 17599
      2.392387426 0.845043572
212684_at, 1565537_at, 1553322_s_at, 219568_x_at, 207968_s_at, 204913_s_at, 211771_s_at,
206074_s_at, 200956_s_at, 216889_s_at, 206035_at, 217415_at,
PANTHER_MF_ALL MF00283: Ubiquitin-protein ligase 21 4.86% 0.012468189
      354 959 29414 1.819497711 0.259080277
212057_at, 233543_s_at, 201959_s_at, 1563620_at, 235788_at, 237905_at, 238801_at,
205655_at, 213250_at, 222389_s_at, 238941_at, 1561365_at, 232725_s_at, 208884_s_at,
1554778_at, 213038_at, 205388_at, 217621_at, 207934_at, 217621_at, 235004_at,
GOTERM_MF_ALL GO: 0004674~protein serine/threonine kinase activity 15 3.47%
      0.012635306 270 449 16968 2.099480327 0.999994963
221667_s_at, 220434_at, 240998_at, 237548_at, 202695_s_at, 211208_s_at, 228712_at,
237093_at, 210981_s_at, 240221_at, 1565628_at, 237942_at, 204708_at, 202332_at, 230239_at,
PANTHER_MF_ALL MF00215: Cysteine protease 13 3.01% 0.012880988 354
      475 29414 2.274052929 0.245490091
219960_s_at, 233351_at, 240171_at, 1563620_at, 1552703_s_at, 200838_at, 1552703_s_at,
211925_s_at, 207253_s_at, 221766_s_at, 208357_x_at, 1555404_a_at, 227928_at,
PANTHER_BP_ALL BP00072: Protein complex assembly 9 2.08% 0.013151694
      354 258 29414 2.898502168 0.166428232 218493_at, 228712_at,
230988_at, 223709_s_at, 206213_at, 226986_at, 243118_at, 233722_at, 220120_s_at,
PANTHER_MF_ALL MF00264: Microtubule family cytoskeletal protein 20 4.63%
      0.013353892 354 902 29414 1.842359102 0.234906182 220434_at,
206180_x_at, 230966_at, 226623_at, 211208_s_at, 240966_at, 238045_at, 240612_at,
1568687_s_at, 227694_at, 236905_at, 222389_s_at, 1561365_at, 229402_at, 1554778_at,
234741_at, 239357_at, 221766_s_at, 226295_at, 206286_s_at, 217415_at,
SP_PIR_KEYWORDS egf-like domain 10 2.31% 0.01349376 275 241 17599
      2.655450773 0.836392486 1556037_s_at, 240786_at, 204029_at, 206775_at,
242426_at, 205774_at, 240171_at, 211571_s_at, 206286_s_at, 1557558_s_at,
GOTERM_BP_ALL GO: 0030154~cell differentiation 43 9.95% 0.013860152
      251 1835 15360 1.43400241 0.952898106 200783_s_at, 240171_at,
237548_at, 205655_at, 1554890_a_at, 201819_at, 1555349_a_at, 223122_s_at, 1561365_at,
202931_x_at, 229408_at, 231548_at, 208570_at, 233937_at, 206286_s_at, 230239_at,
208710_s_at, 237311_at, 1556037_s_at, 240786_at, 233318_at, 222826_at, 242868_at,
212684_at, 202695_s_at, 211194_s_at, 1552703_s_at, 202510_s_at, 211180_x_at, 200838_at,
219568_x_at, 1552703_s_at, 211771_s_at, 203167_at, 213753_x_at, 209960_at, 241849_at,
237942_at, 234741_at, 239357_at, 208255_s_at, 207968_s_at, 225858_s_at, 1570093_at,
GOTERM_BP_ALL GO: 0048869~cellular developmental process 43 9.95%
      0.013860152 251 1835 15360 1.43400241 0.952898106 200783_s_at,
240171_at, 237548_at, 205655_at, 1554890_a_at, 201819_at, 1555349_a_at, 223122_s_at,
1561365_at, 202931_x_at, 229408_at, 231548_at, 208570_at, 233937_at, 206286_s_at,
230239_at, 208710_s_at, 237311_at, 1556037_s_at, 240786_at, 233318_at, 222826_at,
242868_at, 212684_at, 202695_s_at, 211194_s_at, 1552703_s_at, 202510_s_at, 211180_x_at,
200838_at, 219568_x_at, 1552703_s_at, 211771_s_at, 203167_at, 213753_x_at, 209960_at,
241849_at, 237942_at, 234741_at, 239357_at, 208255_s_at, 207968_s_at, 225858_s_at,
1570093_at,
INTERPRO IPR000210: BTB/POZ-like 8 1.85% 0.014114397 288 158
      17845 3.13730661 0.999999999 204182_s_at, 229164_s_at, 1569794_at,
1554740_a_at, 204177_s_at, 239834_at, 214595_at, 207976_at,
PANTHER_BP_ALL BP00301: Non-vertebrate process 10 2.31% 0.014393571
      354 315 29414 2.637790333 0.171073087
243160_at, 206775_at, 202695_s_at, 214752_x_at, 228712_at, 226995_at, 223709_s_at,
206213_at, 226986_at, 1555393_s_at,
GOTERM_BP_ALL GO: 0009893~positive regulation of metabolic process 15 3.47%
      0.014417764 251 445 15360 2.062760195 0.946853579
240786_at, 242868_at, 1565735_at, 211194_s_at, 211180_x_at, 211771_s_at, 216889_s_at,
206035_at, 1553322_s_at, 231548_at, 221517_s_at, 207968_s_at, 206074_s_at, 206286_s_at,
235473_at,
PANTHER_MF_ALL MF00115: Carbohydrate phosphatase 5 1.16% 0.014510196
      354 78 29414 5.326307403 0.235641766
206775_at, 234480_at, 226200_at, 211771_s_at, 240900_at,
INTERPRO IPR006209: EGF-like 8 1.85% 0.014568391 288 159 17845
      3.117575122 0.999999971
240786_at, 204029_at, 206775_at, 242426_at, 205774_at, 240171_at, 211571_s_at,
1557558_s_at,
INTERPRO IPR001660: Sterile alpha motif SAM 6 1.39% 0.015061757 288
      90 17845 4.130787037 0.999999685
229402_at, 211194_s_at, 237548_at, 227449_at, 244440_at, 206973_at,
GOTERM_BP_ALL GO: 0065007~biological regulation 103 23.84% 0.015277567
      251 5238 15360 1.203342415 0.950005618
240171_at, 237548_at, 1557248_at, 200956_s_at, 202638_s_at, 200991_s_at, 1561365_at,
234251_at, 202931_x_at, 229408_at, 205388_at, 217621_at, 207253_s_at, 206074_s_at,
206286_s_at, 222826_at, 201959_s_at, 211180_x_at, 209960_at, 213753_x_at, 221066_at,
237942_at, 223792_at, 203099_s_at, 207968_s_at, 204913_s_at, 214883_at, 208698_s_at,
235815_at, 1555349_a_at, 231713_s_at, 226200_at, 225939_at, 213442_x_at, 234544_at,
208710_s_at, 208039_at, 233318_at, 206180_x_at, 204014_at, 200838_at, 222756_s_at,
213444_at, 226013_at, 206775_at, 1565537_at, 1557300_s_at, 234741_at, 239357_at,
1558208_at, 235885_at, 232187_at, 243160_at, 214013_s_at, 238276_at, 218744_s_at,
214955_at, 216889_s_at, 215112_x_at, 1553172_at, 206035_at, 204182_s_at, 205774_at,
231548_at, 230239_at, 240786_at, 242868_at, 1552703_s_at, 219568_x_at, 1552703_s_at,
233754_x_at, 241849_at, 203167_at, 1558000_at, 211925_s_at, 225858_s_at, 226114_at,
1565735_at, 205900_at, 214915_at, 205655_at, 1554890_a_at, 1553322_s_at, 1559633_a_at,
221517_s_at, 223357_s_at, 217415_at, 1556037_s_at, 212684_at, 1560997_at, 206563_s_at,
211194_s_at, 202695_s_at, 226125_at, 214752_x_at, 228712_at, 211771_s_at, 210981_s_at,
1566163_at, 220129_at, 211064_at, 239716_at, 228831_s_at, 235473_at,
INTERPRO IPR011333: BTB/POZ fold 8 1.85% 0.015507501 288 161
      17845 3.078847481 0.999998178
204182_s_at, 229164_s_at, 1569794_at, 1554740_a_at, 204177_s_at, 239834_at, 214595_at,
207976_at,
GOTERM_BP_ALL GO: 0030195~negative regulation of blood coagulation 3 0.69%
      0.015638798 251 12 15360 15.29880478 0.948061606
205774_at, 205900_at, 214955_at,
PANTHER_BP_ALL BP00076: Electron transport 22 5.09% 0.016143976 354
      1049 29414 1.74260124 0.180388917
209163_at, 203045_at, 220434_at, 230009_at, 239719_at, 206563_s_at, 235098_at, 234480_at,
230966_at, 1563620_at, 211208_s_at, 216669_at, 234701_at, 243636_s_at, 205774_at,
204300_at, 236185_at, 210156_s_at, 226200_at, 206153_at, 214420_s_at, 218980_at,
PANTHER_BP_ALL BP00199: Neurogenesis 23 5.32% 0.016161339 354 1116
      29414 1.712436466 0.171931938
218416_s_at, 219960_s_at, 214914_at, 239719_at, 206413_s_at, 1563620_at, 211208_s_at,
230447_at, 203164_at, 234701_at, 218557_at, 238801_at, 216889_s_at, 213250_at, 205075_at,
206035_at, 205938_at, 240221_at, 1554778_at, 208688_x_at, 235004_at, 243118_at,
1570093_at,
GOTERM_BP_ALL GO: 0044238~primary metabolic process 153 35.42%
      0.016738987 251 8291 15360 1.12928097 0.953033052
221667_s_at, 200783_s_at, 216251_s_at, 240171_at, 217340_at, 237548_at, 1557248_at,
200956_s_at, 200991_s_at, 234251_at, 229408_at, 1554345_a_at, 207253_s_at, 206074_s_at,
206286_s_at, 236613_at, 240998_at, 201959_s_at, 224330_s_at, 232857_at, 211180_x_at,
205527_s_at, 209960_at, 213753_x_at, 217340_at, 237942_at, 223792_at, 218585_s_at,
203099_s_at, 207968_s_at, 208255_s_at, 243414_at, 204913_s_at, 202332_at, 214883_at,
213835_x_at, 219960_s_at, 208698_s_at, 211208_s_at, 216669_at, 36994_at, 225215_s_at,
235815_at, 1555349_a_at, 240221_at, 232063_x_at, 231713_s_at, 208884_s_at, 213949_s_at,
225939_at, 226200_at, 224683_at, 207934_at, 208688_x_at, 210034_s_at, 217621_at,
213442_x_at, 208710_s_at, 206180_x_at, 238037_at, 204014_at, 200838_at, 227449_at,
213444_at, 226013_at, 229164_s_at, 206775_at, 1565628_at, 1565537_at, 222556_at,
204300_at, 211212_s_at, 1557300_s_at, 1558208_at, 210156_s_at, 234741_at, 239357_at,
218493_at, 216242_x_at, 236098_at, 236805_at, 243160_at, 1570505_at, 1563620_at,
238276_at, 214955_at, 235792_x_at, 216889_s_at, 201819_at, 1553172_at, 206035_at,
204182_s_at, 205774_at, 231548_at, 220233_at, 202415_s_at, 230239_at, 240786_at,
242868_at, 211016_x_at, 237182_at, 204385_at, 1552703_s_at, 235788_at, 219568_x_at,
1552703_s_at, 200710_at, 233722_at, 203167_at, 241849_at, 233754_x_at, 1558000_at,
211925_s_at, 213038_at, 219919_s_at, 226114_at, 219419_at, 1565735_at, 205900_at,
214915_at, 205655_at, 1554890_a_at, 213893_x_at, 1557558_s_at, 1559633_a_at,
1553322_s_at, 203022_at, 221517_s_at, 204177_s_at, 238505_at, 223357_s_at, 217415_at,
212684_at, 234472_at, 202695_s_at, 230966_at, 211194_s_at, 214752_x_at, 228712_at,
1560752_at, 211771_s_at, 237093_at, 210981_s_at, 205938_at, 222603_at, 1566163_at,
220129_at, 230492_s_at, 211064_at, 239716_at, 204708_at, 229131_at, 235473_at, 217340_at,
GOTERM_BP_ALL GO: 0016055~Wnt receptor signaling pathway 7 1.62%
      0.017080164 251 126 15360 3.399734396 0.951059337
223122_s_at, 240221_at, 1563620_at, 208570_at, 219683_at, 223709_s_at, 206213_at,
SP_PIR_KEYWORDS Serine/threonine-protein kinase 13 3.01% 0.017235741 275
      383 17599 2.172200332 0.872453691
220434_at, 240998_at, 237548_at, 202695_s_at, 228712_at, 210981_s_at, 237093_at,
240221_at, 1565628_at, 237942_at, 204708_at, 202332_at, 230239_at,
SP_PIR_KEYWORDS Coiled coil 36 8.33% 0.017950005 275 1560 17599
      1.476839161 0.854975446
225830_at, 200783_s_at, 212057_at, 233543_s_at, 208698_s_at, 205900_at, 237905_at,
218744_s_at, 235815_at, 1557558_s_at, 1553172_at, 238941_at, 222389_s_at, 211740_at,
202931_x_at, 1554778_at, 207253_s_at, 234544_at, 230239_at, 243118_at, 211422_at,
222826_at, 1560997_at, 235955_at, 212793_at, 235788_at, 236388_at, 213250_at, 229164_s_at,
1556282_at, 243142_at, 239716_at, 218475_at, 243414_at, 206973_at, 1570093_at,
GOTERM_MF_ALL GO: 0008135~translation factor activity, nucleic acid binding 7
      1.62% 0.018121812 270 131 16968 3.358100085 0.99999807
229164_s_at, 204300_at, 225939_at, 208688_x_at, 225215_s_at, 223357_s_at, 213753_x_at,
GOTERM_BP_ALL GO: 0030193~regulation of blood coagulation 3 0.69%
      0.018285889 251 13 15360 14.12197364 0.95618806
205774_at, 205900_at, 214955_at,
GOTERM_BP_ALL GO: 0042060~wound healing 7 1.62% 0.018958187 251
      129 15360 3.320670805 0.956828775
203045_at, 205774_at, 205900_at, 235885_at, 214955_at, 216889_s_at, 209960_at,
PANTHER_MF_ALL MF00053: Other RNA-binding protein 16 3.70% 0.019901241
      354 688 29414 1.932334779 0.290481564
236613_at, 226410_at, 212057_at, 226623_at, 1554890_a_at, 200956_s_at, 224108_at,
236905_at, 1555393_s_at, 1561365_at, 1565537_at, 1556282_at, 226200_at, 203022_at,
218831_s_at, 226986_at,
PANTHER_MF_ALL MF00102: Protease inhibitor 15 3.47% 0.019928375 354
      627 29414 1.987808504 0.274382305
240786_at, 234480_at, 228712_at, 205075_at, 203167_at, 202638_s_at, 211740_at, 236185_at,
213038_at, 203099_s_at, 216242_x_at, 225858_s_at, 221920_s_at, 1570093_at, 1552461_at,
PANTHER_BP_ALL BP00268: Antioxidation and free radical removal9 2.08%
      0.021238192 354 282 29414 2.651821132 0.210327237
225830_at, 210297_s_at, 206413_s_at, 205380_at, 223792_at, 1560587_s_at, 200838_at,
223296_at, 1554690_a_at,
PANTHER_BP_ALL BP00034: DNA metabolism 19 4.40% 0.021596072 354
      885 29414 1.783861598 0.204451376
226410_at, 239719_at, 206413_s_at, 200838_at, 213361_at, 213893_x_at, 222389_s_at,
1566163_at, 210297_s_at, 211212_s_at, 229131_at, 216007_at, 218585_s_at, 208476_s_at,
224683_at, 235343_at, 226295_at, 236098_at, 214883_at,
SP_PIR_KEYWORDS cytoskeleton  12 2.78% 0.021659546 275 350 17599
      2.194161039 0.880216858
219919_s_at, 236713_at, 1554740_a_at, 208476_s_at, 214752_x_at, 230988_at, 205885_s_at,
230239_at, 210330_at, 220120_s_at, 1555349_a_at, 1570093_at,
KEGG_PATHWAY hsa05217: Basal cell carcinoma 5 1.16% 0.021839586 82
      56 4214 4.588414634 0.772240988
1556037_s_at, 208570_at, 219683_at, 223709_s_at, 206213_at,
PANTHER_BP_ALL BP00141: Transport 29 6.71% 0.021889114 354 1555
      29414 1.5495958 0.198541714
239719_at, 1570505_at, 242913_at, 214915_at, 244820_at, 36994_at, 240900_at, 216889_s_at,
240612_at, 200991_s_at, 206765_at, 208476_s_at, 230571_at, 208039_at, 226125_at,
200838_at, 239834_at, 213444_at, 203167_at, 229164_s_at, 222556_at, 231338_at,
219919_s_at, 234741_at, 239357_at, 221114_at, 223296_at, 214595_at, 202332_at,
221920_s_at,
SMART SM00181: EGF 10 2.31% 0.022676127 181 227 9899
      2.409277874 0.999999102
1556037_s_at, 240786_at, 204029_at, 206775_at, 242426_at, 205774_at, 240171_at,
211571_s_at, 206286_s_at, 1557558_s_at,
GOTERM_BP_ALL GO: 0007596~blood coagulation 6 1.39% 0.022798983 251
      99 15360 3.708801159 0.974571711
205774_at, 205900_at, 235885_at, 214955_at, 216889_s_at, 209960_at,
INTERPRO IPR000742: EGF-like, type 3 9 2.08% 0.023412817 288 215
      17845 2.593750.999999976
1556037_s_at, 240786_at, 204029_at, 206775_at, 242426_at, 205774_at, 240171_at,
211571_s_at, 206286_s_at,
GOTERM_BP_ALL GO: 0010467~gene expression 75 17.36% 0.023699673 251
      3686 15360 1.245155028 0.975434009
217340_at, 238276_at, 1557248_at, 216889_s_at, 200956_s_at, 1553172_at, 206035_at,
204182_s_at, 234251_at, 229408_at, 231548_at, 207253_s_at, 206074_s_at, 240786_at,
242868_at, 236613_at, 237182_at, 201959_s_at, 224330_s_at, 232857_at, 219568_x_at,
211180_x_at, 205527_s_at, 233722_at, 233754_x_at, 213753_x_at, 1558000_at, 217340_at,
223792_at, 203099_s_at, 207968_s_at, 204913_s_at, 226114_at, 214883_at, 219419_at,
213835_x_at, 208698_s_at, 214915_at, 205655_at, 1554890_a_at, 225215_s_at, 235815_at,
232063_x_at, 231713_s_at, 1553322_s_at, 225939_at, 226200_at, 221517_s_at, 208688_x_at,
210034_s_at, 217621_at, 213442_x_at, 223357_s_at, 217415_at, 212684_at, 206180_x_at,
211194_s_at, 214752_x_at, 211771_s_at, 213444_at, 237093_at, 226013_at, 229164_s_at,
1565537_at, 1566163_at, 220129_at, 204300_at, 1557300_s_at, 1558208_at, 218493_at,
211064_at, 239716_at, 216242_x_at, 235473_at, 217340_at,
SMART SM00454: SAM 6 1.39% 0.023889197 181 90 9899
      3.646040516 0.999349896
229402_at, 211194_s_at, 237548_at, 227449_at, 244440_at, 206973_at,
INTERPRO IPR000504: RNA recognition motif, RNP-1 9 2.08% 0.023988783
      288 216 17845 2.581741898 0.999999884
236613_at, 208698_s_at, 1558208_at, 218475_at, 238801_at, 208688_x_at, 1554890_a_at,
235004_at, 233722_at,
GOTERM_BP_ALL GO: 0050819~negative regulation of coagulation 3 0.69%
      0.024096494 251 15 15360 12.23904382 0.97430663
205774_at, 205900_at, 214955_at,
INTERPRO IPR011510: Sterile alpha motif homology 2 4 0.93% 0.024566665
      288 39 17845 6.35505698 0.999999596
211194_s_at, 237548_at, 227449_at, 206973_at,
GOTERM_BP_ALL GO: 0050817~coagulation 6 1.39% 0.024606117 251
      101 15360 3.635359552 0.973643658
205774_at, 205900_at, 235885_at, 214955_at, 216889_s_at, 209960_at,
PANTHER_BP_ALL BP00242: Embryogenesis 5 1.16% 0.024979725 354
      92 29414 4.515782363 0.214921391
240221_at, 216007_at, 218585_s_at, 211771_s_at, 227694_at,
PANTHER_MF_ALL MF00137: Glycosyltransferase 26 6.02% 0.025441365 354
      1370 29414 1.576898016 0.319514135
235098_at, 1554681_a_at, 244820_at, 1557248_at, 1568949_at, 232063_x_at, 1554778_at,
226200_at, 218831_s_at, 235343_at, 205665_at, 208357_x_at, 227928_at, 1552461_at,
240786_at, 242426_at, 234472_at, 201959_s_at, 227449_at, 220364_at, 222556_at,
230492_s_at, 243142_at, 226295_at, 221920_s_at, 1570093_at,
SMART SM00225: BTB 8 1.85% 0.025949453 181 159 9899
      2.751728691 0.995106092
204182_s_at, 229164_s_at, 1569794_at, 1554740_a_at, 204177_s_at, 239834_at, 214595_at,
207976_at,
GOTERM_MF_ALL GO: 0004672~protein kinase activity 19 4.40% 0.025957363
      270 687 16968 1.73805596 0.999999734
236805_at, 221667_s_at, 220434_at, 242868_at, 240998_at, 202695_s_at, 237548_at,
211208_s_at, 228712_at, 227449_at, 237093_at, 210981_s_at, 240221_at, 1561365_at,
1565628_at, 237942_at, 204708_at, 230239_at, 202332_at,
PANTHER_BP_ALL BP00103: Cell surface receptor mediated signal transduction 45
      10.42% 0.026064859 354 2721 29414 1.374152075 0.215019866
221667_s_at, 1565735_at, 206413_s_at, 218429_s_at, 216669_at, 234701_at, 205655_at,
240966_at, 240900_at, 1568949_at, 215112_x_at, 201819_at, 202638_s_at, 229444_at,
1561365_at, 236185_at, 217621_at, 208476_s_at, 206972_s_at, 208357_x_at, 214420_s_at,
234544_at, 208039_at, 208710_s_at, 1552461_at, 233318_at, 242868_at, 208389_s_at,
204118_at, 213444_at, 205075_at, 240172_at, 206190_at, 1566163_at, 221066_at, 231338_at,
1558208_at, 223792_at, 228831_s_at, 235885_at, 239716_at, 203099_s_at, 219683_at,
214883_at, 221920_s_at,
INTERPRO IPR006651: Kelch 3 0.69% 0.026661782 288 16 17845
      11.61783854 0.999999516
1554740_a_at, 204177_s_at, 207976_at,
GOTERM_BP_ALL GO: 0050818~regulation of coagulation 3 0.69% 0.027247516
      251 16 15360 11.47410359 0.980215117
205774_at, 205900_at, 214955_at,
GOTERM_BP_ALL GO: 0007599~hemostasis 6 1.39% 0.02748541 251
      104 15360 3.530493411 0.978792957
205774_at, 205900_at, 235885_at, 214955_at, 216889_s_at, 209960_at,
PANTHER_MF_ALL MF00231: Microtubule binding motor protein 27 6.25%
      0.027616059 354 1449 29414 1.548268239 0.32545106
243160_at, 220434_at, 200783_s_at, 233543_s_at, 239719_at, 205900_at, 240966_at,
1554890_a_at, 229035_s_at, 227420_at, 222389_s_at, 202931_x_at, 229402_at, 1559633_a_at,
1554778_at, 226200_at, 221517_s_at, 207934_at, 203045_at, 1560997_at, 208334_at,
236388_at, 213444_at, 206775_at, 234741_at, 239357_at, 226295_at, 207374_at,
GOTERM_BP_ALL GO: 0050789~regulation of biological process 93 21.53%
      0.027695359 251 4759 15360 1.195872111 0.977261657
243160_at, 214013_s_at, 237548_at, 238276_at, 218744_s_at, 1557248_at, 214955_at,
216889_s_at, 200956_s_at, 215112_x_at, 206035_at, 1553172_at, 204182_s_at, 202638_s_at,
200991_s_at, 1561365_at, 234251_at, 202931_x_at, 205774_at, 229408_at, 231548_at,
205388_at, 207253_s_at, 206074_s_at, 206286_s_at, 230239_at, 240786_at, 222826_at,
242868_at, 201959_s_at, 1552703_s_at, 211180_x_at, 219568_x_at, 1552703_s_at, 203167_at,
213753_x_at, 241849_at, 233754_x_at, 1558000_at, 211925_s_at, 237942_at, 223792_at,
203099_s_at, 207968_s_at, 204913_s_at, 225858_s_at, 226114_at, 214883_at, 1565735_at,
208698_s_at, 205900_at, 214915_at, 205655_at, 1554890_a_at, 235815_at, 1555349_a_at,
231713_s_at, 1559633_a_at, 1553322_s_at, 225939_at, 226200_at, 221517_s_at, 234544_at,
213442_x_at, 208710_s_at, 223357_s_at, 217415_at, 1556037_s_at, 1560997_at, 212684_at,
206180_x_at, 202695_s_at, 211194_s_at, 226125_at, 204014_at, 200838_at, 214752_x_at,
228712_at, 211771_s_at, 213444_at, 222756_s_at, 210981_s_at, 226013_at, 1565537_at,
1566163_at, 220129_at, 1557300_s_at, 1558208_at, 211064_at, 232187_at, 239716_at,
228831_s_at, 235473_at,
GOTERM_BP_ALL GO: 0044237~cellular metabolic process 151 34.95% 0.027845137
      251 8269 15360 1.11748435 0.975506448
221667_s_at, 200783_s_at, 216251_s_at, 240171_at, 217340_at, 237548_at, 1557248_at,
200956_s_at, 200991_s_at, 234251_at, 229408_at, 1554345_a_at, 207253_s_at, 206074_s_at,
206286_s_at, 236613_at, 240998_at, 201959_s_at, 224330_s_at, 232857_at, 211180_x_at,
205527_s_at, 209960_at, 213753_x_at, 217340_at, 237942_at, 223792_at, 218585_s_at,
203099_s_at, 207968_s_at, 208255_s_at, 243414_at, 204913_s_at, 202332_at, 214883_at,
213835_x_at, 219960_s_at, 208698_s_at, 211208_s_at, 216669_at, 36994_at, 225215_s_at,
235815_at, 1555349_a_at, 240221_at, 232063_x_at, 231713_s_at, 208884_s_at, 213949_s_at,
225939_at, 226200_at, 224683_at, 207934_at, 208688_x_at, 210034_s_at, 217621_at,
213442_x_at, 208710_s_at, 206180_x_at, 238037_at, 204014_at, 200838_at, 227449_at,
213444_at, 226013_at, 229164_s_at, 206775_at, 1565628_at, 1565537_at, 222556_at,
204300_at, 211212_s_at, 1557300_s_at, 1558208_at, 210156_s_at, 234741_at, 239357_at,
218493_at, 216242_x_at, 236098_at, 215766_at, 236805_at, 243160_at, 1563620_at, 238276_at,
214955_at, 235792_x_at, 216889_s_at, 201819_at, 1553172_at, 206035_at, 204182_s_at,
205774_at, 231548_at, 220233_at, 202415_s_at, 230239_at, 240786_at, 242868_at,
211016_x_at, 237182_at, 204385_at, 1552703_s_at, 235788_at, 219568_x_at, 1552703_s_at,
200710_at, 233722_at, 203167_at, 241849_at, 233754_x_at, 1558000_at, 213038_at,
219919_s_at, 226114_at, 219419_at, 1565735_at, 205900_at, 214915_at, 205655_at,
1554890_a_at, 213893_x_at, 1559633_a_at, 1553322_s_at, 203022_at, 221517_s_at,
204177_s_at, 238505_at, 223357_s_at, 217415_at, 212684_at, 234472_at, 202695_s_at,
230966_at, 211194_s_at, 214752_x_at, 228712_at, 1560752_at, 211771_s_at, 237093_at,
210981_s_at, 205938_at, 222603_at, 1566163_at, 220129_at, 230492_s_at, 211064_at,
239716_at, 204708_at, 229131_at, 235473_at, 217340_at,
GOTERM_MF_ALL GO: 0045182~translation regulator activity 7 1.62%
      0.028147475 270 145 16968 3.033869732 0.999998872
229164_s_at, 204300_at, 225939_at, 208688_x_at, 225215_s_at, 223357_s_at, 213753_x_at,
SP_PIR_KEYWORDS Transcription 39 9.03% 0.028564917 275 1783 17599
      1.39980829 0.923805353
208698_s_at, 238276_at, 214915_at, 1557248_at, 216889_s_at, 235815_at, 200956_s_at,
1553172_at, 206035_at, 204182_s_at, 231713_s_at, 234251_at, 1553322_s_at, 229408_at,
231548_at, 221517_s_at, 206074_s_at, 213442_x_at, 217415_at, 240786_at, 242868_at,
212684_at, 211194_s_at, 232857_at, 219568_x_at, 211180_x_at, 211771_s_at, 233754_x_at,
1558000_at, 1557300_s_at, 1558208_at, 223792_at, 239716_at, 203099_s_at, 207968_s_at,
204913_s_at, 226114_at, 235473_at, 214883_at,
PANTHER_BP_ALL BP00101: Sulfur metabolism 14 3.24% 0.02869374 354
      595 29414 1.955068129 0.226011602
244440_at, 213893_x_at, 205885_s_at, 213753_x_at, 227694_at, 223523_at, 211740_at,
1556282_at, 1554778_at, 1554740_a_at, 208688_x_at, 218980_at, 236098_at, 1554188_at,
INTERPRO IPR013069: BTB/POZ 6 1.39% 0.029319056 288 107 17845
      3.474493769 0.999999578
204182_s_at, 229164_s_at, 1569794_at, 1554740_a_at, 204177_s_at, 207976_at,
GOTERM_BP_ALL GO: 0043283~biopolymer metabolic process 103 23.84%
      0.029342843 251 5361 15360 1.175733552 0.977994188 216251_s_at,
237548_at, 1557248_at, 200956_s_at, 234251_at, 229408_at, 1554345_a_at, 207253_s_at,
206074_s_at, 206286_s_at, 236613_at, 240998_at, 201959_s_at, 232857_at, 211180_x_at,
205527_s_at, 237942_at, 223792_at, 218585_s_at, 203099_s_at, 207968_s_at, 243414_at,
204913_s_at, 202332_at, 214883_at, 213835_x_at, 219960_s_at, 208698_s_at, 211208_s_at,
235815_at, 1555349_a_at, 240221_at, 231713_s_at, 232063_x_at, 208884_s_at, 213949_s_at,
226200_at, 224683_at, 207934_at, 217621_at, 213442_x_at, 206180_x_at, 204014_at,
227449_at, 213444_at, 226013_at, 1565537_at, 1565628_at, 222556_at, 1557300_s_at,
211212_s_at, 218493_at, 210156_s_at, 1558208_at, 236098_at, 236805_at, 1563620_at,
238276_at, 235792_x_at, 216889_s_at, 1553172_at, 206035_at, 204182_s_at, 231548_at,
220233_at, 230239_at, 240786_at, 242868_at, 235788_at, 219568_x_at, 233754_x_at,
233722_at, 1558000_at, 219919_s_at, 213038_at, 226114_at, 219419_at, 1565735_at,
214915_at, 205655_at, 213893_x_at, 1553322_s_at, 1559633_a_at, 203022_at, 221517_s_at,
204177_s_at, 238505_at, 217415_at, 212684_at, 234472_at, 202695_s_at, 211194_s_at,
228712_at, 211771_s_at, 1560752_at, 237093_at, 210981_s_at, 205938_at, 1566163_at,
211064_at, 204708_at, 239716_at, 235473_at,
PANTHER_MF_ALL MF00100: G-protein modulator 33 7.64% 0.030433569 354
      1884 29414 1.455405016 0.336593125
206413_s_at, 214013_s_at, 218429_s_at, 214915_at, 237905_at, 234701_at, 216889_s_at,
243636_s_at, 240612_at, 215112_x_at, 204182_s_at, 200991_s_at, 1561365_at, 211740_at,
232063_x_at, 1569025_s_at, 236185_at, 226986_at, 234544_at, 208710_s_at, 237311_at,
1556037_s_at, 234480_at, 213444_at, 233722_at, 211925_s_at, 231338_at, 213038_at,
218493_at, 206413_s_at, 218585_s_at, 216242_x_at, 206973_at,
PANTHER_MF_ALL MF00071: Translation factor 11 2.55% 0.031770299 354
      421 29414 2.171007958 0.333771757
240998_at, 239719_at, 225939_at, 204385_at, 205655_at, 208688_x_at, 225215_s_at,
223357_s_at, 213753_x_at, 220120_s_at, 1570093_at,
INTERPRO IPR013302: Wnt-10 protein 2 0.46% 0.031908099 288 2
      17845 61.96180556 0.999999613
223709_s_at, 206213_at,
PANTHER_BP_ALL BP00196:Oogenesis 14 3.24% 0.03274719 354 607
      29414 1.916417688 0.24552274
220434_at, 216251_s_at, 209574_s_at, 211771_s_at, 213444_at, 201819_at, 1555349_a_at,
218585_s_at, 208255_s_at, 208357_x_at, 214420_s_at, 235473_at, 214595_at, 202332_at,
PANTHER_MF_ALL MF00072: Translation initiation factor 26 6.02% 0.033432161
      354 1406 29414 1.536522249 0.333919879
219960_s_at, 239719_at, 206413_s_at, 211571_s_at, 234701_at, 205655_at, 1556017_at,
1559633_a_at, 225939_at, 208476_s_at, 208688_x_at, 223357_s_at, 220120_s_at, 238093_at,
240998_at, 218416_s_at, 204385_at, 231643_s_at, 1560752_at, 236834_at, 213753_x_at,
211212_s_at, 204300_at, 213038_at, 229131_at, 1570093_at,
INTERPRO IPR008271: Serine/threonine protein kinase, active site 12 2.78%
      0.034251118 288 364 17845 2.042696886 0.999999601
240998_at, 240221_at, 1565628_at, 237942_at, 202695_s_at, 237548_at, 204708_at, 228712_at,
237093_at, 230239_at, 202332_at, 210981_s_at,
UP_SEQ_FEATURE region of interest: L, hydrophobic 2 0.46% 0.034372417
      210 2 12056 57.40952381 1
226125_at, 208039_at,
UP_SEQ_FEATURE region of interest: B (M2) hydrophobic 2 0.46% 0.034372417
      210 2 12056 57.40952381 1
226125_at, 208039_at,
UP_SEQ_FEATURE region of interest: A (M1) hydrophobic 2 0.46% 0.034372417
      210 2 12056 57.40952381 1
226125_at, 208039_at,
GOTERM_CC_ALL GO: 0019897~extrinsic to plasma membrane 4 0.93%
      0.034415239 271 42 15857 5.572658584 0.999960248
206775_at, 228831_s_at, 222756_s_at, 206286_s_at,
SP_PIR_KEYWORDS chromosomal rearrangement 9 2.08% 0.034685501 275
      240 17599 2.399863636 0.944684863
1556282_at, 208698_s_at, 231338_at, 206413_s_at, 231548_at, 239716_at, 211180_x_at,
206074_s_at, 234544_at,
SP_PIR_KEYWORDS Transcription regulation 38 8.80% 0.034942498 275 1754
      17599 1.386466259 0.933344523
208698_s_at, 238276_at, 214915_at, 1557248_at, 216889_s_at, 235815_at, 200956_s_at,
1553172_at, 206035_at, 204182_s_at, 231713_s_at, 234251_at, 1553322_s_at, 229408_at,
231548_at, 221517_s_at, 206074_s_at, 213442_x_at, 240786_at, 242868_at, 212684_at,
211194_s_at, 219568_x_at, 211180_x_at, 211771_s_at, 233754_x_at, 1558000_at, 1565537_at,
1557300_s_at, 1558208_at, 223792_at, 239716_at, 203099_s_at, 207968_s_at, 204913_s_at,
226114_at, 235473_at, 214883_at,
PANTHER_MF_ALL MF00101: Guanyl-nucleotide exchange factor 46 10.65%
      0.035379055 354 2853 29414 1.339697929 0.336311628
212057_at, 239719_at, 242913_at, 207566_at, 218429_s_at, 237905_at, 234701_at,
218744_s_at, 240966_at, 243636_s_at, 216889_s_at, 215112_x_at, 1556017_at, 204182_s_at,
200991_s_at, 202638_s_at, 222389_s_at, 229444_at, 232063_x_at, 1561365_at, 213949_s_at,
236185_at, 208476_s_at, 218070_s_at, 208357_x_at, 234544_at, 237311_at, 208710_s_at,
1556037_s_at, 226410_at, 242426_at, 201959_s_at, 227449_at, 233722_at, 241849_at,
220364_at, 220129_at, 221066_at, 231338_at, 219919_s_at, 218493_at, 218585_s_at,
216242_x_at, 1552531_a_at, 206213_at, 1570093_at,
PANTHER_BP_ALL BP00102: Signal transduction 66 15.28% 0.035461533 354
      4356 29414 1.258945386 0.254867698
204029_at, 200783_s_at, 1563620_at, 211571_s_at, 1557248_at, 215112_x_at, 1568687_s_at,
1556017_at, 202638_s_at, 223122_s_at, 1561365_at, 205388_at, 208570_at, 206972_s_at,
207253_s_at, 205665_at, 208357_x_at, 227928_at, 235004_at, 243118_at, 242426_at,
222826_at, 240998_at, 204118_at, 203799_at, 244440_at, 1554690_a_at, 1552925_at,
244220_at, 221066_at, 216007_at, 203099_s_at, 223709_s_at, 206213_at, 214883_at,
1570093_at, 213835_x_at, 232802_at, 211208_s_at, 240966_at, 225215_s_at, 210644_s_at,
1555349_a_at, 229444_at, 240221_at, 231713_s_at, 1559633_a_at, 208710_s_at, 237311_at,
1552461_at, 233318_at, 1560997_at, 238037_at, 206563_s_at, 214752_x_at, 211771_s_at,
222756_s_at, 229164_s_at, 206190_at, 206413_s_at, 218493_at, 230492_s_at, 232187_at,
235885_at, 228831_s_at, 221766_s_at,
INTERPRO IPR015915: Kelch-type beta propeller 4 0.93% 0.035572137 288
      45 17845 5.507716049 0.999999379
1554740_a_at, 204177_s_at, 229035_s_at, 207976_at,
SP_PIR_KEYWORDS activator 13 3.01% 0.035901148 275 427 17599
      1.948367043 0.925600059
240786_at, 242868_at, 212684_at, 208698_s_at, 211194_s_at, 219568_x_at, 211771_s_at,
1553322_s_at, 231548_at, 207968_s_at, 221517_s_at, 213442_x_at, 235473_at,
SP_PIR_KEYWORDS Direct protein sequencing 51 11.81% 0.035956274 275
      2501 17599 1.305003817 0.912814388
243160_at, 200783_s_at, 211571_s_at, 205885_s_at, 202638_s_at, 200991_s_at, 223122_s_at,
205774_at, 205388_at, 208357_x_at, 206074_s_at, 206286_s_at, 230239_at, 211016_x_at,
204118_at, 244440_at, 1552703_s_at, 205075_at, 241849_at, 209960_at, 213753_x_at,
203167_at, 217340_at, 244220_at, 1560587_s_at, 219683_at, 230988_at, 201079_at, 239719_at,
208698_s_at, 205900_at, 1555349_a_at, 1553322_s_at, 218831_s_at, 221517_s_at, 214420_s_at,
210034_s_at, 223357_s_at, 1560997_at, 230966_at, 200838_at, 214752_x_at, 228712_at,
210297_s_at, 1557300_s_at, 204300_at, 210156_s_at, 1558208_at, 236713_at, 235473_at,
215766_at,
INTERPRO IPR005817: Wnt superfamily 3 0.69% 0.036815837 288 19
      17845 9.783442982 0.999997872
208570_at, 223709_s_at, 206213_at,
INTERPRO IPR005816: Secreted growth factor Wnt protein 3 0.69% 0.036815837
      288 19 17845 9.783442982 0.999997872
208570_at, 223709_s_at, 206213_at,
INTERPRO IPR013761: Sterile alpha motif-type 5 1.16% 0.037005913 288
      78 17845 3.971910613 0.99999588
229402_at, 211194_s_at, 237548_at, 227449_at, 244440_at,
GOTERM_MF_ALL GO: 0015405~P-P-bond-hydrolysis-driven transmembrane transporter
activity 7 1.62% 0.037198901 270 155 16968 2.838136201 0.999998805
215319_at, 1570505_at, 237182_at, 234741_at, 239357_at, 36994_at, 241448_at, 1568687_s_at,
GOTERM_MF_ALL GO: 0015399~primary active transmembrane transporter activity 7
      1.62% 0.037198901 270 155 16968 2.838136201 0.999998805
215319_at, 1570505_at, 237182_at, 234741_at, 239357_at, 36994_at, 241448_at, 1568687_s_at,
GOTERM_BP_ALL GO: 0030097~hemopoiesis 8 1.85% 0.037838646 251
      192 15360 2.549800797 0.991975958
240786_at, 237942_at, 229408_at, 231548_at, 211180_x_at, 230988_at, 208710_s_at,
241849_at,
GOTERM_BP_ALL GO: 0032502~developmental process 66 15.28% 0.039992523
      251 3262 15360 1.238162006 0.993173386
204029_at, 200783_s_at, 240171_at, 237548_at, 211571_s_at, 214955_at, 205885_s_at,
201819_at, 223122_s_at, 1561365_at, 202931_x_at, 229408_at, 231548_at, 208570_at,
206286_s_at, 230239_at, 240786_at, 242868_at, 222826_at, 1552703_s_at, 202510_s_at,
219568_x_at, 211180_x_at, 1552703_s_at, 203167_at, 241849_at, 209960_at, 213753_x_at,
237942_at, 219683_at, 207968_s_at, 208255_s_at, 230988_at, 204913_s_at, 223709_s_at,
225858_s_at, 206213_at, 1570093_at, 205900_at, 205655_at, 1554890_a_at, 235815_at,
210330_at, 1557558_s_at, 1555349_a_at, 1559633_a_at, 233937_at, 213442_x_at, 208710_s_at,
237311_at, 1556037_s_at, 203045_at, 233318_at, 1560997_at, 212684_at, 202695_s_at,
211194_s_at, 200838_at, 214752_x_at, 211771_s_at, 222556_at, 1557300_s_at, 234741_at,
239357_at, 232187_at, 243142_at, 221114_at,
PANTHER_BP_ALL BP00033: Pyrimidine metabolism 9 2.08% 0.040845742
      354 321 29414 2.329637256 0.27939871
226410_at, 211740_at, 1558208_at, 208334_at, 218493_at, 205388_at, 206074_s_at,
243636_s_at, 236905_at,
PANTHER_BP_ALL BP00062: Protein folding 13 3.01% 0.041013971 354
      564 29414 1.915204151 0.272179648
221667_s_at, 211016_x_at, 233351_at, 240293_at, 216889_s_at, 243636_s_at, 232725_s_at,
220129_at, 208255_s_at, 238581_at, 202415_s_at, 214883_at, 220120_s_at,
PANTHER_BP_ALL BP00119: Other intracellular signaling cascade 18 4.17%
      0.041793648 354 893 29414 1.674834399 0.268805357
214914_at, 1570505_at, 201959_s_at, 231643_s_at, 237905_at, 218744_s_at, 200710_at,
229164_s_at, 240221_at, 1565537_at, 1559633_a_at, 229402_at, 226200_at, 230492_s_at,
223103_at, 204177_s_at, 208710_s_at, 232213_at,
SP_PIR_KEYWORDS nucleus 71 16.44% 0.042126223 275 3710 17599
      1.224728253
0.932714814 237548_at, 214013_s_at, 238276_at, 1557248_at, 234701_at, 235792_x_at,
216889_s_at, 200956_s_at, 1553172_at, 206035_at, 204182_s_at, 222389_s_at, 234251_at,
202931_x_at, 229408_at, 231548_at, 207253_s_at, 206074_s_at, 227928_at, 235004_at,
240786_at, 242868_at, 232857_at, 219568_x_at, 211180_x_at, 205527_s_at, 233722_at,
233754_x_at, 213753_x_at, 1558000_at, 237942_at, 219919_s_at, 223792_at, 203099_s_at,
207968_s_at, 243414_at, 204913_s_at, 226114_at, 214883_at, 208698_s_at, 214915_at,
205655_at, 235815_at, 236905_at, 231713_s_at, 208884_s_at, 1553322_s_at, 203022_at,
224683_at, 221517_s_at, 210034_s_at, 217621_at, 213442_x_at, 217415_at, 212684_at,
202695_s_at, 211194_s_at, 204014_at, 211771_s_at, 237093_at, 205938_at, 1554821_a_at,
1565537_at, 1566163_at, 211212_s_at, 1557300_s_at, 1558208_at, 218493_at, 239716_at,
235473_at, 236098_at,
PANTHER_MF_ALL MF00179: Extracellular matrix structural protein 11 2.55%
      0.042665915 354 443 29414 2.063192664 0.37729696
239719_at, 233467_s_at, 218493_at, 232422_at, 218585_s_at, 202510_s_at, 204014_at,
208476_s_at, 215112_x_at, 1557558_s_at, 1570093_at,
GOTERM_BP_ALL GO: 0006512~ubiquitin cycle 15 3.47% 0.043318351 251
      515 15360 1.782385023 0.994948063
219960_s_at, 201959_s_at, 1563620_at, 235788_at, 1557248_at, 1560752_at, 208884_s_at,
213038_at, 239716_at, 218585_s_at, 220233_at, 224683_at, 243414_at, 207934_at,
204177_s_at,
GOTERM_BP_ALL GO: 0007389~pattern specification process 7 1.62%
      0.043535713 251 157 15360 2.72844926 0.994466879
1556037_s_at, 240786_at, 233318_at, 223122_s_at, 222556_at, 208255_s_at, 206286_s_at,
PANTHER_BP_ALL BP00273: Chromatin packaging and remodeling 19 4.40%
      0.043674815 354 964 29414 1.63767377 0.271612855
240786_at, 203045_at, 226410_at, 201959_s_at, 231643_s_at, 242913_at, 228712_at,
205655_at, 206035_at, 239345_at, 229408_at, 216007_at, 203099_s_at, 206972_s_at,
208688_x_at, 204177_s_at, 214420_s_at, 237311_at, 1570093_at,
GOTERM_MF_ALL GO: 0008092~cytoskeletal protein binding 13 3.01%
      0.043689413 270 433 16968 1.886784706 0.999999375
200783_s_at, 212793_at, 202898_at, 218744_s_at, 214752_x_at, 226013_at, 1558208_at,
208476_s_at, 1554740_a_at, 230988_at, 204177_s_at, 218980_at, 220120_s_at,
PANTHER_MF_ALL MF00214: Non-receptor tyrosine protein kinase 20 4.63%
      0.043896237 354 1031 29414 1.611840843 0.37277724
240786_at, 233318_at, 214914_at, 233543_s_at, 204385_at, 230447_at, 236388_at, 227449_at,
1554690_a_at, 200710_at, 202638_s_at, 217340_at, 211740_at, 1558208_at, 226200_at,
205388_at, 208476_s_at, 219683_at, 223709_s_at, 214595_at,
GOTERM_CC_ALL GO: 0005622~intracellular 194 44.91% 0.044532622 271
      10544 15857 1.076584364 0.999949085
221667_s_at, 212057_at, 200783_s_at, 233351_at, 240171_at, 237548_at, 217340_at,
1557248_at, 1561365_at, 233467_s_at, 205388_at, 208476_s_at, 222826_at, 201959_s_at,
224330_s_at, 244220_at, 217340_at, 231338_at, 1560587_s_at, 218585_s_at, 207968_s_at,
201079_at, 1570093_at, 213835_x_at, 237905_at, 235815_at, 240221_at, 226200_at, 224683_at,
233937_at, 208688_x_at, 210034_s_at, 217621_at, 213442_x_at, 237311_at, 208710_s_at,
238037_at, 204014_at, 200838_at, 213361_at, 222756_s_at, 241448_at, 206775_at, 1565537_at,
1565628_at, 222556_at, 1557300_s_at, 218493_at, 234741_at, 239357_at, 216242_x_at,
215766_at, 214013_s_at, 226623_at, 203164_at, 238276_at, 218744_s_at, 216889_s_at,
1553172_at, 206035_at, 1554740_a_at, 218980_at, 235004_at, 242868_at, 211016_x_at,
237182_at, 204385_at, 235788_at, 219568_x_at, 244440_at, 1554690_a_at, 200710_at,
205075_at, 233754_x_at, 241849_at, 225858_s_at, 226114_at, 1565735_at, 232802_at,
205900_at, 205655_at, 236905_at, 238941_at, 211740_at, 1553322_s_at, 1554778_at,
206153_at, 214420_s_at, 223357_s_at, 220120_s_at, 217415_at, 211194_s_at, 230966_at,
214752_x_at, 211771_s_at, 1560752_at, 1554821_a_at, 205938_at, 1555393_s_at, 1556282_at,
239716_at, 236713_at, 229131_at, 235098_at, 234701_at, 1568949_at, 200956_s_at,
200991_s_at, 222389_s_at, 234251_at, 202931_x_at, 229408_at, 207253_s_at, 206074_s_at,
236613_at, 240998_at, 232857_at, 211180_x_at, 205527_s_at, 236388_at, 213753_x_at,
237942_at, 223792_at, 203099_s_at, 208255_s_at, 243414_at, 204913_s_at, 223296_at,
202332_at, 214883_at, 219960_s_at, 208698_s_at, 211208_s_at, 242913_at, 36994_at,
225215_s_at, 231713_s_at, 232063_x_at, 1569025_s_at, 208884_s_at, 225939_at, 234544_at,
233318_at, 206180_x_at, 208334_at, 213444_at, 229164_s_at, 226013_at, 211212_s_at,
204300_at, 210156_s_at, 1558208_at, 232187_at, 243142_at, 236098_at, 221920_s_at,
1563620_at, 235792_x_at, 215112_x_at, 204182_s_at, 205380_at, 231548_at, 227928_at,
230239_at, 240786_at, 225484_at, 1552703_s_at, 1552703_s_at, 233722_at, 1558000_at,
211925_s_at, 219919_s_at, 230988_at, 219419_at, 214915_at, 1554890_a_at, 210330_at,
203022_at, 221517_s_at, 204177_s_at, 1556037_s_at, 212684_at, 234472_at, 202695_s_at,
228712_at, 237093_at, 240172_at, 222603_at, 210297_s_at, 220129_at, 1566163_at, 211064_at,
206973_at, 235473_at, 217340_at,
SMART SM00097: WNT1 3 0.69% 0.045916882 181 19 9899
      8.635359116 0.999201624
208570_at, 223709_s_at, 206213_at,
GOTERM_MF_ALL GO: 0022804~active transmembrane transporter activity 12 2.78%
      0.046584676 270 389 16968 1.938646101 0.99999891
215319_at, 239345_at, 1570505_at, 208389_s_at, 237182_at, 234741_at, 239357_at, 226125_at,
36994_at, 241448_at, 208039_at, 240612_at, 1568687_s_at,
PANTHER_MF_ALL MF00261: Actin binding cytoskeletal protein 32 7.41%
      0.046823268 354 1884 29414 1.411301834 0.379699549
235098_at, 239719_at, 234701_at, 236905_at, 1561365_at, 211740_at, 1554345_a_at,
1554740_a_at, 204177_s_at, 227928_at, 218980_at, 243118_at, 1552461_at, 233318_at,
212793_at, 234480_at, 204385_at, 201959_s_at, 214752_x_at, 205527_s_at, 227449_at,
220364_at, 1565537_at, 211925_s_at, 1557300_s_at, 236713_at, 228831_s_at, 218585_s_at,
216242_x_at, 230988_at, 221766_s_at, 207976_at,
INTERPRO IPR000719: Protein kinase, core 15 3.47% 0.046983376 288 529
      17845 1.756951008 0.999999692
236805_at, 240998_at, 237548_at, 202695_s_at, 211208_s_at, 228712_at, 227449_at,
237093_at, 210981_s_at, 240221_at, 1565628_at, 237942_at, 204708_at, 202332_at, 230239_at,
INTERPRO IPR001147: Ribosomal protein L21e 2 0.46% 0.047479604 288
      3 17845 41.30787037 0.99999944
217340_at, 217340_at,
PANTHER_MF_ALL MF00098: Large G-protein 18 4.17% 0.048484938 354
      907 29414 1.64898249 0.378195618
209163_at, 235098_at, 206413_s_at, 226623_at, 204385_at, 1552703_s_at, 1552703_s_at,
205885_s_at, 240221_at, 222556_at, 210156_s_at, 228831_s_at, 216007_at, 203099_s_at,
238581_at, 208357_x_at, 225858_s_at, 223296_at,
PANTHER_BP_ALL BP00224: Cell proliferation and differentiation 22 5.09%
      0.048674226 354 1184 29414 1.543909375 0.290399873
242426_at, 218416_s_at, 212793_at, 206563_s_at, 240171_at, 202898_at, 205655_at,
216889_s_at, 1554690_a_at, 215112_x_at, 240172_at, 1555349_a_at, 1561365_at, 211925_s_at,
1554778_at, 206413_s_at, 218070_s_at, 221766_s_at, 223709_s_at, 210034_s_at, 235004_at,
218980_at,
PANTHER_BP_ALL BP00134: Mitochondrial transport 10 2.31% 0.048950301
      354 394 29414 2.108893286 0.284370751
1560997_at, 210156_s_at, 218493_at, 228375_at, 235788_at, 236388_at, 214752_x_at,
216889_s_at, 241448_at, 228403_at,
GOTERM_BP_ALL GO: 0007223~Wnt receptor signaling pathway, calcium modulating
pathway 3 0.69% 0.049224243 251 22 15360 8.344802608
      0.996865721
208570_at, 223709_s_at, 206213_at,
GOTERM_BP_ALL GO: 0032984~macromolecular complex disassembly 3 0.69%
      0.049224243 251 22 15360 8.344802608 0.996865721
206074_s_at, 225215_s_at, 223357_s_at,
PANTHER_MF_ALL MF00036: Transcription factor 36 8.33% 0.050018493 354
      2184 29414 1.369621904 0.376048533
238276_at, 214915_at, 1557248_at, 235815_at, 200956_s_at, 1553172_at, 206035_at,
204182_s_at, 234251_at, 1553322_s_at, 231548_at, 221517_s_at, 235004_at, 213442_x_at,
242868_at, 212684_at, 206180_x_at, 211194_s_at, 219568_x_at, 211180_x_at, 211771_s_at,
213444_at, 233754_x_at, 1554821_a_at, 1558000_at, 1565748_at, 1565537_at, 1566163_at,
1558208_at, 223792_at, 211064_at, 239716_at, 207968_s_at, 204913_s_at, 226114_at,
235473_at,
UP_SEQ_FEATURE splice variant 77 17.82% 0.050139639 210 3722 12056
      1.187676876 1
243160_at, 233351_at, 1570505_at, 240171_at, 237548_at, 1563620_at, 238276_at,
211571_s_at, 202898_at, 214955_at, 216889_s_at, 201819_at, 202931_x_at, 229408_at,
206972_s_at, 208357_x_at, 202415_s_at, 226986_at, 240786_at, 222826_at, 240998_at,
225484_at, 232857_at, 211180_x_at, 236388_at, 1552703_s_at, 200710_at, 233722_at,
209960_at, 241849_at, 236834_at, 1558000_at, 244220_at, 211925_s_at, 219919_s_at,
1560587_s_at, 203099_s_at, 207968_s_at, 243414_at, 230988_at, 214883_at, 219419_at,
232802_at, 219960_s_at, 242913_at, 1554890_a_at, 240966_at, 229444_at, 238941_at,
1569025_s_at, 1554778_at, 217621_at, 234544_at, 208710_s_at, 1556037_s_at, 211422_at,
206563_s_at, 234472_at, 234480_at, 228712_at, 1560752_at, 211771_s_at, 222756_s_at,
210981_s_at, 206190_at, 1566163_at, 232725_s_at, 210297_s_at, 1557300_s_at, 210156_s_at,
234741_at, 239357_at, 243142_at, 218475_at, 221114_at, 1552531_a_at, 236098_at, 214595_at,
GOTERM_BP_ALL GO: 0050878~regulation of body fluid levels 6 1.39%
      0.050627754 251 123 15360 2.98513264 0.996609683
205774_at, 205900_at, 235885_at, 214955_at, 216889_s_at, 209960_at,
SP_PIR_KEYWORDS host-virus interaction 8 1.85% 0.050678206 275 214
      17599 2.392387426 0.954040595
202638_s_at, 202931_x_at, 1563620_at, 207253_s_at, 36994_at, 208255_s_at, 206074_s_at,
201819_at,
GOTERM_BP_ALL GO: 0048534~hemopoietic or lymphoid organ development 8
      1.85% 0.052113096 251 206 15360 2.376513364 0.996780933
240786_at, 237942_at, 229408_at, 231548_at, 211180_x_at, 230988_at, 208710_s_at,
241849_at,
GOTERM_BP_ALL GO: 0006412~translation 17 3.94% 0.052117178 251
      629 15360 1.653924841 0.996391066
217340_at, 237182_at, 224330_s_at, 1554890_a_at, 225215_s_at, 206035_at, 213753_x_at,
229164_s_at, 217340_at, 232063_x_at, 204300_at, 226200_at, 225939_at, 208688_x_at,
210034_s_at, 223357_s_at, 217340_at,
PANTHER_BP_ALL BP00182: Sensory perception 13 3.01% 0.052787442 354
      589 29414 1.833913652 0.295953848
224881_at, 240998_at, 226995_at, 238801_at, 222756_s_at, 236905_at, 1555393_s_at,
206775_at, 210156_s_at, 216007_at, 235343_at, 208255_s_at, 206286_s_at,
GOTERM_BP_ALL GO: 0007275~multicellular organismal development 49 11.34%
      0.053433758 251 2349 15360 1.276528624 0.996507901
204029_at, 200783_s_at, 211571_s_at, 214955_at, 205885_s_at, 223122_s_at, 1561365_at,
202931_x_at, 229408_at, 231548_at, 208570_at, 206286_s_at, 240786_at, 242868_at,
202510_s_at, 219568_x_at, 211180_x_at, 241849_at, 203167_at, 237942_at, 219683_at,
207968_s_at, 208255_s_at, 204913_s_at, 230988_at, 206213_at, 223709_s_at, 1570093_at,
205900_at, 235815_at, 1557558_s_at, 210330_at, 1555349_a_at, 1559633_a_at, 233937_at,
213442_x_at, 208710_s_at, 203045_at, 233318_at, 1556037_s_at, 212684_at, 1560997_at,
211194_s_at, 214752_x_at, 222556_at, 1557300_s_at, 234741_at, 239357_at, 243142_at,
221114_at,
PANTHER_BP_ALL BP00124: Cell adhesion 14 3.24% 0.05374277 354 655
      29414 1.775977919 0.293355873
203045_at, 206413_s_at, 228375_at, 202510_s_at, 202898_at, 214955_at, 205885_s_at,
238045_at, 205938_at, 202638_s_at, 226295_at, 206973_at, 226986_at, 221920_s_at,
PANTHER_MF_ALL MF00197: Miscellaneous function 20 4.63% 0.053769434
      354 1057 29414 1.572192914 0.386903609
233318_at, 203045_at, 212057_at, 200783_s_at, 230447_at, 216669_at, 236388_at, 244440_at,
1560752_at, 222756_s_at, 1554690_a_at, 227420_at, 240172_at, 1565628_at, 232187_at,
221114_at, 244029_at, 230571_at, 240646_at, 213835_x_at,
GOTERM_MF_ALL GO: 0016874~ligase activity 12 2.78% 0.055472284 270
      401 16968 1.880631754 0.999999672
232063_x_at, 208884_s_at, 216251_s_at, 204300_at, 213038_at, 226200_at, 201959_s_at,
1563620_at, 239716_at, 243414_at, 1557248_at, 1560752_at,
SP_PIR_KEYWORDS proto-oncogene 8 1.85% 0.05591947 275 219 17599
      2.337766708 0.960383539
206413_s_at, 237548_at, 231548_at, 208570_at, 238276_at, 211180_x_at, 234544_at,
206035_at,
GOTERM_BP_ALL GO: 0007243~protein kinase cascade 12 2.78% 0.05723935
      251 393 15360 1.868556309 0.997408438
233318_at, 240998_at, 231713_s_at, 202695_s_at, 237548_at, 204014_at, 214752_x_at,
228712_at, 1552703_s_at, 206286_s_at, 203167_at, 206035_at,
PANTHER_BP_ALL BP00287: Cell motility 16 3.70% 0.057719462 354 797
      29414 1.668063146 0.304635583
211422_at, 212793_at, 230966_at, 226125_at, 218744_s_at, 214752_x_at, 229035_s_at,
215112_x_at, 236905_at, 222389_s_at, 1561365_at, 1569025_s_at, 211925_s_at, 231548_at,
243414_at, 234481_at,
GOTERM_BP_ALL GO: 0031323~regulation of cellular metabolic process 58 13.43%
      0.058201417 251 2873 15360 1.235406442 0.99737937
238276_at, 1557248_at, 216889_s_at, 200956_s_at, 1553172_at, 206035_at, 204182_s_at,
234251_at, 229408_at, 231548_at, 207253_s_at, 206074_s_at, 206286_s_at, 240786_at,
242868_at, 201959_s_at, 219568_x_at, 211180_x_at, 233754_x_at, 213753_x_at, 203167_at,
1558000_at, 223792_at, 203099_s_at, 207968_s_at, 204913_s_at, 226114_at, 214883_at,
1565735_at, 208698_s_at, 214915_at, 205655_at, 1554890_a_at, 235815_at, 1555349_a_at,
231713_s_at, 1553322_s_at, 226200_at, 225939_at, 221517_s_at, 213442_x_at, 223357_s_at,
217415_at, 212684_at, 206180_x_at, 211194_s_at, 214752_x_at, 211771_s_at, 213444_at,
226013_at, 1565537_at, 1566163_at, 220129_at, 1557300_s_at, 1558208_at, 211064_at,
239716_at, 235473_at,
SMART SM00360: RRM9 2.08% 0.058353258 181 230 9899
      2.140067259 0.999323911
236613_at, 208698_s_at, 1558208_at, 218475_at, 238801_at, 208688_x_at, 1554890_a_at,
235004_at, 233722_at,
GOTERM_MF_ALL GO: 0016773~phosphotransferase activity, alcohol group as acceptor
      20 4.63% 0.058961456 270 813 16968 1.545988793 0.999999532
236805_at, 221667_s_at, 220434_at, 242868_at, 240998_at, 202695_s_at, 237548_at,
211208_s_at, 228712_at, 227449_at, 235792_x_at, 237093_at, 210981_s_at, 240221_at,
1561365_at, 1565628_at, 237942_at, 204708_at, 230239_at, 202332_at,
PANTHER_BP_ALL BP00112: Calcium mediated signaling 11 2.55% 0.05970158
      354 472 29414 1.936428708 0.306511385
222389_s_at, 240221_at, 211212_s_at, 226125_at, 208255_s_at, 228712_at, 208357_x_at,
218557_at, 206074_s_at, 227928_at, 210330_at,
PANTHER_BP_ALL BP00014: Amino acid biosynthesis 13 3.01% 0.059845733
      354 602 29414 1.794310866 0.300420591
211422_at, 206413_s_at, 234472_at, 1554681_a_at, 234701_at, 244820_at, 244440_at,
213250_at, 202638_s_at, 222556_at, 211212_s_at, 218980_at, 230239_at,
PANTHER_MF_ALL MF00269: SNARE protein 6 1.39% 0.060587398 354
      175 29414 2.848813559 0.413443526
206180_x_at, 206413_s_at, 218831_s_at, 226986_at, 227694_at, 236905_at,
PANTHER_BP_ALL BP00051: mRNA end-processing and stability 5 1.16%
      0.061163091 354 123 29414 3.377658353 0.299544805
222389_s_at, 206413_s_at, 206413_s_at, 218070_s_at, 205665_at,
INTERPRO IPR006652: Kelch repeat type 1 4 0.93% 0.061200664 288 56
      17845 4.425843254 0.999999981
1554740_a_at, 204177_s_at, 229035_s_at, 207976_at,
PANTHER_MF_ALL MF00107: Kinase 14 3.24% 0.062320524 354 670
      29414 1.736217219 0.41157679
236805_at, 237548_at, 202695_s_at, 211208_s_at, 228712_at, 235792_x_at, 237093_at,
210981_s_at, 240221_at, 1565628_at, 237942_at, 204708_at, 202332_at, 230239_at,
SP_PIR_KEYWORDS Developmental protein 16 3.70% 0.062577017 275 622
      17599 1.646208711 0.968072323
240786_at, 204029_at, 242868_at, 212684_at, 200783_s_at, 211194_s_at, 202510_s_at,
235815_at, 223122_s_at, 202931_x_at, 208570_at, 219683_at, 233937_at, 223709_s_at,
206213_at, 1570093_at,
PANTHER_MF_ALL MF00114: Protein phosphatase 12 2.78% 0.062686225 354
      541 29414 1.843040195 0.402941117
230009_at, 231338_at, 219919_s_at, 1563620_at, 203099_s_at, 218585_s_at, 36994_at,
226986_at, 205938_at, 219650_at, 217415_at, 1570093_at,
PANTHER_BP_ALL BP00012: Other carbohydrate metabolism 7 1.62%
      0.062974086 354 233 29414 2.496277976 0.30074785
230009_at, 205774_at, 237548_at, 200838_at, 207968_s_at, 205655_at, 206213_at,
PANTHER_BP_ALL BP00132: Receptor mediated endocytosis 11 2.55%
      0.063416015 354 477 29414 1.916130713 0.296403123
218416_s_at, 211016_x_at, 216251_s_at, 213949_s_at, 202695_s_at, 1558208_at, 236713_at,
242913_at, 230239_at, 214883_at, 208710_s_at,
GOTERM_CC_ALL GO: 0044424~intracellular part 182 42.13% 0.065945304 271
      9906 15857 1.075040435 0.999992812
200783_s_at, 233351_at, 235098_at, 240171_at, 217340_at, 237548_at, 234701_at, 1557248_at,
200956_s_at, 1568949_at, 200991_s_at, 222389_s_at, 202931_x_at, 234251_at, 1561365_at,
229408_at, 205388_at, 207253_s_at, 208476_s_at, 206074_s_at, 240998_at, 236613_at,
222826_at, 201959_s_at, 224330_s_at, 232857_at, 236388_at, 205527_s_at, 211180_x_at,
213753_x_at, 217340_at, 244220_at, 237942_at, 231338_at, 223792_at, 1560587_s_at,
203099_s_at, 218585_s_at, 243414_at, 208255_s_at, 207968_s_at, 204913_s_at, 223296_at,
214883_at, 202332_at, 201079_at, 213835_x_at, 1570093_at, 219960_s_at, 208698_s_at,
211208_s_at, 242913_at, 237905_at, 36994_at, 225215_s_at, 235815_at, 240221_at,
232063_x_at, 231713_s_at, 208884_s_at, 225939_at, 226200_at, 224683_at, 208688_x_at,
233937_at, 217621_at, 210034_s_at, 234544_at, 213442_x_at, 208710_s_at, 237311_at,
233318_at, 238037_at, 208334_at, 204014_at, 200838_at, 213361_at, 241448_at, 222756_s_at,
226013_at, 229164_s_at, 206775_at, 1565628_at, 1565537_at, 222556_at, 204300_at,
211212_s_at, 1557300_s_at, 1558208_at, 210156_s_at, 234741_at, 239357_at, 218493_at,
243142_at, 232187_at, 216242_x_at, 236098_at, 215766_at, 221920_s_at, 226623_at,
214013_s_at, 1563620_at, 238276_at, 203164_at, 218744_s_at, 235792_x_at, 216889_s_at,
206035_at, 1553172_at, 204182_s_at, 205380_at, 231548_at, 1554740_a_at, 227928_at,
235004_at, 218980_at, 230239_at, 240786_at, 242868_at, 211016_x_at, 225484_at, 204385_at,
237182_at, 235788_at, 219568_x_at, 1552703_s_at, 244440_at, 1554690_a_at, 205075_at,
200710_at, 233722_at, 233754_x_at, 1558000_at, 211925_s_at, 219919_s_at, 230988_at,
225858_s_at, 226114_at, 219419_at, 232802_at, 1565735_at, 205900_at, 214915_at, 205655_at,
1554890_a_at, 210330_at, 236905_at, 211740_at, 1553322 s_at, 1554778_at, 203022_at,
206153_at, 221517_s_at, 204177_s_at, 214420_s_at, 223357_s_at, 217415_at, 220120_s_at,
1556037_s_at, 212684_at, 234472_at, 202695_s_at, 230966_at, 211194_s_at, 214752_x_at,
228712_at, 1560752_at, 211771_s_at, 237093_at, 205938_at, 1554821_a_at, 240172_at,
1555393_s_at, 222603_at, 1556282_at, 1566163_at, 220129_at, 210297_s_at, 239716_at,
236713_at, 206973_at, 235473_at, 217340_at,
PIR_SUPERFAMILY PIRSF013835: Caenorhabditis elegans hypothetical protein F55A12.9
      2 0.46% 0.065957349 118 4 6919 29.31779661 1
221766_s_at, 1552461_at,
GOTERM_BP_ALL GO: 0002520~immune system development 8 1.85%
      0.066458335 251 218 15360 2.245696115 0.998758004
240786_at, 237942_at, 229408_at, 231548_at, 211180_x_at, 230988_at, 208710_s_at,
241849_at,
GOTERM_BP_ALL GO: 0006468~protein amino acid phosphorylation 18 4.17%
      0.068234643 251 704 15360 1.564650489 0.998830664
236805_at, 240998_at, 1565735_at, 202695_s_at, 237548_at, 211208_s_at, 228712_at,
227449_at, 237093_at, 210981_s_at, 1555349_a_at, 240221_at, 1565628_at, 237942_at,
204708_at, 206286_s_at, 230239_at, 202332_at,
GOTERM_BP_ALL GO: 0016070~RNA metabolic process 60 13.89% 0.069157214
      251 3020 15360 1.215799055 0.99879792
238276_at, 1557248_at, 216889_s_at, 200956_s_at, 1553172_at, 206035_at, 204182_s_at,
234251_at, 229408_at, 231548_at, 207253_s_at, 206074_s_at, 240786_at, 242868_at,
236613_at, 201959_s_at, 232857_at, 219568_x_at, 211180_x_at, 205527_s_at, 233722_at,
233754_x_at, 1558000_at, 223792_at, 203099_s_at, 207968_s_at, 204913_s_at, 226114_at,
214883_at, 219419_at, 213835_x_at, 208698_s_at, 214915_at, 205655_at, 235815_at,
231713_s_at, 232063_x_at, 1553322_s_at, 208884_s_at, 226200_at, 203022_at, 221517_s_at,
217621_at, 213442_x_at, 217415_at, 212684_at, 206180_x_at, 211194_s_at, 211771_s_at,
213444_at, 237093_at, 226013_at, 1565537_at, 1566163_at, 1557300_s_at, 218493_at,
1558208_at, 211064_at, 239716_at, 235473_at,
INTERPRO IPR002286: P2 purinoceptor 3 0.69% 0.069530138 288 27
      17845 6.884645062 0.999999996
206190_at, 235885_at, 217621_at,
GOTERM_BP_ALL GO: 0050794~regulation of cellular process 84 19.44%
      0.070485647 251 4422 15360 1.162460065 0.998814253
214013_s_at, 237548_at, 238276_at, 218744_s_at, 1557248_at, 216889_s_at, 200956_s_at,
215112_x_at, 1553172_at, 206035_at, 204182_s_at, 202638_s_at, 200991_s_at, 1561365_at,
234251_at, 202931_x_at, 229408_at, 231548_at, 207253_s_at, 206074_s_at, 206286_s_at,
230239_at, 240786_at, 242868_at, 201959_s_at, 1552703_s_at, 219568_x_at, 211180_x_at,
1552703_s_at, 203167_at, 241849_at, 233754_x_at, 213753_x_at, 1558000_at, 211925_s_at,
237942_at, 223792_at, 203099_s_at, 207968_s_at, 204913_s_at, 225858_s_at, 226114_at,
214883_at, 1565735_at, 208698_s_at, 214915_at, 205655_at, 1554890_a_at, 235815_at,
1555349_a_at, 231713_s_at, 1553322_s_at, 225939_at, 226200_at, 221517_s_at, 234544_at,
213442_x_at, 223357_s_at, 217415_at, 1556037_s_at, 1560997_at, 212684_at, 206180_x_at,
202695_s_at, 211194_s_at, 226125_at, 204014_at, 200838_at, 214752_x_at, 228712_at,
211771_s_at, 213444_at, 222756_s_at, 210981_s_at, 226013_at, 1565537_at, 1566163_at,
220129_at, 1557300_s_at, 1558208_at, 211064_at, 239716_at, 228831_s_at, 235473_at,
PANTHER_MF_ALL MF00124: Oxygenase 13 3.01% 0.070665343 354 617
      29414 1.750689046 0.431647973
235955_at, 218416_s_at, 240171_at, 234701_at, 36994_at, 240966_at, 209960_at, 222389_s_at,
1554778_at, 206153_at, 244029_at, 214420_s_at, 238093_at,
PANTHER_MF_ALL MF00243: DNA helicase 30 6.94% 0.071368818 354
      1812 29414 1.375668799 0.424786109
216251_s_at, 235098_at, 226623_at, 1569794_at, 205655_at, 1554890_a_at, 235792_x_at,
229035_s_at, 205885_s_at, 202931_x_at, 229402_at, 217621_at, 218831_s_at, 224683_at,
214420_s_at, 210034_s_at, 235004_at, 212684_at, 228375_at, 226125_at, 236388_at,
213250_at, 219650_at, 205938_at, 210156_s_at, 229131_at, 1552531_a_at, 201079_at,
236098_at, 1570093_at,
PANTHER_BP_ALL BP00246: Ectoderm development 12 2.78% 0.071819729
      354 553 29414 1.803046557 0.323208218
218416_s_at, 208884_s_at, 206563_s_at, 232187_at, 230447_at, 203164_at, 243414_at,
213250_at, 235004_at, 243118_at, 205938_at, 206035_at,
PANTHER_BP_ALL BP00111: Intracellular signaling cascade 20 4.63% 0.071982894
      354 1098 29414 1.513486256 0.317649761
206413_s_at, 226125_at, 218744_s_at, 228712_at, 200710_at, 210330_at, 229164_s_at,
244220_at, 240221_at, 222389_s_at, 1561365_at, 1559633_a_at, 206413_s_at, 230492_s_at,
235885_at, 223103_at, 208357_x_at, 206074_s_at, 227928_at, 208710_s_at,
SP_PIR_KEYWORDS dna-binding 36 8.33% 0.072684287 275 1748 17599
      1.318002912 0.97830241
208698_s_at, 214915_at, 1557248_at, 216889_s_at, 235815_at, 200956_s_at, 1553172_at,
206035_at, 204182_s_at, 234251_at, 1553322_s_at, 231548_at, 224683_at, 206074_s_at,
227928_at, 213442_x_at, 217415_at, 242868_at, 212684_at, 211194_s_at, 219568_x_at,
211180_x_at, 211771_s_at, 233722_at, 233754_x_at, 1554821_a_at, 1558000_at, 1565537_at,
1566163_at, 1558208_at, 223792_at, 239716_at, 207968_s_at, 204913_s_at, 226114_at,
214883_at,
PANTHER_BP_ALL BP00046: Other mRNA transcription 10 2.31% 0.073279757
      354 426 29414 1.950478767 0.316492555
226013_at, 222389_s_at, 217340_at, 229402_at, 1569794_at, 211194_s_at, 232187_at,
208255_s_at, 229035_s_at, 205938_at,
UP_SEQ_FEATURE repeat: Kelch 6 3 0.69% 0.074074123 210 26 12056
      6.624175824 1
1554740_a_at, 204177_s_at, 207976_at,
GOTERM_BP_ALL GO: 0008152~metabolic process 162 37.50% 0.074295604 251
      9181 15360 1.079798007 0.999081974
221667_s_at, 200783_s_at, 216251_s_at, 240171_at, 217340_at, 237548_at, 1557248_at,
200956_s_at, 200991_s_at, 234251_at, 236185_at, 229408_at, 1554345_a_at, 207253_s_at,
206074_s_at, 206286_s_at, 209163_at, 236613_at, 240998_at, 201959_s_at, 224330_s_at,
232857_at, 211180_x_at, 205527_s_at, 209960_at, 213753_x_at, 217340_at, 237942_at,
223792_at, 218585_s_at, 203099_s_at, 243414_at, 207968_s_at, 208255_s_at, 204913_s_at,
214883_at, 202332_at, 213835_x_at, 219960_s_at, 208698_s_at, 211208_s_at, 216669_at,
36994_at, 225215_s_at, 235815_at, 1555349_a_at, 240221_at, 232063_x_at, 231713_s_at,
208884_s_at, 213949_s_at, 225939_at, 226200_at, 224683_at, 218070_s_at, 207934_at,
208688_x_at, 217621_at, 210034_s_at, 213442_x_at, 208710_s_at, 237311_at, 206180_x_at,
238037_at, 204014_at, 200838_at, 227449_at, 213444_at, 226013_at, 229164_s_at, 206775_at,
1565628_at, 1565537_at, 222556_at, 204300_at, 211212_s_at, 1557300_s_at, 1558208_at,
210156_s_at, 234741_at, 239357_at, 218493_at, 218475_at, 216242_x_at, 236098_at,
215766_at, 236805_at, 243160_at, 1570505_at, 1563620_at, 238276_at, 214955_at, 218557_at,
235792_x_at, 216889_s_at, 201819_at, 1553172_at, 206035_at, 204182_s_at, 205774_at,
231548_at, 220233_at, 202415_s_at, 230239_at, 240786_at, 242868_at, 211016_x_at,
204385_at, 237182_at, 1552703_s_at, 235788_at, 219568_x_at, 1552703_s_at, 200710_at,
233722_at, 203167_at, 241849_at, 233754_x_at, 1558000_at, 211925_s_at, 213038_at,
219919_s_at, 226114_at, 219419_at, 1565735_at, 205900_at, 214915_at, 205655_at,
1554890_a_at, 213893_x_at, 1557558_s_at, 1559633_a_at, 1553322_s_at, 203022_at,
206153_at, 221517_s_at, 204177_s_at, 214420_s_at, 238505_at, 223357_s_at, 217415_at,
212684_at, 234472_at, 202695_s_at, 230966_at, 211194_s_at, 214752_x_at, 228712_at,
1560752_at, 211771_s_at, 237093_at, 210981_s_at, 205938_at, 222603_at, 1566163_at,
220129_at, 230492_s_at, 211064_at, 239716_at, 204708_at, 229131_at, 235473_at, 217340_at,
SP_PIR_KEYWORDS initiation factor 4 0.93% 0.075029453 275 63 17599
      4.063261183 0.977157533
225939_at, 208688_x_at, 223357_s_at, 213753_x_at,
PANTHER_MF_ALL MF00188: Select calcium binding protein 17 3.94%
      0.075759635 354 896 29414 1.576491879 0.434802851
206563_s_at, 1563620_at, 211771_s_at, 216889_s_at, 1554690_a_at, 215112_x_at, 219650_at,
233467_s_at, 206413_s_at, 216007_at, 218585_s_at, 244029_at, 208255_s_at, 243414_at,
221766_s_at, 1552461_at, 217415_at,
GOTERM_MF_ALL GO: 0005548~phospholipid transporter activity 3 0.69%
      0.076844853 270 29 16968 6.501149425 0.999999979
215319_at, 1568949_at, 207374_at,
GOTERM_BP_ALL GO: 0008380~RNA splicing 8 1.85% 0.07866584 251
      227 15360 2.156659705 0.99932186 236613_at, 208698_s_at, 1558208_at,
218493_at, 205527_s_at, 217621_at, 237093_at, 233722_at,
INTERPRO IPR008253: Marvel 3 0.69% 0.078779433 288 29 17845
      6.409841954 0.999999999
235955_at, 230571_at, 201079_at,
PANTHER_BP_ALL BP00194: Gametogenesis 18 4.17% 0.079372464 354
      972 29414 1.538711027 0.3325627
216251_s_at, 209574_s_at, 205797_s_at, 224122_at, 229035_s_at, 205885_s_at, 213444_at,
201819_at, 1555393_s_at, 215673_at, 239716_at, 232187_at, 218585_s_at, 208255_s_at,
231147_at, 206286_s_at, 237311_at, 214595_at,
PANTHER_BP_ALL BP00032: Purine metabolism 13 3.01% 0.079504464 354
      630 29414 1.714563716 0.327132105
224881_at, 1556037_s_at, 240171_at, 234480_at, 214752_x_at, 219650_at, 240172_at,
205388_at, 232187_at, 218475_at, 205665_at, 208688_x_at, 238505_at,
PANTHER_MF_ALL MF00040: Cell adhesion molecule 13 3.01% 0.080713105
      354 633 29414 1.706437822 0.446544748
203045_at, 238037_at, 228375_at, 205655_at, 205885_s_at, 213444_at, 1555349_a_at,
1552925_at, 233467_s_at, 216007_at, 205665_at, 226295_at, 208039_at,
GOTERM_MF_ALL GO: 0042802~identical protein binding 11 2.55% 0.08267059
      270 381 16968 1.814406533 0.99999998
221667_s_at, 206775_at, 222826_at, 208698_s_at, 211212_s_at, 210156_s_at, 211194_s_at,
237548_at, 205885_s_at, 230239_at, 241849_at,
GOTERM_BP_ALL GO: 0006974~response to DNA damage stimulus 10 2.31%
      0.083878687 251 324 15360 1.888741331 0.999534039
208884_s_at, 208698_s_at, 226200_at, 237548_at, 218585_s_at, 224683_at, 200956_s_at,
213893_x_at, 236098_at, 202332_at,
GOTERM_MF_ALL GO: 0004857~enzyme inhibitor activity 9 2.08% 0.084675006
      270 285 16968 1.984561404 0.999999958
239719_at, 233351_at, 228712_at, 225858_s_at, 213444_at, 202415_s_at, 222756_s_at,
205075_at, 203167_at,
SP_PIR_KEYWORDS ubl conjugation 11 2.55% 0.0861322 275 391 17599
      1.800409207 0.984617409
233318_at, 202638_s_at, 236613_at, 242868_at, 229408_at, 211194_s_at, 207968_s_at,
239834_at, 200956_s_at, 235815_at, 200710_at,
KEGG_PATHWAY hsa04510: Focal adhesion 8 1.85% 0.086234962 82
      199 4214 2.065939453 0.989236947
1560997_at, 1565735_at, 214752_x_at, 225858_s_at, 205885_s_at, 230239_at, 237311_at,
209960_at,
GOTERM_BP_ALL GO: 0006915~apoptosis 19 4.40% 0.089721303 251 784
      15360 1.483047402 0.999695461
202695_s_at, 237548_at, 211194_s_at, 1552703_s_at, 200838_at, 1552703_s_at, 205655_at,
1554890_a_at, 241849_at, 213753_x_at, 201819_at, 1555349_a_at, 237942_at, 231548_at,
208255_s_at, 225858_s_at, 206286_s_at, 230239_at, 237311_at,
SP_PIR_KEYWORDS atp-binding 25 5.79% 0.091292378 275 1156 17599
      1.384004404 0.985765564
236805_at, 1570505_at, 237548_at, 240221_at, 232063_x_at, 226200_at, 224683_at, 230239_at,
240998_at, 211016_x_at, 234480_at, 202695_s_at, 227449_at, 228712_at, 210981_s_at,
237093_at, 1565628_at, 237942_at, 211212_s_at, 234741_at, 239357_at, 204708_at, 236713_at,
1552531_a_at, 202332_at, 236098_at,
PANTHER_MF_ALL MF00132: Nucleotidyltransferase 7 1.62% 0.091868174
      354 258 29414 2.254390575 0.482134778
204029_at, 211740_at, 203099_s_at, 211571_s_at, 204014_at, 218070_s_at, 240612_at,
PANTHER_BP_ALL BP00057: Regulation of nucleoside, nucleotide metabolism 5
      1.16% 0.092160508 354 142 29414 2.925718151 0.36401523
211422_at, 1561365_at, 226295_at, 205885_s_at, 1568949_at,
INTERPRO IPR012937: Protein of unknown function DUF1693 2 0.46%
      0.092712378 288 6 17845 20.65393519 1
221766_s_at, 1552461_at,
INTERPRO IPR012346: p53 and RUNT-type transcription factor, DNA-binding 2
      0.46% 0.092712378 288 6 17845 20.65393519 1
211194_s_at, 211180_x_at,
INTERPRO IPR000315: Zinc finger, B-box 4 0.93% 0.093269311 288 67
      17845 3.699212272 1
238941_at, 212057_at, 239716_at, 235788_at,
PANTHER_BP_ALL BP00031: Nucleoside, nucleotide and nucleic acid metabolism 56
      12.96% 0.093352063 354 3842 29414 1.211104151 0.361843389
212057_at, 238276_at, 1557248_at, 200956_s_at, 206035_at, 204182_s_at, 229408_at,
1554345_a_at, 206074_s_at, 235004_at, 242868_at, 236613_at, 232857_at, 219568_x_at,
219650_at, 1558000_at, 1565748_at, 223792_at, 207968_s_at, 204913_s_at, 226114_at,
202332_at, 214883_at, 213835_x_at, 208698_s_at, 214915_at, 1554890_a_at, 213893_x_at,
235815_at, 240221_at, 1553322_s_at, 203022_at, 224683_at, 221517_s_at, 217621_at,
213442_x_at, 217415_at, 212684_at, 206180_x_at, 211194_s_at, 211771_s_at, 213361_at,
213444_at, 1554821_a_at, 1565537_at, 1566163_at, 1557300_s_at, 211212_s_at, 1558208_at,
211064_at, 239716_at, 229131_at, 218475_at, 216242_x_at, 235473_at, 236098_at,
PANTHER_BP_ALL BP00151: MHCII-mediated immunity 46 10.65% 0.093735015
      354 3074 29414 1.243382626 0.357187115
215319_at, 204029_at, 219960_s_at, 216251_s_at, 207566_at, 205655_at, 243636_s_at,
225215_s_at, 205885_s_at, 216889_s_at, 1555349_a_at, 229444_at, 1561365_at, 205774_at,
1554778_at, 226200_at, 1554345_a_at, 231548_at, 206153_at, 244029_at, 208688_x_at,
206074_s_at, 218980_at, 230239_at, 1556037_s_at, 240786_at, 212793_at, 234480_at,
230966_at, 208389_s_at, 231643_s_at, 235788_at, 204014_at, 236388_at, 205527_s_at,
214752_x_at, 1560752_at, 213361_at, 1559611_at, 205938_at, 209960_at, 241849_at,
1552925_at, 1555393_s_at, 1556282_at, 207976_at,
BBID 60.IL-15_rheumatoid-arthritis_synovitis 2 0.46% 0.09503797 8 5
      356 17.8 0.999993097
202638_s_at, 1555349_a_at,
GOTERM_MF_ALL GO: 0016772~transferase activity, transferring phosphorus-containing
groups 26 6.02% 0.095067023 270 1193 16968 1.369619074 0.999999984
221667_s_at, 236805_at, 220434_at, 237548_at, 211208_s_at, 235792_x_at, 240221_at,
1561365_at, 1554345_a_at, 218070_s_at, 230239_at, 217415_at, 242868_at, 240998_at,
234480_at, 202695_s_at, 232857_at, 227449_at, 228712_at, 237093_at, 210981_s_at,
1565628_at, 237942_at, 204708_at, 216242_x_at, 202332_at,
GOTERM_BP_ALL GO: 0012501~programmed cell death 19 4.40% 0.09582727
      251 791 15360 1.469923089 0.999803818
202695_s_at, 237548_at, 211194_s_at, 1552703_s_at, 200838_at, 1552703_s_at, 205655_at,
1554890_a_at, 241849_at, 213753_x_at, 201819_at, 1555349_a_at, 237942_at, 231548_at,
208255_s_at, 225858_s_at, 206286_s_at, 230239_at, 237311_at,
UP_SEQ_FEATURE domain: EGF-like 4 0.93% 0.095923857 210 63
      12056 3.645049131 1
242426_at, 240171_at, 206286_s_at, 1557558_s_at,
INTERPRO IPR001810: Cyclin-like F-box 4 0.93% 0.096471391 288 68
      17845 3.644812092 1
1563620_at, 220233_at, 224683_at, 1560752_at,
GOTERM_BP_ALL GO: 0019222~regulation of metabolic process 58 13.43%
      0.097880517 251 2975 15360 1.19304965 0.999814149
238276_at, 1557248_at, 216889_s_at, 200956_s_at, 1553172_at, 206035_at, 204182_s_at,
234251_at, 229408_at, 231548_at, 207253_s_at, 206074_s_at, 206286_s_at, 240786_at,
242868_at, 201959_s_at, 219568_x_at, 211180_x_at, 233754_x_at, 213753_x_at, 203167_at,
1558000_at, 223792_at, 203099_s_at, 207968_s_at, 204913_s_at, 226114_at, 214883_at,
1565735_at, 208698_s_at, 214915_at, 205655_at, 1554890_a_at, 235815_at, 1555349_a_at,
231713_s_at, 1553322_s_at, 226200_at, 225939_at, 221517_s_at, 213442_x_at, 223357_s_at,
217415_at, 212684_at, 206180_x_at, 211194_s_at, 214752_x_at, 211771_s_at, 213444_at,
226013_at, 1565537_at, 1566163_at, 220129_at, 1557300_s_at, 1558208_at, 211064_at,
239716_at, 235473_at,
UP_SEQ_FEATURE domain: F-box 4 0.93% 0.099419582 210 64 12056
      3.588095238 1
1563620_at, 220233_at, 224683_at, 1560752_at,
UP_SEQ_FEATURE domain: RRM 2 4 0.93% 0.099419582 210 64 12056
      3.588095238 1
208698_s_at, 1558208_at, 1554890_a_at, 233722_at,
UP_SEQ_FEATURE domain: RRM 1 4 0.93% 0.099419582 210 64 12056
      3.588095238 1
208698_s_at, 1558208_at, 1554890_a_at, 233722_at,
```

TABLE 6
Full DAVID results for the Leading Edge Set

| | | | | | | | Benjamini- | |
|---|---|---|---|---|---|---|---|---|
| Annotation | | | p-value | | Pop hits/ | Fold | Hochberg | |
| category¹ | Term | Count (%) | (EASE) | List total | pop total | enrichment | FDR | Genes |
| P_{BP} | BP00301: | 11 | 6.8E−09 | 75 | 315/29414 | 13.7 | 1.5E−06 | C21ORF59, C21ORF90, |
| | Non-vertebrate | (14.1%) | | | | | | PDXK, C21ORF77, |
| | process | | | | | | | C21ORF45, C21ORF89, |
| | | | | | | | | C21ORF51, C21ORF121, |
| | | | | | | | | C21ORF86, C21ORF84, |
| | | | | | | | | C21ORF56 |
| P_{MF} | MF00198: | 11 | 1.8E−08 | 75 | 350/29414 | 12.3 | 4.4E−06 | C21ORF90, PDXK, |
| | Structural | (14.1%) | | | | | | C21ORF45, C21ORF89, |
| | protein | | | | | | | RIPK4, C21ORF51, |
| | | | | | | | | C21ORF121, C21ORF86, |
| | | | | | | | | CRYAA, C21ORF84, |
| | | | | | | | | KRTAP13-1 |
| P_{BP} | BP00182: | 13 | 2.6E−08 | 75 | 589/29414 | 8.7 | 2.9E−06 | C21ORF45, C21ORF89, |
| | Sensory | (16.7%) | | | | | | C21ORF51, C21ORF86, |
| | perception | | | | | | | C21ORF84, C21ORF56, |
| | | | | | | | | SIM2, C21ORF90, |
| | | | | | | | | C21ORF59, PDXK, |
| | | | | | | | | C21ORF77, C21ORF121, |
| | | | | | | | | CRYAA |
| P_{MF} | MF00216: | 13 | 7.0E−06 | 75 | 999/29414 | 5.1 | 8.3E−04 | TMPRSS2, C21ORF45, |
| | Serine protease | (16.7%) | | | | | | C21ORF89, C21ORF51, |
| | | | | | | | | C21ORF86, C21ORF84, |
| | | | | | | | | C21ORF56, C21ORF90, |
| | | | | | | | | C21ORF59, ITSN1, PDXK, |
| | | | | | | | | C21ORF77, C21ORF121 |
| G_{MF} | GO: 0019961~ | 3 | 9.5E−05 | 54 | 5/16968 | 188.5 | 0.2 | IFNAR1, IFNAR2, |
| | interferon | (3.9%) | | | | | | IFNGR2 |
| | binding | | | | | | | |
| G_{MF} | GO: 0004904~ | 3 | 9.5E−05 | 54 | 5/16968 | 188.5 | 0.2 | IFNAR1, IFNAR2, |
| | interferon | (3.9%) | | | | | | IFNGR2 |
| | receptor activity | | | | | | | |
| G_{CC} | GO: 0005622~ | 52 | 4.5E−04 | 60 | 10544/15857 | 1.3 | 0.3 | TFF3, DYRK1A, SOD1, |
| | intracellular | (66.7%) | | | | | | PCNT, C21ORF7, PFKL, |
| | | | | | | | | H2BFS, CRYZL1, |
| | | | | | | | | SNF1LK, PRDM15, |
| | | | | | | | | KCNE2, PCP4, KCNE1, |
| | | | | | | | | B3GALT5, RUNX1, |
| | | | | | | | | DIP2A, KRTAP13-1, |
| | | | | | | | | ITSN1, HMGN1, POFUT2, |
| | | | | | | | | CHAF1B, ADARB1, |
| | | | | | | | | BRWD1, C21ORF66, |
| | | | | | | | | OLIG2, PDE9A, CLIC6, |
| | | | | | | | | SON, NDUFV3, |
| | | | | | | | | DONSON, DSCR3, |
| | | | | | | | | KRTAP19-1, DSCR6, |
| | | | | | | | | MX2, TMEM1, KRTAP10- |
| | | | | | | | | 12, C21ORF33, HLCS, |
| | | | | | | | | GART, ZNF295, SIM2, |
| | | | | | | | | TMEM50B, C21ORF59, |
| | | | | | | | | CLDN14, PDXK, |
| | | | | | | | | SH3BGR, CBR1, RIPK4, |
| | | | | | | | | IFNGR2, UBE2G2, |
| | | | | | | | | CRYAA, CSTB |
| G_{CC} | GO: 0044424~ | 50 | 4.9E−04 | 60 | 9906/15857 | 1.3 | 0.2 | TFF3, DYRK1A, SOD1, |
| | intracellular part | (64.1%) | | | | | | PCNT, C21ORF7, PFKL, |
| | | | | | | | | H2BFS, CRYZL1, |
| | | | | | | | | SNF1LK, PRDM15, |
| | | | | | | | | KCNE2, PCP4, KCNE1, |
| | | | | | | | | B3GALT5, RUNX1, |
| | | | | | | | | DIP2A, KRTAP13-1, |
| | | | | | | | | HMGN1, POFUT2, |
| | | | | | | | | CHAF1B, BRWD1, |
| | | | | | | | | C21ORF66, OLIG2, |
| | | | | | | | | PDE9A, CLIC6, SON, |
| | | | | | | | | NDUFV3, DONSON, |
| | | | | | | | | DSCR3, KRTAP19-1, |
| | | | | | | | | DSCR6, MX2, TMEM1, |
| | | | | | | | | KRTAP10-12, C21ORF33, |
| | | | | | | | | HLCS, GART, ZNF295, |
| | | | | | | | | SIM2, TMEM50B, |
| | | | | | | | | C21ORF59, CLDN14, |
| | | | | | | | | PDXK, SH3BGR, CBR1, |
| | | | | | | | | RIPK4, IFNGR2, UBE2G2, |
| | | | | | | | | CRYAA, CSTB |
| SP | cytokine receptor | 3 | 1.5E−03 | 60 | 17/17599 | 51.8 | 0.8 | IFNAR1, IFNAR2, |
| | | (3.9%) | | | | | | IFNGR2 |
| G_{CC} | GO: 0005737~ | 35 | 3.2E−03 | 60 | 6219/15857 | 1.5 | 0.6 | BRWD1, TFF3, |
| | cytoplasm | (44.9%) | | | | | | C21ORF66, SOD1, PCNT, |
| | | | | | | | | OLIG2, PDE9A, CLIC6, |
| | | | | | | | | NDUFV3, C21ORF7, |
| | | | | | | | | PFKL, CRYZL1, SNF1LK, |
| | | | | | | | | KCNE2, MX2, KCNE1, |
| | | | | | | | | PCP4, TMEM1, |
| | | | | | | | | B3GALT5, C21ORF33, |
| | | | | | | | | HLCS, GART, TMEM50B, |
| | | | | | | | | C21ORF59, PDXK, |
| | | | | | | | | CLDN14, SH3BGR, |
| | | | | | | | | POFUT2, CHAF1B, |
| | | | | | | | | RIPK4, CBR1, IFNGR2, |
| | | | | | | | | UBE2G2, CRYAA, CSTB |
| SP | acetylation | 8 | 3.8E−03 | 60 | 599/17599 | 3.9 | 0.9 | PDXK, SOD1, H2BFS, |
| | | (10.3%) | | | | | | CBR1, C21ORF33, SON, |
| | | | | | | | | CRYAA, CSTB |
| G_{BP} | GO: 0051704~ | 6 | 4.1E−03 | 52 | 320/15360 | 5.5 | 1.0 | IFNAR1, IFNAR2, SOD1, |
| | multi-organism | (7.7%) | | | | | | MX2, H2BFS, IFNGR2 |
| | process | | | | | | | |
| G_{BP} | GO: 0009615~ | 4 | 4.3E−03 | 52 | 99/15360 | 11.9 | 1.0 | IFNAR1, IFNAR2, MX2, |
| | response to virus | (5.1%) | | | | | | IFNGR2 |
| G_{BP} | GO: 0051707~ | 5 | 4.4E−03 | 52 | 201/15360 | 7.3 | 1.0 | IFNAR1, IFNAR2, MX2, |
| | response to other | (6.4%) | | | | | | H2BFS, IFNGR2 |
| | organism | | | | | | | |
| I | IPR000369: | 2 | 1.2E−02 | 54 | 4/17845 | 165.2 | 1.0 | KCNE2, KCNE1 |
| | Potassium | (2.6%) | | | | | | |
| | channel, voltage- | | | | | | | |
| | dependent, beta | | | | | | | |
| | subunit, KCNE | | | | | | | |
| G_{BP} | GO: 0060047~ | 3 | 1.2E−02 | 52 | 51/15360 | 17.4 | 1.0 | SOD1, KCNE2, KCNE1 |
| | heart contraction | (3.9%) | | | | | | |
| G_{BP} | GO: 0003015~ | 3 | 1.2E−02 | 52 | 51/15360 | 17.4 | 1.0 | SOD1, KCNE2, KCNE1 |
| | heart process | (3.9%) | | | | | | |
| P_{BP} | BP00149: | 19 | 1.3E−02 | 75 | 4138/29414 | 1.8 | 0.6 | IFNAR1, BRWD1, |
| | T-cell mediated | (24.4%) | | | | | | PRDM15, DYRK1A, MX2, |
| | immunity | | | | | | | TMEM1, BACE2, |
| | | | | | | | | NDUFV3, C21ORF84, |
| | | | | | | | | TRPM2, SIM2, |
| | | | | | | | | C21ORF90, IFNAR2, |
| | | | | | | | | HMGN1, ITSN1, |
| | | | | | | | | SH3BGR, CBR1, |
| | | | | | | | | ADARB1, SNF1LK |
| P_{BP} | BP00285: | 10 | 1.9E−02 | 75 | 1608/29414 | 2.4 | 0.7 | BRWD1, CLDN14, ITSN1, |
| | Cell structure | (12.8%) | | | | | | PCNT, CBR1, KRTAP10- |
| | and motility | | | | | | | 12, SLC19A1, ZNF295, |
| | | | | | | | | KRTAP13-1, KRTAP19-1, |
| G_{BP} | GO: 0009607~ | 5 | 2.0E−02 | 52 | 313/15360 | 4.7 | 1.0 | IFNAR1, IFNAR2, MX2, |
| | response to | (6.4%) | | | | | | H2BFS, IFNGR2 |
| | biotic stimulus | | | | | | | |
| P_{MF} | MF00212: | 18 | 2.1E−02 | 75 | 4033/29414 | 1.8 | 0.8 | DYRK1A, MX2, |
| | Other G-protein | (23.1%) | | | | | | KRTAP10-12, B3GALT5, |
| | modulator | | | | | | | BACE2, RUNX1, ZNF295, |
| | | | | | | | | DIP2A, TMEM50B, PFKL, |
| | | | | | | | | TTC3, LRRC3, HMGN1, |
| | | | | | | | | ITSN1, CHAF1B, |
| | | | | | | | | C21ORF29, ADARB1, |
| | | | | | | | | UBE2G2 |
| G_{CC} | GO: 0043231~ | 36 | 2.1E−02 | 60 | 7191/15857 | 1.3 | 1.0 | BRWD1, TFF3, |
| | intracellular | (46.2%) | | | | | | C21ORF66, SOD1, |
| | membrane- | | | | | | | DYRK1A, OLIG2, SON, |
| | bound organelle | | | | | | | NDUFV3, DONSON, |
| | | | | | | | | C21ORF7, DSCR3, |
| | | | | | | | | H2BFS, SNF1LK, |
| | | | | | | | | PRDM15, DSCR6, |
| | | | | | | | | KCNE2, MX2, TMEM1, |
| | | | | | | | | KCNE1, PCP4, B3GALT5, |
| | | | | | | | | C21ORF33, RUNX1, |
| | | | | | | | | HLCS, ZNF295, DIP2A, |
| | | | | | | | | TMEM50B, SIM2, |
| | | | | | | | | C21ORF59, CLDN14, |
| | | | | | | | | HMGN1, POFUT2, |
| | | | | | | | | CHAF1B, IFNGR2, |
| | | | | | | | | UBE2G2, CSTB |
| G_{CC} | GO: 0043227~ | 36 | 2.2E−02 | 60 | 7194/15857 | 1.3 | 1.0 | BRWD1, TFF3, |
| | membrane- | (46.2%) | | | | | | C21ORF66, SOD1, |
| | bound organelle | | | | | | | DYRK1A, OLIG2, SON, |
| | | | | | | | | NDUFV3, DONSON, |
| | | | | | | | | C21ORF7, DSCR3, |
| | | | | | | | | H2BFS, SNF1LK, |
| | | | | | | | | PRDM15, DSCR6, |
| | | | | | | | | KCNE2, MX2, TMEM1, |
| | | | | | | | | KCNE1, PCP4, B3GALT5, |
| | | | | | | | | C21ORF33, RUNX1, |
| | | | | | | | | HLCS, ZNF295, DIP2A, |
| | | | | | | | | TMEM50B, SIM2, |
| | | | | | | | | C21ORF59, CLDN14, |
| | | | | | | | | HMGN1, POFUT2, |
| | | | | | | | | CHAF1B, IFNGR2, |
| | | | | | | | | UBE2G2, CSTB |
| G_{CC} | GO: 0043229~ | 40 | 2.2E−02 | 60 | 8288/15857/ | 1.3 | 1.0 | BRWD1, TFF3, |
| | intracellular | (51.3%) | | | | | | C21ORF66, SOD1, |
| | organelle | | | | | | | DYRK1A, PCNT, OLIG2, |
| | | | | | | | | SON, NDUFV3, |
| | | | | | | | | DONSON, C21ORF7, |
| | | | | | | | | DSCR3, H2BFS, |
| | | | | | | | | KRTAP19-1, SNF1LK, |
| | | | | | | | | PRDM15, DSCR6, MX2, |
| | | | | | | | | KCNE2, TMEM1, KCNE1, |
| | | | | | | | | PCP4, KRTAP10-12, |
| | | | | | | | | B3GALT5, C21ORF33, |
| | | | | | | | | RUNX1, HLCS, ZNF295, |
| | | | | | | | | KRTAP13-1, DIP2A, |
| | | | | | | | | TMEM50B, SIM2, |
| | | | | | | | | C21ORF59, CLDN14, |
| | | | | | | | | HMGN1, POFUT2, |
| | | | | | | | | CHAF1B, IFNGR2, |
| | | | | | | | | UBE2G2, CSTB |
| G_{CC} | GO: 0043226~ | 40 | 2.2E−02 | 60 | 8292/15857 | 1.3 | 0.9 | BRWD1, TFF3, |
| | organelle | (51.3%) | | | | | | C21ORF66, SOD1, |
| | | | | | | | | DYRK1A, PCNT, OLIG2, |
| | | | | | | | | SON, NDUFV3, |
| | | | | | | | | DONSON, C21ORF7, |
| | | | | | | | | DSCR3, H2BFS, |
| | | | | | | | | KRTAP19-1, SNF1LK, |
| | | | | | | | | PRDM15, DSCR6, MX2, |
| | | | | | | | | KCNE2, TMEM1, KCNE1, |
| | | | | | | | | PCP4, KRTAP10-12, |
| | | | | | | | | B3GALT5, C21ORF33, |
| | | | | | | | | RUNX1, HLCS, ZNF295, |
| | | | | | | | | KRTAP13-1, DIP2A, |
| | | | | | | | | TMEM50B, SIM2, |
| | | | | | | | | C21ORF59, CLDN14, |
| | | | | | | | | HMGN1, POFUT2, |
| | | | | | | | | CHAF1B, IFNGR2, |
| | | | | | | | | UBE2G2, CSTB |
| SP | long qt syndrome | 2 | 2.3E−02 | 60 | 7/17599 | 83.8 | 1.0 | KCNE2, KCNE1 |
| | | (2.6%) | | | | | | |
| S | SM00018: PD | 2 | 2.3E−02 | 27 | 9/9899 | 81.5 | 1.0 | TFF3, TFF2 |
| | | (2.6%) | | | | | | |
| I | IPRO00519: | 2 | 2.6E−02 | 54 | 9/17845 | 73.4 | 1.0 | TFF3, TFF2 |
| | P-type trefoil | (2.6%) | | | | | | |
| SP | phosphoprotein | 24 | 2.7E−02 | 60 | 4705/17599 | 1.5 | 1.0 | IFNAR1, BRWD1, |
| | | (30.8%) | | | | | | C21ORF66, DYRK1A, |
| | | | | | | | | TMEM1, KCNE1, CLIC6, |
| | | | | | | | | SON, RUNX1, GART, |
| | | | | | | | | ZNF295, PFKL, |
| | | | | | | | | C21ORF59, IFNAR2, |
| | | | | | | | | CLDN14, HMGN1, ITSN1, |
| | | | | | | | | H2BFS, CHAF1B, RIPK4, |
| | | | | | | | | IFNGR2, ADARB1, |
| | | | | | | | | CRYAA, SNF1LK |
| P_{MF} | MF00107: | 6 | 2.7E−02 | 75 | 670/29414 | 3.5 | 0.8 | PDXK, DYRK1A, RIPK4, |
| | Kinase | (7.7%) | | | | | | C21ORF7, SNF1LK, PFKL |
| SP | alternative | 27 | 2.8E−02 | 60 | 5530/17599 | 1.4 | 1.0 | IFNAR1, BRWD1, |
| | splicing | (34.6%) | | | | | | C21ORF66, DYRK1A, |
| | | | | | | | | PDE9A, CLIC6, SON, |
| | | | | | | | | DONSON, C21ORF7, |
| | | | | | | | | TRPM2, PFKL, |
| | | | | | | | | C21ORF90, C21ORF29, |
| | | | | | | | | CRYZL1, DSCR6, |
| | | | | | | | | C21ORF33, RUNX1, |
| | | | | | | | | PIGP, GART, SIM2, TTC3, |
| | | | | | | | | IFNAR2, ITSN1, PDXK, |
| | | | | | | | | POFUT2, RIPK4, |
| | | | | | | | | ADARB1 |
| P_{BP} | BP00070: | 4 | 2.8E−02 | 75 | 261/29414 | 6.0 | 0.7 | HMGN1, SON, SLC19A1, |
| | Protein-lipid | (5.1%) | | | | | | PIGP |
| | modification | | | | | | | |
| P_{BP} | BP00036: | 9 | 3.1E−02 | 75 | 1465/29414 | 2.4 | 0.7 | IFNAR1, IFNAR2, MX2, |
| | DNA repair | (11.5%) | | | | | | OLIG2, KCNE1, |
| | | | | | | | | ADARB1, HLCS, |
| | | | | | | | | CRYZL1, TRPM2 |
| G_{MF} | GO: 0019955~ | 3 | 3.3E−02 | 54 | 91/16968 | 10.4 | 1.0 | IFNAR1, IFNAR2, |
| | cytokine binding | (3.6%) | | | | | | IFNGR2 |
| B | h_ifnaPathway: | 2 | 4.5E−02 | 10 | 8/1557 | 38.9 | 1.0 | IFNAR1, IFNAR2 |
| | IFN alpha | (2.6%) | | | | | | |
| | signaling | | | | | | | |
| | pathway | | | | | | | |
| SP | blocked amino | 3 | 4.8E−02 | 60 | 105/17599 | 8.4 | 1.0 | CBR1, CRYAA, CSTB |
| | end | (3.9%) | | | | | | |
| I | IPR014720: | 2 | 5.5E−02 | 54 | 19/17845 | 34.8 | 1.0 | SON, ADARB1 |
| | Double-stranded | (2.6%) | | | | | | |
| | RNA-binding- | | | | | | | |
| | like | | | | | | | |
| S | SM00358: | 2 | 6.4E−02 | 27 | 25/9899 | 29.3 | 1.0 | SON, ADARB1 |
| | DSRM | (2.6%) | | | | | | |
| P_{MF} | MF00024: | 4 | 6.8E−02 | 75 | 373/29414 | 4.2 | 1.0 | KCNE2, KCNE1, CLIC6, |
| | Ion channel | (5.1%) | | | | | | TRPM2 |
| P_{BP} | BP00137: | 4 | 6.9E−02 | 75 | 375/29414 | 4.2 | 0.9 | POFUT2, KCNE2, PCNT, |
| | Protein targeting | (5.1%) | | | | | | PCP4 |
| | and localization | | | | | | | |
| P_{BP} | BP00063: | 13 | 7.1E−02 | 75 | 3004/29414 | 1.7 | 0.9 | BRWD1, DYRK1A, PCP4, |
| | Protein | (16.7%) | | | | | | B3GALT5, SON, PIGP, |
| | modification | | | | | | | SLC19A1, HLCS, TTC3, |
| | | | | | | | | ITSN1, RIPK4, ADARB1, |
| | | | | | | | | UBE2G2 |
| I | IPR001159: | 2 | 7.2E−02 | 54 | 25/17845 | 26.4 | 1.0 | SON, ADARB1 |
| | Double-stranded | (2.6%) | | | | | | |
| | RNA binding | | | | | | | |
| G_{BP} | GO: 0006357~ | 5 | 7.3E−02 | 52 | 476/15360 | 3.1 | 1.0 | BRWD1, IFNAR2, OLIG2, |
| | regulation of | (6.4%) | | | | | | RUNX1, SIM2 |
| | transcription | | | | | | | |
| | from RNA | | | | | | | |
| | polymerase II | | | | | | | |
| | promoter | | | | | | | |
| G_{BP} | GO: 0007605~ | 3 | 7.5E−02 | 52 | 136/15360 | 6.5 | 1.0 | CLDN14, SOD1, KCNE1 |
| | sensory | (3.9%) | | | | | | |
| | perception of | | | | | | | |
| | sound | | | | | | | |
| G_{BP} | GO: 0050954~ | 3 | 7.6E−02 | 52 | 137/15360 | 6.5 | 1.0 | CLDN14, SOD1, KCNE1 |
| | sensory | (3.9%) | | | | | | |
| | perception of | | | | | | | |
| | mechanical | | | | | | | |
| | stimulus | | | | | | | |
| P | BP00103: | 12 | 7.8E−02 | 75 | 2721/29414 | 1.7 | 0.9 | IFNAR1, LRRC3, |
| | Cell surface | (15.4%) | | | | | | PRDM15, ITSN1, TFF3, |
| | receptor | | | | | | | OLIG2, CBR1, TFF2, |
| | mediated signal | | | | | | | DONSON, LOC284837, |
| | transduction | | | | | | | CRYZL1, PLAC4 |
| B | h_ranklPathway: | 2 | 7.8E−02 | 10 | 14/1557 | 22.2 | 1.0 | IFNAR1, IFNAR2 |
| | Bone | (2.6%) | | | | | | |
| | Remodelling | | | | | | | |
| G_{MF} | GO: 0046873~metal | 4 | 7.9E−02 | 54 | 322/16968 | 3.9 | 1.0 | KCNE2, TMEM1, KCNE1, |
| | ion | (5.1%) | | | | | | TRPM2 |
| | transmembrane | | | | | | | |
| | transporter | | | | | | | |
| | activity | | | | | | | |
| SP | Keratin | 3 | 8.1E−02 | 60 | 141/17599 | 6.2 | 1.0 | KRTAP10-12, KRTAP13- |
| | | (3.9%) | | | | | | 1, KRTAP19-1 |
| G_{MF} | GO: 0005515~ | 29 | 8.4E−02 | 54 | 7212/16968 | 1.3 | 1.0 | BRWD1, IFNAR1, SOD1, |
| | protein binding | (37.2%) | | | | | | DYRK1A, PCNT, OLIG2, |
| | | | | | | | | SON, C21ORF56, |
| | | | | | | | | C21ORF7, SNF1LK, |
| | | | | | | | | DSCR6, RUNX1, GART, |
| | | | | | | | | DIP2A, ZNF295, SIM2, |
| | | | | | | | | TTC3, LRRC3, ITSN1, |
| | | | | | | | | CLDN14, IFNAR2, |
| | | | | | | | | SH3BGR, CHAF1B, |
| | | | | | | | | CBR1, RIPK4, IFNGR2, |
| | | | | | | | | UBE2G2, CRYAA, CSTB |
| K | hsa04650: | 3 | 8.4E−02 | 17 | 129/4214 | 5.8 | 1.0 | IFNAR1, IFNAR2, |
| | Natural killer | (3.9%) | | | | | | IFNGR2 |
| | cell mediated | | | | | | | |
| | cytotoxicity | | | | | | | |
| P_{BP} | BP00067: | 9 | 8.8E−02 | 75 | 1828/29414 | 1.9 | 0.9 | PRDM15, ITSN1, PCP4, |
| | Protein | (11.5%) | | | | | | B3GALT5, BACE2, |
| | glycosylation | | | | | | | NDUFV3, GART, CSTB, |
| | | | | | | | | C21ORF56 |
| SP | voltage-gated | 3 | 8.8E−02 | 60 | 148/17599 | 5.9 | 1.0 | KCNE2, KCNE1, CLIC6 |
| | channel | (3.9%) | | | | | | |
| I | IPR001969: | 2 | 8.8E−02 | 54 | 31/17845 | 21.3 | 1.0 | RIPK4, BACE2 |
| | Peptidase | (2.6%) | | | | | | |
| | aspartic, active | | | | | | | |
| | site | | | | | | | |
| P_{MF} | MF00042: | 28 | 8.9E−02 | 75 | 8438/29414 | 1.3 | 1.0 | BRWD1, TFF3, |
| | Nucleic acid | (35.9%) | | | | | | C21ORF66, OLIG2, SON, |
| | binding | | | | | | | NDUFV3, SLC19A1, |
| | | | | | | | | PLAC4, H2BFS, TFF2, |
| | | | | | | | | CRYZL1, TMPRSS2, |
| | | | | | | | | MX2, TMEM1, B3GALT5, |
| | | | | | | | | BACE2, LOC284837, |
| | | | | | | | | ZNF295, SIM2, ITSN1, |
| | | | | | | | | HMGN1, CLDN14, |
| | | | | | | | | POFUT2, CHAF1B, |
| | | | | | | | | RIPK4, CBR1, ADARB1, |
| | | | | | | | | CRYAA |
| SP | Ionic channel | 4 | 9.0E−02 | 60 | 316/17599 | 3.7 | 1.0 | KCNE2, KCNE1, CLIC6, |
| | | (5.1%) | | | | | | TRPM2 |
| SP | disease mutation | 9 | 9.3E−02 | 60 | 1395/17599 | 1.9 | 1.0 | CLDN14, SOD1, KCNE2, |
| | | (11.5%) | | | | | | KCNE1, IFNGR2, RUNX1, |
| | | | | | | | | HLCS, CRYAA, CSTB |
| SP | transferase | 9 | 9.4E−02 | 60 | 1398/17599 | 1.9 | 1.0 | PDXK, POFUT2, |
| | | (11.5%) | | | | | | DYRK1A, RIPK4, |
| | | | | | | | | B3GALT5, PIGP, GART, |
| | | | | | | | | SNF1LK, PFKL |
| G_{BP} | GO: 0042552~ | 2 | 9.5E−02 | 52 | 30/15360 | 19.7 | 1.0 | SOD1, OLIG2 |
| | myelination | (2.6%) | | | | | | |
| P_{BP} | BP00273: | 6 | 9.5E−02 | 75 | 964/29414 | 2.4 | 0.9 | TMEM1, CHAF1B, |
| | Chromatin | (7.7%) | | | | | | H2BFS, CBR1, CLIC6, |
| | packaging and | | | | | | | SLC19A1 |
| | remodeling | | | | | | | |

TABLE 7
Over-represented functional terms among lists of
differentially expressed genes in trisomy 21 samples

| | | | | | |
|---|---|---|---|---|---|
| | | | | Raw | BH adj |
| List | Ontology term | Source | # | p-value | p-value |

G-protein signaling

| | | | | | |
|---|---|---|---|---|---|
| Indiv | G-protein mediated signaling | Panther | 81 | 3.9e−6 | 0.00085 |
| | | BP | | | |
| Indiv | G-protein modulator | Panther | 33 | 0.03 | 0.34 |
| | | MF | | | |
| Indiv | Large G-protein | Panther | 18 | 0.048 | 0.38 |
| | | MF | | | |
| 21 | Other G-protein modulator | Panther | 19 | 0.017 | 0.74 |
| | | MF | | | |

Ion Transport

| | | | | | |
|---|---|---|---|---|---|
| Indiv | Ion Transport | Panther | 52 | 0.00013 | 0.0057 |
| | | BP | | | |
| Indiv | Proteolysis (possibly relevant | Panther | 95 | 5.4e−5 | 0.0054 |
| | to transmembrane ion | BP | | | |
| | transport) | | | | |
| Indiv | Cation Transport | Panther | 80 | 0.0002 | 0.0062 |
| | | BP | | | |
| Indiv | Calcium mediated signaling | Panther | 11 | 0.06 | 0.31 |
| | | BP | | | |
| 21 | Ion Channel | Panther | 4 | 0.077 | 0.96 |
| | | MF | | | |
| 21 | Voltage gated channel | PIR kwds | 3 | 0.098 | 1.0 |
| 21 | Potassium channel, voltage | Interpro | 2 | 0.013 | 1.0 |
| | dependent, beta subunit, | | | | |
| | KCNE | | | | |
| 21 | Metal ion transmembrane | GO MF | 4 | 0.086 | 1.0 |
| | transporter activity | | | | |

Oxidative Stress

| | | | | | |
|---|---|---|---|---|---|
| Indiv | Oxidative Phosphorylation | Panther | 30 | 0.0089 | 0.13 |
| | | BP | | | |
| Indiv | Antioxidation and free radical | Panther | 9 | 0.021 | 0.21 |
| | removal | BP | | | |
| Indiv | Oxygenase (relevant?) | Panther | 13 | 0.071 | 0.42 |
| | | MF | | | |
| Indiv | Glycosyltransferase | Panther | 26 | 0.025 | 0.32 |
| | (relevant?) | MF | | | |
| Indiv | Phospholipid transporter | GO MF | 3 | 0.077 | 1.0 |
| | activity | | | | |
| 21 | Protein-lipid modification | Panther | 5 | 0.0052 | 0.32 |
| | | BP | | | |

Cell structure

| | | | | | |
|---|---|---|---|---|---|
| Indiv | Cell structure | Panther | 68 | 0.00014 | 0.0054 |
| | | BP | | | |
| Indiv | Extracellular matrix structural | Panther | 11 | 0.043 | 0.38 |
| | protein | MF | | | |
| Indiv | Actin bining cytoskeletal | Panther | 32 | 0.047 | 0.38 |
| | protein | MF | | | |
| Indiv | Microtubule family | Panther | 20 | 0.013 | 0.23 |
| | cytoskeletal protein | MF | | | |
| Indiv | Cytoskeleton | PIR kwds | 12 | 0.022 | 0.88 |
| 21 | Structural protein | Panther | 12 | 2.2e−9 | 5.3e−7 |
| | | MF | | | |
| 21 | Keratin | PIR kwds | 3 | 0.091 | 1.0 |

Circulatory system functions

| | | | | | |
|---|---|---|---|---|---|
| Indiv | Blood vessel morphogenesis | GO BP | 10 | 0.0016 | 0.82 |
| Indiv | Blood vessel development | GO BP | 10 | 0.0037 | 0.89 |
| Indiv | Angiogenesis | GO BP | 8 | 0.009 | 0.91 |
| Indiv | Negative regulation of blood | GO BP | 3 | 0.016 | 0.95 |
| | coagulation | | | | |
| Indiv | Blood coagulation | GO BP | 6 | 0.023 | 0.97 |
| Indiv | Vasculature development | GO BP | 10 | 0.0041 | 0.84 |
| 21 | Heart contraction | GO BP | 3 | 0.014 | 1 |
| 21 | Heart process | GO BP | 3 | 0.014 | 1 |

Immune and stress response

| | | | | | |
|---|---|---|---|---|---|
| Indiv | Response to stress | GO BP | 33 | 0.00073 | 0.72 |
| Indiv | Response to wounding | GO BP | 15 | 0.011 | 0.93 |
| Indiv | Response to DNA damage | GO BP | 10 | 0.084 | 1.0 |
| | stimulus | | | | |
| Indiv | MHCI-mediated immunity | Panther | 84 | 0.011 | 0.14 |
| | | BP | | | |
| Indiv | Immune system development | GO BP | 8 | 0.066 | 1.0 |
| Indiv | MHCII-mediated immunity | Panther | 46 | 0.094 | 0.36 |
| | | BP | | | |
| Indiv | Wound healing | GO BP | 7 | 0.019 | 0.96 |
| Indiv | Host-virus interaction | PIR kwds | 8 | 0.051 | 0.95 |
| 21 | Interferon binding/interferon | GO MF | 3 | 0.0001 | 0.26 |
| | receptor activity | | | | |
| 21 | Cytokine receptor | PIR kwds | 3 | 0.0017 | 0.83 |
| 21 | Response to virus | GO BP | 4 | 0.0051 | 1.0 |
| 21 | Response to other organism | GO BP | 5 | 0.0054 | 1.0 |
| 21 | T-cell mediated immunity | Panther | 20 | 0.011 | 0.44 |
| | | BP | | | |
| 21 | Response to biotic stimulus | GO BP | 5 | 0.024 | 1.0 |
| 21 | DNA repair | Panther | 10 | 0.015 | 0.49 |
| | | BP | | | |
| 21 | Natural killer cell mediated | KEGG | 3 | 0.094 | 1.0 |
| | cytotoxicity | | | | |

Sensory perception

| | | | | | |
|---|---|---|---|---|---|
| Indiv | Sensory perception | Panther | 13 | 0.053 | 0.3 |
| | | BP | | | |
| 21 | Sensory perception | Panther | 14 | 4.9e−9 | 5.4e−7 |
| | | BP | | | |
| 21 | Sensory perception of sound | GO BP | 3 | 0.083 | 1.0 |
| 21 | Sensory perception of | GO BP | 3 | 0.084 | 1.0 |
| | mechanical stimulus | | | | |

Developmental processes

| | | | | | |
|---|---|---|---|---|---|
| Indiv | Wnt signaling pathway | KEGG | 12 | 0.00012 | 0.024 |
| Indiv | Hedgehog signaling pathway | KEGG | 7 | 0.00069 | 0.067 |
| Indiv | Developmental processes | Panther | 42 | 0.0014 | 0.028 |
| | | BP | | | |
| Indiv | Anatomical structure | GO BP | 10 | 0.0027 | 0.91 |
| | formation | | | | |
| Indiv | Mesoderm development | Panther | 22 | 0.0033 | 0.059 |
| | | BP | | | |
| Indiv | Organ morphogenesis | GO BP | 16 | 0.0033 | 0.89 |
| Indiv | Blood vessel development | GO BP | 10 | 0.0037 | 0.89 |
| Indiv | Neurogenesis | Panther | 23 | 0.016 | 0.17 |
| | | BP | | | |
| Indiv | Embryogenesis | Panther | 5 | 0.022 | 0.21 |
| | | BP | | | |
| Indiv | Oogenesis | Panther | 14 | 0.033 | 0.25 |
| | | BP | | | |
| Indiv | Hemopoiesis | GO BP | 8 | 0.038 | 0.99 |
| Indiv | Developmental process | GO BP | 66 | 0.04 | 0.99 |
| Indiv | Wnt receptor signaling | GO BP | 3 | 0.049 | 1.0 |
| | pathway, calcium modulating | | | | |
| | pathway | | | | |
| Indiv | Hemopoietic or lymphoid | GO BP | 8 | 0.052 | 1.0 |
| | organ development | | | | |
| Indiv | Multicellular organismal | GO BP | 49 | 0.053 | 1.0 |
| | development | | | | |
| Indiv | Immune system development | GO BP | 8 | 0.066 | 1.0 |
| Indiv | Ectoderm development | Panther | 12 | 0.072 | 0.32 |
| | | BP | | | |
| Indiv | Gametogenesis | Panther | 18 | 0.079 | 0.33 |
| | | BP | | | |
| 21 | Bone Remodeling | Biocarta | 2 | 0.078 | 1.0 |

List = either the list of 414 genes identified in analyses of individual genes that are differentially expressed (″Indiv″) or the 82-gene leading edge subste of genes on Chr21q22 identified by GSEA analysis (″21″).
Source = database from which the functional category was derived
Raw p-value = ″EASE″ value used by the DAVID software (Hosack et al. ″Identifying biological themes within lists of genes with EASE.″ Genome Biol., 2003,, 4(10): R70, the contents of which are herein incorporated by reference in their entirety.) The authors of the DAVID software suggest that adjustment using the Benjamin-Hochberg approach may be too strict, and suggest that all terms with raw p-values below 0.1 have potential signfiicance.
BH adjusted p-val = significance (adjusted for multiple testing using the Benjamin-Hochberg false discovery rate approach)

### Example 5

### Identification of Potential Drugs for Down Syndrome

In this Example, compounds that may be useful in treatments for Down Syndrome were identified using genomic approaches. The lists of differentially-expressed genes in trisomy 21 fetuses obtained in Example 3 was used in conjunction with the Connectivity Map (Lamb et al. (2006) and Lamb et al. (2007), the contents of each which are herein incorporated by reference in their entirety), a publicly available reference collection of gene expression profiles of human cells treated with bioactive small molecules.

### Materials and Methods

To identify compounds with molecular signatures that might mimic or mitigate the effects of DS, we used Connectivity Map build 1.0, which contains a database of 564 expression profiles representing the effects of 164 compounds on 4 cancer cell lines, using the Affymetrix U133A microarrays (Lamb et al. (2006)). Since the U133 Plus 2.0 arrays used in the present study contain a superset of the probe sets on the U133A arrays, the Connectivity Map analysis was run using only those probe sets that were common to both arrays.

### Results and Discussion

To further confirm the importance of functional processes identified in Example 4, the Connectivity Map was used to identify compounds whose molecular signatures either mimic or counteract that of DS. Four compounds with average connectivity scores above 0.7 (indicating a high correlation with the DS molecular signature), and 9 compounds with average connectivity scores below −0.7 (indicating a high negative correlation) were found.

The full results of the Connectivity Map analysis appear in Table 8.

TABLE 8
Compound scores for genes differentially-expressed in Down Syndrome

| # | Compound name | Score | n | instances |
|---|---|---|---|---|
| 1 | fisetin | 0.879 | 1 | 0.879 50 μM PC3 (86) [579] |
| 2 | DL-PPMP | 0.807 | 1 | 0.807 2 μM MCF7 (514) [1121] |
| 3 | tolbutamide | 0.773 | 1 | 0.773 100 μM MCF7 (6) [142] |
| 4 | phentolamine | 0.726 | 1 | 0.726 12 μM MCF7 (514) [1138] |
| 5 | chlorpropamide | 0.639 | 2 | 0.695 100 μM MCF7 (6) [141] |
| | | | | 0.583 100 μM MCF7 (6) [144] |
| 6 | dexverapamil | 0.629 | 1 | 0.629 10 μM MCF7 (7) [164] |
| 7 | yohimbine | 0.623 | 1 | 0.623 23 μM MCF7 (514) [1119] |
| 8 | prednisolone | 0.548 | 1 | 0.548 1 μM MCF7 (21) [265] |
| 9 | oxamic acid | 0.427 | 1 | 0.427 10 mM MCF7 (58) [439] |
| 10 | 4,5- | 0.404 | 2 | 0.807 10 μM PC3 (86) [578] |
| | dianilinophthalimide | | | 0.0 10 μM MCF7 (107) [624] |
| 11 | quercetin | 0.392 | 2 | 0.785 1 μM MCF7 (22a) [283] |
| | | | | 0.0 1 μM MCF7 (505) [917] |
| 12 | tacrolimus | 0.389 | 3 | 0.801 1 μM MCF7 (22a) [284] |
| | | | | 0.717 1 μM MCF7 (39) [378] |
| | | | | 0.351 1 μM MCF7 (8) [169] |
| 13 | 5253409 | 0.372 | 2 | 0.745 17 μM MCF7 (504) [844] |
| | | | | 0.0 17 μM MCF7 (502) [961] |
| 14 | clozapine | 0.336 | 2 | 0.673 10 μM MCF7 (46) [416] |
| | | | | 0.0 10 μM MCF7 (506) [1009] |
| 15 | exisulind | 0.316 | 2 | 0.632 50 μM MCF7 (24) [314] |
| | | | | 0.0 50 μM MCF7 (23) [309] |
| 16 | iloprost | 0.310 | 3 | 0.81 1 μM SKMEL5 (69) [496] |
| | | | | 0.651 1 μM MCF7 (54) [427] |
| | | | | −0.532 1 μM MCF7 (67) [488] |
| 17 | NU-1025 | 0.308 | 2 | 0.617 100 μM MCF7 (24) [313] |
| | | | | 0.0 100 μM MCF7 (98) [608] |
| 18 | imatinib | 0.307 | 2 | 0.614 10 μM PC3 (66) [483] |
| | | | | 0.0 10 μM MCF7 (36) [366] |
| 19 | benserazide | 0.279 | 2 | 0.558 10 μM MCF7 (112) [641] |
| | | | | 0.0 10 μM SKMEL5 (109) [631] |
| 20 | monastrol | 0.274 | 8 | 1.0 100 μM MCF7 (24) [311] |
| | | | | 0.639 100 μM PC3 (116) [668] |
| | | | | 0.567 20 μM MCF7 (98) [605] |
| | | | | 0.52 100 μM MCF7 (108) [627] |
| | | | | 0.0 100 μM MCF7 (101) [610] |
| | | | | 0.0 100 μM MCF7 (95) [596] |
| | | | | 0.0 20 μM MCF7 (103) [614] |
| | | | | −0.532 100 μM MCF7 (117) [681] |
| 21 | tamoxifen | 0.251 | 3 | 0.754 1 μM MCF7 (39) [380] |
| | | | | 0.0 1 μM ssMCF7 (38) [375] |
| | | | | 0.0 1 μM MCF7 (6) [143] |
| 22 | LM-1685 | 0.234 | 3 | 0.701 10 μM MCF7 (18) [253] |
| | | | | 0.0 10 μM MCF7 (101) [612] |
| | | | | 0.0 10 μM MCF7 (16) [207] |
| 23 | tretinoin | 0.224 | 8 | 0.931 1 μM PC3 (60) [447] |
| | | | | 0.81 1 μM MCF7 (17) [224] |
| | | | | 0.607 1 μM MCF7 (504) [849] |
| | | | | 0.583 1 μM MCF7 (40) [384] |
| | | | | 0.0 1 μM MCF7 (513) [1049] |
| | | | | 0.0 1 μM MCF7 (506) [991] |
| | | | | −0.567 1 μM MCF7 (502) [966] |
| | | | | −0.57 1 μM HL60 (41) [390] |
| 24 | chlorpromazine | 0.208 | 4 | 0.769 1 μM MCF7 (54) [426] |
| | | | | 0.757 10 μM MCF7 (46) [419] |
| | | | | 0.0 1 μM MCF7 (513) [1055] |
| | | | | −0.693 1 μM MCF7 (506) [997] |
| 25 | pirinixic acid | 0.153 | 5 | 0.654 100 μM SKMEL5 (69) [495] |
| | | | | 0.651 100 μM PC3 (66) [481] |
| | | | | 0.0 100 μM MCF7 (36) [368] |
| | | | | 0.0 100 μM PC3 (65) [464] |
| | | | | −0.538 100 μM MCF7 (67) [487] |
| 26 | SC-58125 | 0.124 | 4 | 0.495 10 μM MCF7 (18) [254] |
| | | | | 0.0 10 μM MCF7 (16) [208] |
| | | | | 0.0 10 μM SKMEL5 (73) [507] |
| | | | | 0.0 10 μM HL60 (75) [542] |
| 27 | celecoxib | 0.088 | 5 | 0.717 10 μM MCF7 (39) [377] |
| | | | | 0.611 10 μM MCF7 (18) [252] |
| | | | | 0.0 10 μM MCF7 (505) [922] |
| | | | | −0.316 10 μM PC3 (66) [482] |
| | | | | −0.573 10 μM MCF7 (16) [206] |
| 28 | staurosporine | 0.085 | 4 | 0.972 1 μM MCF7 (24) [312] |
| | | | | 0.0 10 nM SKMEL5 (73) [508] |
| | | | | 0.0 10 nM MCF7 (54) [425] |
| | | | | −0.632 100 nM MCF7 (53) [423] |
| 29 | sulindac | 0.060 | 2 | 0.601 50 μM MCF7 (23) [307] |
| | | | | −0.48 100 μM MCF7 (8) [168] |
| 30 | monorden | 0.045 | 10 | 0.829 100 nM MCF7 (26b) [325] |
| | | | | 0.67 100 nM SKMEL5 (69) [493] |
| | | | | 0.511 100 nM PC3 (66) [484] |
| | | | | 0.461 100 nM MCF7 (68) [489] |
| | | | | 0.0 100 nM MCF7 (513) [1057] |
| | | | | 0.0 100 nM HL60 (75) [544] |
| | | | | 0.0 100 nM PC3 (61) [449] |
| | | | | −0.596 100 nM MCF7 (506) [999] |
| | | | | −0.678 100 nM MCF7 (502) [953] |
| | | | | −0.746 100 nM MCF7 (504) [836] |
| 31 | valproic acid | 0.036 | 18 | 0.91 50 μM MCF7 (513) [1060] |
| | | | | 0.757 50 μM MCF7 (506) [1002] |
| | | | | 0.52 500 μM MCF7 (513) [1078] |
| | | | | 0.486 1 mM SKMEL5 (109) [629] |
| | | | | 0.0 1 mM MCF7 (2) [23] |
| | | | | 0.0 1 mM PC3 (56) [433] |
| | | | | 0.0 500 μM MCF7 (33) [347] |
| | | | | 0.0 1 mM HL60 (44) [409] |
| | | | | 0.0 10 mM MCF7 (33) [345] |
| | | | | 0.0 1 mM PC3 (63) [458] |
| | | | | 0.0 2 mM MCF7 (33) [346] |
| | | | | 0.0 50 μM MCF7 (33) [348] |
| | | | | 0.0 200 μM MCF7 (506) [994] |
| | | | | 0.0 10 mM HL60 (44) [410] |
| | | | | 0.0 500 μM MCF7 (506) [1020] |
| | | | | −0.626 1 mM MCF7 (506) [989] |
| | | | | −0.637 1 mM ssMCF7 (70) [497] |
| | | | | −0.766 1 mM MCF7 (513) [1047] |
| 32 | rosiglitazone | 0.025 | 4 | 0.676 10 μM MCF7 (513) [1071] |
| | | | | 0.0 10 μM HL60 (37) [369] |
| | | | | 0.0 10 μM PC3 (55) [430] |
| | | | | −0.576 10 μM MCF7 (506) [1013] |
| 33 | alpha-estradiol | 0.024 | 6 | 0.916 10 nM PC3 (110b) [702] |
| | | | | 0.0 10 nM ssMCF7 (42) [403] |
| | | | | 0.0 10 nM MCF7 (5) [122] |
| | | | | 0.0 10 nM MCF7 (506) [990] |
| | | | | 0.0 10 nM MCF7 (513) [1048] |
| | | | | −0.775 10 nM MCF7 (119) [762] |
| 34 | depudecin | 0.019 | 2 | 0.629 1 μM MCF7 (504) [874] |
| | | | | −0.591 1 μM MCF7 (502) [982] |
| 35 | metformin | 0.017 | 5 | 0.664 1 mM MCF7 (1) [4] |
| | | | | 0.0 10 μM MCF7 (1) [2] |
| | | | | 0.0 10 μM MCF7 (1) [1] |
| | | | | 0.0 100 nM MCF7 (1) [3-0.579 10 μM MCF7 |
| | | | | (2a) [61] |
| 36 | tyrphostin AG-825 | 0.000 | 1 | 0.0 25 μM MCF7 (514) [1114] |
| 37 | phenformin | 0.000 | 1 | 0.0 10 μM MCF7 (2) [21] |
| 38 | pararosaniline | 0.000 | 1 | 0.0 10 μM MCF7 (505) [893] |
| 39 | doxycycline | 0.000 | 1 | 0.0 14 μM MCF7 (514) [1113] |
| 40 | sulfasalazine | 0.000 | 1 | 0.0 100 μM MCF7 (13) [204] |
| 41 | thalidomide | 0.000 | 2 | 0.0 100 μM MCF7 (21) [266] |
| | | | | 0.0 100 μM MCF7 (98) [606] |
| 42 | 3-aminobenzamide | 0.000 | 1 | 0.0 10 mM MCF7 (94) [590] |
| 43 | sulindac sulfide | 0.000 | 1 | 0.0 50 μM MCF7 (23) [308] |
| 44 | butirosin | 0.000 | 1 | 0.0 10 μM PC3 (116) [666] |
| 45 | mesalazine | 0.000 | 1 | 0.0 100 μM MCF7 (5) [124] |
| 46 | splitomicin | 0.000 | 1 | 0.0 20 μM PC3 (90) [661] |
| 47 | phenanthridinone | 0.000 | 1 | 0.0 51 μM MCF7 (514) [1115] |
| 48 | tomelukast | 0.000 | 1 | 0.0 1 μM MCF7 (17) [222] |
| 49 | (−)-catechin | 0.000 | 1 | 0.0 11 μM MCF7 (514) [1101] |
| 50 | probucol | 0.000 | 1 | 0.0 10 μM MCF7 (94) [592] |
| 51 | 5252917 | 0.000 | 2 | 0.0 14 μM MCF7 (502) [944] |
| | | | | 0.0 14 μM MCF7 (504) [828] |
| 52 | 5248896 | 0.000 | 2 | 0.0 11 μM MCF7 (502) [955] |
| | | | | 0.0 11 μM MCF7 (504) [838] |
| 53 | arachidonyltrifluoromethane | 0.000 | 2 | 0.0 10 μM MCF7 (26b) [327] |
| | | | | 0.0 10 μM MCF7 (95) [594] |
| 54 | fasudil | 0.000 | 2 | 0.0 10 μM PC3 (56) [436] |
| | | | | 0.0 10 μM MCF7 (31) [343] |
| 55 | ciclosporin | 0.000 | 2 | 0.0 1 μM MCF7 (96) [602] |
| | | | | 0.0 1 μM MCF7 (20) [261] |
| 56 | MK-886 | 0.000 | 2 | 0.0 1 μM MCF7 (96) [601] |
| | | | | 0.0 1 μM MCF7 (20) [264] |
| 57 | 5211181 | 0.000 | 2 | 0.0 12 μM MCF7 (502) [950] |
| | | | | 0.0 12 μM MCF7 (504) [834] |
| 58 | 5230742 | 0.000 | 2 | 0.0 17 μM MCF7 (502) [970] |
| | | | | 0.0 17 μM MCF7 (504) [862] |
| 59 | TTNPB | 0.000 | 2 | 0.0 100 nM MCF7 (17) [223] |
| | | | | 0.0 100 nM PC3 (61) [451] |
| 60 | demecolcine | 0.000 | 1 | 0.0 12 μM MCF7 (514) [1103] |
| 61 | amitriptyline | 0.000 | 1 | 0.0 1 μM MCF7 (8) [167] |
| 62 | phenyl biguanide | 0.000 | 1 | 0.0 10 μM MCF7 (2) [22] |
| 63 | N-phenylanthranilic | 0.000 | 1 | 0.0 10 μM MCF7 (25) [317] |
| | acid | | | |
| 64 | colchicine | 0.000 | 2 | 0.0 1 μM SKMEL5 (109) [630] |
| | | | | 0.0 100 nM MCF7 (112) [644] |
| 65 | monensin | 0.000 | 1 | 0.0 11 μM MCF7 (514) [1105] |
| 66 | fludrocortisone | 0.000 | 2 | 0.0 1 μM MCF7 (22a) [282] |
| | | | | 0.0 1 μM MCF7 (22a) [281] |
| 67 | 12,13-EODE | 0.000 | 1 | 0.0 200 nM MCF7 (514) [1108] |
| 68 | minocycline | 0.000 | 1 | 0.0 11 μM MCF7 (514) [1135] |
| 69 | tyrphostin AG-1478 | 0.000 | 1 | 0.0 32 μM MCF7 (514) [1141] |
| 70 | 5149715 | 0.000 | 1 | 0.0 10 μM MCF7 (505) [890] |
| 71 | azathioprine | 0.000 | 1 | 0.0 100 μM MCF7 (29) [338] |
| 72 | tioguanine | 0.000 | 1 | 0.0 10 μM MCF7 (112) [642] |
| 73 | 5186324 | 0.000 | 1 | 0.0 2 μM MCF7 (505) [900] |
| 74 | 5286656 | 0.000 | 1 | 0.0 50 μM MCF7 (505) [889] |
| 75 | clofibrate | 0.000 | 2 | 0.0 100 μM MCF7 (59) [444] |
| | | | | 0.0 150 μM MCF7 (20) [263] |
| 76 | 5151277 | 0.000 | 1 | 0.0 14 μM MCF7 (505) [903] |
| 77 | dopamine | 0.000 | 1 | 0.0 1 μM MCF7 (68) [491] |
| 78 | BW-B70C | 0.000 | 1 | 0.0 32 μM MCF7 (514) [1132] |
| 79 | quinpirole | 0.000 | 1 | 0.0 1 μM MCF7 (62) [456] |
| 80 | 2-deoxy-D-glucose | 0.000 | 1 | 0.0 10 mM MCF7 (31) [344] |
| 81 | topiramate | 0.000 | 1 | 0.0 3 μM MCF7 (505) [915] |
| 82 | 5666823 | 0.000 | 1 | 0.0 100 μM MCF7 (101) [609] |
| 83 | 1,5-isoquinolinediol | 0.000 | 1 | 0.0 100 μM HL60 (75) [543] |
| 84 | gefitinib | 0.000 | 1 | 0.0 10 μM HL60 (75) [541] |
| 85 | clotrimazole | 0.000 | 1 | 0.0 50 μM MCF7 (505) [905] |
| 86 | 5186223 | 0.000 | 1 | 0.0 12 μM MCF7 (505) [885] |
| 87 | pentamidine | 0.000 | 1 | 0.0 100 μM MCF7 (111) [639] |
| 88 | ikarugamycin | −0.001 | 3 | 0.816 2 μM MCF7 (502) [974] |
| | | | | 0.0 2 μM MCF7 (504) [866] |
| | | | | −0.819 2 μM MCF7 (505) [918] |
| 89 | ionomycin | −0.005 | 3 | 0.791 2 μM MCF7 (502) [979] |
| | | | | 0.0 2 μM MCF7 (505) [882] |
| | | | | −0.807 2 μM MCF7 (504) [871] |
| 90 | arachidonic acid | −0.023 | 3 | 0.489 10 μM MCF7 (96) [604] |
| | | | | 0.0 10 μM MCF7 (59) [441] |
| | | | | −0.558 10 μM MCF7 (59) [443] |
| 91 | trifluoperazine | −0.035 | 3 | 0.695 10 μM MCF7 (505) [910] |
| | | | | 0.0 10 μM MCF7 (53) [421] |
| | | | | −0.801 10 μM MCF7 (506) [1004] |
| 92 | tetraethylenepentamine | −0.049 | 6 | 0.745 100 μM HL60 (44) [412] |
| | | | | 0.0 100 μM PC3 (63) [457] |
| | | | | 0.0 10 μM MCF7 (43) [405] |
| | | | | 0.0 100 μM MCF7 (43) [406] |
| | | | | −0.462 100 μM MCF7 (82) [574] |
| | | | | −0.579 100 μM ssMCF7 (70) [498] |
| 93 | wortmannin | −0.063 | 8 | 0.564 10 nM MCF7 (513) [1081] |
| | | | | 0.0 1 μM MCF7 (502) [977] |
| | | | | 0.0 10 nM MCF7 (506) [1023] |
| | | | | 0.0 1 μM MCF7 (504) [869] |
| | | | | 0.0 10 nM HL60 (41) [389] |
| | | | | 0.0 10 nM MCF7 (505) [911] |
| | | | | −0.526 10 nM SKMEL5 (73) [506] |
| | | | | −0.541 10 nM ssMCF7 (42) [404] |
| 94 | haloperidol | −0.101 | 6 | 0.807 10 μM MCF7 (513) [1082] |
| | | | | 0.0 10 μM MCF7 (46) [418] |
| | | | | 0.0 10 μM MCF7 (68) [492] |
| | | | | 0.0 10 μM MCF7 (506) [983] |
| | | | | −0.652 10 μM MCF7 (513) [1041] |
| | | | | −0.76 10 μM MCF7 (506) [1024] |
| 95 | trichostatin A | −0.106 | 12 | 0.71 100 nM PC3 (60) [448] |
| | | | | 0.492 1 μM MCF7 (513) [1072] |
| | | | | 0.0 1 μM MCF7 (504) [873] |
| | | | | 0.0 100 nM HL60 (35) [364] |
| | | | | 0.0 100 nM MCF7 (514) [1112] |
| | | | | 0.0 1 μM MCF7 (502) [981] |
| | | | | 0.0 100 nM MCF7 (506) [992] |
| | | | | 0.0 100 nM MCF7 (513) [1050] |
| | | | | −0.57 100 nM ssMCF7 (45) [413] |
| | | | | −0.576 1 μM MCF7 (506) [1014] |
| | | | | −0.626 100 nM MCF7 (28) [332] |
| | | | | −0.699 100 nM MCF7 (28) [331] |
| 96 | dexamethasone | −0.111 | 3 | 0.854 1 μM ssMCF7 (38) [374] |
| | | | | −0.573 1 μM MCF7 (19) [255] |
| | | | | −0.614 1 μM MCF7 (5) [123] |
| 97 | genistein | −0.111 | 7 | 0.838 10 μM PC3 (110b) [703] |
| | | | | 0.0 1 μM MCF7 (21) [267] |
| | | | | 0.0 1 μM MCF7 (21) [268] |
| | | | | 0.0 10 μM MCF7 (40) [382] |
| | | | | 0.0 10 μM MCF7 (513) [1073] |
| | | | | −0.787 10 μM MCF7 (111) [638] |
| | | | | −0.83 10 μM MCF7 (506) [1015] |
| 98 | estradiol | −0.113 | 10 | 0.732 10 nM ssMCF7 (38) [373] |
| | | | | 0.604 10 nM ssMCF7 (45) [414] |
| | | | | 0.0 10 nM MCF7 (5) [121] |
| | | | | 0.0 100 nM MCF7 (36) [365] |
| | | | | 0.0 10 nM PC3 (116) [665] |
| | | | | 0.0 10 nM HL60 (41) [387] |
| | | | | −0.477 10 nM HL60 (120) [782] |
| | | | | −0.482 10 nM MCF7 (506) [1021] |
| | | | | −0.635 100 nM MCF7 (506) [988] |
| | | | | −0.871 10 nM MCF7 (513) [1079] |
| 99 | cobalt chloride | −0.127 | 3 | 0.794 100 μM MCF7 (39) [379] |
| | | | | −0.471 100 μM MCF7 (40) [383] |
| | | | | −0.705 100 μM MCF7 (62) [454] |
| 100 | fulvestrant | −0.136 | 7 | 0.664 1 μM PC3 (110b) [704] |
| | | | | 0.0 1 μM MCF7 (36) [367] |
| | | | | 0.0 10 nM MCF7 (23) [310] |
| | | | | 0.0 1 μM MCF7 (506) [985] |
| | | | | 0.0 1 μM MCF7 (513) [1043] |
| | | | | −0.719 10 nM MCF7 (513) [1076] |
| | | | | −0.895 1 μM ssMCF7 (74) [523] |
| 101 | thioridazine | −0.157 | 4 | 0.0 10 μM MCF7 (506) [1010] |
| | | | | 0.0 10 μM MCF7 (46) [417] |
| | | | | 0.0 1 μM MCF7 (53) [422] |
| | | | | −0.629 10 μM MCF7 (513) [1068] |
| 102 | rofecoxib | −0.162 | 6 | 0.0 10 μM HL60 (37) [371] |
| | | | | 0.0 10 μM MCF7 (7) [166] |
| | | | | 0.0 10 μM MCF7 (19) [256] |
| | | | | 0.0 10 μM PC3 (65) [463] |
| | | | | −0.474 10 μM MCF7 (16) [205] |
| | | | | −0.497 10 μM MCF7 (18) [251] |
| 103 | troglitazone | −0.167 | 6 | 0.0 10 μM HL60 (37) [370] |
| | | | | 0.0 10 μM MCF7 (513) [1070] |
| | | | | 0.0 10 μM PC3 (65) [462] |
| | | | | 0.0 10 μM PC3 (55) [431] |
| | | | | 0.0 10 μM MCF7 (506) [1012] |
| | | | | −1.0 10 μM SKMEL5 (71) [504] |
| 104 | novobiocin | −0.182 | 6 | 0.0 100 μM MCF7 (82) [576] |
| | | | | 0.0 100 μM PC3 (56) [435] |
| | | | | 0.0 100 μM SKMEL5 (109) [632] |
| | | | | 0.0 100 μM MCF7 (31) [342] |
| | | | | −0.43 100 μM ssMCF7 (70) [499] |
| | | | | −0.661 100 μM MCF7 (58) [437] |
| 105 | deferoxamine | −0.197 | 3 | 0.0 100 μM PC3 (63) [460] |
| | | | | 0.0 100 μM MCF7 (82) [573] |
| | | | | −0.591 100 μM MCF7 (67) [485] |
| 106 | raloxifene | −0.198 | 3 | 0.0 100 nM ssMCF7 (38) [376] |
| | | | | 0.0 100 nM HL60 (41) [388] |
| | | | | −0.594 100 nM MCF7 (13) [202] |
| 107 | geldanamycin | −0.203 | 6 | 0.0 1 μM MCF7 (502) [972] |
| | | | | 0.0 1 μM MCF7 (513) [1066] |
| | | | | 0.0 1 μM MCF7 (95) [593] |
| | | | | 0.0 1 μM MCF7 (504) [864] |
| | | | | −0.585 1 μM MCF7 (506) [1008] |
| | | | | −0.635 1 μM MCF7 (101) [611] |
| 108 | bucladesine | −0.206 | 3 | 0.0 2 μM MCF7 (502) [959] |
| | | | | 0.0 20 μM MCF7 (94) [591] |
| | | | | −0.617 2 μM MCF7 (504) [842] |
| 109 | sodium | −0.231 | 7 | 0.408 1 mM MCF7 (43) [408] |
| | phenylbutyrate | | | 0.0 1 mM HL60 (44) [411] |
| | | | | 0.0 100 μM MCF7 (31) [341] |
| | | | | 0.0 1 mM MCF7 (43) [407] |
| | | | | −0.5 100 μM HL60 (35) [363] |
| | | | | −0.749 1 mM PC3 (56) [434] |
| | | | | −0.778 200 μM SKMEL5 (71) [502] |
| 110 | W-13 | −0.240 | 2 | 0.0 10 μM MCF7 (58) [440] |
| | | | | −0.48 10 μM MCF7 (112) [643] |
| 111 | LY-294002 | −0.249 | 17 | 0.741 10 μM MCF7 (26b) [328] |
| | | | | 0.0 100 nM MCF7 (513) [1054] |
| | | | | 0.0 10 μM MCF7 (25) [318] |
| | | | | 0.0 10 μM MCF7 (513) [1065] |
| | | | | 0.0 10 μM MCF7 (506) [1016] |
| | | | | 0.0 10 μM MCF7 (513) [1074] |
| | | | | 0.0 10 μM PC3 (65) [461] |
| | | | | 0.0 10 μM MCF7 (506) [1019] |
| | | | | 0.0 100 nM MCF7 (506) [996] |
| | | | | −0.526 10 μM SKMEL5 (71) [501] |
| | | | | −0.541 10 μM ssMCF7 (42) [401] |
| | | | | −0.547 10 μM PC3 (55) [429] |
| | | | | −0.649 10 μM HL60 (35) [361] |
| | | | | −0.667 10 μM MCF7 (513) [1077] |
| | | | | −0.667 10 μM MCF7 (19) [258] |
| | | | | −0.684 10 μM MCF7 (506) [1007] |
| | | | | −0.687 100 nM MCF7 (53) [424] |
| 112 | copper sulfate | −0.262 | 4 | 0.0 100 μM MCF7 (82) [575] |
| | | | | 0.0 100 μM MCF7 (58) [438] |
| | | | | −0.48 100 μM ssMCF7 (70) [500] |
| | | | | −0.567 100 μM PC3 (63) [459] |
| 113 | 15-delta prostaglandin | −0.266 | 5 | 0.0 10 μM PC3 (60) [446] |
| | J2 | | | 0.0 10 μM SKMEL5 (79) [564] |
| | | | | 0.0 10 μM MCF7 (513) [1069] |
| | | | | −0.488 10 μM MCF7 (506) [1011] |
| | | | | −0.842 10 μM MCF7 (13) [201] |
| 114 | blebbistatin | −0.274 | 2 | 0.0 17 μM MCF7 (504) [837] |
| | | | | −0.547 17 μM MCF7 (502) [954] |
| 115 | prochlorperazine | −0.276 | 3 | 0.0 10 μM MCF7 (513) [1053] |
| | | | | 0.0 10 μM MCF7 (506) [995] |
| | | | | −0.827 10 μM MCF7 (62) [455] |
| 116 | 17-dimethylamino- | −0.278 | 2 | 0.0 100 nM MCF7 (506) [993] |
| | geldanamycin | | | −0.556 100 nM MCF7 (513) [1051] |
| 117 | butein | −0.285 | 2 | 0.0 10 μM PC3 (87) [582] |
| | | | | −0.57 10 μM MCF7 (98) [607] |
| 118 | nordihydroguaiaretic | −0.290 | 5 | 0.0 1 μM MCF7 (506) [1003] |
| | acid | | | 0.0 1 μM MCF7 (513) [1061] |
| | | | | 0.0 1 μM ssMCF7 (45) [415] |
| | | | | −0.675 1 μM ssMCF7 (74) [524] |
| | | | | −0.775 1 μM MCF7 (13) [203] |
| 119 | acetylsalicylic acid | −0.298 | 3 | 0.0 100 μM MCF7 (506) [984] |
| | | | | −0.339 100 μM MCF7 (25) [315] |
| | | | | −0.556 100 μM MCF7 (513) [1042] |
| 120 | 5182598 | −0.300 | 2 | 0.0 25 μM MCF7 (502) [976] |
| | | | | −0.599 25 μM MCF7 (504) [868] |
| 121 | sirolimus | −0.302 | 1 | 0.586 100 nM MCF7 (26b) [326] |
| | | | 0 | 0.0 100 nM ssMCF7 (42) [402] |
| | | | | 0.0 100 nM MCF7 (506) [1022] |
| | | | | 0.0 100 nM HL60 (35) [362] |
| | | | | −0.389 100 nM MCF7 (513) [1080] |
| | | | | −0.529 100 nM MCF7 (513) [1045] |
| | | | | −0.579 100 nM MCF7 (513) [1059] |
| | | | | −0.649 100 nM MCF7 (506) [987] |
| | | | | −0.678 100 nM MCF7 (505) [921] |
| | | | | −0.787 100 nM MCF7 (506) [1001] |
| 122 | docosahexaenoic acid | −0.304 | 2 | 0.0 100 μM PC3 (90) [664] |
| | ethyl ester | | | −0.608 100 μM MCF7 (505) [881] |
| 123 | diclofenac | −0.322 | 2 | 0.0 10 μM PC3 (60) [445] |
| | | | | −0.643 10 μM MCF7 (28) [333] |
| 124 | mercaptopurine | −0.330 | 2 | 0.0 10 μM PC3 (116) [667] |
| | | | | −0.661 100 μM MCF7 (28) [334] |
| 125 | indometacin | −0.347 | 4 | 0.0 20 μM MCF7 (20) [262] |
| | | | | 0.0 100 μM PC3 (61) [452] |
| | | | | −0.693 100 μM SKMEL5 (71) [503] |
| | | | | −0.696 100 μM MCF7 (62) [453] |
| 126 | 5279552 | −0.356 | 2 | 0.0 22 μM MCF7 (504) [843] |
| | | | | −0.711 22 μM MCF7 (502) [960] |
| 127 | 17-allylamino- | −0.416 | 1 | 0.523 100 nM MCF7 (17) [221] |
| | geldanamycin | | 8 | 0.0 1 μM MCF7 (513) [1064] |
| | | | | 0.0 1 μM MCF7 (506) [1005] |
| | | | | 0.0 1 μM MCF7 (506) [1006] |
| | | | | 0.0 1 μM MCF7 (505) [916] |
| | | | | 0.0 1 μM PC3 (61) [450] |
| | | | | −0.444 1 μM MCF7 (40) [381] |
| | | | | −0.518 1 μM MCF7 (502) [947] |
| | | | | −0.57 1 μM MCF7 (506) [998] |
| | | | | −0.579 1 μM MCF7 (513) [1063] |
| | | | | −0.594 1 μM MCF7 (506) [986] |
| | | | | −0.626 1 μM MCF7 (513) [1044] |
| | | | | −0.661 1 μM MCF7 (54) [428] |
| | | | | −0.684 1 μM ssMCF7 (74) [521] |
| | | | | −0.731 1 μM MCF7 (513) [1056] |
| | | | | −0.763 1 μM SKMEL5 (73) [505] |
| | | | | −0.904 1 μM MCF7 (504) [831] |
| | | | | −0.942 1 μM PC3 (55) [432] |
| 128 | rottlerin | −0.421 | 3 | 0.0 10 μM MCF7 (504) [825] |
| | | | | −0.497 10 μM MCF7 (502) [941] |
| | | | | −0.766 10 μM MCF7 (505) [914] |
| 129 | paclitaxel | −0.436 | 1 | −0.436 100 nM MCF7 (111) [640] |
| 130 | pyrvinium | −0.446 | 2 | 0.0 1 μM MCF7 (502) [978] |
| | | | | −0.892 1 μM MCF7 (504) [870] |
| 131 | flufenamic acid | −0.450 | 1 | −0.45 10 μM MCF7 (25) [316] |
| 132 | oligomycin | −0.462 | 1 | −0.462 1 μM MCF7 (59) [442] |
| 133 | 5114445 | −0.480 | 1 | −0.48 10 μM MCF7 (505) [901] |
| 134 | resveratrol | −0.480 | 5 | 0.0 10 μM MCF7 (502) [958] |
| | | | | −0.459 50 μM PC3 (90) [662] |
| | | | | −0.515 50 μM MCF7 (95) [595] |
| | | | | −0.617 10 μM MCF7 (504) [841] |
| | | | | −0.81 50 μM MCF7 (107) [622] |
| 135 | Y-27632 | −0.486 | 2 | −0.485 3 μM MCF7 (504) [832] |
| | | | | −0.488 3 μM MCF7 (502) [948] |
| 136 | carbamazepine | −0.495 | 3 | 0.0 100 nM MCF7 (502) [952] |
| | | | | −0.713 100 nM MCF7 (504) [835] |
| | | | | −0.772 100 nM MCF7 (505) [919] |
| 137 | nitrendipine | −0.503 | 1 | −0.503 10 μM MCF7 (29) [336] |
| 138 | fluphenazine | −0.512 | 4 | 0.0 10 μM SKMEL5 (69) [494] |
| | | | | −0.664 10 μM MCF7 (506) [1017] |
| | | | | −0.675 10 μM MCF7 (68) [490] |
| | | | | −0.708 10 μM MCF7 (513) [1075] |
| 139 | 5152487 | −0.523 | 1 | −0.523 10 μM MCF7 (505) [896] |
| 140 | prazosin | −0.532 | 2 | −0.529 10 μM MCF7 (502) [942] |
| | | | | −0.535 10 μM MCF7 (504) [826] |
| 141 | 5140203 | −0.558 | 1 | −0.558 15 μM MCF7 (505) [908] |
| 142 | cytochalasin B | −0.567 | 1 | −0.567 21 μM MCF7 (514) [1122] |
| 143 | vorinostat | −0.570 | 2 | −0.564 10 μM MCF7 (506) [1000] |
| | | | | −0.576 10 μM MCF7 (513) [1058] |
| 144 | MG-132 | −0.570 | 1 | −0.57 21 μM MCF7 (514) [1140] |
| 145 | HNMPA-(AM)3 | −0.573 | 1 | −0.573 5 μM PC3 (87) [583] |
| 146 | decitabine | −0.573 | 1 | −0.573 100 nM MCF7 (505) [920] |
| 147 | U0125 | −0.576 | 1 | −0.576 1 μM PC3 (90) [663] |
| 148 | nocodazole | −0.585 | 1 | −0.585 1 μM MCF7 (107) [621] |
| 149 | 5224221 | −0.592 | 2 | −0.588 12 μM MCF7 (502) [956] |
| | | | | −0.596 12 μM MCF7 (504) [839] |
| 150 | 3-hydroxy-DL- | −0.599 | 1 | −0.599 9 μM MCF7 (514) [1109] |
| | kynurenine | | | |
| 151 | 5162773 | −0.614 | 1 | −0.614 7 μM MCF7 (505) [892] |
| 152 | oxaprozin | −0.627 | 2 | −0.605 300 μM MCF7 (504) [863] |
| | | | | −0.649 300 μM MCF7 (502) [971] |
| 153 | colforsin | −0.644 | 2 | −0.588 50 μM HL60 (120) [783] |
| | | | | −0.699 50 μM MCF7 (505) [913] |
| 154 | nifedipine | −0.676 | 2 | −0.55 10 μM MCF7 (29) [335] |
| | | | | −0.801 10 μM MCF7 (96) [603] |
| 155 | exemestane | −0.678 | 1 | −0.678 10 nM MCF7 (7) [165] |
| 156 | felodipine | −0.706 | 3 | −0.33 10 μM MCF7 (29) [337] |
| | | | | −0.827 10 μM MCF7 (504) [848] |
| | | | | −0.962 10 μM MCF7 (502) [965] |
| 157 | HC toxin | −0.734 | 1 | −0.734 100 nM MCF7 (505) [909] |
| 158 | verapamil | −0.749 | 1 | −0.749 10 μM MCF7 (7a) [161] |
| 159 | 5213008 | −0.751 | 1 | −0.751 18 μM MCF7 (505) [898] |
| 160 | dimethyloxalylglycine | −0.789 | 1 | −0.789 1 mM PC3 (87) [584] |
| 161 | 5109870 | −0.795 | 1 | −0.795 25 μM MCF7 (505) [904] |
| 162 | calmidazolium | −0.811 | 2 | −0.713 5 μM MCF7 (505) [906] |
| | | | | −0.909 5 μM MCF7 (67) [486] |
| 163 | celastrol | −0.839 | 1 | −0.839 3 μM MCF7 (505) [887] |
| 164 | 5255229 | −0.868 | 2 | −0.839 13 μM MCF7 (502) [949] |
| | | | | −0.898 13 μM MCF7 (504) [833] |

Compounds identified as potentially capable of reversing an observed DS molecular phenotype (and thus might be candidates for further hypothesis testing in vitro) include without limitation NSC-5255229, celastrol, calmidazolium, NSC-5109870, dimethyloxalylglycine, NSC-5213008, verapamil, HC toxin, and felodipine. Celastrol is an antioxidant and anti-inflammatory agent that has been suggested for use in treating Alzheimer disease, which prematurely affects many DS patients (Allison et al. (2001), the contents of which are herein incorporated by reference in their entirety). Calmidazolium is a calmodulin inhibitor, which decreases sensitivity to calcium ion signaling, and has been considered for use in treating osteoporosis (Seales et al. (2006), the contents of which are herein incorporated by reference in their entirety). Verapamil and felodipine are both calcium channel blockers, while dimethyloxalylglycine is a hydroxylase inhibitor thought to increase resistance to oxidative stress (Cummins et al. (2008) and Zaman et al. (1999), the contents of each of which are herein incorporated by reference in their entirety).

The four compounds that (according to this analysis) most mimic the DS phenotype also relate to potassium and calcium signaling or oxidation. These results also implicate oxidative stress and ion transport, providing a third level of confirmation of the importance of these functional classes.

Without wishing to be bound by any particular theory, it is contemplated that compounds identified as having molecular signatures that counteract that of DS may be useful in therapeutic interventions for Down Syndrome. In some embodiments, such interventions are commenced before birth of the affected fetus. In some embodiments, derivatives of compounds identified as having molecular signatures that counteract that of DS are used. In some embodiments, combinations of two or more compounds (and/or derivatives thereof) whose molecular signatures counteract that of DS are used in therapeutic intervention(s).

### Discussion

### Examples 2-5

Results described in Examples 2-5 demonstrate that transcriptional profiling of RNA in uncultured amniotic fluid provides a unique molecular window into developmental disorders in the living human fetus. In addition to identifying genes relevant to the DS phenotype, functional profiling was undertaken to identify significantly disrupted biological pathways.

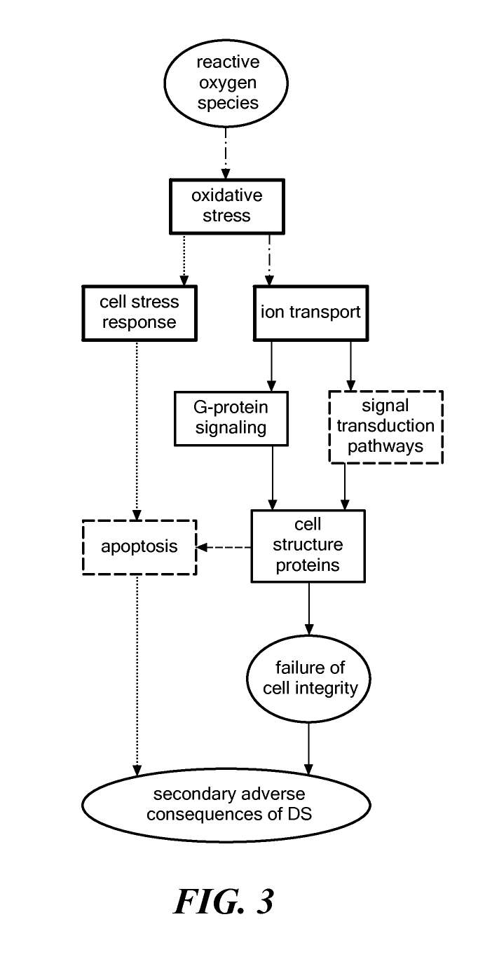

Without wishing to be bound by any particular theory, it is contemplated that among the functional pathway groups identified by both the individual and gene set analyses, several are amenable to a single explanation. Reactive oxygen species, especially hydrogen peroxide, are known to disrupt ion transport mechanisms, leading to problems with signal transduction through cell membranes, cell dysfunction, structural failure of membrane integrity, and ultimately to pathological symptoms, particularly in neural and cardiac tissues (Kourie et al. (1998)). Consistent evidence of several of these steps was observed, including dysregulation of oxidative stress response genes, phospholipids, ion transport molecules, heart muscle genes, structural proteins, and DNA damage repair genes, in both the Individual and the Leading Edge gene sets (Table 7 and FIG. 3).

It has previously been suggested that oxidative stress plays an important role in DS (Zaman et al. (1999)). Since individuals with DS demonstrate pathology consistent with Alzheimer's disease at an early age (Bush et al. (2004)), links to the role of oxidative stress in Alzheimer's have been explored (Zana et al, (2007)). Lockstone et al. (2007) found that oxidative stress response genes were over-represented in adult but not fetal DS tissue, and suggested that this response might reflect adult-onset DS pathologies such as Alzheimer disease. More recently, a few groups have found oxidative stress response markers in fetal DS tissues, although neither study emphasized this particular result or considered the potential relationship between oxidative stress and other functional pathways (Rozovski et al. (2007) and Mao et al. (2005)). Esposito et al. (2008) identified oxidative stress and apoptosis genes in neural progenitor cell lines generated from the frontal cortex of second trimester DS fetuses. They suggested that up-regulation of the chromosome 21 gene S100B causes an increase in reactive oxygen species and stress-response kinases, leading to an increase in programmed cell death. Using a biochemical approach, other investigators demonstrated increased levels of isoprostanes, a marker of oxidative stress, in second trimester amniotic fluid samples from DS fetuses (Perrone et al. (2007)).

The present application discloses the first functional analysis of the DS fetus that implicates not only oxidative stress, but potential intermediate consequences, such as defects in ion transport and G-protein signaling.

In mouse models, at least one G-protein coupled potassium channel protein (GIRK2) has been implicated in DS pathology (Best et al. (2007) and Harashima et al. (2006)). Another study using adult mouse models has suggested a role for two other G-protein dependent pathways in DS and Alzheimer disease (Lumbreras et al. (2006)). The inventors' results described in Examples 3-5, however, suggests a wider and more fundamental role for G-protein signaling, involving a large number of proteins and appearing as early as the second trimester.

These results also contribute to an ongoing debate regarding the extent of transcriptional changes due to trisomy 21. Despite the many prior studies of gene expression in DS, the precise mechanism by which the additional set of chromosome 21 genes disrupts normal development and results in the phenotype of DS remains unknown. Consistent with several previous studies (Mao et al. (2005), Amano et al. (2004), and Dauphinot et al. (2005)), it was observed that trisomic genes generally showed increased expression in DS, with average up-regulation centered near 1.5-fold (FIG. 2a). However, this effect is highly variable, with nearly a third of trisomic genes actually down-regulated on average (but few significantly so). Widespread differential expression of genes from the other diploid chromosomes was observed (FIG. 2b), consistent with the results of Gardiner (2006) and Lockstone et al. (2007).

In the present disclosure, hierarchical clustering of samples based on expression levels of the 409 Individual genes not located on chromosome 21 completely separates the DS samples from the controls (as seen in FIG. 1). In contrast, Mao et al. (2005), who studied gene expression from frozen fetal heart and brain tissue, found that explicit classification of their samples worked only when based on the expression levels of trisomic genes. Without wishing to be bound by any particular theory, it is proposed that differences in expression patterns between their work and the present study may reflect the different tissues involved.

While previous reports identified significant differential expression of trisomic genes, the present analysis surprisingly did not. It is noted that, since most of the amniotic fluid from which RNA was obtained for these studies is cell-free, care should be taken when comparing these results to previously published transcriptomic profiles of material that used fetal cells or tissue (Altug-Teber et al. (2007), Chung et al. (2005), FitzPatrick et al. (2002), Rozovski et al. (2007), Mao et al. (2005), Gross et al. (2002), and Li et al. (2006)). Without wishing to be bound by any particular theory, it is proposed that this discrepancy may also be due the some of the other data being derived from mouse models of DS, which are more genetically homogeneous than the human population samples used in the present studies. It is further proposed, without wishing to be bound by any particular theory, that most likely the discrepancy is due to the use of a strict statistical cutoff for differential expression in the present studies, including adjustment for multiple testing of over 54,000 probe sets. So relatively few chromosome 21 genes were found with such consistent expression in the diverse sample population that the evidence for their moderate up-regulation exceeded this strict significance cutoff Fortunately, GSEA was developed precisely to detect such consistent but modest expression changes. With no a priori bias, the GSEA tool identified the DS critical region as the only strongly (q<0.05) up-regulated chromosomal band in the DS samples.

Addition of the Connectivity Map analysis not only confirmed the pathways implicated by DAVID, but also suggested possible testable hypotheses to develop novel treatments for DS, starting with an in vitro approach to explore the effects of compounds suggested by the Connectivity Map analysis and/or of other compounds with similar effects on oxidation or ion transport. Work described here serves as proof of concept that gene expression profiles from living second trimester human fetuses with developmental disorders can lead to a better understanding of the early etiology of disease as well as the secondary consequences of congenital anomalies, and may suggest future innovative approaches to treatment.

### Example 6

### Gene Expression Profiling of Fetuses with Structural Abnormalities Such as Gastroschisis and Congenital Diaphragmatic Hernia (CDH)

In the present Example, gene expression profiles of fetuses with structural abnormalities such as gastroschisis and congenital diaphragmatic hernia (CDH) are obtained and analyzed. Such profiling is predicted to enable development of in utero therapies for these conditions via an increased understanding of the genetic mechanisms underlying these conditions.

### Experimental Design and Methods

Pregnant women are recruited into the study at the time of sonographic confirmation of the defect. Amniotic fluid supernatant samples that would otherwise be discarded are obtained from the cytogenetics laboratory involved. Approaches to isolation, amplification, and hybridization of fetal cell-free mRNA from amniotic fluid, as well as computational approaches, are the same as described in Examples 2 and 3.

Gene expression profiles may be used, for example, to identify possible therapeutic regimens and/or agents. For example, gene expression profiles of fetuses with structural abnormalities may be used in conjunction with the Connectivity Map (see Example 5) to identify a list of candidate compounds that may have therapeutic uses for these conditions.

### Example 7

### Assessing the Effect of Current Fetal Treatment Using Gene Expression Analyses

In this Example, gene expression analyses are performed before and after treatment to explore the effect of current fetal treatments. Twin-to-twin transfusion syndrome (TTTS) is used in this particular example. It is understood that insights gained from this Example may also be useful for developing and/or assessing treatments for other fetal anomalies, conditions, and diseases (such as Down Syndrome.)

Treatment of TTTS has changed dramatically with the introduction of endoscopic laser ablation of communicating placental vessels. While occlusion of these anastomoses has been shown to halt the transfusion syndrome in almost all cases (as documented by return of diuresis and amniotic fluid in the donor twin within hours or days), survival rates are less than expected, and intrauterine demise of both twins is still observed in 20-25% of cases. In another 30%, only one twin will ultimately survive. Of note, these results appear independent of the individual surgeon and medical center, and have not significantly improved since the introduction of the technique 15 years ago.

It is still unclear why only 15-20% of all identical twins develop the syndrome (although almost all monochorionic twins have placental anastomoses), or why some exhibit a rapid deterioration, while others improve spontaneously. With the availability of an effective treatment, the ability to differentiate the more aggressive subgroup of this disease would be remarkably useful in improving survival, without placing pregnant women and fetuses at undue risk if the disease is predicted to follow a more benign course. Current diagnostic methods are not sensitive enough to distinguish this group, despite very detailed sonographic and Doppler descriptions of the different clinical stages of TTTS.

In the present Example, samples of amniotic fluid are obtained pre and post intervention to examine specific changes in gene expression that result from treatment. Amniotic fluid is routinely collected at the time of the fetoscopic procedure. To analyze the effects of the intervention, the study includes an amniocentesis at a later date at a time when the risk of preterm labor associated with the procedure has subsided. Methods of isolating, amplifying, and hybridizing fetal cell-free mRNA from amniotic fluid, as well as computational methods, are similar to those described in Example 3. The list of genes up-regulated in amniotic fluid at the time of the procedure are compared to the same fetus following laser ablation at a later date.

### Example 8

### Development of Baseline Gene Expression Data as a Function of Gestational Age from Whole Blood

In this Example, baseline gene expression data are obtained from normal fetuses at various ages during the second trimester, a time during which medical intervention could occur in order to prevent the development of symptoms of the fetus. Such baseline gene expression data would be useful for comparison purposes when analyzing gene expression patterns in fetuses with chromosomal, structural, and/or growth abnormalities such as Down Syndrome fetuses.

In this example, RNA samples are obtained from maternal whole blood samples rather than amniotic fluid. Although amniotic fluid is a source of pure fetal mRNA, it can only be obtained through an invasive procedure. Maternal blood can be available through less invasive procedures, though it presents challenges because it contains a mixture of both maternal and fetal nucleic acids. Nevertheless, the inventors had previously shown that they can identify fetal gene expression in maternal whole blood samples (Maron et al. 2007, the entire contents of which are herein incorporated by reference in their entirety).

Preliminary studies had provided baseline gene expression data on 10 fetuses between 36 and 39 weeks of gestation (i.e., at term). This time period was selected because of ease of coordinating antepartum and postpartum maternal and infant blood samples. This study proved that it is possible to isolate mRNA from whole blood and perform a comparison genomic analysis to identify genes that were differentially up-regulated in the maternal antepartum samples, representing candidate fetal transcripts.

In this Example, women who are undergoing elective termination of pregnancy are enrolled. An antenatal sample is then obtained during pregnancy, as well as a fetal cord blood sample (technically feasible after 18 weeks of gestation) and a post-termination sample.

Methods to Isolate Fetal mRNA from Whole Maternal Blood

Whole blood samples are obtained from women in the first, second and third trimesters of pregnancy, as well as before and after term delivery. All blood samples are obtained in PaxGene specimen tubes (PreAnalytiX) and stored at room temperature for 6 to 36 hours prior to RNA extraction.

RNA is extracted using the PaxGene blood RNA kit (PreAnalytiX) according the manufacturer's instructions. Following extraction, a portion of the eluted RNA sample is analyzed on the Bioanalyzer 2100 (Agilent) to assess quantity and quality of each sample. Samples with distinct peaks representing 18S ribosomal RNA and a minimum quantity of 1 μg of total RNA are selected for further processing. Extracted total RNA are then amplified and converted to cDNA using the commercially available One Step Amplification Kit (Affymetrix) according to Van Gelder et al. (1990). Following amplification, cDNA is then assessed with the Bioanalyzer 2100 for quantity and quality. When possible, 15 μg of amplified labeled cDNA is then fragmented and hybridized to the GeneChip® Human Genome U133 Plus 2.0 Array, which allows analysis of over 47,000 transcripts and variants derived from over 38,500 human genes.

### Computational Methods

Gene expression data analysis methods known to be relevant to time series analysis are used to identify genes that are changing significantly and consistently during normal development. Such methods include analysis of variance (ANOVA), Fourier-transform methods (Wichert et al. 2004; Aach and Church 2001) and spline-fitting methods (Bar-Joseph, et al. 2003).

### Example 9

### Comparison of Gene Expression Data Between Abnormal Fetuses and Gestationally Age-Matched Normal Fetuses

In this Example, gene expression data from abnormal fetuses is compared to that of gestationally age-matched normal fetuses. Once a developmental gene expression profile is established for normal fetuses in the second trimester and at term, cases of gestational-age matched pregnancies that are complicated by fetal chromosomal or anatomic abnormalities are sought for analyses. Studies in this Example focus on trisomy 21 because of the interest in providing noninvasive prenatal diagnosis (as opposed to screening) for this condition. Additional analyses on diseased fetuses such as those with trisomy 21 (using baseline gene expression profiles from Example 5) may provide an opportunity to explore new hypotheses, such as whether fetuses with severe intrauterine growth restriction at term manifest neurodevelopmental abnormalities in utero.

Preliminary studies showed that it is possible to successfully isolate mRNA from whole blood, hybridize to gene expression arrays, and create gene lists of up-regulated genes that are involved in fetal development at term. Methods for processing maternal blood samples are the same as in Example 5. Expression data obtained from “diseased” fetuses are compared to that of “healthy” fetuses at the same gestational age to determine differences in expression.

### Example 10

### Development of Custom Microarrays for Prenatal Diagnostic Applications

In this Example, custom microarrays for prenatal diagnostic applications are developed using gene expression data from Examples 7 and 8. Custom microarrays could be developed for a variety of disorders affecting fetuses depending on the gene expression data that becomes available from Examples 7 and 8.

Using trisomy 21 as an example, affected fetuses are shown to have differences in expression of genes related to cardiac function depending on the extent of their underlying cardiac malformation(s). Custom gene expression microarrays are designed specifically to include genes identified as being differentially regulated for a particular condition.

Such custom microarrays could help identify those infants likely to manifest cardiac failure in the perinatal period, which will influence location of delivery. Similarly, a “neurodevelopmental” custom array, used with blood samples from pregnant women with complicated pregnancies, could identify those fetuses with the highest likelihood of abnormal neurologic development. Such determinations may influence decisions regarding route of delivery or how long to allow the pregnant woman to labor.

Several companies already offer creation of custom microarrays for reasonable costs via an online system. In particular, Agilent Technologies allows uploading of probe sequences, a selection of various formats, and printing using well-validated technology. Ultimately sequences most likely to distinguish between normal and abnormal fetuses are selected and custom arrays with such sequences are ordered. For gene expression profiling, methods described in Example 7 are used, except that custom arrays are used instead of Affymetrix arrays.

### Example 11

### Design of Novel Fetal Treatment Approaches Using Pathway and Network Analyses

Analyses discussed in this Example are directed to developing novel fetal treatment approaches that could potentially allow intervention earlier than existing therapies allow. Current fetal therapies are generally offered when signs and symptoms of disease have already developed, or the clinical significance of the condition is well-known. Although in some cases fetal treatment is necessary to prevent fetal demise (for example, laser ablation of shared vessels in twin-to-twin transfusion syndrome, TTTS), in many cases the treatment is too late. The development of treatments that can be offered earlier may be facilitated by understanding what biological pathways are involved in normal fetal development and by the ability to identify fetuses that are developing abnormally whether or not they show symptoms. Such treatments might reduce or ameliorate symptoms before birth. Although the following discussion focuses on TTTS, it is to be understood that novel treatment approaches could similarly be designed for Down Syndrome.

The metabolic and hormonal aspects of TTTS have been extensively studied. It is now believed that the incidence of hydrops in the recipient twin is higher than one would expect based solely on a theory of fluid overload. It has been speculated, without wishing to be bound by any particular theory, that the (appropriate) up-regulation of the renin-angiotensin system in the chronically hypovolemic donor causes inappropriate vasoconstriction and fluid retention in the recipient, as long as both fetuses remain connected by vascular anastomoses. It has been suggested that activation of renin in the donor kidney may be secondary to chronic hypoxemia because of elevated placental vascular resistance. A large percentage of donor fetuses have a significantly smaller placental share, which may cause intrauterine growth restriction (IUGR) and increased vascular resistance. Other putative factors and pathways have been proposed as well, lending credence to the theory that fetal well-being and/or stress affect placental and maternal metabolism. Analysis of amniotic fluid and maternal serum from subjects with varying degrees of severity of TTTS, and their differential analysis before and after surgical treatment, could potentially offer new insight in the pathophysiology of the disease, methods of early detection of severe or rapidly evolving forms, and opportunities to offer non-operative treatment.

### Preliminary Studies

The inventors had previously demonstrated that microarray gene expression profiling of amniotic fluid provides important information about fetal well-being, development, and potential disease status (Larrabee et al. (2005), the contents of which are herein incorporated by reference in their entirety). Two fetuses with TTTS and 2 fetuses with hydrops fetalis were compared to pooled information from 6 normal fetuses. At the time of publication of Larrabee et al. (2005), pathway analysis had not been performed. Subsequently, data from the preliminary study's gene set was analyzed using Ingenuity pathway analysis software. Results are presented in Table 9. Although statistical significance was limited due to the small numbers of samples involved, the results suggest a strong involvement of carbohydrate metabolism pathways in the pathophysiology of TTTS and hydrops fetalis.

Results of pathway analyses are reviewed for consistent trends. Should specific biologic pathways be identified in specific diseases, consultants with knowledge of the disease and experts in pharmacology are identified to develop specific suggestions of drugs that would be safe to administer to the fetus and that would warrant controlled study.

All literature and similar material cited in this application, including, patents, patent applications, articles, books, treatises, dissertations and web pages, regardless of the format of such literature and similar materials, are expressly incorporated by reference in their entirety. In the event that one or more of the incorporated literature and similar materials differs from or contradicts this application, including defined terms, term usage, described techniques, or the like, this application controls.

The section headings used herein are for organizational purposes only and are not to be construed as limiting the subject matter described in any way.

### REFERENCES

1. Aach and Church. “Aligning gene expression time series with time warping algorithms” Bioinformatics. 2001. 17:495-508.
2. Adams et al., “Complementary DNA sequencing: expressed sequence tags and human genome project.” Science. 1991, 252: 1651-1656
3. Aikens et al., “Solid-state imagers for microscopy.”Meth. Cell Biol. 1989, 29: 291-313
4. Alberts et al., “Molecular Biology of the Cell.” 1994 (3rd Ed.), Garland Publishing, Inc.: New York, N.Y.
5. Allison et al. (2001) “Celastrol, a potent antioxidant and anti-inflammatory drug, as a possible treatment for Alzheimer's disease.” Prog Neuropsychopharmacol Biol Psychiatry. 25: 1341-1357.
6. Altug-Teber et al. (2007) “Specific transcriptional changes in human fetuses with autosomal trisomies.” Cytogenet Genome Res. 119:171-184.
7. Amano et al. (2004) “Dosage-dependent over-expression of genes in the trisomic region of Ts1Cje mouse model for Down syndrome.” Hum Mol. Genet. 13:1333-1340.
8. Anderson et al., Monthly Vital Statistics Report. 1997. 45(11 suppl. 2): 55
9. Anderson (Ed.), “Nucleic Acid Hybridization.” 1999, Springer Verlag: New York, N.Y.
10. Anfinsen et al., “Studies on the gross structure, cross-linkages, and terminal sequences in ribonuclease.” J. Biol. Chem. 1954, 207: 201-210
11. Apweiler et al., “InterPro—an integrated documentation resource for protein families, domains and functional sites.” Bioinformatics. 2000. 16:1145-1150
12. Ashburner et al., “Gene ontology: tool for the unification of biology. The Gene Ontology Consortium.” Nat. Genet. 2000. 25:25-29
13. Ausubel (Ed.), “Short Protocols in Molecular Biology.” 2002, 5th Ed., John Wiley & Sons
14. Bar-Joseph et al, “Continuous representations of time series gene expression data.” J. Comp. Biol. 2003. 10:341-356.
15. Barker et al., “The protein information resource (PIR).” Nucleic Acids Res. 2000. 28:41-44
16. Barone et al., “Photolithographic synthesis of high-density oligonucleotide probe arrays.” Nucleosides Nucleotides Nucleic Acids. 2001. 20: 525-531
17. Barringer et al., “Blunt-end and single-strand ligations by Escherichia coli ligase: influence on an in vitro amplification scheme.” Gene. 1990, 89: 117-122
18. Bassett et al., “Gene expression informatics—it's all in your mine” Nat. Genet. 1999, 21: 51-55
19. Bayer et al., “The use of the avidin-biotin complex as a tool in molecular biology.” Methods of Biochem. Analysis. 1980, 26: 1-45
20. Becker-Andre and Hahlbrock, “Absolute mRNA quantification using the polymerase chain reaction (PCR). A novel approach by a PCR aided transcript titration assay (PATTY).” Nucl. Acids Res. 1989, 17: 9437-9346
21. Belosludtsev et al., “DNA microarrays based on noncovalent oligonucleotide attachment and hybridization in two dimensions.” Anal Biochem. 2001. 292: 250-256
22. Belousov et al., “Sequence-specific targeting and covalent modification of human genomic DNA.” Nucleic Acids Res. 1997. 25: 3440-3444
23. Benjamini and Hochberg, “Controlling the false discovery rate: A practical and powerful approach to multiple testing.” J Roy Statist Soc Ser B. 1995. 57:289-300.
24. Best et al. “Ts65Dn, a mouse model of Down syndrome, exhibits increased GABAB-induced potassium current.” J. Neurophysiol. 2007. 97(1):892-900
25. Blommers et al., “Effects of the introduction of L-nucleotides into DNA. Solution structure of the heterochiral duplex d(G-C-G-(L)T-G-C-G).d(C-G-C-A-C-G-C) studied by NMR spectroscopy.” Biochemistry. 1994. 33: 7886-7896
26. Blackburn et al., “Ribonuclease inhibitor from human placenta. Purification and properties.” J. Biol. Chem. 1977, 252: 5904-5910
27. Bowtell, “Options available—from start to finish—for obtaining expression data by microarray.” Nature Gen. 1999. Supp. 21:25-32
28. Bravo et al., “A versatile negative-staining ribonuclease zymogram.” Anal. Biochem. 1994, 219: 82-86
29. Brazma and Vilo, “Gene expression data analysis.” FEBS Lett. 2000, 480: 17-24
30. Brison et al., “General method for cloning amplified DNA by differential screening with genomic probes.” Mol. Cell. Biol. 1982, 2: 578-587
31. Brigati et al., “Detection of viral genomes in cultured cells and paraffin-embedded tissue sections using biotin-labeled hybridization probes.” Virol. 1983, 126: 32-50
32. Britten et al., “Analysis of repeating DNA sequences by reassociation.” Methods in Enzymol., 1974, 29: 363-418
33. Brown et al., “Chemical synthesis and cloning of a tyrosine tRNA gene.” Methods in Enzymology. 1979. 68: 109-151
34. Broker et al., “Electron microscopic visualization of tRNA genes with ferritin-avidin: biotin labels.” Nucl. Acids Res. 1978, 5: 363-384
35. Burg et al., “Single molecule detection of RNA reporter probes by amplification with Q beta replicase.”Mol. Cell. Probes. 1996, 10: 257-271
36. Butte, “The use and analysis of microarray data.” Nat. Rev. Drug Discov. 2002, 1: 951-960
37. Butler et al., “In situ synthesis of oligonucleotide arrays by using surface tension.” J Am Chem. Soc. 2001. 123: 8887-8894
38. Bush et al., “Risk factors for dementia in people with Down Syndrome: Issues in assessment and diagnosis.” Am J Ment Retard. 2004. 109:83-97.
39. Centers for Disease Control, Morb Mortal Weekly Rep. 1989, 38: 264-267
40. Chen et al., “4,4-Difluoro-4-bora-3a,4a-diaza-s-indacene (BODIPY) Dyes Modified for Extended Conjugation and Restricted Bond Rotations.” J. Org. Chem. 2000, 65: 2900-2906
41. Chen et al., “Profiling Expression Patterns and Isolating Differentially Expressed Genes by cDNA Microarray System with Colorimetry Detection.” Genomics, 1998, 51: 313-324
42. Chen et al., “Probing the cathepsin D using a BODIPY FL-pepstatin A: applications in fluorescence polarization and microscopy.” J. Biochem. Biophys. Methods, 2000, 42: 137-151
43. Cheung et al., “Integration of cytogenetic landmarks into the draft sequence of the human genome.” Nature. 2001. 409(6822): 953-958
44. Cheung et al., “Making and reading microarrays.” Nature Genet. 1999, 21: 15-19
45. Chiu et al., “Mass spectrometry of single-stranded restriction fragments captured by an undigested complementary sequence.” Nuc. Acids Res. 2000. 28: E31
46. Chomczynski et al., “Single-step method of RNA isolation by acid guanidinium thiocyanate-phenol-chloroform extraction.” Anal. Biochem. 1987, 162: 156-159
47. Chiang et al., “Visualizing associations between genome sequences and gene expression data using genome-mean expression profiles.” Bioinformatics, 2001, 17: S49-S55
48. Chirgwin et al., Biochem. 1979, 18: 5294-529
49. Chung et al. “Gene expression analysis of cultured amniotic fluid cell with Down syndrome by DNA microarray.” J Korean Med. Sci. 2005. 20:82-87.
50. Connoly and P. Rider, “Chemical synthesis of oligonucleotides containing a free sulphydryl group and subsequent attachment of thiol specific probes.” Nucl. Acids. Res. 1985, 13: 4485-4502
51. Cummins et al. “The hydroxylase inhibitor dimethyloxalylglycine is protective in a murine model of colitis.” Gastroenterology. 2008. 134: 156-165.
52. Cunniff et al., “Health care utilization and perceptions of health among adolescents and adults with Turner syndrome.” Clinical Genetics. 1995, 48:17-22
53. Cuzin, “DNA chips: a new tool for genetic analysis and diagnostics.” Transfus. Clin. Biol. 2001. 8:291-296
54. Czeizel et al., “The load of genetic and partially genetic disorders in man. I. Congenital anomalies: estimates of detriment in terms of years of life lost and years of impaired life.” Mutation Research. 1984, 128: 73-103
55. Dauphinot et al. “The cerebellar transcriptome during postnatal development of the Ts1Cje mouse, segmental trisomy model for Down syndrome.” 2005. Hum Mol. Genet. 14: 373-384.
56. Deininger, “Random subcloning of sonicated DNA: application to shotgun DNA sequence analysis.” Anal Biochem. 1983. 129: 216-223
57. Dennis, Jr. et al. “DAVID: Database for Annotation, Visualization, and Integrated Discovery.” Genome Biology. 2003. 4:P3.
58. Dewey, “From microarrays to networks: mining expression time series.” Drug Discov. Today, 2002, 7: S170-S175
59. Divane et al., “Rapid prenatal diagnosis of aneuploidy from uncultured amniotic fluid cells using 5 colour fluorescence in situ hybridization.” Prenat. Diagn. 1994, 14: 1061-1069
60. Duggan et al., “Expression profiling using cDNA microarrays.” Nat. Genet. 1999, 21: 10-14
61. Ekins and Chu, “Microarrays: their origins and applications.” Trends in Biotech. 1999, 17: 217-218
62. Esposito, et al. “Genomic and functional profiling of human Down syndrome neural progenitors implicates S100B and aquaporin 4 in cell injury.” Human Molecular Genetics. 2008. 14:440-457.
63. FitzPatrick et al. “Transcriptome analysis of human autosomal trisomy.” Hum Mol. Genet. 2002. 11:3249-56
64. Freeman et al., “Fundamentals of DNA hybridization arrays for gene expression analysis.” Biotechniques. 2000. 29: 1042-1046 and 1048-1055
65. Frenkel et al., “7,12-dimethylbenz[a]anthracene induces oxidative DNA modification in vivo.” Free Radic Bio. Med. 1995. 19: 373-380
66. Gabig and Wegrzyn, “An introduction to DNA chips: principles, technology, applications and analysis.” Acta Biochim. Pol. 2001. 48: 615-622
67. Gardiner, “Transcriptional dysregulation in Down syndrome: Predictions for altered protein complex stoichiometries and post-translatonal modifications, and consequences for learning/behavior genes ELK, CREB, and the estrogen and gluococorticoid receptors.” Behav Genet. 2006. 36:439-453.
68. Gentleman et al. “Bioconductor: open software development for computational biology and bioinformatics.” Genome Biol. 2004 5: R(80).
69. Gilliland et al., “Analysis of cytokine mRNA and DNA: detection and quantitation by competitive polymerase chain reaction.” Proc. Natl. Acad. Sci. 1990, 87: 2725-2729
70. Glisin et al., “Ribonucleic acid isolated by cesium chloride centrifugation.” Biochem. 1974, 13: 2633-2637
71. Greijer et al., “Quantitative competitive NASBA for measuring mRNA expression levels of the immediate early 1, late pp 67, and immune evasion genes US3, US6, and US11 in cells infected with human cytomegalovirus.” J. Virol. Methods. 2001, 96: 133-147
72. Greiner-Stoeffele et al., “A General Ribonuclease Assay Using Methylene Blue.” Anal. Biochem. 1996, 240: 24-28
73. Gross et al. “Gene expression profile of trisomy 21 placentas: a potential approach for designing noninvasive techniques of prenatal diagnosis.” Am J Obstet. Gynecol. 2002. 187:457-62.
74. Grunert et al., “Trp59 to Tyr substitution enhances the catalytic activity of RNase T1 and of the Tyr to Trp variants in positions 24, 42 and 45.” Protein Eng. 1993, 6: 739-744
75. Guatelli et al., “Isothermal, in vitro amplification of nucleic acids by a multienzyme reaction modeled after retroviral replication.” Proc. Natl. Acad. Sci. 1990, 87: 1874-1878
76. Guschin et al., “Manual manufacturing of oligonucleotide, DNA, and protein microchips.” Anal Biochem. 1997. 250: 203-211
77. Hamaguchi et al., “Aptamer Beacons for the Direct Detection of Proteins.” Anal. Biochem. 2001, 294: 126-131
78. Harashima et al. “Elevated expression of the G-protein-activated inwardly rectifying potassium channel 2 (GIRK2) in cerebellar unipolar brush cells of a Down syndrome mouse model.” Cell Mol. Neurobiol. 2006. 26:719-734.
79. Haugland, “Molecular Probes: Handbook of Fluorescent Probes and Research Chemicals 1992-1994”, 5th Ed., 1994, Molecular Probes, Inc.
80. Hess et al., “Microarrays: Handling the deluge of data and extracting reliable information.” Trends Biotechnol. 2001, 19: 463-468
81. Hiraoka et al., “The Use of a Charge-Coupled Device for Quantitative Optical Microscopy of Biological Structures.” Science, 1987, 238: 36-41
82. Hopman et al., “Mercurated nucleic acid probes, a new principle for non-radioactive in situ hybridization.” Exp. Cell Res. 1987, 169: 357-368
83. Ikehara et al., “Inquiries into the structure-function relationship of ribonuclease T1 using chemically synthesized coding sequences.” Proc. Natl. Acad. Sci. USA, 1986, 83: 4695-4699
84. Innis (Ed.), “PCR Protocols: A Guide to Methods and Applications.” Academic Press: New York, 1990
85. Innis (Ed.), “PCR Strategies.” Academic Press: New York, 1995
86. Jalal et al., “Prenatal detection of aneuploidy by directly labeled multicolored probes and interphase fluorescence in situ hybridization.” Mayo Clin. Proc. 1998, 73:132-137
87. Johnston, “Gene chips: array of hope for understanding gene regulation.” Curr Biol. 1998. 8: R171-R174
88. Joos et al., “Mapping and chromosome analysis: the potential of fluorescence in situ hybridization.” J. Biotechnol. 1994, 35: 135-153
89. Kaboev et al., “PCR hot start using primers with the structure of molecular beacons (hairpin-like structure).” Nucl. Acids Res. 2000, 28: E94
90. Kanehisa and Goto, “KEGG: kyoto encyclopedia of genes and genomes.” Nucleic Acids Res. 2000. 28:27-30
91. Kaplan, “Natural adult survival patterns.” J Am Coll Cardiol. 1991. 18: 319-320
92. Kern and Hampton, “Direct hybridization of large-insert genomic clones on high-density gridded cDNA filter arrays.” Biotechniques. 1997. 23:120-124
93. Kerr et al., “Analysis of Variance for Gene Expression Microarray Data.” J. Comput. Biol. 2000, 7: 819-837
94. Khatri et al. “Profiling gene expression using Onto-Express.” Genomics. 2002. 79(2):266-270
95. Kievits et al., “NASBA™ isothermal enzymatic in vitro nucleic acid amplification optimized for the diagnosis of HIV-1 infection.” J. Virol. Methods, 1991, 35: 273-286
96. Kimmel and Berger, “Preparation of cDNA and the generation of cDNA libraries: Overview.” Methods in Enzymol. 1987, 152: 307-316
97. Korn et al., “Analysis of the RNase T1 Mediated Cleavage of an Immobilized Gapped Heteroduplex via Fluorescence Correlation Spectroscopy.” Biol. Chem. 2000, 381: 259-263
98. Kourie, “Interaction of reactive oxygen species with ion transport mechanisms.” American Journal of Physiology. 1998. 275: C1-24.
99. Kricka, “Stains, labels and detection strategies for nucleic acids assays.” Ann. Clin. Biochem. 2002, 39: 114-129
100. Kwoh et al., “Transcription-based amplification system and detection of amplified human immunodeficiency virus type 1 with a bead-based sandwich hybridization format.” Proc. Natl. Acad. Sci. 1989, 86: 1173-1177
101. Lamb et al., “The Connectivity Map: A new tool for biomedical research.” Nat Rev Cancer. 2006. 7: 54-60.
102. Lamb et al. “The Connectivity Map: Using gene-expression signatures to connect small molecules, genes, and disease.” Science. 2007. 313: 1929-1935.
103. Landegren et al., “A ligase-mediated gene detection technique.” Science, 1988, 241: 1077-1080
104. Landegent et al., “2-acetylaminofluorine-modified probes for the hybridocytochemical detection of specific nucleic acid sequences.” Exp. Cell Res. 1984. 15: 61-72
105. Langer et al., “Enzymatic synthesis of biotin-labeled polynucleotides: novel nucleic acid affinity probes.” Proc. Natl. Acad. Sci. 1981. 78: 6633-6637
106. Larrabee et al. “Presence of filterable and nonfilterable cell-free mRNA in amniotic fluid.” Clin Chem. 2005. 51(6):1024-6
107. Lehotsky et al. “Membrane ion transport systems during oxidative stress in rodent brain: Protective effect of stobadine and other antioxidants.” Life Sciences. 1999. 65: 1951-1958.
108. Liang and Pardee, “Differential display of eukaryotic messenger RNA by means of the polymerase chain reaction.” Science. 1992. 257: 967-971
109. Lipshutz et al., “High density synthetic oligonucleotide arrays.” Nat. Genet. 1999. 21: 20-24
110. Livak et al., “Oligonucleotides with Fluorescent Dyes at Opposite Ends Provide a Quenched Probe System Useful for Detecting PCR Product and Nucleic Acid Hybridization.” PCR Methods Appl. 1995. 4: 357-362
111. Lockhart et al., “Expression monitoring by hybridization to high-density oligonucleotide arrays.” Nature Biotech. 1996. 14: 1675-1680
112. Lockhart and Winzeler, “Genomics, gene expression and DNA arrays.” Nature, 2000, 405: 827 836
113. Lockstone et al. “Gene expression profiling in the adult Down Syndrome brain.” Genomics. 2007. 90:647-660.
114. Lumbreras et al. “Brain G-protein-dependent signaling pathways in Down Syndrome and Alzheimer's disease.” Amino Acids. 2006. 31(4):449-56),
115. Mansfield et al., “Nucleic acid detection using non-radioactive labelling methods.” Mol. Cell. Probes. 1995, 9: 145-156
116. Mao et al. “Primary and secondary transcriptional effects in the developing human Down syndrome brain and heart.” Genome Biol. 2005. 6:R107.
117. Marcotte and Date, “Exploiting big biology: integrating large-scale biological data for function inference.” Brief Bioinform. 2001. 2: 363-374 118. Marshall and Hodgson, “DNA chips: an array of possibilities.” Nature Biotech. 1998. 16: 27-31
119. Maron et al. “Gene expression analysis in pregnant women and their infants identifies unique fetal biomarkers that circulate in maternal blood.” J Clin Invest. 2007. 117(10):3007-3019
120. Maskos and Southern, “Oligonucleotide hybridizations on glass supports: a novel linker for oligonucleotide synthesis and hybridization properties of oligonucleotides synthesised in situ.” Nucleic Acids Res. 1992. 20: 1679-1684
121. Mates et al. “Intracellular redox status and oxidative stress: Implications for cell proliferation, apoptosis, and carcinogenesis.” Archives of Toxicology. 2008. 82:273-299.
122. Matson et al., “Biopolymer synthesis on polypropylene supports: oligonucleotide arrays.” Anal Biochem. 1995. 224: 110-116
123. McClelland et al., “Arbitrary primed PCR fingerprinting of RNA applied to mapping differentially expressed genes.” EXS. 1993. 67: 103-115
124. McGall et al., “Photolithographic synthesis of high-density oligonucleotide arrays.” Methods Mol. Biol. 2001. 170: 71-101
125. Narang et al., “Improved phosphotriester method for the synthesis of gene fragments.” Methods in Enzymology. 1979. 68: 90-98;
126. Neves et al., “Novel method for covalent fluorescent labeling of plasmid DNA that maintains structural integrity of the plasmid.” Bioconjugate Chem. 2000. 11: 51-55
127. Nuwaysir et al., “Gene expression analysis using oligonucleotide arrays produced by maskless photolithography.” Genome Res. 2002. 12: 1749-1755
128. Oefner et al., “Efficient random subcloning of DNA sheared in a recirculating point-sink flow system.” Nucleic Acids Res. 1996. 24: 3879-3886
129. Ordahl et al., “Sheared DNA fragment sizing: comparison of techniques.” Nucleic Acids Res. 1976. 3: 2985-2999
130. Pease et al., “Light-generated oligonucleotide arrays for rapid DNA sequence analysis.” Proc Natl Acad Sci USA. 1994. 91: 5022-5026
131. Perrone et al. “Early oxidative stress in amniotic fluid of pregnancies with Down syndrome.” Clin Biochem. 2007. 40: 177-180.
132. Pirrung et al., “3′-nitrophenylpropyloxycarbonyl (NPPOC) protecting groups for high-fidelity automated 5′- ->3′ photochemical DNA synthesis.” Org. Lett. 2001. 3: 1105-1108
133. Poddar and Le, “Bordetella pertussis detection by spectrofluorometry using polymerase chain reaction (PCR) and a molecular beacon probe.” Mol. Cell. Probes. 2001. 15: 161-167
134. Podyminogin et al., “Attachment of benzaldehyde-modified oligodeoxynucleotide probes to semicarbazide-coated glass.” Nucleic Acids Res. 2001. 29: 5090-5098
135. Quaas et al., “Indicator plates for rapid detection of ribonuclease T1 secreting Escherichia coli clones.” Nucl. Acids Res. 1989. 17: 3318
136. Ramsay, “DNA chips: state-of-the art.” Nature Biotech. 1998. 16: 40-4
137. Richardson et al., “Biotin and fluorescent labeling of RNA using T4 RNA ligase.” Nucl. Acids Res. 1983. 11: 6167-6184
138. Robinson and Linden, “Clinical Genetic Handbook”, 1993, Blackwell Scientific Publications; Boston, Mass.
139. Rogers et al., “Immobilization of oligonucleotides onto a glass support via disulfide bonds: A method for preparation of DNA microarrays.” Anal Biochem. 1999. 266: 23-30
140. Rozovski et al. “Genome-wide expression analysis of human autosomal trisomy.” Hum Mol Genet. 2007. 11:3249-3256.
141. Sambrook et al., “Molecular Cloning: A Laboratory Manual”, 1989, 2nd Ed., Cold Spring Harbour Laboratory Press: New York
142. Schena et al., “Quantitative monitoring of gene expression patterns with a complementary DNA microarray.” Science. 1995. 270: 467-470
143. Schena et al., “Parallel human genome analysis: microarray-based expression monitoring of 1000 genes.” Proc. Natl. Acad. Sci. 1996. 93: 10614-10619
144. Schummer et al., “Inexpensive handheld device for the construction of high-density nucleic acid arrays.” Biotechniques. 1997. 23:1087-1092
145. Seales et al. “Calmodulin is a critical regulator of osteoclastic differentiation, function, and survival.” J Biol Psychiatry. 2006. 25: 1341-1357.
146. Sebestyen et al., “DNA vector chemistry: the covalent attachment of signal peptides to plasmid DNA.” Nat. Biotechnol. 1998. 16: 568-576
147. Siles and Collier, “Analysis of DNA fragmentation using a dynamic size-sieving polymer solution in capillary electrophoresis.” J. Chromatogr. A. 1997. 771: 319-329
148. Singh-Gasson et al., “Maskless fabrication of light-directed oligonucleotide microarrays using a digital micromirror array.” Nat. Biotech. 1999. 17: 974-978
149. Smith et al., “Detection of Mycobacterium tuberculosis directly from sputum by using a prototype automated Q-beta replicase assay.” J. Clin. Microbiol. 1997. 35(6): 1477-1491
150. Smith et al., “The synthesis of oligonucleotides containing an aliphatic amino group at the 5′ terminus: synthesis of fluorescent DNA primers for use in DNA sequence analysis.” Nucl. Acids Res. 1985. 13: 2399-2412
151. Solinas-Toldo et al., “Matrix-based comparative genomic hybridization: biochips to screen for genomic imbalances.” Genes, Chromosomes & Cancer. 1997. 20: 399-407
152. Sokol et al., “Real time detection of DNA•RNA hybridization in living cells.” Proc. Natl. Acad. Sci., 1998. 95: 11538-11543
153. Stern and Newton, “Isolation of plant mitochondrial RNA.” Methods in Enzymol. 1986. 118: 488
154. Su et al., “High-throughput RT-PCR analysis of multiple transcripts using a microplate RNA isolation procedure.” BioTechniques, 1997. 22: 1107-1113
155. Subramanian et al. “Gene set enrichment analysis: a knowledge-based approach for interpreting genome-wide expression profiles.” Proc Natl Acad Sci USA. 2005. 102(43): 15545-50
156. Tamames et al., “Bioinformatics methods for the analysis of expression arrays: data clustering and information extraction.” J. Biotechnol. 2002. 98: 269-283
157. Tchen et al., “Chemically modified nucleic acids as immunodetectable probes in hybridization experiments.” Proc. Natl Acad. Sci. 1984. 81: 3466-3470
158. Temsamani and Agrawal, “Enzymatic labeling of nucleic acids.” Mol. Biotechnol. 1996. 5: 223-232
159. Thomas et al. “PANTHER: A library of protein families and subfamilies indexed by function.” Genome Res. 2003. 13:2129-2141.
160. Thorstenson et al., “An automated hydrodynamic process for controlled, unbiased DNA shearing.” Genome Res. 1998. 8: 848-855.
161. Tijssen “Hybridization with Nucleic Acid Probes—Laboratory Techniques in Biochemistry and Molecular Biology (Parts I and II).” 1993. Elsevier Science.
162. van Gijlswijk et al., “Universal Linkage System: versatile nucleic acid labeling technique.” Expert Rev. Mol. Diagn. 2001. 1: 81-91
163. Velculescu et al., “Serial analysis of gene expression.” Science. 1995. 270: 484-487
164. Wang et al., “Quantitation of mRNA by the polymerase chain reaction.” Proc. Natl. Acad. Sci. 1989. 86: 9717-9721
165. Watson et al., “Technology for microarray analysis of gene expression.” Curr. Opin. Biotechnol. 1998. 9: 609-614
166. Watson and Akil, “Gene chips and arrays revealed: a primer on their power and their uses.” Biol Psychiatry. 1999. 45: 533-543
167. Welsh et al., “Arbitrarily primed PCR fingerprinting of RNA.” Nucl. Acids Res. 1992. 20: 4965-497
168. Wichert et al. “Identifying periodically expressed transcripts in microarray time series data.” Bioinformatics. 2004. 20: 5-20.
169. Wiegant et al. “ULS: a versatile method of labeling nucleic acids for FISH based on a monofunctional reaction of cisplatin derivatives with guanine moieties.” Cytogenet cell Genet. 1999. 87: 47-52.
170. Wilfinger et al., “Effect of pH and ionic strength on the spectrophotometric assessment of nucleic acid purity.” Biotechniques. 1997. 22: 474-481
171. Weinstein et al., “The bioinformatics of microarray gene expression profiling.” Cytometry. 2002. 47: 46-49
172. Xiang et al., “Microarray expression profiling: analysis and applications.” Curr. Opin. Drug Discov. Devel. 2003. 6: 384-395
173. Yamamoto et al., “Molecular beacon aptamer fluoresces in the presence of Tat protein of HIV-1.” Genes Cells. 2000. 5: 389-396
174. Zaman et al. “Protection from oxidative stress-induced apoptosis in cortical neuronal cultures by iron chelators is associated with enhanced DNA binding of hypoxia-inducible factor-1 and ATF-1/CREB and increased expression of glycolytic enzumes, p21(waf1/cip1), and erythropoietin.” J Neurosci. 1999. 19:9821-9830.
175. Zana et al. “Oxidatve stress: A bridge between Down's syndrome and Alzheimer's disease.” Neurobiol Aging. 2007. 28(5): 648-676
176. Zarrinkar et al., “Arrays of Arrays for High-Throughput Gene Expression Profiling.” Genome Res. 2001. 11: 1256-1261
177. Zhang et al., “Gene expression profiles in normal and cancer cells.” Science. 1997. 276: 1268-1272

### Other Embodiments

Other embodiments of the invention will be apparent to those skilled in the art from a consideration of the specification or practice of the invention disclosed herein. It is intended that the specification and examples be considered as exemplary only, with the true scope of the invention being indicated by the following claims.

1. A method comprising steps of
obtaining a reference genomic profile;
obtaining a test genomic profile from a sample of amniotic fluid and/or maternal blood, wherein the sample is obtained from a subject suffering from or carrying a fetus suffering from a fetal disease or condition;
determining differences between the test genomic profile and the reference genomic profile;
inputting the test genomic profile into a first computing machine;
accessing a storage repository on a second computing machine, wherein the storage repository contains a set of stored genomic profiles of one or more cell line(s) that have each been contacted with a different agent, wherein each stored genomic profile is mapped to data representing a corresponding agent;
generating, by a correlator executing on the first or the second computing machine, a correlation between each stored genomic profile and the test genomic profile; and
selecting at least one agent whose corresponding genomic profile has a negative correlation score with the test genomic profile, the selected agent being likely to reduce the differences between the test genomic profile and the reference genomic profile.

2. The method of claim 1, wherein the genomic profiles comprise information selected from the group consisting of mRNA levels, protein expression levels, DNA methylation patterns, metabolite profiles, and combinations thereof.

3. The method of claim 2, wherein the genomic profiles comprise mRNA levels.

4. The method of claim 3, wherein the reference genomic profile comprises a reference gene expression profile, the test genomic profile comprises a test gene expression profile, and the database comprises expression profiles.

5. The method of claim 1, further comprising testing activity of at least one identified agent for therapeutic effects in a fetal disease or condition.

6. The method of claim 5, wherein the at least one selected agent is tested in a model for the fetal disease or condition.

7. The method of claim 5, wherein the at least one selected agent is tested for medical applications in utero.

8. The method of claim 1, wherein the fetal disease or condition is selected from the group consisting of twin-to-twin transfusion syndrome (TTTS), gastroschisis, Down Syndrome, fetal structural anomalies, fetal congenital heart anomaly, fetal kidney anomalies, neural tube defects, and congenital diaphragmatic hernia.

9. The method of claim 8, wherein the fetal disease or condition is Down Syndrome.

10. The method of claim 1, wherein the first computing machine and the second computing machine are the same.

11. The method of claim 1, wherein the first computing machine and the second computing machine are different.

12. A method comprising a step of:
administering to a patient suffering from a fetal disease or condition an effective dose of an agent selected by the method of claim 1, such that symptoms of the fetal disease or condition are ameliorated.

13. The method of claim 12, wherein the effective dose of the compound is administered in utero.

14. The method of claim 12, wherein the fetal disease or condition is selected from the group consisting of twin-to-twin transfusion syndrome (TTTS), gastroschisis, Down Syndrome, fetal structural anomalies, fetal congenital heart anomaly, fetal kidney anomalies, neural tube defects, and congenital diaphragmatic hernia.

15. The method of claim 14, wherein the fetal disease or condition is Down Syndrome.

16. The method of claim 15, wherein the agent is selected from the group consisting of anti-oxidants, ion channel modulators, G-protein signaling modulators, and combinations thereof.

17. The method of claim 16, wherein the agent is an anti-oxidant.

18. The method of claim 17, wherein the agent is celastrol.

19. The method of claim 15, wherein the agent is a calcium channel blocker.

20. The method of claim 19, wherein the agent is selected from the group consisting of verapamil, felodipine, nifedipine, and combinations thereof.

21. The method of claim 15, wherein the agent is selected from the group consisting of copper sulfate, 15-delta prostaglandian J2, blebbistatin, prochlorperazine, 17-dimethylamino-geldanamycin, butein, nordihydroguaiaretic acid, acetylsalicyclic acid, 51825898, sirolimus, docosahexaenoic acid ethyl ester, diclofenac, mercaptopurine, indometacin, 5279552, 17-allylamino-geldanamycin, rottlerin, paclitaxel, pyrvinium, flufenamic acid, oligomycin, 5114445, resveratrol, Y-27632, carbamazepine, nitrendipine, fluphenazine, 5152487, prazosin, 5140203, cytochalasin B, vorinostate, MG-132, HNMPA-(AM)₃, decitabine, U0125, nocodazole, 5224221, 3-hydroxy-DL-kynurenine, 5162773, oxaprozin, colforsin, exemestane, felodipine, HC toxin, 5213008, dimethyloxalylglycine, 5109870, calmidazolium, 5255229, derivatives thereof, and combinations thereof.

22. A method comprising steps of:
(a) hybridizing RNA from an amniotic fluid and/or maternal blood sample from a subject suffering from or carrying a fetus suffering from a fetal disease or condition to at least one polynucleotide probe for at least one predetermined gene such that expression levels of at least one predetermined gene are obtained, wherein the sample is obtained from a subject to which the agent in step (b) has not been administered;
(b) administering an agent to a subject suffering from the fetal disease or condition;
(c) hybridizing RNA from an amniotic fluid and/or maternal blood sample from a subject suffering from or carrying a fetus suffering from a fetal disease or condition to at least one genetic probe for the same predetermined gene(s) from step (a) such that expression levels of the predetermined gene(s) are obtained, wherein the sample is obtained from a subject to which the agent has been administered;
(d) comparing the gene expression levels of the predetermined genes obtained from steps (a) and (c); and
(e) determining, based on the comparison, efficacy of the agent as a treatment for the fetal disease or condition.

23. The method of claim 22, wherein the fetal disease or condition is selected from the group consisting of twin-to-twin transfusion syndrome (TTTS), gastroschisis, Down Syndrome, fetal structural anomalies, fetal congenital heart anomaly, fetal kidney anomalies, neural tube defects, and congenital diaphragmatic hernia.

24. The method of claim 23, wherein the fetal disease is Down Syndrome.

25. A method comprising steps of:
providing a test sample comprising fetal RNA, wherein the fetal RNA is obtained from amniotic fluid and/or maternal blood obtained from a woman pregnant with a fetus with a known gender and gestational age, and wherein the test sample comprises a plurality of nucleic acid segments labeled with a detectable agent;
providing a gene-expression array comprising a plurality of genetic probes, wherein each genetic probe is immobilized to a discrete spot on a substrate surface to form the array;
providing a database comprising levels of mRNA expression established for trisomy 21 male and female fetuses at different gestational ages;
contacting the array with the test sample under conditions to allow the nucleic acid segments in the sample to specifically hybridize to the genetic probes on the array;
determining the binding of individual nucleic acid segments of the test sample to individual genetic probes immobilized on the array to obtain a binding pattern;
establishing a gene expression pattern for the fetus;
comparing the gene expression pattern of the fetus to the levels of mRNA expression in the database; and
providing, based on the comparison, a diagnosis with respect to Down Syndrome.

26. A method comprising steps of:
providing an amniotic fluid and/or maternal blood sample from a pregnant woman;
hybridizing RNA from the sample to at least ten genetic probes for at least ten genes that are differentially expressed in trisomy 21 fetuses such that expression levels of the at least ten genes are obtained; and
determining, based on the expression levels of the at least ten genes, a diagnosis with respect to Down Syndrome.

27. The method of claim 26, wherein the at least ten genes are selected from genes listed in Tables 2 and 4.

28-33. (canceled)
